(19) United States
(12) Patent Application Publication
(10) Pub. No.: US 2021/0009656 A1
(43) Pub. Date: Jan. 14, 2021

(54) C-TERMINALLY FUSED TNF FAMILY LIGAND TRIMER-CONTAINING ANTIGEN BINDING MOLECULES

(71) Applicant: Hoffmann-La Roche Inc., Little Falls, NJ (US)

(72) Inventors: Peter BRUENKER, Hittnau (CH); Claudia Ferrara KOLLER, Zug (CH); Sandra GRAU-RICHARDS, Birmensdorf (CH); Christian KLEIN, Bonstetten (CH); Ekkehard MOESSNER, Kreuzlingen (CH); Pablo UMANA, Wollerau (CH); Maria AMANN, Duebendorf (CH); Christina CLAUS, Ennetbaden (CH); Wei XU, Urdorf (CH)

(73) Assignee: Hoffmann-La Roche Inc., Little Falls, NJ (US)

(21) Appl. No.: 17/030,251

(22) Filed: Sep. 23, 2020

Related U.S. Application Data
(63) Continuation of application No. 16/184,147, filed on Nov. 8, 2018.
Application No. 16/184,147 is a continuation of application No. PCT/EP2017/060863, filed on May 8, 2017.

(30) Foreign Application Priority Data
May 11, 2016 (EP) 16169237.1

(51) Int. Cl. C07K 14/705; C07K 16/28; C07K 14/715; A61K 47/68; A61K 39/395; C07K 16/14; C07K 16/24; C07K 16/30; C07K 16/40

(52) U.S. Cl. CPC C07K 14/70578; C07K 2317/92; C07K 14/70575; C07K 14/7151; A61K 47/6813; A61K 39/395; C07K 16/14; C07K 16/241; A61K 47/6835; C07K 16/3007; C07K 16/40; C07K 2319/30; C07K 2317/24; C07K 2317/55; C07K 16/2803

(57) ABSTRACT

The invention relates to novel TNF family ligand trimer-containing antigen binding molecules comprising (a) one Fab domain capable of specific binding to a target cell antigen, (b) a Fc domain composed of a first and a second subunit capable of stable association, and (c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus of one of the subunits of the Fc domain and wherein the second polypeptide is fused at its N-terminus to the C-terminus of the other subunit of the Fc domain, wherein the TNF family ligand trimer-containing antigen binding molecule is monovalent for the binding to the target cell antigen.

### CROSS REFERENCE TO RELATED APPLICATIONS

This application is a Continuation of International Patent Application PCT/EP/2017/060863 filed on May 8, 2017 which is entitled to the benefit of EP Patent Application No. 16169237.1 filed on May 11, 2016, the disclosures of which are incorporated herein by reference.

### SEQUENCE LISTING

The present application contains a Sequence Listing which has been submitted in ASCII format via EFS-Web and is hereby incorporated by reference in its entirety. Said ASCII copy, created on Nov. 7, 2018, is named P33591-US_ST25.txt and is 663,074 in size.

### FIELD OF THE INVENTION

The invention relates to novel TNF family ligand trimer-containing antigen binding molecules comprising (a) a Fab domain capable of specific binding to a target cell antigen, (b) a Fc domain composed of a first and a second subunit capable of stable association, and (c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the TNF family ligand trimer-containing antigen binding molecule is monovalent for the binding to the target cell antigen. The invention further relates to methods of producing these molecules and to methods of using the same.

### BACKGROUND

Ligands interacting with molecules of the TNF (tumor necrosis factor) receptor superfamily have pivotal roles in the organization and function of the immune system. While regulating normal functions such as immune responses, hematopoiesis and morphogenesis, the TNF family ligands (also called cytokines) play a role in tumorgenesis, transplant rejection, septic shock, viral replication, bone resorption, rheumatoid arthritis and diabetes (Aggarwal, 2003). The TNF ligand family comprises 18 genes encoding 19 type II (i.e. intracellular N terminus and extracellular C-terminus) transmembrane proteins, characterized by the presence of #781063 a conserved C-terminal domain coined the ‘TNF homology domain’ (THD). This domain is responsible for receptor binding and is thus critical for the biological activity of the TNF ligand family members. The sequence identity between family members is ˜20-30% (Bodmer, 2002).

Members of the TNF ligand family exert their biological function as self-assembling, noncovalent trimers (Banner et al, 1993). Thus, the TNF family ligands form a trimer that is able to bind to and to activate the corresponding receptors of TNFR superfamily. 4-1BB (CD137), a member of the TNF receptor superfamily, has been first identified as a molecule whose expression is induced by T-cell activation (Kwon and Weissman, 1989). Subsequent studies demonstrated expression of 4-1BB in T- and B-lymphocytes (Snell et al., 2011; Zhang et al., 2010), NK-cells (Lin et al., 2008), NKT-cells (Kim et al., 2008), monocytes (Kienzle and von Kempis, 2000; Schwarz et al., 1995), neutrophils (Heinisch et al., 2000), mast (Nishimoto et al., 2005) and dendritic cells as well as cells of non-hematopoietic origin such as endothelial and smooth muscle cells (Broll et al., 2001; Olofsson et al., 2008). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002; von Kempis et al., 1997; Zhang et al., 2010).

Expression of 4-1BB ligand (4-1BBL or CD137L) is more restricted and is observed on professional antigen presenting cells (APC) such as B-cells, dendritic cells (DCs) and macrophages. Inducible expression of 4-1BBL is characteristic for T-cells, including both αβ and γδ T-cell subsets, and endothelial cells (reviewed in Shao and Schwarz, 2011).

CD137 signaling is known to stimulate IFNγ secretion and proliferation of NK cells (Buechele et al., 2012; Lin et al., 2008; Melero et al., 1998) as well as to promote DC activation as indicated by their increased survival and capacity to secret cytokines and upregulate co-stimulatory molecules (Choi et al., 2009; Futagawa et al., 2002; Wilcox et al., 2002). However, CD137 is best characterized as a co-stimulatory molecule which modulates TCR-induced activation in both the CD4⁺ and CD8⁺ subsets of T-cells. In combination with TCR triggering, agonistic 4-1BB-specific antibodies enhance proliferation of T-cells, stimulate lymphokine secretion and decrease sensitivity of T-lymphocytes to activation-induced cells death (reviewed in (reviewed in Snell et al., 2011).

In line with these co-stimulatory effects of 4-1BB antibodies on T-cells in vitro, their administration to tumor bearing mice leads to potent anti-tumor effects in many experimental tumor models (Melero et al., 1997; Narazaki et al., 2010). However, 4-1BB usually exhibits its potency as an anti-tumor agent only when administered in combination with other immunomodulatory compounds (Curran et al., 2011; Guo et al., 2013; Morales-Kastresana et al., 2013; Teng et al., 2009; Wei et al., 2013), chemotherapeutic reagents (Ju et al., 2008; Kim et al., 2009), tumor-specific vaccination (Cuadros et al., 2005; Lee et al., 2011) or radiotherapy (Shi and Siemann, 2006). In vivo depletion experiments demonstrated that CD8⁺ T-cells play the most critical role in anti-tumoral effect of 4-1BB-specific antibodies. However, depending on the tumor model or combination therapy, which includes anti-4-1BB, contributions of other types of cells such as DCs, NK-cells or CD4⁺ T-cells have been reported (Melero et al., 1997; Murillo et al., 2009; Narazaki et al., 2010; Stagg et al., 2011).

In addition to their direct effects on different lymphocyte subsets, 4-1BB agonists can also induce infiltration and retention of activated T-cells in the tumor through 4-1BB-mediated upregulation of intercellular adhesion molecule 1 (ICAM1) and vascular cell adhesion molecule (VCAM1) on tumor vascular endothelium (Palazon et al., 2011).

4-1BB triggering may also reverse the state of T-cell anergy induced by exposure to soluble antigen that may contribute to disruption of immunological tolerance in the tumor micro-environment or during chronic infections (Wilcox et al., 2004).

It appears that the immunomodulatory properties of 4-1BB agonistic antibodies in vivo require the presence of the wild type Fc-portion on the antibody molecule thereby implicating Fc-receptor binding as an important event required for the pharmacological activity of such reagents as has been described for agonistic antibodies specific to other apoptosis-inducing or immunomodulatory members of the TNFR-superfamily (Li and Ravetch, 2011; Teng et al., 2009). However, systemic administration of 4-1BB-specific agonistic antibodies with the functionally active Fc domain also induces expansion of CD8⁺ T-cells associated with liver toxicity (Dubrot et al., 2010) that is diminished or significantly ameliorated in the absence of functional Fc-receptors in mice. In human clinical trials (ClinicalTrials.gov, NCT00309023), Fc-competent 4-1BB agonistic antibodies (BMS-663513) administered once every three weeks for 12 weeks induced stabilization of the disease in patients with melanoma, ovarian or renal cell carcinoma. However, the same antibody given in another trial (NCT00612664) caused grade 4 hepatitis leading to termination of the trial (Simeone and Ascierto, 2012).

Collectively, the available pre-clinical and clinical data clearly demonstrate that there is a high clinical need for effective 4-1BB agonists. However, new generation drug candidates should not only effectively engage 4-1BB on the surface of hematopoietic and endothelial cells but also be capable of achieving that through mechanisms other than binding to Fc-receptors in order to avoid uncontrollable side effects. The latter may be accomplished through preferential binding to and oligomerization on tumor-specific or tumor-associated moieties.

Fusion proteins composed of one extracellular domain of a 4-1BB ligand and a single chain antibody fragment (Mueller et al., 2008; Hornig et al., 2012) or a single 4-1BB ligand fused to the C-terminus of a heavy chain (Zhang et al, 2007) have been made. WO 2010/010051 discloses the generation of fusion proteins that consist of three TNF ligand ectodomains linked to each other and fused to an antibody part.

However, there is still a need of new antigen binding molecules that combine a Fab domain capable of preferred binding to tumor-specific or tumor-associated targets with a moiety capable of forming a costimulatory TNF ligand trimer and that have at the same time sufficient stability to be pharmaceutically useful. The antigen binding molecules of the present invention comprise both and surprisingly they provide a trimeric and thus biologically active TNF ligand, although one of the trimerizing TNF ligand ectodomains is located on another polypeptide than the other two TNF ligand ectodomains of the molecule. Surprisingly, it has been found that one Fab domain capable of specific binding to a target cell antigen is sufficient to provide the preferred targeting properties. The antigen binding molecules of this invention are particularly stable and robust.

### SUMMARY OF THE INVENTION

In one aspect, the invention provides a TNF family ligand trimer-containing antigen binding molecule comprising

(a) one Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the TNF family ligand trimer-containing antigen binding molecule is monovalent for the binding to the target cell antigen.

In a particular aspect, the TNF ligand family member is one that costimulates human T-cell activation. Thus, the TNF family ligand trimer-containing antigen binding molecule comprises (a) one Fab domain capable of specific binding to a target cell antigen, (b) a Fc domain composed of a first and a second subunit capable of stable association, and

(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the TNF family ligand trimer-containing antigen binding molecule is monovalent for the binding to the target cell antigen and wherein the TNF ligand family member costimulates human T-cell activation. More particularly, the TNF ligand family member is selected from 4-1BBL and OX40L.

In one aspect, the TNF ligand family member is 4-1BBL.

In a further aspect, the ectodomain of a TNF ligand family member comprises the amino acid sequence selected from the group consisting of SEQ ID NO:1, SEQ ID NO: 2, SEQ ID NO:3, SEQ ID NO:4, SEQ ID NO:5, SEQ ID NO:6, SEQ ID NO:7 and SEQ ID NO:8, particularly the amino acid sequence of SEQ ID NO:1 or SEQ ID NO:5.

In a further aspect, the TNF family ligand trimer-containing antigen binding molecule of the invention comprises

(a) one Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain and wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5. In one aspect, the first polypeptide comprising two ectodomains of a TNF ligand family member or fragments thereof comprises an amino acid sequence SEQ ID NO:9 and the second polypeptide comprising one ectodomain of said TNF ligand family member or a fragment thereof comprises the amino acid sequence of SEQ ID NO:1. In a particular aspect, the first polypeptide comprising two ectodomains of a TNF ligand family member or fragments thereof comprises an amino acid sequence of SEQ ID NO:10 and the second polypeptide comprising one ectodomain of said TNF ligand family member or a fragment thereof comprises the amino acid sequence of SEQ ID NO:5.

In a further aspect, provided is aTNF family ligand trimer-containing antigen binding molecule as described herein before, wherein the TNF ligand family member is OX40L. In one aspect, provided is TNF family ligand trimer-containing antigen binding molecule, wherein the ectodomain of a TNF ligand family member comprises the amino acid sequence selected from the group consisting of SEQ ID NO:11 and SEQ ID NO:12.

In one aspect, the invention provides a TNF family ligand trimer-containing antigen binding molecule comprising

(a) one Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain. The invention thus relates to a TNF family ligand trimer-containing antigen binding molecule, wherein TNF family ligand trimer-containing antigen binding molecule is monovalent for the binding to the target cell antigen.

In another aspect, provided is a TNF family ligand trimer-containing antigen binding molecule of the invention, wherein the target cell antigen is selected from the group consisting of Fibroblast Activation Protein (FAP), CD19, Carcinoembryonic Antigen (CEA), Melanoma-associated Chondroitin Sulfate Proteoglycan (MCSP), Epidermal Growth Factor Receptor (EGFR), CD20 and CD33.

In a particular aspect, the target cell antigen is Fibroblast Activation Protein (FAP).

In one aspect, the invention provides a TNF family ligand trimer-containing antigen binding molecule, wherein the Fab domain capable of specific binding to FAP comprises

(a) a VH domain comprising (i) CDR-H1 comprising the amino acid sequence of SEQ ID NO:13, (ii) CDR-H2 comprising the amino acid sequence of SEQ ID NO:14 and (iii) CDR-H3 comprising the amino acid sequence of SEQ ID NO:15, and a VL domain comprising (iv) CDR-L1 comprising the amino acid sequence of SEQ ID NO:16, (v) CDR-L2 comprising the amino acid sequence of SEQ ID NO:17 and (vi) CDR-L3 comprising the amino acid sequence of SEQ ID NO:18 or
(b) a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.

In a particular aspect, the invention provides a TNF family ligand trimer-containing antigen binding molecule, wherein the Fab domain capable of specific binding to FAP comprises a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.

In a further aspect, provided is a TNF family ligand trimer-containing antigen binding molecule as described herein before, wherein the Fab domain capable of specific binding to FAP comprises a variable heavy chain comprising an amino acid sequence of SEQ ID NO:25 and a variable light chain comprising an amino acid sequence of SEQ ID NO:26 or wherein the Fab domain capable of specific binding to FAP comprises a variable heavy chain comprising an amino acid sequence of SEQ ID NO:27 and a variable light chain comprising an amino acid sequence of SEQ ID NO:28.

In another particular aspect, the target cell antigen is CD19.

In one aspect, the invention provides a TNF family ligand trimer-containing antigen binding molecule, wherein the Fab domain capable of specific binding to CD19 comprises

(a) a VH domain comprising (i) CDR-H1 comprising the amino acid sequence of SEQ ID NO:29, (ii) CDR-H2 comprising the amino acid sequence of SEQ ID NO:30 and (iii) CDR-H3 comprising the amino acid sequence of SEQ ID NO:31, and a VL domain comprising (iv) CDR-L1 comprising the amino acid sequence of SEQ ID NO:32, (v) CDR-L2 comprising the amino acid sequence of SEQ ID NO:33 and (vi) CDR-L3 comprising the amino acid sequence of SEQ ID NO:34 or
(b) a VH domain comprising (i) CDR-H1 comprising the amino acid sequence of SEQ ID NO:35, (ii) CDR-H2 comprising the amino acid sequence of SEQ ID NO:36 and (iii) CDR-H3 comprising the amino acid sequence of SEQ ID NO:37, and a VL domain comprising (iv) CDR-L1 comprising the amino acid sequence of SEQ ID NO:38, (v) CDR-L2 comprising the amino acid sequence of SEQ ID NO:39 and (vi) CDR-L3 comprising the amino acid sequence of SEQ ID NO:40.

In a particular aspect, the invention provides a TNF family ligand trimer-containing antigen binding molecule, wherein the Fab domain capable of specific binding to CD19 comprises a VH domain comprising (i) CDR-H1 comprising the amino acid sequence of SEQ ID NO:35, (ii) CDR-H2 comprising the amino acid sequence of SEQ ID NO:36 and (iii) CDR-H3 comprising the amino acid sequence of SEQ ID NO:37, and a VL domain comprising (iv) CDR-L1 comprising the amino acid sequence of SEQ ID NO:38, (v) CDR-L2 comprising the amino acid sequence of SEQ ID NO:39 and (vi) CDR-L3 comprising the amino acid sequence of SEQ ID NO:40.

In a further aspect, provided is a TNF family ligand trimer-containing antigen binding molecule as described herein before, wherein the Fab domain capable of specific binding to CD19 comprises a variable heavy chain comprising an amino acid sequence of SEQ ID NO:41 and a variable light chain comprising an amino acid sequence of SEQ ID NO:42 or wherein the Fab domain capable of specific binding to FAP comprises a variable heavy chain comprising an amino acid sequence of SEQ ID NO:43 and a variable light chain comprising an amino acid sequence of SEQ ID NO:44.

In another particular aspect, the target cell antigen is CEA.

In one aspect, the invention provides a TNF family ligand trimer-containing antigen binding molecule, wherein the Fab domain capable of specific binding to CEA comprises a VH domain comprising (i) CDR-H1 comprising the amino acid sequence of SEQ ID NO:45, (ii) CDR-H2 comprising the amino acid sequence of SEQ ID NO:46 and (iii) CDR-H3 comprising the amino acid sequence of SEQ ID NO:47, and a VL domain comprising (iv) CDR-L1 comprising the amino acid sequence of SEQ ID NO:48, (v) CDR-L2 comprising the amino acid sequence of SEQ ID NO:49 and (vi) CDR-L3 comprising the amino acid sequence of SEQ ID NO:50.

In a particular aspect, provided is a TNF family ligand trimer-containing antigen binding molecule as described herein before, wherein the Fab domain capable of specific binding to CEA comprises a variable heavy chain comprising an amino acid sequence of SEQ ID NO:51 and a variable light chain comprising an amino acid sequence of SEQ ID NO:52.

In a further aspect, the Fc domain is an IgG, particularly an IgG1 Fc domain or an IgG4 Fc domain. More particularly, the Fc domain is an IgG1 Fc domain.

In particular, the TNF family ligand trimer-containing antigen binding molecule of the invention comprises a Fab domain capable of specific binding to a target cell antigen, wherein the VH and CH1 domain of the Fab domain are fused at the C-terminus to the N-terminus of a CH2 domain in the Fc domain.

In another aspect, the invention is concerned with a TNF family ligand trimer-containing antigen binding molecule as defined herein before, comprising

(b) an Fc domain composed of a first and a second subunit capable of stable association, wherein the Fc domain comprises one or more amino acid substitution that reduces binding to an Fc receptor, in particular towards Fcγ receptor.

In particular, the Fc domain comprises amino acid substitutions at positions 234 and 235 (numbering according to Kabat EU index) and/or 329 (numbering according to Kabat EU index) of the IgG heavy chains. More particularly, provided is a trimeric TNF family ligand-containing antigen binding molecule according to the invention which comprises an IgG1 Fc domain with the amino acid substitutions L234A, L235A and P329G (numbering according to Kabat EU index).

In a further aspect, the Fc domain comprises a modification promoting the association of the first and second subunit of the Fc domain. In a particular aspect, provided is a TNF family ligand trimer-containing antigen binding molecule as defined herein before, wherein the first subunit of the Fc domain comprises the amino acid substitutions S354C and T366W (numbering according to Kabat EU index) of the knob and the second subunit of the Fc domain comprises the amino acid substitutions Y349C, T366S, L368A and Y407V (numbering according to Kabat EU index) of the hole.

In a further aspect, provided is a TNF family ligand trimer-containing antigen binding molecule as defined herein before, wherein the first polypeptide comprising two ectodomains of a TNF ligand family member or fragments thereof that are connected to each other by a peptide linker is fused at its N-terminus to the C-terminus of the second subunit of the Fc domain (hole) and the second polypeptide comprising one ectodomain of said TNF ligand family member or a fragment thereof is fused at its N-terminus to the C-terminus of the first subunit of the Fc domain (knob).

In another aspect, provided is a TNF family ligand trimer-containing antigen binding molecule as defined herein before, wherein the first polypeptide comprising two ectodomains of a TNF ligand family member or fragments thereof that are connected to each other by a peptide linker is fused at its N-terminus to the C-terminus of the first subunit of the Fc domain (knob) and the second polypeptide comprising one ectodomain of said TNF ligand family member or a fragment thereof is fused at its N-terminus to the C-terminus of the second subunit of the Fc domain (hole).

In one aspect, the invention provides a TNF family ligand trimer-containing antigen binding molecule, wherein the Fab domain capable of specific binding to a target cell antigen is fused at the C-terminus to the N-terminus of the first subunit of the Fc domain (knob). In another aspect, provided is a TNF family ligand trimer-containing antigen binding molecule, wherein the Fab domain capable of specific binding to a target cell antigen is fused at the C-terminus to the N-terminus of the second subunit of the Fc domain (hole).

In a particular aspect, provided is a TNF family ligand trimer-containing antigen binding molecule, wherein the Fab domain capable of specific binding to a target cell antigen is fused at the C-terminus to the N-terminus of the first subunit of the Fc domain (knob), the first polypeptide comprising two ectodomains of a TNF ligand family member or fragments thereof that are connected to each other by a peptide linker is fused at its N-terminus to the C-terminus of the second subunit of the Fc domain (hole) and the second polypeptide comprising one ectodomain of said TNF ligand family member or a fragment thereof is fused at its N-terminus to the C-terminus of the first subunit of the Fc domain (knob).

In a further aspect, provided is a TNF family ligand trimer-containing antigen binding molecule, comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fc domain comprising the hole mutations and wherein the VH and CH1 domain of the Fab domain capable of specific binding to a target cell antigen is fused at the C-terminus to the N-terminus of the second subunit of the Fc domain comprising the knob mutations.

In another aspect, provided is a TNF family ligand trimer-containing antigen binding molecule, comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fc domain comprising the hole mutations and wherein the VH and CH1 domain of the Fab domain capable of specific binding to a target cell antigen is fused at the C-terminus to the N-terminus of the first subunit of the Fc domain comprising the hole mutations.

In yet another aspect, provided is a TNF family ligand trimer-containing antigen binding molecule, comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fe domain comprising the hole mutations and wherein the second polypeptide is fused at its N-terminus to the C-terminus of the second subunit of the Fe domain comprising the knob mutations and wherein the VH and CH1 domain of the Fab domain capable of specific binding to a target cell antigen is fused at the C-terminus to the N-terminus of the second subunit of the Fc domain comprising the knob mutations.

In addition, provided is a TNF family ligand trimer-containing antigen binding molecule, comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fc domain comprising the hole mutations and wherein the second polypeptide is fused at its N-terminus to the C-terminus of the second subunit of the Fc domain comprising the knob mutations and wherein the VH and CH1 domain of the Fab domain capable of specific binding to a target cell antigen is fused at the C-terminus to the N-terminus of the first subunit of the Fc domain comprising the hole mutations.

In a further aspect, the invention provides a TNF family ligand trimer-containing antigen binding molecule as defined before further comprising (d) a Fab domain that is not capable of specific binding to a target cell antigen. Thus, the invention provides a TNF family ligand trimer-containing antigen binding molecule comprising

(a) a Fab domain capable of specific binding to a target cell antigen,
(b) a Fc domain composed of a first and a second subunit capable of stable association,
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, and
(d) a Fab domain that is not capable of specific binding to a target cell antigen.

According to another aspect of the invention, there is provided an isolated polynucleotide encoding a TNF family ligand trimer-containing antigen binding molecule as defined herein before. The invention further provides a vector, particularly an expression vector, comprising the isolated polynucleotide of the invention and a host cell comprising the isolated polynucleotide or the vector of the invention. In some embodiments the host cell is a eukaryotic cell, particularly a mammalian cell.

In another aspect, provided is a method for producing the TNF family ligand trimer-containing antigen binding molecule of the invention, comprising the steps of (i) culturing the host cell of the invention under conditions suitable for expression of the antigen binding molecule, and (ii) recovering the antigen binding molecule. The invention also encompasses a TNF family ligand trimer-containing antigen binding molecule produced by the method of the invention.

The invention further provides a pharmaceutical composition comprising the TNF family ligand trimer-containing antigen binding molecule of the invention and at least one pharmaceutically acceptable excipient.

Also encompassed by the invention is the TNF family ligand trimer-containing antigen binding molecule of the invention, or the pharmaceutical composition of the invention, for use as a medicament. In one aspect is provided the TNF family ligand trimer-containing antigen binding molecule of the invention, or the pharmaceutical composition of the invention, for use in the treatment of a disease in an individual in need thereof. The present invention also provides the TNF family ligand trimer-containing antigen binding molecule of the invention, or the pharmaceutical composition of the invention, for use (i) in stimulating T cell response, (ii) in supporting survival of activated T cells, (iii) in the treatment of infections, (iv) in the treatment of cancer, (v) in delaying progression of cancer, or (vi) in prolonging the survival of a patient suffering from cancer. In a specific embodiment, provided is the TNF family ligand trimer-containing antigen binding molecule of the invention, or the pharmaceutical composition of the invention, for use in the treatment of cancer.

Also provided is the use of the TNF family ligand trimer-containing antigen binding molecule of the invention for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the TNF family ligand trimer-containing antigen binding molecule of the invention in a pharmaceutically acceptable form. In a specific embodiment, the disease is cancer. The present invention also provides the use of the TNF family ligand trimer-containing antigen binding molecule of the invention, or the pharmaceutical composition of the invention, in the manufacture of a medicament for up-regulating or prolonging cytotoxic T cell activity. Also provided is a method of up-regulating or prolonging cytotoxic T cell activity in an individual having cancer, comprising administering to the individual an effective amount of the TNF family ligand trimer-containing antigen binding molecule of the invention, or the pharmaceutical composition of the invention. In any of the above embodiments the individual is preferably a mammal, particularly a human.

### BRIEF DESCRIPTION OF THE DRAWINGS

FIGS. 1A to 1D show the components for the assembly of split trimeric human 4-1BB ligands. Figure (TA) shows the polypeptide comprising the dimeric 4-1BB ligand that is fused at the N-terminus to the C-terminus of a human CH3 domain by a peptide linker (G4S)₂ and FIG. (1B) shows the polypeptide comprising the monomeric 4-1BB ligand that is fused at the N-terminus to the C-terminus of the other CH3 domain within a Fc domain by a peptide linker (G4S)₂. FIG. (1C) shows a dimeric OX40 ligand that is fused at the N-terminus by a peptide linker (G4S)₂ to the C-terminus of a human CH3 domain and FIG. (1D) shows a monomeric ligand fused at the N-terminus to the C-terminus of the other human CH3 domain within the Fc domain by a peptide linker (G4S)₂.

FIGS. 2A to 2D are schematic drawings of the structures of the monovalent targeted 4-1BBL-trimer-containing antigen binding molecules of the invention. In FIG. 2A is shown the antigen binding molecule as construct of type x.11, wherein the Fab domain capable of specific binding to a target cell antigen is attached to the Fc knob chain, the dimeric 4-1BBL is fused to the C-terminus of the Fc hole chain and the monomeric 4-1BBL is fused to the C-terminus of the Fc knob chain. Constructs 2.11, 1.11, 3.11, 4.11, 5.11 and Control H are corresponding examples. A construct of type x.12 is shown in FIG. 2B. The Fab domain capable of specific binding to a target cell antigen is attached to the Fc knob chain, the dimeric 4-1BBL is also fused to the C-terminus of the Fc knob chain and the monomeric 4-1BBL is fused to the C-terminus of the Fc hole chain. Constructs 2.12, 1.12, 3.12, 4.12, 5.12 and Control I are examples for these antigen binding molecules. In FIG. 2C is shown the antigen binding molecule as construct of type x.13, wherein the Fab domain capable of specific binding to a target cell antigen is attached to the Fc hole chain, the dimeric 4-1BBL is fused to the C-terminus of the Fc hole chain and the monomeric 4-1BBL is fused to the C-terminus of the Fc knob chain. Constructs 2.13, 1.13, 3.13, 4.13, 5.13 and Control J are corresponding examples. FIG. 2D relates to constructs of type x.14, wherein the Fab domain capable of specific binding to a target cell antigen is attached to the Fc hole chain, the dimeric 4-1BBL is also fused to the C-terminus of the Fc knob chain and the monomeric 4-1BBL is fused to the C-terminus of the Fc hole chain. Constructs 2.14, 1.14, 3.14, 4.14, 5.14 and Control K are examples therefore. The preparation of all these constructs is described in Example 1. The VH, VL, CL and CH domains symbolize the Fab domain, the thick black point stands for the knob-into-hole modification.

FIGS. 3A to 3D are schematic drawings of further molecules described herein. FIG. 3A shows Control F, a hu IgG1 with two DP47 (germline) untargeted Fab domains. FIG. 3B shows Control D, an untargeted 4-1BBL trimer-containing antigen binding molecule, wherein the dimeric 4-1BBL is fused to the N-terminus of CL domain in the Fe knob heavy chain and the monomeric 4-1BBL is fused to the N-terminus of a CH1 domain of a light chain. * symbolizes amino acid modifications in the CH1 and CL domain (so-called charged residues). FIG. 3C shows the same construct as FIG. 3B, however the DP47 Fab domain is replaced by FAP(4B9) Fab domain. The preparation and production of these constructs is described in Example 1.11. The thick black point stands for the knob-into-hole modification. FIG. 3D is a drawing of the monomeric 4-1BB Fc(kih) construct as prepared in Example 3.1.

FIG. 4A shows the setup of the SPR experiments for simultaneous binding of the monovalent targeted split trimeric C-terminal 4-1BB ligand-containing antigen binding molecules of the invention. The simultaneous binding to human 4-1BB and human FAP of the FAP (4B9)-targeted split 4-1BBL (71-248) Fc kih antigen binding molecule Construct 2.11 is shown in FIG. 4B. Simultaneous binding to human 4-1BB and human CD19 of CD19(8B8-018)-targeted split 4-1BBL (71-248) Fc kih antigen binding molecule (Construct 3.11) is shown in FIG. 4C. Simultaneous binding to human 4-1BB and human CD19 of the CD19(8B8-2B11)-targeted split 4-1BBL (71-248) Fc kih antigen binding molecule (construct 4.11) is shown in FIG. 4D.

FIGS. 5A to 5D are schematic drawings of the structures of the monovalent targeted 4-1BBL-trimer-containing antigen binding molecules of the invention that comprise a further Fab domain that is not capable for binding to a target cell antigen. In FIG. 5A is shown the antigen binding molecule as construct of type x.15, wherein the Fab domain not capable of specific binding to a target cell antigen (DP47 germline) is attached to the Fc knob chain, the Fab domain capable of specific binding to the antigen (FAP) is connected to the Fc hole chain, the dimeric 4-1BBL is fused to the C-terminus of the Fc hole chain and the monomeric 4-1BBL is fused to the C-terminus of the Fc knob chain. Construct 2.15 is an example thereof. A construct of type x.16 is shown in FIG. 5B. The Fab domain not capable of specific binding to a target cell antigen (DP47 germline) is attached to the Fc knob chain, the Fab domain capable of specific binding to the antigen (FAP) is connected to the Fc hole chain, the dimeric 4-1BBL is also fused to the C-terminus of the Fc knob chain and the monomeric 4-1BBL is fused to the C-terminus of the Fc hole chain. Construct 2.16 is an example thereof. In FIG. 5C is shown the antigen binding molecule as construct of type x.17, wherein the Fab domain capable of specific binding to a target cell antigen is attached to the Fc knob chain, the Fab domain not capable of specific binding to a target cell antigen (DP47 germline) is connected to the Fc hole chain, the dimeric 4-1BBL is fused to the C-terminus of the Fc hole chain and the monomeric 4-1BBL is fused to the C-terminus of the Fc knob chain. Construct 2.17 is an example thereof. FIG. 5D relates to constructs of type x.18, wherein the Fab domain capable of specific binding to a target cell antigen is attached to the Fc knob chain, the Fab domain not capable of specific binding to a target cell antigen (DP47 germline) is attached to the Fc hole chain, the dimeric 4-1BBL is also fused to the C-terminus of the Fc knob chain and the monomeric 4-1BBL is fused to the C-terminus of the Fc hole chain. Construct 2.18 is an example thereof. The preparation of all these constructs is described in Example 4.

FIG. 6A shows the setup of the SPR experiments for simultaneous binding of the DP47/monovalent FAP targeted split trimeric C-terminal 4-1BB ligand-containing antigen binding molecules of the invention. The simultaneous binding to human 4-1BB and human FAP of the DP47/FAP (4B9)-targeted split 4-1BBL (71-254) Fc kih antigen binding molecule Construct 2.15 is shown in FIG. 6B.

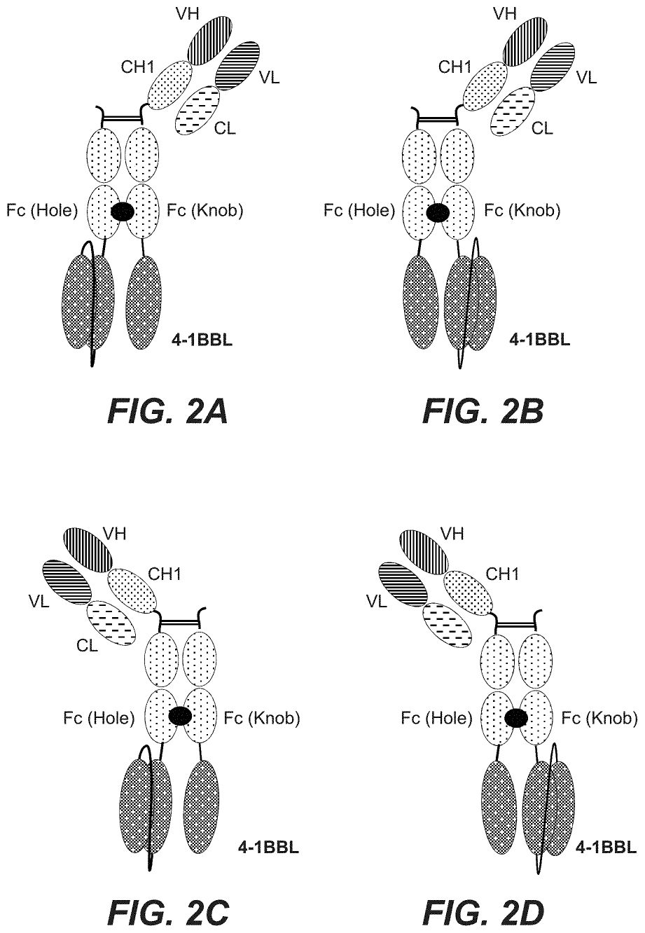

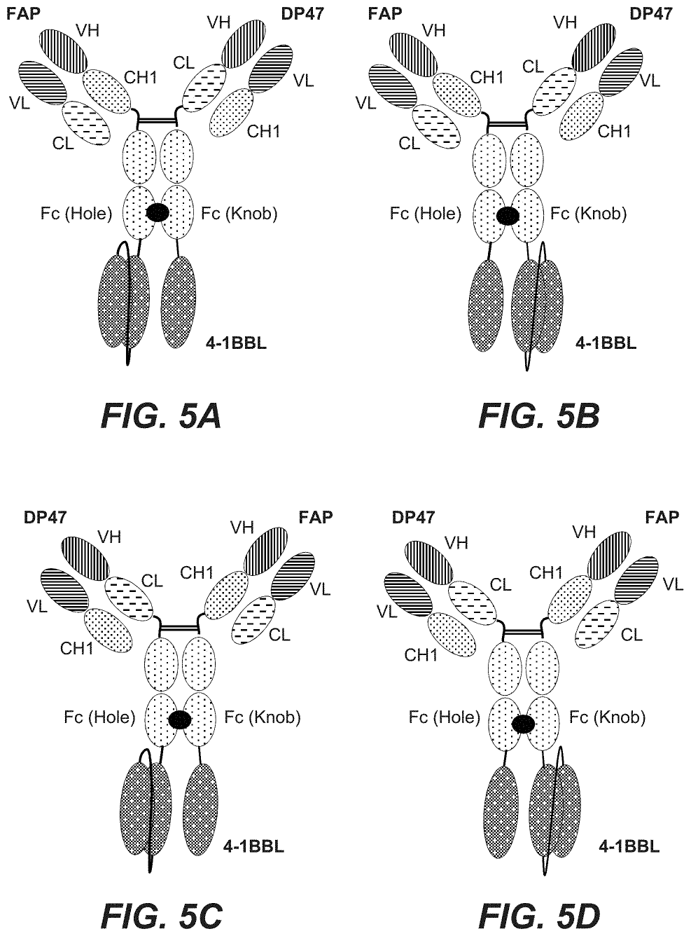

In FIGS. 7A to 7H is shown the binding of FAP-targeted split trimeric 4-1BB ligand Fc fusion antigen binding molecules to freshly isolated PBMCs. Shown is the geometric (geo) mean of fluorescence intensity of PE-conjugated secondary detection antibody versus the concentration of tested constructs. As negative controls control F and control D and as positive control construct 2.4 (described in Example 1.11) were used. In FIGS. 7A to 7D the binding of Construct 2.11 and in FIGS. 7E to 7H the binding of Construct 2.15 is illustrated. The structure of construct 2.11 is shown in FIG. 2A and the structure of Construct 2.15 is shown in FIG. 5A. Binding was monitored on fresh human CD45⁺ CD3⁺ CD8^{neg} CD4⁺ T cells (FIG. 7A or 7E), fresh human CD45⁺ CD3⁺ CD4^{neg} CD8⁺ T cells (FIG. 7B or 7F), fresh human CD45⁺ CD3⁺ CD4^{neg} CD8^{neg} γδ T cells (FIG. 7C or 7G) and CD45⁺ CD3^{neg} CD19⁺ B cells (FIG. 7D or 7H). Because the majority of fresh isolated PBMCs do not express 4-1BB or FAP, no binding could be detected.

FIGS. 8A to 8F illustrate the binding of FAP-targeted split trimeric 4-1BB ligand Fc fusion antigen binding molecules to activated human PBMCs. Shown is the geo mean of fluorescence intensity of PE-conjugated secondary detection antibody versus the concentration of tested constructs. As negative controls control F and control D and as positive control construct 2.4 as described in Example 1.11 were used. In FIGS. 8A to 8C the binding of Construct 2.11 and in FIGS. 8D to 8F the binding of Construct 2.15 is illustrated. After activation with agonistic anti-human CD28 and anti-human CD3 antibody 4-1BB expression is upregulated on the majority of CD3⁺ CD8⁺ T cells and to a lesser extend to the majority CD3⁺ CD4⁺ and CD3⁺ CD4^{neg} CD8^{neg} γδ T cells. All constructs containing a fused 4-1BB split trimeric ligand (control D, construct 2.4, constructs 2.11 and 2.15) bind with a quite similar affinity to human 4-1BB, independent if they are FAP (4B9)-targeted (construct 2.4 and 2.11) or DP47-untargeted (control D), whereas the untargeted molecule without a fused 4-1BB split trimeric ligand (control F) does not bind to the activated human T cells.

FIGS. 9A to 9D show the binding of FAP-targeted or untargeted split trimeric human 4-1BB ligand Fc (kih) fusion antigen binding molecules to human-FAP expressing human melanoma MV-3 cells and WM-266-4 cells. Binding was detected with R-Phycoerythrin-fluorochrome conjugated anti-human IgG Fcγ-specific goat IgG F(ab′)2 fragment. As negative controls control F and D and as positive control Construct 2.4 as described in Example 1.11 were used. Shown is the geo mean of fluorescence intensity versus the concentration of tested constructs and controls. In FIGS. 9A (MV-3 cells) and 9B (WM-266-4 cells) the binding of Construct 2.11 in comparison to the control molecules is shown and in FIGS. 9C (MV-3 cells) and 9D (WM-266-4 cells) the binding of Construct 2.15 in comparison to the control molecules is shown. All FAP-targeted constructs bind with a similar affinity to human FAP.

FIG. 10 shows a scheme that illustrates the general principal of the NFkB activity assay described in Example 6.3 using a reporter cell line. Shown is the activation assay set up with human 4-1BB expressing HeLa reporter cell line. A crosslinking of 4-1BB expressed on the reporter cells induces NFκB activation and NFκB-mediated Luciferase expression. After lysis of the cells Luciferase can catalyze the oxidation of Luciferin to Oxyluciferin. This chemical reaction correlates positively with the strength of NFκB-mediated luciferase expression and can be measured by the strength of light emission (units of released light).

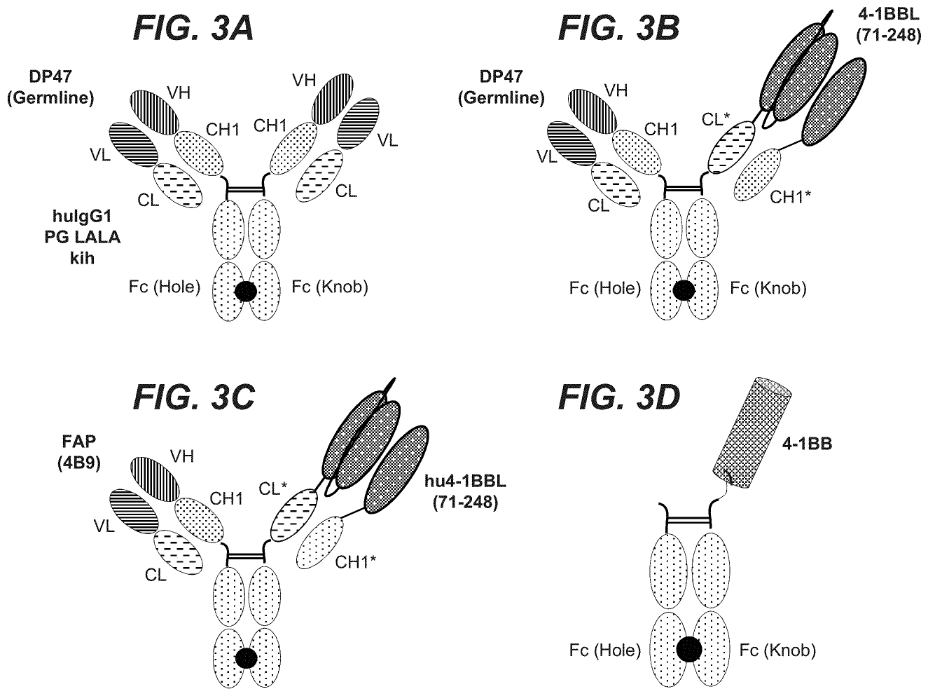

FIGS. 11A to 11C shows that the activation of the NFkB signaling pathway by FAP-targeted 4-1BB ligand trimer-containing Fc(kih) fusion antigen binding molecule is strictly dependent on its binding to FAP-expressing target cells. Human 4-1BB expressing NFkB reporter HeLa cells were co-cultured with the indicated tumor cells exhibiting different levels of cell surface FAP expression. Units of released light (URL) are measured for 0.5 s/well and plotted against the used concentration of Construct 2.11 or Construct 2.15 or (as positive control) Construct 2.4 or (as negative controls) Control D or F. Human 4-1BB-expressing HeLa-reporter cells were incubated for 6 h in the absence (FIG. 11A) or presence of crosslinking human-FAP expressing human melanoma cell line MV-3 (see FIG. 11B) or 3T3-huFAP clone 19 (see FIG. 11C) and then Luciferase activity was assessed as described in Example 6.3. The cell ratio of 4-1BB-expressing HeLa reporter cell to tumor cells are indicated in each set up.

FIG. 12 shows a schematic scheme of the human PBMC Activation assas as described in Example 6.4. Activation of PBMCs is achieved in the presence of FAP-expressing Fibroblasts, agonistic anti-human CD3 antibody and bispecific anti-FAP split trimeric 4-1BBL fusion antigen binding molecule construct 2.11. The content per well in the plate was: 45 Gy irradiated 2×10⁴ NIH/3T3-human FAP clone 19, 7.5×10⁶ CFSE-labeled human PBMCs, 0.5 nM agonistic anti-human CD3 human IgG wt (clone V9) and 10⁻⁵ nM to 10 nM titrated concentrations of construct 2.11, construct 2.4, control D or control F. The incubation time was 4 days.

In FIGS. 13 A and 13B results in the human PBMC activation assay in the presence of FAP+ fibroblasts, agonistic CD3 antibody and FAP-targeted split trimeric 4-1BBL fusion molecules are shown. The percentage of CD25 and CD137 (4-1BB) expressing CD8⁺ T cells are plotted against the used concentration of construct 2.11 or as positive control construct 2.4 or as negative control molecules control D and control F (see Example 1.11).

FIG. 14 shows the schematic set-up of the NLV-specific CD8+ T cells activation in the presence of NLV-peptide pulsed FAP-expressing MV-3 human melanoma cells and bispecific anti-FAP split trimeric 4-1BBL fusion molecule construct 2.11. The content per well in the plate was: 5×10⁴ human melanoma cells MV-3, pulsed with no, 10- or 10⁻⁸ M NLV-peptide, 6250 CFSE-labeled NLV-specific CD8 T cells and 10⁻⁵ nM to 10 nM titrated concentrations of construct 2.11, construct 2.4, control D or control F. The incubation time was 24 h+4 h in the presence of Brefeldin A and Monesin.

In FIGS. 15A to 15F results are shown from the activation assay with HLA-A2-NLV-specific CD8 T cells and NLV-pulsed HLA-A2⁺ FAP⁺ human melanoma cell line MV-3 in the presence of titrated concentration of different FAP-targeted or untargeted split trimeric human 4-1BB ligand Fc (kih) antigen binding molecules. The percentage of CD137 (4-1BB) and IFNγ expressing NLV-specific CD8⁺ T cells are plotted against the used concentration of the test compounds (construct 2.11 or as positive control construct 2.4 or as negative controls control D and control F). All FAP-targeted split trimeric human 4-1BB ligand Fc (kih) antigen binding molecules show a similar activation improvement of HLA-A2-NLV-peptide specific CD8 T cells shown in FIGS. 15A-C as CD137 (4-1BB)-upregulation (positive feedback loop) and in FIGS. 15D-F as IFNγ expression after 24 h of stimulation. Differences of curves lie in the range of normal error deviation and are not significant.

FIGS. 16A and 16B show the binding of CD19-targeted split trimeric 4-1BBL Fc (kih) antigen binding molecules to 4-1BB expressing activated human CD4 and CD8 T cells from human PBMCs, respectively (see Example 7.1). Cells were gated on CD4 and CD8, and the binding curves were expressed by the mean fluorescent intensity of the secondary antibody against human Fc.

FIG. 17 shows the binding of CD19-targeted split trimeric 4-1BBL fusion molecules to CD19-expressing B lymphoma cell line (Example 7.2). The binding curves of the tested compounds (CD19-targeted split trimeric 4-1BBL Fc (kih) antigen binding molecules and untargeted Control D) to WSU-DLCL2 cells were expressed by the mean fluorescent intensity of the secondary antibody against human Fc.

FIG. 18 shows a scheme that illustrates the general principal of the NFkB activity assay described in Example 7.3 using a reporter cell line and CD19 expressing tumor cell lines. Shown is the activation assay set up with human 4-1BB expressing HeLa reporter cell line. A crosslinking of 4-1BB expressed on the reporter cells induces NFκB activation and NFκB-mediated Luciferase expression. After lysis of the cells Luciferase can catalyze the oxidation of Luciferin to Oxyluciferin. This chemical reaction correlates positively with the strength of NFκB-mediated luciferase expression and can be measured by the strength of light emission (units of released light).

FIGS. 19A to 19D show the NFκB-activation of luciferase in the 4-1BB expressing reporter cell line in the presence of CD19 expressing tumor cell lines and CD19-targeted split trimeric 4-1BBL Fc (kih) fusion antigen binding molecules as measured with the assay described in Example 7.3. Different test compounds containing either the anti-human CD19-binder clone 8B8-018 (construct 3.3 and 3.11) or the anti-human CD19 binder clone 8B8-2B11 (construct 4.1 and 4.11) were incubated at different concentrations (shown as nM on the x-axis) with 4-1BB expressing reporter cell line and no CD19-expressing cells (FIG. 19A) or CD19-expressing tumor cell lines OCI-Ly18, Nalm6 and SU-DHL-8 (FIG. 19B, FIG. 19C and FIG. 19D, respectively) at a E:T ratio 1:5 for 6 hours. The monovalent CD19-targeted split trimeric 4-1BBL Fc (kih) fusion antigen binding molecules (Constructs 3.11 and 4.11) could induce a nice Luciferase activity measured by the units of released light (URL) measured for 0.5s/well (shown on the y-axis) similar to the positive CD19-targeted control molecules Constructs 3.4 and 4.1 (as described in Example 1.11). The non-CD19-targeted control molecules Control F and D induce no Luciferase activity. All values were baseline corrected by substracting the blank control (no construct added or control added).

FIG. 20 shows IFNγ release in activated PBMCs via agonistic CD3 antibody in combination with CD19-targeted split trimeric 4-1BBL fusion molecules. IFN-γ releases were measured by ELISA in the culture supernatant of human PBMCs in the presence of different targeted- or untargeted-CD19-targeted split trimeric 4-1BBL Fc (kih) antigen binding molecules.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

As used herein, the term “moiety capable of specific binding to a target cell antigen” or “antigen binding domain capable of specific binding to a target cell antigen” refers to a polypeptide molecule that specifically binds to an antigenic determinant. In one aspect, the antigen binding moiety is able to activate signaling through its target cell antigen. In a particular aspect, the antigen binding moiety is able to direct the entity to which it is attached (e.g. the TNF family ligand trimer) to a target site, for example to a specific type of tumor cell or tumor stroma bearing the antigenic determinant. Moieties capable of specific binding to a target cell antigen include antibodies and fragments thereof as further defined herein. In addition, moieties capable of specific binding to a target cell antigen include scaffold antigen binding proteins as further defined herein, e.g. binding domains which are based on designed repeat proteins or designed repeat domains (see e.g. WO 2002/020565).

As used herein, the term “Fab domain capable of specific binding to a target cell antigen” refers to a Fab domain that specifically binds to an antigenic determinant. In one aspect, the antigen binding moiety is able to activate signaling through its target cell antigen. In a particular aspect, the antigen binding moiety is able to direct the entity to which it is attached (e.g. the TNF family ligand trimer) to a target site, for example to a specific type of tumor cell or tumor stroma bearing the antigenic determinant.

In relation to an antibody or Fab domain, the term “moiety capable of specific binding to a target cell antigen” refers to the part of the molecule that comprises the area which specifically binds to and is complementary to part or all of an antigen. A moiety capable of specific antigen binding may be provided, for example, by one or more antibody variable domains (also called antibody variable regions). Particularly, a moiety capable of specific antigen binding comprises an antibody light chain variable region (VL) and an antibody heavy chain variable region (VH).

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule.

The term “monovalent to an antigen” as used within the current application denotes the presence of only one binding site for said antigen in the antigen binding molecule. The term “monovalent to a target cell antigen” as used within the current application denotes the presence of only one binding site for said target cell antigen in the antigen binding molecule.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “Fab domain” as used herein denotes a Fab fragment or cross-Fab fragment as defined herein after.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab _{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin).

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “target cell antigen” as used herein refers to an antigenic determinant presented on the surface of a target cell, for example a cell in a tumor such as a cancer cell or a cell of the tumor stroma. In certain embodiments, the target cell antigen is an antigen on the surface of a tumor cell. In one embodiment, target cell antigen is selected from the group consisting of Fibroblast Activation Protein (FAP), Carcinoembryonic Antigen (CEA), Melanoma-associated Chondroitin Sulfate Proteoglycan (MCSP), Epidermal Growth Factor Receptor (EGFR), CD19, CD20 and CD33. In particular, the target cell antigen is Fibroblast Activation Protein (FAP).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:53), or NCBI (www.ncbi.nlm.nih.gov/) RefSeq NP_004451.2. The extracellular domain (ECD) of human FAP extends from amino acid position 26 to 760. The amino acid and nucleotide sequences of a His-tagged human FAP ECD is shown in SEQ ID NOs 54 and 55, respectively. The amino acid sequence of mouse FAP is shown in UniProt accession no. P97321 (version 126, SEQ ID NO:56), or NCBI RefSeq NP_032012.1. The extracellular domain (ECD) of mouse FAP extends from amino acid position 26 to 761. SEQ ID NOs 57 and 58 show the amino acid and nucleotide sequences, respectively, of a His-tagged mouse FAP ECD. SEQ ID NOs 59 and 60 show the amino acid and nucleotide sequences, respectively, of a His-tagged cynomolgus FAP ECD. Preferably, an anti-FAP binding molecule of the invention binds to the extracellular domain of FAP. Exemplary anti-FAP binding molecules are described in International Patent Application No. WO 2012/020006 A2.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:61). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarstrom S., Semin Cancer Biol. 9(2):67-81 (1999)), making it inaccessible to antibody in the blood stream. In contrast to normal tissue, CEA tends to be expressed over the entire surface of cancerous cells (Hammarstrom S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:62). The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:63).

The term “CD19” refers to B-lymphocyte antigen CD19, also known as B-lymphocyte surface antigen B4 or T-cell surface antigen Leu-12 and includes any native CD19 from any vertebrate source, including mammals such as primates (e.g. humans) non-human primates (e.g. cynomolgus monkeys) and rodents (e.g. mice and rats), unless otherwise indicated. The amino acid sequence of human CD19 is shown in Uniprot accession no. P15391 (version 160, SEQ ID NO:64). The term encompasses “full-length” unprocessed human CD19 as well as any form of human CD19 that results from processing in the cell as long as the antibody as reported herein binds thereto. CD19 is a structurally distinct cell surface receptor expressed on the surface of human B cells, including, but not limited to, pre-B cells, B cells in early development {i.e., immature B cells), mature B cells through terminal differentiation into plasma cells, and malignant B cells. CD19 is expressed by most pre-B acute lymphoblastic leukemias (ALL), non-Hodgkin's lymphomas, B cell chronic lymphocytic leukemias (CLL), pro-lymphocytic leukemias, hairy cell leukemias, common acute lymphocytic leukemias, and some Null-acute lymphoblastic leukemias. The expression of CD19 on plasma cells further suggests it may be expressed on differentiated B cell tumors such as multiple myeloma. Therefore, the CD19 antigen is a target for immunotherapy in the treatment of non-Hodgkin's lymphoma, chronic lymphocytic leukemia and/or acute lymphoblastic leukemia.

“CD20” refers to B-lymphocyte antigen CD20, also known as membrane-spanning 4-domains subfamily A member 1 (MS4A1), B-lymphocyte surface antigen B1 or Leukocyte surface antigen Leu-16, and includes any native CD20 from any vertebrate source, including mammals such as primates (e.g. humans) non-human primates (e.g. cynomolgus monkeys) and rodents (e.g. mice and rats), unless otherwise indicated. The amino acid sequence of human CD20 is shown in Uniprot accession no. P11836 (version 149, SEQ ID NO:65). “CD33” refers to Myeloid cell surface antigen CD33, also known as SIGLEC3 or gp67, and includes any native CD33 from any vertebrate source, including mammals such as primates (e.g. humans) non-human primates (e.g. cynomolgus monkeys) and rodents (e.g. mice and rats), unless otherwise indicated. The amino acid sequence of human CD33 is shown in Uniprot accession no. P20138 (version 157, SEQ ID NO:66).

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, “Sequences of Proteins of Immunological Interest” (1983) and by Chothia et al., J Mol. Biol. 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table A as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE A
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to Clq binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fe region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, Md., 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcaRI (CD89). A particular activating Fc receptor is human FcγRIIIa (see UniProt accession no. P08637, version 141).

The term “TNF ligand family member” or “TNF family ligand” refers to a proinflammatory cytokine. Cytokines in general, and in particular the members of the TNF ligand family, play a crucial role in the stimulation and coordination of the immune system. At present, nineteen cyctokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors.

Members of the TNF ligand family are selected from the group consisting of Lymphotoxin a (also known as LTA or TNFSF1), TNF (also known as TNFSF2), LTP (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated. In specific embodiments of the invention, the TNF ligand family member is selected from the group consisting of OX40L, FasL, CD27L, TRAIL, 4-1BBL, CD40L and GITRL. Ina particular embodiment, the TNF ligand family member is selected from 4-1BBL and OX40L.

Further information, in particular sequences, of the TNF ligand family members may be obtained from publically accessible databases such as Uniprot (www.uniprot.org). For instance, the human TNF ligands have the following amino acid sequences: human Lymphotoxin a (UniProt accession no. P01374, SEQ ID NO:67), human TNF (UniProt accession no. P01375, SEQ ID NO:68), human Lymphotoxin f (UniProt accession no. Q06643, SEQ ID NO:69), human OX40L (UniProt accession no. P23510, SEQ ID NO:70), human CD40L (UniProt accession no. P29965, SEQ ID NO:71), human FasL (UniProt accession no. P48023, SEQ ID NO:72), human CD27L (UniProt accession no. P32970, SEQ ID NO:73), human CD30L (UniProt accession no. P32971, SEQ ID NO:74), 4-1BBL (UniProt accession no. P41273, SEQ ID NO:75), TRAIL (UniProt accession no. P50591, SEQ ID NO:76), RANKL (UniProt accession no. 014788, SEQ ID NO:77), TWEAK (UniProt accession no. 043508, SEQ ID NO:78), APRIL (UniProt accession no. 075888, SEQ ID NO:79), BAFF (UniProt accession no. Q9Y275, SEQ ID NO:80), LIGHT (UniProt accession no. 043557, SEQ ID NO:81), TL1A (UniProt accession no. 095150, SEQ ID NO:82), GITRL (UniProt accession no. Q9UNG2, SEQ ID NO:83) and ectodysplasin A (UniProt accession no. Q92838, SEQ ID NO:84).

An “ectodomain” is the domain of a membrane protein that extends into the extracellular space (i.e. the space outside the target cell). Ectodomains are usually the parts of proteins that initiate contact with surfaces, which leads to signal transduction. The ectodomain of TNF ligand family member as defined herein thus refers to the part of the TNF ligand protein that extends into the extracellular space (the extracellular domain), but also includes shorter parts or fragments thereof that are responsible for the trimerization and for the binding to the corresponding TNF receptor. The term “ectodomain of a TNF ligand family member or a fragment thereof” thus refers to the extracellular domain of the TNF ligand family member that forms the extracellular domain or to parts thereof that are still able to bind to the receptor (receptor binding domain).

The term “costimulatory TNF ligand family member” or “costimulatory TNF family ligand” refers to a subgroup of TNF ligand family members, which are able to costimulate proliferation and cytokine production of T-cells. These TNF family ligands can costimulate TCR signals upon interaction with their corresponding TNF receptors and the interaction with their receptors leads to recruitment of TNFR-associated factors (TRAF), which initiate signalling cascades that result in T-cell activation. Costimulatory TNF family ligands are selected from the group consisting of 4-1BBL, OX40L, GITRL, CD70, CD30L and LIGHT, more particularly the costimulatory TNF ligand family member is selected from 4-1BBL and OX40L.

As described herein before, 4-1BBL is a type II transmembrane protein and one member of the TNF ligand family. Complete or full length 4-1BBL having the amino acid sequence of SEQ ID NO:42 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO:85) form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO:4 (amino acids 52-254 of human 4-1BBL), SEQ ID NO:1 (amino acids 71-254 of human 4-1BBL), SEQ ID NO:3 (amino acids 80-254 of human 4-1BBL) and SEQ ID NO:2 (amino acids 85-254 of human 4-1BBL) or a polypeptide having an amino acid sequence selected from SEQ ID NO:5 (amino acids 71-248 of human 4-1BBL), SEQ ID NO:8 (amino acids 52-248 of human 4-1BBL), SEQ ID NO:7 (amino acids 80-248 of human 4-1BBL) and SEQ ID NO:6 (amino acids 85-248 of human 4-1BBL), but also other fragments of the ectodomain capable of trimerization are included herein.

As described herein before, OX40L is another type II transmembrane protein and a further member of the TNF ligand family. Complete or full length human OX40L has the amino acid sequence of SEQ ID NO:70. The amino acids 51-183 of the human OX40L sequence (SEQ ID NO:11) form the extracellular domain of OX40L, but even fragments thereof that are able to form the trimers. In specific embodiments of the invention, the term “ectodomain of OX40L or a fragment thereof” refers to a polypeptide having an amino acid sequence selected from SEQ ID NO:11 (amino acids 51-183 of human OX40L) or SEQ ID NO:12 (amino acids 52-183 of human OX40L), but also other fragments of the ectodomain capable of trimerization are included herein.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 1 and 4, in particular 2, i.e. the peptides selected from the group consisting of GGGGS (SEQ ID NO:86), GGGGSGGGGS (SEQ ID NO:87), SGGGGSGGGG (SEQ ID NO:88), (G₄S)₃ or GGGGSGGGGSGGGGS (SEQ ID NO:89), GGGGSGGGGSGGGG or G4(SG₄)₂ (SEQ ID NO:90), and (G₄S)₄ or GGGGSGGGGSGGGGSGGGGS (SEQ ID NO:91), but also include the sequences GSPGSSSSGS (SEQ ID NO:92), GSGSGSGS (SEQ ID NO:93), GSGSGNGS (SEQ ID NO:94), GGSGSGSG (SEQ ID NO:95), GGSGSG (SEQ ID NO:96), GGSG (SEQ ID NO:97), GGSGNGSG (SEQ ID NO:98), GGNGSGSG (SEQ ID NO:99) and GGNGSG (SEQ ID NO:100). Peptide linkers of particular interest are (G4S)₁ or GGGGS (SEQ ID NO:86), (G₄S)₂ or GGGGSGGGGS (SEQ ID NO:87) and GSPGSSSSGS (SEQ ID NO:92), more particularly (G₄S)₂ or GGGGSGGGGS (SEQ ID NO:87).

The term “amino acid” as used within this application denotes the group of naturally occurring carboxy a-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “single chain fusion protein” as used herein refers to a single chain polypeptide composed of one or two ectodomains of said TNF ligand family member fused to a part of antigen binding moiety or Fc part. The fusion may occur by directly linking the N or C-terminal amino acid of the antigen binding moiety via a peptide linker to the C- or N-terminal amino acid of the ectodomain of said TNF ligand family member.

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the TNF ligand trimer-containing antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE B

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include TNF family ligand trimer-containing antigen binding molecule with an N-terminal methionyl residue. Other insertional variants of the molecule include the fusion to the N- or C-terminus to a polypeptide which increases the serum half-life of the TNF ligand trimer-containing antigen binding molecules.

In certain embodiments, the TNF family ligand trimer-containing antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of TNF family ligand trimer-containing antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the TNF family ligand trimer-containing antigen binding molecules of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the TNF family ligand trimer-containing antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the TNF family ligand trimer-containing antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

In another aspect, immunoconjugates of the TNF family ligand trimer-containing antigen binding molecules provided herein maybe obtained. An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, carcinoma, lymphoma, blastoma, sarcoma, leukemia, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colorectal cancer (CRC), pancreatic cancer, breast cancer, triple-negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, melanoma, multiple myeloma, B-cell cancer (lymphoma), chronic lymphocytic leukemia (CLL), acute lymphoblastic leukemia (ALL), hairy cell leukemia, chronic myeloblastic leukemia, including refractory versions of any of the above cancers, or a combination of one or more of the above cancers.

TNF Family Ligand Trimer-Containing Antigen Binding Molecules of the Invention

The invention provides novel TNF family ligand trimer-containing antigen binding molecules with particularly advantageous properties such as producibility, stability, robustness, binding affinity, biological activity, targeting efficiency and reduced toxicity.

In a first aspect, the invention provides a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fe domain.

In a particular aspect, the invention provides a costimulatory TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a costimulatory TNF ligand family member or fragments thereof that are connected to each other by a peptide linker and a second polypeptide comprising one ectodomain of said costimulatory TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain. Thus, the TNF family ligand trimer-containing antigen binding molecule comprises (a) a Fab domain capable of specific binding to a target cell antigen, (b) a Fc domain composed of a first and a second subunit capable of stable association, and (c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the TNF ligand family member costimulates human T-cell activation.

In another particular aspect, the TNF family ligand trimer-containing antigen binding molecule (a) a Fab domain capable of specific binding to a target cell antigen,

(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the ectodomains of a TNF ligand family member are identical in all instances.

In a particular aspect, the TNF family ligand trimer-containing antigen binding molecule comprises a TNF ligand family member that costimulates human T-cell activation which is selected from 4-1BBL and OX40L. More particularly, the TNF ligand family member is 4-1BBL.

In a further aspect, the ectodomain of a TNF ligand family member comprises the amino acid sequence selected from the group consisting of SEQ ID NO:1, SEQ ID NO: 2, SEQ ID NO:3, SEQ ID NO:4, SEQ ID NO:5, SEQ ID NO:6, SEQ ID NO:7 and SEQ ID NO:8, particularly the amino acid sequence of SEQ ID NO:1 or SEQ ID NO:5.

In a particular aspect, the ectodomain of a TNF ligand family member comprises the amino acid sequence selected from the group consisting of SEQ ID NO:5, SEQ ID NO:6, SEQ ID NO:7 and SEQ ID NO:8. More particularly, the ectodomain of a TNF ligand family member comprises the amino acid sequence of SEQ ID NO:5.

In a further aspect, the TNF family ligand trimer-containing antigen binding molecule of the invention comprises

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain and wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5. In one aspect, the first polypeptide comprising two ectodomains of a TNF ligand family member or fragments thereof comprises an amino acid sequence SEQ ID NO:9 and the second polypeptide comprising one ectodomain of said TNF ligand family member or a fragment thereof comprises the amino acid sequence of SEQ ID NO:1. In a particular aspect, the first polypeptide comprising two ectodomains of a TNF ligand family member or fragments thereof comprises an amino acid sequence of SEQ ID NO:10 and the second polypeptide comprising one ectodomain of said TNF ligand family member or a fragment thereof comprises the amino acid sequence of SEQ ID NO:5.

In a further aspect, provided is aTNF family ligand trimer-containing antigen binding molecule as described herein before, wherein the TNF ligand family member is OX40L. In one aspect, provided is TNF family ligand trimer-containing antigen binding molecule, wherein the ectodomain of a TNF ligand family member comprises the amino acid sequence selected from the group consisting of SEQ ID NO:11 and SEQ ID NO:12.

In one aspect, the invention provides a TNF family ligand trimer-containing antigen binding molecule comprising

(a) one Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain. The invention thus relates to a TNF family ligand trimer-containing antigen binding molecule, wherein TNF family ligand trimer-containing antigen binding molecule is monovalent for the binding to the target cell antigen.

In another aspect, provided is a TNF family ligand trimer-containing antigen binding molecule of the invention, wherein the target cell antigen is selected from the group consisting of Fibroblast Activation Protein (FAP), CD19, Carcinoembryonic Antigen (CEA), Melanoma-associated Chondroitin Sulfate Proteoglycan (MCSP), Epidermal Growth Factor Receptor (EGFR), CD20 and CD33. In one aspect, the target cell antigen is selected from the group consisting of Fibroblast Activation Protein (FAP), CD19 and Carcinoembryonic Antigen (CEA).

In a particular aspect, provided is a TNF family ligand trimer-containing antigen binding molecule, wherein the Fab domain capable of specific binding to a target cell antigen is a Fab or a crossover Fab capable of specific binding to a target cell antigen.

In a particular aspect, the target cell antigen is Fibroblast Activation Protein (FAP).

In one aspect, the invention provides a TNF family ligand trimer-containing antigen binding molecule, wherein the Fab domain capable of specific binding to FAP comprises

(a) a VH domain comprising (i) CDR-H1 comprising the amino acid sequence of SEQ ID NO:13, (ii) CDR-H2 comprising the amino acid sequence of SEQ ID NO:14 and (iii) CDR-H3 comprising the amino acid sequence of SEQ ID NO:15, and a VL domain comprising (iv) CDR-L1 comprising the amino acid sequence of SEQ ID NO:16, (v) CDR-L2 comprising the amino acid sequence of SEQ ID NO:17 and (vi) CDR-L3 comprising the amino acid sequence of SEQ ID NO:18 or
(b) a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.

In a particular aspect, the invention provides a TNF family ligand trimer-containing antigen binding molecule, wherein the Fab domain capable of specific binding to FAP comprises a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.

In a further aspect, provided is a TNF family ligand trimer-containing antigen binding molecule as described herein before, wherein the Fab domain capable of specific binding to FAP comprises a variable heavy chain comprising an amino acid sequence of SEQ ID NO:25 and a variable light chain comprising an amino acid sequence of SEQ ID NO:26 or wherein the Fab domain capable of specific binding to FAP comprises a variable heavy chain comprising an amino acid sequence of SEQ ID NO:27 and a variable light chain comprising an amino acid sequence of SEQ ID NO:28.

In another particular aspect, the target cell antigen is CD19.

In one aspect, the invention provides a TNF family ligand trimer-containing antigen binding molecule, wherein the Fab domain capable of specific binding to CD19 comprises

(a) a VH domain comprising (i) CDR-H1 comprising the amino acid sequence of SEQ ID NO:29, (ii) CDR-H2 comprising the amino acid sequence of SEQ ID NO:30 and (iii) CDR-H3 comprising the amino acid sequence of SEQ ID NO:31, and a VL domain comprising (iv) CDR-L1 comprising the amino acid sequence of SEQ ID NO:32, (v) CDR-L2 comprising the amino acid sequence of SEQ ID NO:33 and (vi) CDR-L3 comprising the amino acid sequence of SEQ ID NO:34 or
(b) a VH domain comprising (i) CDR-H1 comprising the amino acid sequence of SEQ ID NO:35, (ii) CDR-H2 comprising the amino acid sequence of SEQ ID NO:36 and (iii) CDR-H3 comprising the amino acid sequence of SEQ ID NO:37, and a VL domain comprising (iv) CDR-L1 comprising the amino acid sequence of SEQ ID NO:38, (v) CDR-L2 comprising the amino acid sequence of SEQ ID NO:39 and (vi) CDR-L3 comprising the amino acid sequence of SEQ ID NO:40.

In a particular aspect, the invention provides a TNF family ligand trimer-containing antigen binding molecule, wherein the Fab domain capable of specific binding to CD19 comprises a VH domain comprising (i) CDR-H1 comprising the amino acid sequence of SEQ ID NO:35, (ii) CDR-H2 comprising the amino acid sequence of SEQ ID NO:36 and (iii) CDR-H3 comprising the amino acid sequence of SEQ ID NO:37, and a VL domain comprising (iv) CDR-L1 comprising the amino acid sequence of SEQ ID NO:38, (v) CDR-L2 comprising the amino acid sequence of SEQ ID NO:39 and (vi) CDR-L3 comprising the amino acid sequence of SEQ ID NO:40.

In a further aspect, provided is a TNF family ligand trimer-containing antigen binding molecule as described herein before, wherein the Fab domain capable of specific binding to CD19 comprises a variable heavy chain comprising an amino acid sequence of SEQ ID NO:41 and a variable light chain comprising an amino acid sequence of SEQ ID NO:42 or wherein the Fab domain capable of specific binding to FAP comprises a variable heavy chain comprising an amino acid sequence of SEQ ID NO:43 and a variable light chain comprising an amino acid sequence of SEQ ID NO:44.

In another particular aspect, the target cell antigen is CEA.

In one aspect, the invention provides a TNF family ligand trimer-containing antigen binding molecule, wherein the Fab domain capable of specific binding to CEA comprises a VH domain comprising (i) CDR-H1 comprising the amino acid sequence of SEQ ID NO:45, (ii) CDR-H2 comprising the amino acid sequence of SEQ ID NO:46 and (iii) CDR-H3 comprising the amino acid sequence of SEQ ID NO:47, and a VL domain comprising (iv) CDR-L1 comprising the amino acid sequence of SEQ ID NO:48, (v) CDR-L2 comprising the amino acid sequence of SEQ ID NO:49 and (vi) CDR-L3 comprising the amino acid sequence of SEQ ID NO:50.

In a particular aspect, provided is a TNF family ligand trimer-containing antigen binding molecule as described herein before, wherein the Fab domain capable of specific binding to CEA comprises a variable heavy chain comprising an amino acid sequence of SEQ ID NO:51 and a variable light chain comprising an amino acid sequence of SEQ ID NO:52.

In a further aspect, the Fc domain is an IgG, particularly an IgG1 Fc domain or an IgG4 Fc domain. More particularly, the Fc domain is an IgG1 Fc domain. In a particular aspect, the Fc domain comprises a modification promoting the association of the first and second subunit of the Fc domain.

In particular, the TNF family ligand trimer-containing antigen binding molecule of the invention comprises a Fab domain capable of specific binding to a target cell antigen, wherein the Fab heavy chain is fused at the C-terminus to the N-terminus of a CH2 domain in the Fc domain.

In another aspect, the invention is concerned with a TNF family ligand trimer-containing antigen binding molecule as defined herein before, comprising

(b) an Fc domain composed of a first and a second subunit capable of stable association, wherein the Fc domain comprises one or more amino acid substitution that reduces binding to an Fc receptor, in particular towards Fcγ receptor.

Fc Domain Modifications Reducing Fc Receptor Binding and/or Effector Function

The Fc domain of the TNF family ligand trimer-containing antigen binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the TNF family ligand trimer-containing antigen binding molecule of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of a TNF family ligand trimer-containing antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the Fc domain comprises one or more amino acid substitution that reduces binding to an Fc receptor, in particular towards Fcγ receptor.

In one aspect, the Fc domain of the TNF family ligand trimer-containing antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is a trimeric TNF family ligand-containing antigen binding molecule according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions. “EU numbering” refers to the numbering according to EU index of Kabat et al, Sequences of Proteins of Immunological Interest, 5th Ed. Public Health Service, National Institutes of Health, Bethesda, Md., 1991.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fe domain is an IgG4 Fe domain. IgG4 antibodies exhibit reduced binding affinity to Fc receptors and reduced effector functions as compared to IgG antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656(1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

In a particular aspect, the Fc domain comprises a modification promoting the association of the first and second subunit of the Fc domain.

Fc Domain Modifications Promoting Heterodimerization

In one aspect, the TNF family ligand trimer-containing antigen binding molecules of the invention comprise (a) a Fab domain capable of specific binding to a target cell antigen, (b) a Fc domain composed of a first and a second subunit capable of stable association, and (c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, wherein the Fc domain comprises one or more amino acid substitution that reduces binding to an Fc receptor, in particular towards Fcγ receptor. Thus, they comprise different moieties, fused to one or the other of the two subunits of the Fc domain that are typically comprised in two non-identical polypetide chains (“heavy chains”). Recombinant co-expression of these polypeptides and subsequent dimerization leads to several possible combinations of the two polypeptides. To improve the yield and purity of the TNF family ligand trimer-containing antigen binding molecules in recombinant production, it will thus be advantageous to introduce in the Fc domain of the TNF family ligand trimer-containing antigen binding molecules of the invention a modification promoting the association of the desired polypeptides.

Accordingly, the Fc domain of the TNF family ligand trimer-containing antigen binding molecules of the invention comprises a modification promoting the association of the first and the second subunit of the Fc domain. The site of most extensive protein-protein interaction between the two subunits of a human IgG Fc domain is in the CH3 domain of the Fc domain. Thus, said modification is particularly in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, in a particular aspect, the invention relates to a TNF family ligand trimer-containing antigen binding molecule as described herein before which comprises an IgG molecule, wherein the Fc part of the first heavy chain comprises a first dimerization module and the Fc part of the second heavy chain comprises a second dimerization module allowing a heterodimerization of the two heavy chains of the IgG molecule and the first dimerization module comprises knobs and the second dimerization module comprises holes according to the knob into hole technology.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular aspect, in the CH3 domain of the first subunit of the Fc domain of the TNF family ligand trimer-containing antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). More particularly, in the second subunit of the Fc domain additionally the threonine residue at position 366 is replaced with a serine residue (T366S) and the leucine residue at position 368 is replaced with an alanine residue (L368A). More particularly, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). The introduction of these two cysteine residues results in the formation of a disulfide bridge between the two subunits of the Fc domain. The disulfide bridge further stabilizes the dimer (Carter, J Immunol Methods 248, 7-15 (2001)).

Thus, in a particular aspect, the Fe domain comprises a modification promoting the association of the first and second subunit of the Fc domain. In a particular aspect, provided is a TNF family ligand trimer-containing antigen binding molecule as defined herein before, wherein the first subunit of the Fc domain comprises the amino acid substitutions S354C and T366W (numbering according to Kabat EU index) of the knob and the second subunit of the Fc domain comprises the amino acid substitutions Y349C, T366S, L368A and Y407V (numbering according to Kabat EU index) of the hole.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

In a further aspect, provided is a TNF family ligand trimer-containing antigen binding molecule as defined herein before, wherein the first polypeptide comprising two ectodomains of a TNF ligand family member or fragments thereof that are connected to each other by a peptide linker is fused at its N-terminus to the C-terminus of the second subunit of the Fc domain (hole) and the second polypeptide comprising one ectodomain of said TNF ligand family member or a fragment thereof is fused at its N-terminus to the C-terminus of the first subunit of the Fc domain (knob).

In another aspect, provided is a TNF family ligand trimer-containing antigen binding molecule as defined herein before, wherein the first polypeptide comprising two ectodomains of a TNF ligand family member or fragments thereof that are connected to each other by a peptide linker is fused at its N-terminus to the C-terminus of the first subunit of the Fc domain (knob) and the second polypeptide comprising one ectodomain of said TNF ligand family member or a fragment thereof is fused at its N-terminus to the C-terminus of the second subunit of the Fc domain (hole).

In one aspect, the invention provides a TNF family ligand trimer-containing antigen binding molecule, wherein the Fab domain capable of specific binding to a target cell antigen is fused at the C-terminus to the N-terminus of the first subunit of the Fc domain (knob). In another aspect, provided is a TNF family ligand trimer-containing antigen binding molecule, wherein the Fab domain capable of specific binding to a target cell antigen is fused at the C-terminus to the N-terminus of the second subunit of the Fc domain (hole).

In a particular aspect, provided is a TNF family ligand trimer-containing antigen binding molecule, wherein the Fab domain capable of specific binding to a target cell antigen is fused at the C-terminus to the N-terminus of the first subunit of the Fc domain (knob), the first polypeptide comprising two ectodomains of a TNF ligand family member or fragments thereof that are connected to each other by a peptide linker is fused at its N-terminus to the C-terminus of the second subunit of the Fc domain (hole) and the second polypeptide comprising one ectodomain of said TNF ligand family member or a fragment thereof is fused at its N-terminus to the C-terminus of the first subunit of the Fc domain (knob).

In a further aspect, the invention provides a TNF family ligand trimer-containing antigen binding molecule as defined before further comprising (d) a Fab domain that is not capable of specific binding to a target cell antigen. Thus, the invention provides a TNF family ligand trimer-containing antigen binding molecule comprising

(a) a Fab domain capable of specific binding to a target cell antigen,
(b) a Fc domain composed of a first and a second subunit capable of stable association,
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, and
(d) a Fab domain that is not capable of specific binding to a target cell antigen.

These molecules comprising a Fab domain that is not capable of specific binding to a target cell antigen have a full IgG-like structure while at the same time they have the advantage of only one binding site for the target cell antigen (monovalent binding). They may thus have a better Pk profile and/or less side effects caused for instance by anti-drug antibodies.

Modifications in the CH1/CL Domains

To further improve correct pairing in antigen binding molecules comprising two different Fab domains, the TNF family ligand trimer-containing antigen binding molecules can contain different charged amino acid substitutions (so-called “charged residues”). These modifications are introduced in the crossed or non-crossed CH1 and CL domains. In a particular aspect, the invention relates to a TNF family ligand trimer-containing antigen binding molecule, wherein in one of CL domains the amino acid at position 123 (EU numbering) has been replaced by arginine (R) and the amino acid at position 124 (EU numbering) has been substituted by lysine (K) and wherein in one of the CH1 domains the the amino acids at position 147 (EU numbering) and at position 213 (EU numbering) have been substituted by glutamic acid (E).

More particularly, the invention relates to a TNF family ligand trimer-containing antigen binding molecule comprising

(a) a Fab domain capable of specific binding to a target cell antigen,
(b) a Fc domain composed of a first and a second subunit capable of stable association,
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain, and
(d) a Fab domain that is not capable of specific binding to a target cell antigen being a crossover Fab, wherein in the CL domain the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E).

Particular TNF Family Ligand Trimer-Containing Antigen Binding Molecules

In one aspect, provided is a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fc domain comprising the hole mutations.

In a particular aspect, the invention provides a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fe domain comprising the hole mutations and wherein the VH and CH1 domain of the Fab domain capable of specific binding to a target cell antigen is fused at the C-terminus to the N-terminus of the second subunit of the Fc domain comprising the knob mutations.

In another aspect, the invention provides a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fc domain comprising the hole mutations and wherein the VH and CH1 domain of the Fab domain capable of specific binding to a target cell antigen is fused at the C-terminus to the N-terminus of the first subunit of the Fc domain comprising the hole mutations.

In a further aspect, provided is a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fc domain comprising the hole mutations and wherein the second polypeptide is fused at its N-terminus to the C-terminus of the second subunit of the Fc domain comprising the knob mutations.

In a particular aspect, the invention provides a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fc domain comprising the hole mutations and wherein the second polypeptide is fused at its N-terminus to the C-terminus of the second subunit of the Fe domain comprising the knob mutations and wherein the VH and CH1 domain of the Fab domain capable of specific binding to a target cell antigen is fused at the C-terminus to the N-terminus of the second subunit of the Fc domain comprising the knob mutations.

In another aspect, the invention provides a TNF family ligand trimer-containing antigen binding molecule comprising

(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fc domain comprising the hole mutations and wherein the second polypeptide is fused at its N-terminus to the C-terminus of the second subunit of the Fc domain comprising the knob mutations and wherein the VH and CH1 domain of the Fab domain capable of specific binding to a target cell antigen is fused at the C-terminus to the N-terminus of the first subunit of the Fc domain comprising the hole mutations.

TNF Family Ligand Trimer-Containing Antigen Binding Molecules, Wherein the Target Cell Antigen is FAP

In one aspect, provided is a TNF family ligand trimer-containing antigen binding molecule, wherein the target cell antigen is Fibroblast Activation Protein (FAP).

In a particular aspect, provided is a TNF family ligand trimer-containing antigen binding molecule as defined herein before, wherein the Fab domain capable of specific binding to FAP comprises a heavy chain variable region comprising an amino acid sequence that is at least about 95%, 96%, 97%, 98%, 99% or 100% identical to the amino acid sequence of SEQ ID NO:25 and a light chain variable region comprising an amino acid sequence that is at least about 95%, 96%, 97%, 98%, 99% or 100% identical to the amino acid sequence of SEQ ID NO:26.

In another particular aspect, provided is a TNF family ligand trimer-containing antigen binding molecule as defined herein before, wherein the Fab domain capable of specific binding to FAP comprises a heavy chain variable region comprising an amino acid sequence that is at least about 95%, 96%, 97%, 98%, 99% or 100% identical to the amino acid sequence of SEQ ID NO:27 and a light chain variable region comprising an amino acid sequence that is at least about 95%. 96%. 97%. 98%. 99% or 100% identical to the amino acid sequence of SEQ ID NO:28.

In one aspect, the Fab domain capable of specific binding to FAP comprises a heavy chain variable region comprising an amino acid sequence of SEQ ID NO:25 and a light chain variable region comprising an amino acid sequence of SEQ ID NO:26 or a heavy chain variable region comprising an amino acid sequence of SEQ ID NO:27 and a light chain variable region comprising an amino acid sequence of SEQ ID NO:28. In a particular aspect, the Fab domain capable of specific binding to FAP comprises a heavy chain variable region comprising the amino acid sequence of SEQ ID NO:25 and a light chain variable region comprising the amino acid sequence of SEQ ID NO:26. In another particular aspect, the Fab domain capable of specific binding to FAP comprises a heavy chain variable region comprising the amino acid sequence of SEQ ID NO:27 and a light chain variable region comprising the amino acid sequence of SEQ ID NO:28. In a specific aspect, the Fab domain capable of specific binding to FAP comprises a VH domain consisting of amino acid sequence of SEQ ID NO:27 and a VL domain consisting of the amino acid sequence of SEQ ID NO:28.

In a further aspect, the invention provides a TNF family ligand trimer-containing antigen binding molecule comprising

(a) a Fab domain capable of specific binding to FAP comprising a VH domain comprising an amino acid sequence of SEQ ID NO:25 and a VL domain comprising an amino acid sequence of SEQ ID NO:26, or a VH domain comprising an amino acid sequence of SEQ ID NO:27 and a VL domain comprising an amino acid sequence of SEQ ID NO:28,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain.

In another aspect, the invention provides a TNF family ligand trimer-containing antigen binding molecule comprising

(a) a Fab domain capable of specific binding to FAP comprising a VH domain comprising an amino acid sequence of SEQ ID NO:25 and a VL domain comprising an amino acid sequence of SEQ ID NO:26, or a VH domain comprising an amino acid sequence of SEQ ID NO:27 and a VL domain comprising an amino acid sequence of SEQ ID NO:28,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fe domain and wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5.

In a particular aspect, provided is a TNF family ligand trimer-containing antigen binding molecule comprising

(a) a Fab domain capable of specific binding to FAP comprising a VH domain comprising an amino acid sequence of SEQ ID NO:27 and a VL domain comprising an amino acid sequence of SEQ ID NO:28,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain and wherein the first polypeptide comprising two ectodomains of a TNF ligand family member comprising an amino acid sequence of SEQ ID NO:10 and the second polypeptide comprising one ectodomain of said TNF ligand family member comprising the amino acid sequence of SEQ ID NO:5.

In another aspect, provided is a TNF family ligand trimer-containing antigen binding molecule, wherein the antigen binding molecule comprises

(i) a light chain comprising the VL domain of the Fab domain capable of specific binding to FAP,
(ii) a fusion polypeptide comprising a first subunit of a Fc domain and a first polypeptide comprising two ectodomains of a TNF ligand family member or fragments thereof that are connected to each other by a peptide linker, wherein the first polypeptide is fused at its N-terminus to the C-terminus of the first subunit of the Fc domain, and
(iii) a heavy chain comprising the VH domain of the Fab domain capable of specific binding to FAP, a second subunit of the Fc domain and and a second polypeptide comprising one ectodomain of said TNF ligand family member or a fragment thereof, wherein the variable heavy chain of the Fab domain capable of specific binding to FAP is fused at its C-terminus to the N-terminus of the second subunit of the Fc domain and wherein the second polypeptide comprising one ectodomain of said TNF ligand family member or a fragment thereof is fused at its N-terminus to the C-terminus of the second subunit of the Fc domain.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:106,
(ii) a fusion polypeptide comprising an amino acid sequence that is at least about 95%, 96%, 97%, 98%, 99% or 100% identical to the amino acid sequence of SEQ ID NO:104, and
(iii) a heavy chain comprising an amino acid sequence that is at least about 95%, 96%, 97%, 98%, 99% or 100% identical to the amino acid sequence of SEQ ID NO:105.

More particularly, the TNF family ligand trimer-containing antigen binding molecule comprises (i) a light chain comprising the amino acid sequence of SEQ ID NO:106, (ii) a fusion polypeptide comprising the amino acid sequence of SEQ ID NO:104, and (iii) a heavy chain comprising the amino acid sequence of SEQ ID NO:105.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:114,
(ii) a fusion polypeptide comprising an amino acid sequence that is at least about 95%, 96%, 97%, 98%, 99% or 100% identical to the amino acid sequence of SEQ ID NO:104, and
(iii) a heavy chain comprising an amino acid sequence that is at least about 95%, 96%, 97%, 98%, 99% or 100% identical to the amino acid sequence of SEQ ID NO:113.

More particularly, the TNF family ligand trimer-containing antigen binding molecule comprises (i) a light chain comprising the amino acid sequence of SEQ ID NO:114, (ii) a fusion polypeptide comprising the amino acid sequence of SEQ ID NO:104, and (iii) a heavy chain comprising the amino acid sequence of SEQ ID NO:113.

In a further aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:106,
(ii) a fusion polypeptide comprising an amino acid sequence that is at least about 95%, 96%, 97%, 98%, 99% or 100% identical to the amino acid sequence of SEQ ID NO:178, and
(iii) a heavy chain comprising an amino acid sequence that is at least about 95%, 96%, 97%, 98%, 99% or 100% identical to the amino acid sequence of SEQ ID NO:177.

More particularly, the TNF family ligand trimer-containing antigen binding molecule comprises (i) a light chain comprising the amino acid sequence of SEQ ID NO:106, (ii) afusion polypeptide comprising the amino acid sequence of SEQ ID NO: 178, and (iii) a heavy chain comprising the amino acid sequence of SEQ ID NO:177.

In yet another aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:114,
(ii) a fusion polypeptide comprising an amino acid sequence that is at least about 95%, 96%, 97%, 98%, 99% or 100% identical to the amino acid sequence of SEQ ID NO:178, and
(iii) a heavy chain comprising an amino acid sequence that is at least about 95%, 96%, 97%, 98%, 99% or 100% identical to the amino acid sequence of SEQ ID NO:182.

More particularly, the TNF family ligand trimer-containing antigen binding molecule comprises (i) a light chain comprising the amino acid sequence of SEQ ID NO:114, (ii) a fusion polypeptide comprising the amino acid sequence of SEQ ID NO:178, and (iii) a heavy chain comprising the amino acid sequence of SEQ ID NO:182.

In another aspect, provided is a TNF family ligand trimer-containing antigen binding molecule, wherein the antigen binding molecule comprises

(i) a light chain comprising the VL domain of the Fab domain capable of specific binding to FAP,
(ii) a fusion polypeptide comprising a first subunit of a Fc domain and a second polypeptide comprising one ectodomain of said TNF ligand family member or a fragment thereof, wherein the second polypeptide is fused at its N-terminus to the C-terminus of the first subunit of the Fc domain, and
(iii) a heavy chain comprising the VH domain of the Fab domain capable of specific binding to FAP, a second subunit of the Fc domain and and a first polypeptide comprising two ectodomains of a TNF ligand family member or fragments thereof that are connected to each other by a peptide linker, wherein the variable heavy chain of the Fab domain capable of specific binding to FAP is fused at its C-terminus to the N-terminus of the second subunit of the Fc domain and wherein the first polypeptide comprising two ectodomains of a TNF ligand family member or fragments thereof is fused at its N-terminus to the C-terminus of the second subunit of the Fc domain.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:106,
(ii) a fusion polypeptide comprising an amino acid sequence that is at least about 95%, 96%, 97%. 98%, 99% or 100% identical to the amino acid sequence of SEQ ID NO:109, and
(iii) a heavy chain comprising an amino acid sequence that is at least about 95%, 96%, 97%, 98%, 99% or 100% identical to the amino acid sequence of SEQ ID NO:110.

More particularly, the TNF family ligand trimer-containing antigen binding molecule comprises (i) a light chain comprising the amino acid sequence of SEQ ID NO:106, (ii) a fusion polypeptide comprising the amino acid sequence of SEQ ID NO:109, and (iii) a heavy chain comprising the amino acid sequence of SEQ ID NO:110.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:114,
(ii) a fusion polypeptide comprising an amino acid sequence that is at least about 95%, 96%, 97%, 98%, 99% or 100% identical to the amino acid sequence of SEQ ID NO:109, and
(iii) a heavy chain comprising an amino acid sequence that is at least about 95%, 96%, 97%, 98%, 99% or 100% identical to the amino acid sequence of SEQ ID NO:116.

More particularly, the TNF family ligand trimer-containing antigen binding molecule comprises (i) a light chain comprising the amino acid sequence of SEQ ID NO:114, (ii) a fusion polypeptide comprising the amino acid sequence of SEQ ID NO:109, and (iii) a heavy chain comprising the amino acid sequence of SEQ ID NO:116.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:106,
(ii) a fusion polypeptide comprising an amino acid sequence that is at least about 95%, 96%, 97%, 98%, 99% or 100% identical to the amino acid sequence of SEQ ID NO:174, and
(iii) a heavy chain comprising an amino acid sequence that is at least about 95%, 96%, 97%, 98%, 99% or 100% identical to the amino acid sequence of SEQ ID NO:173.

More particularly, the TNF family ligand trimer-containing antigen binding molecule comprises (i) a light chain comprising the amino acid sequence of SEQ ID NO:106, (ii) a fusion polypeptide comprising the amino acid sequence of SEQ ID NO:174, and (iii) a heavy chain comprising the amino acid sequence of SEQ ID NO:173.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:114,
(ii) a fusion polypeptide comprising an amino acid sequence that is at least about 95%, 96%, 97%, 98%, 99% or 100% identical to the amino acid sequence of SEQ ID NO:174, and
(iii) a heavy chain comprising an amino acid sequence that is at least about 95%, 96%, 97%, 98%, 99% or 100% identical to the amino acid sequence of SEQ ID NO:180.

More particularly, the TNF family ligand trimer-containing antigen binding molecule comprises (i) a light chain comprising the amino acid sequence of SEQ ID NO:114, (ii) a fusion polypeptide comprising the amino acid sequence of SEQ ID NO:174, and (iii) a heavy chain comprising the amino acid sequence of SEQ ID NO:180.

In a further aspect, provided is a TNF family ligand trimer-containing antigen binding molecule, wherein the antigen binding molecule comprises

(i) a first light chain comprising the VL domain of the Fab domain capable of specific binding to FAP,
(ii) a first heavy chain comprising the VH domain of the Fab domain capable of specific binding to FAP, a first subunit of a Fc domain and a second polypeptide comprising one ectodomain of said TNF ligand family member or a fragment thereof, wherein the variable heavy chain of the Fab domain capable of specific binding to FAP is fused at its C-terminus to the N-terminus of the second subunit of the Fc domain and wherein the second polypeptide is fused at its N-terminus to the C-terminus of the first subunit of the Fc domain,
(iii) a second heavy chain comprising the VH domain of a Fab domain that is not capable of specific binding to a target cell antigen, a second subunit of the Fc domain and and a first polypeptide comprising two ectodomains of a TNF ligand family member or fragments thereof that are connected to each other by a peptide linker, wherein the variable heavy chain of the Fab domain not capable of specific binding to a target cell antigen is fused at its C-terminus to the N-terminus of the second subunit of the Fc domain and wherein the first polypeptide comprising two ectodomains of a TNF ligand family member or fragments thereof is fused at its N-terminus to the C-terminus of the second subunit of the Fc domain, and
(iv) a second light chain comprising the VL domain of the Fab domain that is not capable of specific binding to a target cell antigen.

In a particular aspect, provided is a TNF family ligand trimer-containing antigen binding molecule, wherein the antigen binding molecule comprises

(i) a first light chain comprising an amino acid sequence that is at least about 95%, 96%, 97%, 98%, 99% or 100% identical to the amino acid sequence of SEQ ID NO:106,
(ii) a first heavy chain comprising an amino acid sequence that is at least about 95%, 96%, 97%, 98%, 99% or 100% identical to the amino acid sequence of SEQ ID NO:217,
(iii) a second heavy chain comprising an amino acid sequence that is at least about 95%, 96%, 97%. 98%. 99% or 100% identical to the amino acid sequence of SEQ ID NO:218, and
(iv) a second light chain comprising an amino acid sequence that is at least about 95%, 96%, 97%, 98%, 99% or 100% identical to the amino acid sequence of SEQ ID NO:214.

More particularly, the TNF family ligand trimer-containing antigen binding molecule comprises (i) a first light chain comprising the amino acid sequence of SEQ ID NO:106, (ii) a first heavy chain comprising the amino acid sequence of SEQ ID NO:217, (iii) a second heavy chain comprising the amino acid sequence of SEQ ID NO:218, and (iv) a second light chain comprising the amino acid sequence of SEQ ID NO:214.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:106,
(ii) a first heavy chain comprising an amino acid sequence that is at least about 95%, 96%, 97%, 98%, 99% or 100% identical to the amino acid sequence of SEQ ID NO:222,
(iii) a second heavy chain comprising an amino acid sequence that is at least about 95%, 96%, 97%, 98%, 99% or 100% identical to the amino acid sequence of SEQ ID NO:221, and
(iv) a second light chain comprising an amino acid sequence that is at least about 95%, 96%, 97%, 98%, 99% or 100% identical to the amino acid sequence of SEQ ID NO:214.

More particularly, the TNF family ligand trimer-containing antigen binding molecule comprises (i) a first light chain comprising the amino acid sequence of SEQ ID NO:106, (ii) a first heavy chain comprising the amino acid sequence of SEQ ID NO:222, (iii) a second heavy chain comprising the amino acid sequence of SEQ ID NO:221, and (iv) a second light chain comprising the amino acid sequence of SEQ ID NO:214.

In another aspect, provided is a TNF family ligand trimer-containing antigen binding molecule, wherein the antigen binding molecule comprises

(i) a first light chain comprising the VL domain of the Fab domain capable of specific binding to FAP,
(ii) a first heavy chain comprising the VH domain of the Fab domain capable of specific binding to FAP, a first subunit of a Fc domain and and a first polypeptide comprising two ectodomains of a TNF ligand family member or fragments thereof that are connected to each other by a peptide linker, wherein the variable heavy chain of the Fab domain capable of specific binding to FAP is fused at its C-terminus to the N-terminus of the second subunit of the Fc domain and wherein the first polypeptide comprising ectodomains of a TNF ligand family member or fragments thereof is fused at its N-terminus to the C-terminus of the first subunit of the Fc domain,
(iii) a second heavy chain comprising the VH domain of a Fab domain that is not capable of specific binding to a target cell antigen, a second subunit of the Fc domain and and a second polypeptide comprising one ectodomain of said TNF ligand family member or a fragment thereof, wherein the variable heavy chain of the Fab domain not capable of specific binding to a target cell antigen is fused at its C-terminus to the N-terminus of the second subunit of the Fc domain and wherein the second polypeptide comprising one ectodomain of a TNF ligand family member or fragments thereof is fused at its N-terminus to the C-terminus of the second subunit of the Fc domain, and
(iv) a second light chain comprising the VL domain of the Fab domain that is not capable of specific binding to a target cell antigen.

In a particular aspect, provided is a TNF family ligand trimer-containing antigen binding molecule, wherein the antigen binding molecule comprises

(i) a first light chain comprising an amino acid sequence that is at least about 95%, 96%, 97%, 98%, 99% or 100% identical to the amino acid sequence of SEQ ID NO:106,
(ii) a first heavy chain comprising an amino acid sequence that is at least about 95%, 96%, 97%, 98%, 99% or 100% identical to the amino acid sequence of SEQ ID NO:212,
(iii) a second heavy chain comprising an amino acid sequence that is at least about 95%, 96%, 97%, 98%, 99% or 100% identical to the amino acid sequence of SEQ ID NO:213, and
(iv) a second light chain comprising an amino acid sequence that is at least about 95%, 96%, 97%, 98%, 99% or 100% identical to the amino acid sequence of SEQ ID NO:214.

More particularly, the TNF family ligand trimer-containing antigen binding molecule comprises (i) a first light chain comprising the amino acid sequence of SEQ ID NO:106, (ii) a first heavy chain comprising the amino acid sequence of SEQ ID NO:212, (iii) a second heavy chain comprising the amino acid sequence of SEQ ID NO:213, and (iv) a second light chain comprising the amino acid sequence of SEQ ID NO:214.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:106,
(ii) a first heavy chain comprising an amino acid sequence that is at least about 95%, 96%, 97%, 98%, 99% or 100% identical to the amino acid sequence of SEQ ID NO:226,
(iii) a second heavy chain comprising an amino acid sequence that is at least about 95%, 96%, 97%, 98%, 99% or 100% identical to the amino acid sequence of SEQ ID NO:225, and
(iv) a second light chain comprising an amino acid sequence that is at least about 95%, 96%, 97%, 98%, 99% or 100% identical to the amino acid sequence of SEQ ID NO:214.

More particularly, the TNF family ligand trimer-containing antigen binding molecule comprises (i) a first light chain comprising the amino acid sequence of SEQ ID NO:106, (ii) a first heavy chain comprising the amino acid sequence of SEQ ID NO:226, (iii) a second heavy chain comprising the amino acid sequence of SEQ ID NO:225, and (iv) a second light chain comprising the amino acid sequence of SEQ ID NO:214.

TNF Family Ligand Trimer-Containing Antigen Binding Molecules, Wherein the Target Cell Antigen is CD19

In one aspect, provided is a TNF family ligand trimer-containing antigen binding molecule, wherein the target cell antigen is CD19.

In a particular aspect, provided is a TNF family ligand trimer-containing antigen binding molecule as defined herein before, wherein the Fab domain capable of specific binding to CD19 comprises a heavy chain variable region comprising an amino acid sequence that is at least about 95%, 96%, 97%, 98%, 99% or 100% identical to the amino acid sequence of SEQ ID NO:41 and a light chain variable region comprising an amino acid sequence that is at least about 95%, 96%, 97%, 98%, 99% or 100% identical to the amino acid sequence of SEQ ID NO:42.

In another particular aspect, provided is a TNF family ligand trimer-containing antigen binding molecule as defined herein before, wherein the Fab domain capable of specific binding to CD19 comprises a heavy chain variable region comprising an amino acid sequence that is at least about 95%, 96%, 97%, 98%, 99% or 100% identical to the amino acid sequence of SEQ ID NO:43 and a light chain variable region comprising an amino acid sequence that is at least about 95%, 96%, 97%, 98%, 99% or 100% identical to the amino acid sequence of SEQ ID NO:44.

In one aspect, the Fab domain capable of specific binding to CD19 comprises a variable heavy chain comprising an amino acid sequence of SEQ ID NO:41 and a variable light chain comprising an amino acid sequence of SEQ ID NO:42 or a variable heavy chain comprising an amino acid sequence of SEQ ID NO:43 and a variable light chain comprising an amino acid sequence of SEQ ID NO:44. In a particular aspect, the Fab domain capable of specific binding to FAP comprises a heavy chain variable region comprising the amino acid sequence of SEQ ID NO:41 and a light chain variable region comprising the amino acid sequence of SEQ ID NO:42. In another particular aspect, the Fab domain capable of specific binding to FAP comprises a heavy chain variable region comprising the amino acid sequence of SEQ ID NO:43 and a light chain variable region comprising the amino acid sequence of SEQ ID NO:44. In a specific aspect, the Fab domain capable of specific binding to FAP comprises a VH domain consisting of amino acid sequence of SEQ ID NO:43 and a VL domain consisting of the amino acid sequence of SEQ ID NO:44.

In a further aspect, the invention provides a TNF family ligand trimer-containing antigen binding molecule comprising

(a) a Fab domain capable of specific binding to CD19 comprising a VH domain comprising an amino acid sequence of SEQ ID NO:41 and a VL domain comprising an amino acid sequence of SEQ ID NO:42, or a VH domain comprising an amino acid sequence of SEQ ID NO:43 and a VL domain comprising an amino acid sequence of SEQ ID NO:44,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain.

In another aspect, the invention provides a TNF family ligand trimer-containing antigen binding molecule comprising

(a) a Fab domain capable of specific binding to CD19 comprising a VH domain comprising an amino acid sequence of SEQ ID NO:41 and a VL domain comprising an amino acid sequence of SEQ ID NO:42, or a VH domain comprising an amino acid sequence of SEQ ID NO:43 and a VL domain comprising an amino acid sequence of SEQ ID NO:44,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain and wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5.

In a particular aspect, provided is a TNF family ligand trimer-containing antigen binding molecule comprising

(a) a Fab domain capable of specific binding to CD19 comprising a VH domain comprising an amino acid sequence of SEQ ID NO:43 and a VL domain comprising an amino acid sequence of SEQ ID NO:44,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fe domain and wherein the second polypeptide is fused at its N-terminus to the C-terminus of the other subunit of the Fe domain and wherein the first polypeptide comprising two ectodomains of a TNF ligand family member comprising an amino acid sequence of SEQ ID NO:10 and the second polypeptide comprising one ectodomain of said TNF ligand family member comprising the amino acid sequence of SEQ ID NO:5.

In another aspect, provided is a TNF family ligand trimer-containing antigen binding molecule, wherein the antigen binding molecule comprises

(i) a light chain comprising the variable light chain of the Fab domain capable of specific binding to CD19,
(ii) a fusion polypeptide comprising a first subunit of a Fc domain and a first polypeptide comprising two ectodomains of a TNF ligand family member or fragments thereof that are connected to each other by a peptide linker, wherein the first polypeptide is fused at its N-terminus to the C-terminus of the first subunit of the Fc domain, and
(iii) a heavy chain comprising the variable heavy chain of the Fab domain capable of specific binding to CD19, a second subunit of the Fc domain and and a second polypeptide comprising one ectodomain of said TNF ligand family member or a fragment thereof, wherein the variable heavy chain of the Fab domain capable of specific binding to CD19 is fused at its C-terminus to the N-terminus of the second subunit of the Fc domain and wherein the second polypeptide comprising one ectodomain of said TNF ligand family member or a fragment thereof is fused at its N-terminus to the C-terminus of the second subunit of the Fc domain.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:120,
(ii) a fusion polypeptide comprising an amino acid sequence that is at least about 95%, 96%, 97%, 98%, 99% or 100% identical to the amino acid sequence of SEQ ID NO:104, and
(iii) a heavy chain comprising an amino acid sequence that is at least about 95%, 96%, 97%, 98%, 99% or 100% identical to the amino acid sequence of SEQ ID NO:119.

More particularly, the TNF family ligand trimer-containing antigen binding molecule comprises (i) a light chain comprising the amino acid sequence of SEQ ID NO:120, (ii) a fusion polypeptide comprising the amino acid sequence of SEQ ID NO:104, and (iii) a heavy chain comprising the amino acid sequence of SEQ ID NO:119.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:126,
(ii) a fusion polypeptide comprising an amino acid sequence that is at least about 95%, 96%, 97%, 98%, 99% or 100% identical to the amino acid sequence of SEQ ID NO:104, and
(iii) a heavy chain comprising an amino acid sequence that is at least about 95%, 96%, 97%, 98%, 99% or 100% identical to the amino acid sequence of SEQ ID NO:125.

More particularly, the TNF family ligand trimer-containing antigen binding molecule comprises (i) a light chain comprising the amino acid sequence of SEQ ID NO:126, (ii) a fusion polypeptide comprising the amino acid sequence of SEQ ID NO:174, and (iii) a heavy chain comprising the amino acid sequence of SEQ ID NO:125.

In a further aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:120,
(ii) a fusion polypeptide comprising an amino acid sequence that is at least about 95%, 96%, 97%, 98%, 99% or 100% identical to the amino acid sequence of SEQ ID NO:178, and
(iii) a heavy chain comprising an amino acid sequence that is at least about 95%, 96%, 97%, 98%, 99% or 100% identical to the amino acid sequence of SEQ ID NO:186.

More particularly, the TNF family ligand trimer-containing antigen binding molecule comprises (i) a light chain comprising the amino acid sequence of SEQ ID NO:120, (ii) a fusion polypeptide comprising the amino acid sequence of SEQ ID NO:178, and (iii) a heavy chain comprising the amino acid sequence of SEQ ID NO:186.

In yet another aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:126,
(ii) a fusion polypeptide comprising an amino acid sequence that is at least about 95%, 96%, 97%, 98%, 99% or 100% identical to the amino acid sequence of SEQ ID NO:178, and
(iii) a heavy chain comprising an amino acid sequence that is at least about 95%, 96%, 97%, 98%, 99% or 100% identical to the amino acid sequence of SEQ ID NO:190.

More particularly, the TNF family ligand trimer-containing antigen binding molecule comprises (i) a light chain comprising the amino acid sequence of SEQ ID NO:126, (ii) a fusion polypeptide comprising the amino acid sequence of SEQ ID NO:178, and (iii) a heavy chain comprising the amino acid sequence of SEQ ID NO:190.

In another aspect, provided is a TNF family ligand trimer-containing antigen binding molecule, wherein the antigen binding molecule comprises

(i) a light chain comprising the variable light chain of the Fab domain capable of specific binding to CD19,
(ii) a fusion polypeptide comprising a first subunit of a Fc domain and a second polypeptide comprising one ectodomain of said TNF ligand family member or a fragment thereof, wherein the second polypeptide is fused at its N-terminus to the C-terminus of the first subunit of the Fc domain, and
(iii) a heavy chain comprising the variable heavy chain of the Fab domain capable of specific binding to CD19, a second subunit of the Fc domain and and a first polypeptide comprising two ectodomains of a TNF ligand family member or fragments thereof that are connected to each other by a peptide linker, wherein the variable heavy chain of the Fab domain capable of specific binding to CD19 is fused at its C-terminus to the N-terminus of the second subunit of the Fc domain and wherein the first polypeptide comprising two ectodomains of a TNF ligand family member or fragments thereof is fused at its N-terminus to the C-terminus of the second subunit of the Fc domain.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:120,
(ii) a fusion polypeptide comprising an amino acid sequence that is at least about 95%, 96%, 97%, 98%, 99% or 100% identical to the amino acid sequence of SEQ ID NO:109, and
(iii) a heavy chain comprising an amino acid sequence that is at least about 95%, 96%, 97%, 98%, 99% or 100% identical to the amino acid sequence of SEQ ID NO:122.

More particularly, the TNF family ligand trimer-containing antigen binding molecule comprises (i) a light chain comprising the amino acid sequence of SEQ ID NO:120, (ii) a fusion polypeptide comprising the amino acid sequence of SEQ ID NO:109, and (iii) a heavy chain comprising the amino acid sequence of SEQ ID NO:122.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:126,
(ii) a fusion polypeptide comprising an amino acid sequence that is at least about 95%, 96%, 97%, 98%, 99% or 100% identical to the amino acid sequence of SEQ ID NO:109, and
(iii) a heavy chain comprising an amino acid sequence that is at least about 95%, 96%, 97%, 98%. 99% or 100% identical to the amino acid sequence of SEQ ID NO:128.

More particularly, the TNF family ligand trimer-containing antigen binding molecule comprises (i) a light chain comprising the amino acid sequence of SEQ ID NO:126, (ii) a fusion polypeptide comprising the amino acid sequence of SEQ ID NO:109, and (iii) a heavy chain comprising the amino acid sequence of SEQ ID NO:128.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:120,
(ii) a fusion polypeptide comprising an amino acid sequence that is at least about 95%, 96%, 97%, 98%, 99% or 100% identical to the amino acid sequence of SEQ ID NO:174, and
(iii) a heavy chain comprising an amino acid sequence that is at least about 95%, 96%, 97%, 98%, 99% or 100% identical to the amino acid sequence of SEQ ID NO:184.

More particularly, the TNF family ligand trimer-containing antigen binding molecule comprises (i) a light chain comprising the amino acid sequence of SEQ ID NO:120, (ii) a fusion polypeptide comprising the amino acid sequence of SEQ ID NO:174, and (iii) a heavy chain comprising the amino acid sequence of SEQ ID NO:184.

In another particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:126,
(ii) a fusion polypeptide comprising an amino acid sequence that is at least about 95%, 96%, 97%, 98%, 99% or 100% identical to the amino acid sequence of SEQ ID NO:174, and
(iii) a heavy chain comprising an amino acid sequence that is at least about 95%, 96%, 97%, 98%, 99% or 100% identical to the amino acid sequence of SEQ ID NO:188.

More particularly, the TNF family ligand trimer-containing antigen binding molecule comprises (i) a light chain comprising the amino acid sequence of SEQ ID NO:126, (ii) a fusion polypeptide comprising the amino acid sequence of SEQ ID NO:174, and (iii) a heavy chain comprising the amino acid sequence of SEQ ID NO:188.

TNF Family Ligand Trimer-Containing Antigen Binding Molecules, Wherein the Target Cell Antigen is CEA

In one aspect, provided is a TNF family ligand trimer-containing antigen binding molecule, wherein the target cell antigen is CEA.

In a particular aspect, provided is a TNF family ligand trimer-containing antigen binding molecule as defined herein before, wherein the Fab domain capable of specific binding to CEA comprises a heavy chain variable region comprising an amino acid sequence that is at least about 95%, 96%, 97%, 98%, 99% or 100% identical to the amino acid sequence of SEQ ID NO:51 and a light chain variable region comprising an amino acid sequence that is at least about 95%, 96%, 97%, 98%, 99% or 100% identical to the amino acid sequence of SEQ ID NO:52.

In one aspect, the Fab domain capable of specific binding to CEA comprises a variable heavy chain comprising an amino acid sequence of SEQ ID NO:51 and a variable light chain comprising an amino acid sequence of SEQ ID NO:52 or a variable heavy chain comprising an amino acid sequence of SEQ ID NO:163 and a variable light chain comprising an amino acid sequence of SEQ ID NO:164. In a particular aspect, the Fab domain capable of specific binding to CEA comprises a heavy chain variable region comprising the amino acid sequence of SEQ ID NO:51 and a light chain variable region comprising the amino acid sequence of SEQ ID NO:52.

In a specific aspect, the Fab domain capable of specific binding to CEA comprises a VH domain consisting of amino acid sequence of SEQ ID NO:51 and a VL domain consisting of the amino acid sequence of SEQ ID NO:52.

In a further aspect, the invention provides a TNF family ligand trimer-containing antigen binding molecule comprising

(a) a Fab domain capable of specific binding to CEA comprising a VH domain comprising an amino acid sequence of SEQ ID NO:51 and a VL domain comprising an amino acid sequence of SEQ ID NO:52,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain.

In another aspect, the invention provides a TNF family ligand trimer-containing antigen binding molecule comprising

(a) a Fab domain capable of specific binding to CEA comprising a VH domain comprising an amino acid sequence of SEQ ID NO:51 and a VL domain comprising an amino acid sequence of SEQ ID NO:52,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fe domain and wherein the second polypeptide is fused at its N-terminus to the C-terminus of the other subunit of the Fe domain and wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5.

In a particular aspect, provided is a TNF family ligand trimer-containing antigen binding molecule comprising

(a) a Fab domain capable of specific binding to CEA comprising a VH domain comprising an amino acid sequence of SEQ ID NO:51 and a VL domain comprising an amino acid sequence of SEQ ID NO:52,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain and wherein the first polypeptide comprising two ectodomains of a TNF ligand family member comprising an amino acid sequence of SEQ ID NO:10 and the second polypeptide comprising one ectodomain of said TNF ligand family member comprising the amino acid sequence of SEQ ID NO:5.

In another aspect, provided is a TNF family ligand trimer-containing antigen binding molecule, wherein the antigen binding molecule comprises

(i) a light chain comprising the variable light chain of the Fab domain capable of specific binding to CEA,
(ii) a fusion polypeptide comprising a first subunit of a Fc domain and a first polypeptide comprising two ectodomains of a TNF ligand family member or fragments thereof that are connected to each other by a peptide linker, wherein the first polypeptide is fused at its N-terminus to the C-terminus of the first subunit of the Fc domain, and
(iii) a heavy chain comprising the variable heavy chain of the Fab domain capable of specific binding to CEA, a second subunit of the Fc domain and and a second polypeptide comprising one ectodomain of said TNF ligand family member or a fragment thereof, wherein the variable heavy chain of the Fab domain capable of specific binding to CEA is fused at its C-terminus to the N-terminus of the second subunit of the Fc domain and wherein the second polypeptide comprising one ectodomain of said TNF ligand family member or a fragment thereof is fused at its N-terminus to the C-terminus of the second subunit of the Fe domain.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:156,
(ii) a fusion polypeptide comprising an amino acid sequence that is at least about 95%, 96%, 97%, 98%, 99% or 100% identical to the amino acid sequence of SEQ ID NO:104, and
(iii) a heavy chain comprising an amino acid sequence that is at least about 95%, 96%, 97%, 98%, 99% or 100% identical to the amino acid sequence of SEQ ID NO:155.

More particularly, the TNF family ligand trimer-containing antigen binding molecule comprises (i) a light chain comprising the amino acid sequence of SEQ ID NO:156, (ii) a fusion polypeptide comprising the amino acid sequence of SEQ ID NO:104, and (iii) a heavy chain comprising the amino acid sequence of SEQ ID NO:155.

In a further aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:156,
(ii) a fusion polypeptide comprising an amino acid sequence that is at least about 95%, 96%, 97%, 98%, 99% or 100% identical to the amino acid sequence of SEQ ID NO:178, and
(iii) a heavy chain comprising an amino acid sequence that is at least about 95%, 96%, 97%, 98%, 99% or 100% identical to the amino acid sequence of SEQ ID NO:194.

More particularly, the TNF family ligand trimer-containing antigen binding molecule comprises (i) a light chain comprising the amino acid sequence of SEQ ID NO:156, (ii) a fusion polypeptide comprising the amino acid sequence of SEQ ID NO:178, and (iii) a heavy chain comprising the amino acid sequence of SEQ ID NO:194.

In another aspect, provided is a TNF family ligand trimer-containing antigen binding molecule, wherein the antigen binding molecule comprises

(i) a light chain comprising the variable light chain of the Fab domain capable of specific binding to CEA,
(ii) a fusion polypeptide comprising a first subunit of a Fc domain and a second polypeptide comprising one ectodomain of said TNF ligand family member or a fragment thereof, wherein the second polypeptide is fused at its N-terminus to the C-terminus of the first subunit of the Fc domain, and
(iii) a heavy chain comprising the variable heavy chain of the Fab domain capable of specific binding to CEA, a second subunit of the Fe domain and and a first polypeptide comprising two ectodomains of a TNF ligand family member or fragments thereof that are connected to each other by a peptide linker, wherein the variable heavy chain of the Fab domain capable of specific binding to CEA is fused at its C-terminus to the N-terminus of the second subunit of the Fc domain and wherein the first polypeptide comprising two ectodomains of a TNF ligand family member or fragments thereof is fused at its N-terminus to the C-terminus of the second subunit of the Fc domain.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:156,
(ii) a fusion polypeptide comprising an amino acid sequence that is at least about 95%, 96%, 97%, 98%, 99% or 100% identical to the amino acid sequence of SEQ ID NO:109, and
(iii) a heavy chain comprising an amino acid sequence that is at least about 95%, 96%, 97%, 98%, 99% or 100% identical to the amino acid sequence of SEQ ID NO:158.

More particularly, the TNF family ligand trimer-containing antigen binding molecule comprises (i) a light chain comprising the amino acid sequence of SEQ ID NO:156, (ii) a fusion polypeptide comprising the amino acid sequence of SEQ ID NO:109, and (iii) a heavy chain comprising the amino acid sequence of SEQ ID NO:158.

In a particular aspect, provided is a TNF family ligand trimer-containing antigen binding molecule, wherein the antigen binding molecule comprises

(i) a light chain comprising an amino acid sequence that is at least about 95%, 96%, 97%, 98%, 99% or 100% identical to the amino acid sequence of SEQ ID NO:156,
(ii) a fusion polypeptide comprising an amino acid sequence that is at least about 95%, 96%, 97%, 98%, 99% or 100% identical to the amino acid sequence of SEQ ID NO:174, and
(iii) a heavy chain comprising an amino acid sequence that is at least about 95%, 96%, 97%, 98%, 99% or 100% identical to the amino acid sequence of SEQ ID NO:192.

More particularly, the TNF family ligand trimer-containing antigen binding molecule comprises (i) a light chain comprising the amino acid sequence of SEQ ID NO:156, (ii) a fusion polypeptide comprising the amino acid sequence of SEQ ID NO:174, and (iii) a heavy chain comprising the amino acid sequence of SEQ ID NO:192.

Polynucleotides

The invention further provides isolated polynucleotides encoding a TNF family ligand trimer-containing antigen binding molecule as described herein or a fragment thereof.

The isolated polynucleotides encoding TNF ligand trimer-containing antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes the entire TNF family ligand trimer-containing antigen binding molecule according to the invention as described herein. In particular, the isolated polynucleotide encodes a polypeptide comprised in the TNF family ligand trimer-containing antigen binding molecule according to the invention as described herein.

In one aspect, the present invention is directed to an isolated polynucleotide encoding a TNF family ligand trimer-containing antigen binding molecule, wherein the polynucleotide comprises (a) a sequence that encodes a Fab domain capable of specific binding to a target cell antigen, (b) a sequence that encodes a first subunit of a Fc domain and first polypeptide comprising two ectodomains of a TNF ligand family member or fragments thereof that are connected to each other by a peptide linker and (c) a sequence that encodes a second subunit of a Fc domain and and a second polypeptide comprising one ectodomain of said TNF ligand family member or a fragment thereof.

In certain aspects, provided an isolated polynucleotide encoding a TNF family ligand trimer-containing antigen binding molecule, wherein the polynucleotide comprises (a) a sequence that encodes a Fab domain capable of specific binding to a target cell antigen, (b) a sequence that encodes a first subunit of a Fc domain and first polypeptide comprising two ectodomains of a TNF ligand family member or fragments thereof that are connected to each other by a peptide linker, (c) a sequence that encodes a second subunit of a Fc domain and and a second polypeptide comprising one ectodomain of said TNF ligand family member or a fragment thereof, and (d) a sequence that encodes a Fab domain that is not capable of specific binding to a target cell antigen.

In another aspect, provided is an isolated polynucleotide encoding a 4-1BB ligand trimer-containing antigen binding molecule, wherein the polynucleotide comprises (a) a sequence that encodes a Fab domain capable of specific binding to a target cell antigen, (b) a sequence that encodes a first subunit of a Fc domain and first polypeptide comprising two ectodomains of a TNF ligand family member or fragments thereof that are connected to each other by a peptide linker and (c) a sequence that encodes a second subunit of a Fc domain and and a second polypeptide comprising one ectodomain of said TNF ligand family member or a fragment thereof.

In a further aspect, the invention is directed to an isolated polynucleotide comprising a sequence that encodes a polypeptide comprising two 4-1BBL fragments comprising an amino acid sequence that is at least about 90%, 95%, 98% or 100% identical to an amino acid sequence shown in SEQ ID NO:1, SEQ ID NO:2, SEQ ID NO:3, SEQ ID NO:4, SEQ ID NO:5, SEQ ID NO:6, SEQ ID NO:7 and SEQ ID NO:8, and to a polynucleotide comprising a sequence that encodes a polypeptide comprising one 4-1BBL fragment comprising an amino acid sequence that is is at least about 90%, 95%, 98% or 100% identical to an amino acid sequence shown in SEQ ID NO:1, SEQ ID NO:2, SEQ ID NO:3, SEQ ID NO:4, SEQ ID NO:5, SEQ ID NO:6, SEQ ID NO:7 and SEQ ID NO:8.

Furthermore, provided is an isolated polynucleotide encoding a OX40 ligand trimer-containing antigen binding molecule, wherein the polynucleotide comprises (a) a sequence that encodes a moiety capable of specific binding to a target cell antigen, (b) a sequence that encodes a polypeptide comprising two ectodomains of OX40L or two fragments thereof that are connected to each other by a peptide linker and (c) a sequence that encodes a polypeptide comprising one ectodomain of OX40L or a fragment thereof.

In another aspect, the invention is directed to an isolated polynucleotide comprising a sequence that encodes a polypeptide comprising two 4-1BBL fragments comprising an amino acid sequence that is at least about 90%, 95%, 98% or 100% identical to an amino acid sequence shown in SEQ ID NO:9 or SEQ ID NO:10, and to a polynucleotide comprising a sequence that encodes a polypeptide comprising one 4-1BBL fragment comprising an amino acid sequence that is is at least about 90%, 95%, 98% or 100% identical to an amino acid sequence shown in SEQ ID NO:1 or SEQ ID NO:5.

In further aspects, the invention relates to the polynucleotides comprising a sequence that is at least about 90%, 95%, 98% or 100% identical to the specific cDNA sequences disclosed herein. In a particular aspect, the invention relates to a polynucleotide comprising a sequence that is identical to one of the specific cDNA sequences disclosed herein.

In certain aspects, the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

TNF family ligand trimer-containing antigen binding molecules of the invention may be obtained, for example, by solid-state peptide synthesis (e.g. Merrifield solid phase synthesis) or recombinant production. For recombinant production one or more polynucleotide encoding the TNF family ligand trimer-containing antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the TNF family ligand trimer-containing antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the TNF family ligand trimer-containing antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the TNF family ligand trimer-containing antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the TNF family ligand trimer-containing antigen binding molecule or polypeptide fragments thereof is desired, DNA encoding a signal sequence may be placed upstream of the nucleic acid encoding a TNF family ligand trimer-containing antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse 0-glucuronidase.

DNA encoding a short protein sequence that could be used to facilitate later purification (e.g. a histidine tag) or assist in labeling the fusion protein may be included within or at the ends of the polynucleotide encoding a TNF family ligand trimer-containing antigen binding molecule of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a TNF family ligand trimer-containing antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES^{T} technology for producing antibodies in transgenic plants). Vertebrate cells may also be used as hosts. For example, mammalian cell lines that are adapted to grow in suspension may be useful. Other examples of useful mammalian host cell lines are monkey kidney CV Iline transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NSO, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a TNF family ligand trimer-containing antigen binding molecule of the invention or polypeptide fragments thereof is provided, wherein the method comprises culturing a host cell comprising polynucleotides encoding the TNF family ligand trimer-containing antigen binding molecule of the invention or polypeptide fragments thereof, as provided herein, under conditions suitable for expression of the TNF family ligand trimer-containing antigen binding molecule of the invention or polypeptide fragments thereof, and recovering the TNF family ligand trimer-containing antigen binding molecule of the invention or polypeptide fragments thereof from the host cell (or host cell culture medium).

In the TNF family ligand trimer-containing antigen binding molecule of the invention, the components (a Fab domain capable of specific binding to a target cell antigen, one polypeptide comprising two ectodomains of a TNF ligand family member or fragments thereof and a polypeptide comprising one ectodomain of said TNF family ligand family member or a fragment thereof) are not genetically fused to each other. The polypeptides are designed such that its components (two ectodomains of a TNF ligand family member or fragments thereof and other components such as CH or CL) are fused to each other directly or through a linker sequence. The composition and length of the linker may be determined in accordance with methods well known in the art and may be tested for efficacy. Examples of linker sequences between different components of the antigen binding molecules of the invention are found in the sequences provided herein. Additional sequences may also be included to incorporate a cleavage site to separate the individual components of the fusion protein if desired, for example an endopeptidase recognition sequence.

In certain embodiments the moieties capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osboum et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the Fab domains capable of specific binding to a target cell antigen (e.g. Fab fragments) comprised in the antigen binding molecules of the present invention are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch.14 (Cold Spring Harbor Laboratory, Cold Spring Harbor, N.Y.).

TNF ligand trimer-containing antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the TNF ligand trimer-containing antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the TNF ligand trimer-containing antigen binding molecule or fragments thereof can be determined by any of a variety of well-known analytical methods including gel electrophoresis, high pressure liquid chromatography, and the like. For example, the TNF ligand trimer-containing antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art.

1. Affinity Assays

The affinity of the TNF family ligand trimer-containing antigen binding molecule provided herein for the corresponding TNF receptor can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the TNF family ligand trimer-containing antigen binding molecule for the target cell antigen can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 3. According to one aspect, K_{D} is measured by surface plasmon resonance using a BIACORE® T100 machine (GE Healthcare) at 25° C.

2. Binding Assays and Other Assays

Binding of the TNF family ligand trimer-containing antigen binding molecule provided herein to the corresponding receptor expressing cells may be evaluated using cell lines expressing the particular receptor or target antigen, for example by flow cytometry (FACS). In one aspect, fresh peripheral blood mononuclear cells (PBMCs) expressing the TNF receptor are used in the binding assay (Example 6.1 and 7.1). These cells are used directly after isolation (naïve PMBCs) or after stimulation (activated PMBCs). In another aspect, activated mouse splenocytes (expressing the TNF receptor molecule) were used to demonstrate the binding of the TNF family ligand trimer-containing antigen binding molecule of the invention to the corresponding TNF receptor expressing cells.

In a further aspect, cancer cell lines expressing the target cell antigen, for example FAP, were used to demonstrate the binding of the antigen binding molecules to the target cell antigen (Example 6.2). In another aspect, cancer cell lines expressing CD19 were used to demonstrate the binding of the antigen binding molecules to CD19 (Example 7.2).

In another aspect, competition assays may be used to identify an antigen binding molecule that competes with a specific antibody or antigen binding molecule for binding to the target or TNF receptor, respectively. In certain embodiments, such a competing antigen binding molecule binds to the same epitope (e.g., a linear or a conformational epitope) that is bound by a specific anti-target antibody or a specific anti-TNF receptor antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying TNF family ligand trimer-containing antigen binding molecules that bind to a specific target cell antigen and to a specific TNF receptor having biological activity. Biological activity may include, e.g., agonistic signalling through the TNF receptor on cells expressing the target cell antigen. TNF family ligand trimer-containing antigen binding molecules identified by the assays as having such biological activity in vitro are also provided.

In certain aspects, a TNF family ligand trimer-containing antigen binding molecule of the invention is tested for such biological activity. Assays for detecting the biological activity of the molecules of the invention are those described in Examples 6 and 7. Furthermore, assays for detecting cell lysis (e.g. by measurement of LDH release), induced apoptosis kinetics (e.g. by measurement of Caspase 3/7 activity) or apoptosis (e.g. using the TUNEL assay) are well known in the art. In addition the biological activity of such complexes can be assessed by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

Pharmaceutical Compositions, Formulations and Routes of Administration

In a further aspect, the invention provides pharmaceutical compositions comprising any of the TNF family ligand trimer-containing antigen binding molecules provided herein, e.g., for use in any of the below therapeutic methods. In one embodiment, a pharmaceutical composition comprises any of the TNF family ligand trimer-containing antigen binding molecules provided herein and at least one pharmaceutically acceptable excipient. In another embodiment, a pharmaceutical composition comprises any of the TNF family ligand trimer-containing antigen binding molecules provided herein and at least one additional therapeutic agent, e.g., as described below.

Pharmaceutical compositions of the present invention comprise a therapeutically effective amount of one or more TNF family ligand trimer-containing antigen binding molecules dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one TNF family ligand trimer-containing antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the TNF family ligand trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the fusion proteins may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the fusion proteins of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The TNF family ligand trimer-containing antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Therapeutic Methods and Compositions

Any of the TNF family ligand trimer-containing antigen binding molecules provided herein may be used in therapeutic methods.

For use in therapeutic methods, TNF family ligand trimer-containing antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, TNF family ligand trimer-containing antigen binding molecules of the invention for use as a medicament are provided. In further aspects, TNF family ligand trimer-containing antigen binding molecules of the invention for use in treating a disease, in particular for use in the treatment of cancer, are provided. In certain aspects, TNF family ligand trimer-containing antigen binding molecules of the invention for use in a method of treatment are provided. In one aspect, the invention provides a TNF family ligand trimer-containing antigen binding molecule as described herein for use in the treatment of a disease in an individual in need thereof. In certain aspects, the invention provides a TNF family ligand trimer-containing antigen binding molecule for use in a method of treating an individual having a disease comprising administering to the individual a therapeutically effective amount of the fusion protein. In certain aspects, the disease to be treated is cancer. Examples of cancers include solid tumors, bladder cancer, renal cell carcinoma, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer, melanoma, B-cell lymphoma, B-cell leukemia, non-Hodgkin lymphoma and acute lymphoblastic leukemia. Thus, a TNF family ligand trimer-containing antigen binding molecule as described herein for use in the treatment of cancer is provided. The subject, patient, or “individual” in need of treatment is typically a mammal, more specifically a human.

In another aspect, provided is a TNF family ligand trimer-containing antigen binding molecule as described herein for use in the treatment of infectious diseases, in particular for the treatment of viral infections. In a further aspect, provided is a TNF family ligand trimer-containing antigen binding molecule as described herein for use in the treatment of autoimmune diseases such as for example Lupus disease.

In one aspect, provided is a TNF family ligand trimer-containing antigen binding molecule according to the invention for use in treating head and neck squamous cell carcinoma (HNSCC), breast cancer, colorectal cancer (CRC), pancreatic cancer (PAC), gastric cancer, non-small-cell lung carcinoma (NSCLC) and Mesothelioma, wherein the target cell antigen is FAP.

In a further aspect, the invention relates to the use of a TNF family ligand trimer-containing antigen binding molecule in the manufacture or preparation of a medicament for the treatment of a disease in an individual in need thereof. In one aspect, the medicament is for use in a method of treating a disease comprising administering to an individual having the disease a therapeutically effective amount of the medicament. In certain embodiments the disease to be treated is a proliferative disorder, particularly cancer. Thus, in one aspect, the invention relates to the use of a TNF family ligand trimer-containing antigen binding molecule of the invention in the manufacture or preparation of a medicament for the treatment of cancer. Examples of cancers include solid tumors, bladder cancer, renal cell carcinoma, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer, melanoma, B-cell lymphoma, B-cell leukemia, non-Hodgkin lymphoma and acute lymphoblastic leukemia. Other cell proliferation disorders that can be treated using a TNF family ligand trimer-containing antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan may recognize that in some cases the TNF family ligand trimer-containing antigen binding molecule may not provide a cure but may only provide partial benefit. In some aspects, a physiological change having some benefit is also considered therapeutically beneficial. Thus, in some aspects, an amount of TNF family ligand trimer-containing antigen binding molecule that provides a physiological change is considered an “effective amount” or a “therapeutically effective amount”.

In a further aspect, the invention relates to the use of a TNF family ligand trimer-containing antigen binding molecule as described herein in the manufacture or preparation of a medicament for the treatment of infectious diseases, in particular for the treatment of viral infections or for the treatment of autoimmune diseases, for example Lupus disease.

In a further aspect, the invention provides a method for treating a disease in an individual, comprising administering to said individual a therapeutically effective amount of a TNF family ligand trimer-containing antigen binding molecule of the invention. In one aspect a composition is administered to said individual, comprising a fusion protein of the invention in a pharmaceutically acceptable form. In certain aspects, the disease to be treated is a proliferative disorder. In a particular aspect, the disease is cancer. In another aspect, the disease is an infectious disease or an autoimmune disease. In certain aspects, the method further comprises administering to the individual a therapeutically effective amount of at least one additional therapeutic agent, e.g. an anti-cancer agent if the disease to be treated is cancer. An “individual” according to any of the above embodiments may be a mammal, preferably a human.

For the prevention or treatment of disease, the appropriate dosage of a TNF family ligand trimer-containing antigen binding molecule of the invention (when used alone or in combination with one or more other additional therapeutic agents) will depend on the type of disease to be treated, the route of administration, the body weight of the patient, the type of fusion protein, the severity and course of the disease, whether the fusion protein is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The TNF family ligand trimer-containing antigen binding molecule is suitably administered to the patient at one time or over a series of treatments. Depending on the type and severity of the disease, about 1 μg/kg to 15 mg/kg (e.g. 0.1 mg/kg-10 mg/kg) of TNF family ligand trimer-containing antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). An initial higher loading dose, followed by one or more lower doses may be administered. However, other dosage regimens may be useful. The progress of this therapy is easily monitored by conventional techniques and assays.

The TNF family ligand trimer-containing antigen binding molecules of the invention will generally be used in an amount effective to achieve the intended purpose. For use to treat or prevent a disease condition, the TNF family ligand trimer-containing antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the TNF family ligand trimer-containing antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the TNF family ligand trimer-containing antigen binding molecule may not be related to plasma concentration. One skilled in the art will be able to optimize therapeutically effective local dosages without undue experimentation.

A therapeutically effective dose of the TNF family ligand trimer-containing antigen binding molecules described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LDso (the dose lethal to 50% of a population) and the EDso (the dose therapeutically effective in 50% of a population). The dose ratio between toxic and therapeutic effects is the therapeutic index, which can be expressed as the ratio LD₅₀/ED₅₀. TNF family ligand trimer-containing antigen binding molecules that exhibit large therapeutic indices are preferred. In one embodiment, the TNF family ligand trimer-containing antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with fusion proteins of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The TNF family ligand trimer-containing antigen binding molecules of the invention may be administered in combination with one or more other agents in therapy. For instance, a fusion protein of the invention may be co-administered with at least one additional therapeutic agent.

The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent or immunotherapy.

Such other agents are suitably present in combination in amounts that are effective for the purpose intended. The effective amount of such other agents depends on the amount of fusion protein used, the type of disorder or treatment, and other factors discussed above. The TNF family ligand trimer-containing antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the TNF family ligand trimer-containing antigen binding molecule of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a TNF ligand trimer-containing antigen binding molecule of the invention.

The label or package insert indicates that the composition is used for treating the condition of choice. Moreover, the article of manufacture may comprise (a) a first container with a composition contained therein, wherein the composition comprises a TNF ligand trimer-containing antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE C
(Sequences):

| SEQ ID NO: | Name | Sequence |
|---|---|---|
| 1 | Human (hu) 4-1BBL | REGPELSPDDPAGLLDLRQGMFAQLVAQNVLLIDG |
| | (71-254) | PLSWYSDPGLAGVSLTGGLSYKEDTKELVVAKAGV |
| | | YYVFFQLELRRVVAGEGSGSVSLALHLQPLRSAAG |
| | | AAALALTVDLPPASSEARNSAFGFQGRLLHLSAGQ |
| | | RLGVHLHTEARARHAWQLTQGATVLGLFRVTPEIP |
| | | AGLPSPRSE |
| 2 | hu 4-1BBL (85-254) | LDLRQGMFAQLVAQNVLLIDGPLSWYSDPGLAGVS |
| | | LTGGLSYKEDTKELVVAKAGVYYVFFQLELRRVVA |
| | | GEGSGSVSLALHLQPLRSAAGAAALALTVDLPPAS |
| | | SEARNSAFGFQGRLLHLSAGQRLGVHLHTEARARH |
| | | AWQLTQGATVLGLFRVTPEIPAGLPSPRSE |
| 3 | hu 4-1BBL (80-254) | DPAGLLDLRQGMFAQLVAQNVLLIDGPLSWYSDPG |
| | | LAGVSLTGGLSYKEDTKELVVAKAGVYYVFFQLEL |
| | | RRVVAGEGSGSVSLALHLQPLRSAAGAAALALTVD |
| | | LPPASSEARNSAFGFQGRLLHLSAGQRLGVHLHTE |
| | | ARARHAWQLTQGATVLGLFRVTPEIPAGLPSPRSE |
| 4 | hu 4-1BBL (52-254) | PWAVSGARASPGSAASPRLREGPELSPDDPAGLLD |
| | | LRQGMFAQLVAQNVLLIDGPLSWYSDPGLAGVSLT |
| | | GGLSYKEDTKELVVAKAGVYYVFFQLELRRVVAGE |
| | | GSGSVSLALHLQPLRSAAGAAALALTVDLPPASSE |
| | | ARNSAFGFQGRLLHLSAGQRLGVHLHTEARARHAW |
| | | QLTQGATVLGLFRVTPEIPAGLPSPRSE |
| 5 | hu 4-1BBL (71-248) | REGPELSPDDPAGLLDLRQGMFAQLVAQNVLLIDG |
| | | PLSWYSDPGLAGVSLTGGLSYKEDTKELVVAKAGV |
| | | YYVFFQLELRRVVAGEGSGSVSLALHLQPLRSAAG |
| | | AAALALTVDLPPASSEARNSAFGFQGRLLHLSAGQ |
| | | RLGVHLHTEARARHAWQLTQGATVLGLFRVTPEIP |
| | | AGL |
| 6 | hu 4-1BBL (85-248) | LDLRQGMFAQLVAQNVLLIDGPLSWYSDPGLAGVS |
| | | LTGGLSYKEDTKELVVAKAGVYYVFFQLELRRVVA |
| | | GEGSGSVSLALHLQPLRSAAGAAALALTVDLPPAS |
| | | SEARNSAFGFQGRLLHLSAGQRLGVHLHTEARARH |
| | | AWQLTQGATVLGLFRVTPEIPAGL |
| 7 | hu 4-1BBL (80-248) | DPAGLLDLRQGMFAQLVAQNVLLIDGPLSWYSDPG |
| | | LAGVSLTGGLSYKEDTKELVVAKAGVYYVFFQLEL |
| | | RRVVAGEGSGSVSLALHLQPLRSAAGAAALALTVD |
| | | LPPASSEARNSAFGFQGRLLHLSAGQRLGVHLHTE |
| | | ARARHAWQLTQGATVLGLFRVTPEIPAGL |
| 8 | hu 4-1BBL (52-248) | PWAVSGARASPGSAASPRLREGPELSPDDPAGLLD |
| | | LRQGMFAQLVAQNVLLIDGPLSWYSDPGLAGVSLT |
| | | GGLSYKEDTKELVVAKAGVYYVFFQLELRRVVAGE |
| | | GSGSVSLALHLQPLRSAAGAAALALTVDLPPASSE |
| | | ARNSAFGFQGRLLHLSAGQRLGVHLHTEARARHAW |
| | | QLTQGATVLGLFRVTPEIPAGL |
| 9 | dimeric hu 4-1BBL (71-254) | REGPELSPDDPAGLLDLRQGMFAQLVAQNVLLIDG |
| | connected by (G4S)₂ linker | PLSWYSDPGLAGVSLTGGLSYKEDTKELVVAKAGV |
| | | YYVFFQLELRRVVAGEGSGSVSLALHLQPLRSAAG |
| | | AAALALTVDLPPASSEARNSAFGFQGRLLHLSAGQ |
| | | RLGVHLHTEARARHAWQLTQGATVLGLFRVTPEIP |
| | | AGLPSPRSEGGGGSGGGGSREGPELSPDDPAGLLD |
| | | LRQGMFAQLVAQNVLLIDGPLSWYSDPGLAGVSLT |
| | | GGLSYKEDTKELVVAKAGVYYVFFQLELRRVVAGE |
| | | GSGSVSLALHLQPLRSAAGAAALALTVDLPPASSE |
| | | ARNSAFGFQGRLLHLSAGQRLGVHLHTEARARHAW |
| | | QLTQGATVLGLFRVTPEIPAGLPSPRSE |
| 10 | dimeric hu 4-1BBL (71-248) | REGPELSPDDPAGLLDLRQGMFAQLVAQNVLLIDG |
| | connected by (G4S)₂ linker | PLSWYSDPGLAGVSLTGGLSYKEDTKELVVAKAGV |
| | | YYVFFQLELRRVVAGEGSGSVSLALHLQPLRSAAG |
| | | AAALALTVDLPPASSEARNSAFGFQGRLLHLSAGQ |
| | | RLGVHLHTEARARHAWQLTQGATVLGLFRVTPEIP |
| | | AGLGGGGSGGGGSREGPELSPDDPAGLLDLRQGMF |
| | | AQLVAQNVLLIDGPLSWYSDPGLAGVSLTGGLSYK |
| | | EDTKELVVAKAGVYYVFFQLELRRVVAGEGSGSVS |
| | | LALHLQPLRSAAGAAALALTVDLPPASSEARNSAF |
| | | GFQGRLLHLSAGQRLGVHLHTEARARHAWQLTQG |
| | | ATVLGLFRVTPEIPAGL |
| 11 | hu OX40L (51-183) | QVSHRYPRIQSIKVQFTEYKKEKGFILTSQKEDEI |
| | | MKVQNNSVIINCDGFYLISLKGYFSQEVNISLHYQ |
| | | KDEEPLFQLKKVRSVNSLMVASLTYKDKVYLNVTT |
| | | DNTSLDDFHVNGGELILIHQNPGEFCVL |
| 12 | hu OX40L (52-183) | VSHRYPRIQSIKVQFTEYKKEKGFILTSQKEDEIM |
| | | KVQNNSVIINCDGFYLISLKGYFSQEVNISLHYQK |
| | | DEEPLFQLKKVRSVNSLMVASLTYKDKVYLNVTTD |
| | | NTSLDDFHVNGGELILIHQNPGEFCVL |
| 13 | FAP(28H1) CDR-H1 | SHAMS |
| 14 | FAP(28H1) CDR-H2 | AIWASGEQYYADSVKG |
| 15 | FAP(28H1) CDR-H3 | GWLGNFDY |
| 16 | FAP(28H1) CDR-L1 | RASQSVSRSYLA |
| 17 | FAP(28H1) CDR-L2 | GASTRAT |
| 18 | FAP(28H1) CDR-L3 | QQGQVIPPT |
| 19 | FAP(4B9) CDR-H1 | SYAMS |
| 20 | FAP(4B9) CDR-H2 | AIIGSGASTYYADSVKG |
| 21 | FAP(4B9) CDR-H3 | GWFGGFNY |
| 22 | FAP(4B9) CDR-L1 | RASQSVTSSYLA |
| 23 | FAP(4B9) CDR-L2 | VGSRRAT |
| 24 | FAP(4B9) CDR-L3 | QQGIMLPPT |
| 25 | FAP(28H1) VH | EVQLLESGGGLVQPGGSLRLSCAASGFTFSSHAMS |
| | | WVRQAPGKGLEWVSAIWASGEQYYADSVKGRFTIS |
| | | RDNSKNTLYLQMNSLRAEDTAVYYCAKGWLGNFDY |
| | | WGQGTLVTVSS |
| 26 | FAP(28H1) VL | EIVLTQSPGTLSLSPGERATLSCRASQSVSRSYLA |
| | | WYQQKPGQAPRLLIIGASTRATGIPDRFSGSGSGT |
| | | DFTLTISRLEPEDFAVYYCQQGQVIPPTFGQGTKV |
| | | EIK |
| 27 | FAP(4B9) VH | EVQLLESGGGLVQPGGSLRLSCAASGFTFSSYAMS |
| | | WVRQAPGKGLEWVSAIIGSGASTYYADSVKGRFTI |
| | | SRDNSKNTLYLQMNSLRAEDTAVYYCAKGWFGGFN |
| | | YWGQGTLVTVSS |
| 28 | FAP(4B9) VL | EIVLTQSPGTLSLSPGERATLSCRASQSVTSSYLA |
| | | WYQQKPGQAPRLLINVGSRRATGIPDRFSGSGSGT |
| | | DFTLTISRLEPEDFAVYYCQQGIMLPPTFGQGTKV |
| | | EIK |
| 29 | CD19 (8B8-018) CDR-H1 | DYIMH |
| 30 | CD19 (8B8-018) CDR-H2 | YINPYNDGSKYTEKFQG |
| 31 | CD19 (8B8-018) CDR-H3 | GTYYYGSALFDY |
| 32 | CD19 (8B8-018) CDR-L1 | KSSQSLENPNGNTYLN |
| 33 | CD19 (8B8-018) CDR-L2 | RVSKRFS |
| 34 | CD19 (8B8-018) CDR-L3 | LQLTHVPYT |
| 35 | CD19 (8B8-2B11) CDR-H1 | DYIMH |
| 36 | CD19 (8B8-2B11) CDR-H2 | YINPYNDGSKYTEKFQG |
| 37 | CD19 (8B8-2B11) CDR-H3 | GTYYYGPQLFDY |
| 38 | CD19 (8B8-2B11) CDR-L1 | KSSQSLETSTGTTYLN |
| 39 | CD19 (8B8-2B11) CDR-L2 | RVSKRFS |
| 40 | CD19 (8B8-2B11) CDR-H3 | LQLLEDPYT |
| 41 | CD19 (8B8-018) VH | QVQLVQSGAEVKKPGASVKVSCKASGYTFTDYIMH |
| | | WVRQAPGQGLEWMGYINPYNDGSKYTEKFQGRVTM |
| | | TSDTSISTAYMELSRLRSDDTAVYYCARGTYYYGS |
| | | ALFDYWGQGTTVTVSS |
| 42 | CD19 (8B8-018) VL | DIVMTQTPLSLSVTPGQPASISCKSSQSLENPNGN |
| | | TYLNWYLQKPGQSPQLLIYRVSKRFSGVPDRFSGS |
| | | GSGTDFTLKISRVEAEDVGVYYCLQLTHVPYTFGQ |
| | | GTKLEIK |
| 43 | CD19 (8B8-2B11) VH | QVQLVQSGAEVKKPGASVKVSCKASGYTFTDYIMH |
| | | WVRQAPGQGLEWMGYINPYNDGSKYTEKFQGRVTM |
| | | TSDTSISTAYMELSRLRSDDTAVYYCARGTYYYGP |
| | | QLFDYWGQGTTVTVSS |
| 44 | CD19 (8B8-2B11) VL | DIVMTQTPLSLSVTPGQPASISCKSSQSLETSTGT |
| | | TYLNWYLQKPGQSPQLLIYRVSKRFSGVPDRFSGS |
| | | GSGTDFTLKISRVEAEDVGVYYCLQLLEDPYTFGQ |
| | | GTKLEIK |
| 45 | CEA CDR-H1 | DTYMH |
| 46 | CEA CDR-H2 | RIDPANGNSKYVPKFQG |
| 47 | CEA CDR-H3 | FGYYVSDYAMAY |
| 48 | CEA CDR-L1 | RAGESVDIFGVGFLH |
| 49 | CEA CDR-L2 | RASNRAT |
| 50 | CEA CDR-L3 | QQTNEDPYT |
| 51 | Humanized CEA binder VH | QVQLVQSGAEVKKPGSSVKVSCKASGFNIKDTYMH |
| | | WVRQAPGQGLEWMGRIDPANGNSKYVPKFQGRVTI |
| | | TADTSTSTAYMELSSLRSEDTAVYYCAPFGYYVSD |
| | | YAMAYWGQGTLVTVSS |
| 52 | Humanized CEA binder VL | EIVLTQSPATLSLSPGERATLSCRAGESVDIFGVG |
| | | FLHWYQQKPGQAPRLLIYRASNRATGIPARFSGSG |
| | | SGTDFTLTISSLEPEDFAVYYCQQTNEDPYTFGQG |
| | | TKLEIK |
| 53 | Human (hu) FAP | UniProt no. Q12884 |
| 54 | hu FAP ectodomain + poly- | RPSRVHNSEENTMRALTLKDILNGTFSYKTFFPNW |
| | lys-tag + his₆-tag | ISGQEYLHQSADNNIVLYNIETGQSYTILSNRTMK |
| | | SVNASNYGLSPDRQFVYLESDYSKLWRYSYTATYY |
| | | IYDLSNGEFVRGNELPRPIQYLCWSPVGSKLAYVY |
| | | QNNIYLKQRPGDPPFQITFNGRENKIFNGIPDWVY |
| | | EEEMLATKYALWWSPNGKFLAYAEFNDTDIPVIAY |
| | | SYYGDEQYPRTINIPYPKAGAKNPVVRIFIIDTTY |
| | | PAYVGPQEVPVPAMIASSDYYFSWLTWVTDERVCL |
| | | QWLKRVQNVSVLSICDFREDWQTWDCPKTQEHIEE |
| | | SRTGWAGGFFVSTPVFSYDAISYYKIFSDKDGYKH |
| | | IHYIKDTVENAIQITSGKWEAINIFRVTQDSLFYS |
| | | SNEFEEYPGRRNIYRISIGSYPPSKKCVTCHLRKE |
| | | RCQYYTASFSDYAKYYALVCYGPGIPISTLHDGRT |
| | | DQEIKILEENKELENALKNIQLPKEEIKKLEVDEI |
| | | TLWYKMILPPQFDRSKKYPLLIQVYGGPCSQSVRS |
| | | VFAVNWISYLASKEGMVIALVDGRGTAFQGDKLLY |
| | | AVYRKLGVYEVEDQITAVRKFIEMGFIDEKRIAIW |
| | | GWSYGGYVSSLALASGTGLFKCGIAVAPVSSWEYY |
| | | ASVYTERFMGLPTKDDNLEHYKNSTVMARAEYFRN |
| | | VDYLLIHGTADDNVHFQNSAQIAKALVNAQVDFQA |
| | | MWYSDQNHGLSGLSTNHLYTHMTHFLKQCFSLSDG |
| | | KKKKKKGHHHHHH |
| 55 | nucleotide sequence of hu | CGCCCTTCAAGAGTTCATAACTCTGAAGAAAATAC |
| | | AATGAGAGCACTCACACTGAAGGATATTTTAAATG |
| | | GAACATTTTCTTATAAAACATTTTTTCCAAACTGG |
| | | ATTTCAGGACAAGAATATCTTCATCAATCTGCAGA |
| | | TAACAATATAGTACTTTATAATATTGAAACAGGAC |
| | | AATCATATACCATTTTGAGTAATAGAACCATGAAA |
| | | AGTGTGAATGCTTCAAATTACGGCTTATCACCTGA |
| | | TCGGCAATTTGTATATCTAGAAAGTGATTATTCAA |
| | | AGCTTTGGAGATACTCTTACACAGCAACATATTAC |
| | | ATCTATGACCTTAGCAATGGAGAATTTGTAAGAGG |
| | | AAATGAGCTTCCTCGTCCAATTCAGTATTTATGCT |
| | | GGTCGCCTGTTGGGAGTAAATTAGCATATGTCTAT |
| | | CAAAACAATATCTATTTGAAACAAAGACCAGGAGA |
| | | TCCACCTTTTCAAATAACATTTAATGGAAGAGAAA |
| | | ATAAAATATTTAATGGAATCCCAGACTGGGTTTAT |
| | | GAAGAGGAAATGCTTGCTACAAAATATGCTCTCTG |
| | | GTGGTCTCCTAATGGAAAATTTTTGGCATATGCGG |
| | | AATTTAATGATACGGATATACCAGTTATTGCCTAT |
| | | TCCTATTATGGCGATGAACAATATCCTAGAACAAT |
| | | AAATATTCCATACCCAAAGGCTGGAGCTAAGAATC |
| | | CCGTTGTTCGGATATTTATTATCGATACCACTTAC |
| | | CCTGCGTATGTAGGTCCCCAGGAAGTGCCTGTTCC |
| | | AGCAATGATAGCCTCAAGTGATTATTATTTCAGTT |
| | | GGCTCACGTGGGTTACTGATGAACGAGTATGTTTG |
| | | CAGTGGCTAAAAAGAGTCCAGAATGTTTCGGTCCT |
| | | GTCTATATGTGACTTCAGGGAAGACTGGCAGACAT |
| | | GGGATTGTCCAAAGACCCAGGAGCATATAGAAGAA |
| | | AGCAGAACTGGATGGGCTGGTGGATTCTTTGTTTC |
| | | AACACCAGTTTTCAGCTATGATGCCATTTCGTACT |
| | | ACAAAATATTTAGTGACAAGGATGGCTACAAACAT |
| | | ATTCACTATATCAAAGACACTGTGGAAAATGCTAT |
| | | TCAAATTACAAGTGGCAAGTGGGAGGCCATAAATA |
| | | TATTCAGAGTAACACAGGATTCACTGTTTTATTCT |
| | | AGCAATGAATTTGAAGAATACCCTGGAAGAAGAAA |
| | | CATCTACAGAATTAGCATTGGAAGCTATCCTCCAA |
| | | GCAAGAAGTGTGTTACTTGCCATCTAAGGAAAGAA |
| | | AGGTGCCAATATTACACAGCAAGTTTCAGCGACTA |
| | | CGCCAAGTACTATGCACTTGTCTGCTACGGCCCAG |
| | | GCATCCCCATTTCCACCCTTCATGATGGACGCACT |
| | | GATCAAGAAATTAAAATCCTGGAAGAAAACAAGGA |
| | | ATTGGAAAATGCTTTGAAAAATATCCAGCTGCCTA |
| | | AAGAGGAAATTAAGAAACTTGAAGTAGATGAAATT |
| | | ACTTTATGGTACAAGATGATTCTTCCTCCTCAATT |
| | | TGACAGATCAAAGAAGTATCCCTTGCTAATTCAAG |
| | | TGTATGGTGGTCCCTGCAGTCAGAGTGTAAGGTCT |
| | | GTATTTGCTGTTAATTGGATATCTTATCTTGCAAG |
| | | TAAGGAAGGGATGGTCATTGCCTTGGTGGATGGTC |
| | | GAGGAACAGCTTTCCAAGGTGACAAACTCCTCTAT |
| | | GCAGTGTATCGAAAGCTGGGTGTTTATGAAGTTGA |
| | | AGACCAGATTACAGCTGTCAGAAAATTCATAGAAA |
| | | TGGGTTTCATTGATGAAAAAAGAATAGCCATATGG |
| | | GGCTGGTCCTATGGAGGATACGTTTCATCACTGGC |
| | | CCTTGCATCTGGAACTGGTCTTTTCAAATGTGGTA |
| | | TAGCAGTGGCTCCAGTCTCCAGCTGGGAATATTAC |
| | | GCGTCTGTCTACACAGAGAGATTCATGGGTCTCCC |
| | | AACAAAGGATGATAATCTTGAGCACTATAAGAATT |
| | | CAACTGTGATGGCAAGAGCAGAATATTTCAGAAAT |
| | | GTAGACTATCTTCTCATCCACGGAACAGCAGATGA |
| | | TAATGTGCACTTTCAAAACTCAGCACAGATTGCTA |
| | | AAGCTCTGGTTAATGCACAAGTGGATTTCCAGGCA |
| | | ATGTGGTACTCTGACCAGAACCACGGCTTATCCGG |
| | | CCTGTCCACGAACCACTTATACACCCACATGACCC |
| | | ACTTCCTAAAGCAGTGTTTCTCTTTGTCAGACGGC |
| | | AAAAAGAAAAAGAAAAAGGGCCACCACCATCACCA |
| | | TCAC |
| 56 | mouse FAP | UniProt no. P97321 |
| 57 | Murine FAP | RPSRVYKPEGNTKRALTLKDILNGTFSYKTYFPNW |
| | ectodomain + poly-lys- | ISEQEYLHQSEDDNIVFYNIETRESYIILSNSTMK |
| | tag + his₆-tag | SVNATDYGLSPDRQFVYLESDYSKLWRYSYTATYY |
| | | IYDLQNGEFVRGYELPRPIQYLCWSPVGSKLAYVY |
| | | QNNIYLKQRPGDPPFQITYTGRENRIFNGIPDWVY |
| | | EEEMLATKYALWWSPDGKFLAYVEFNDSDIPIIAY |
| | | SYYGDGQYPRTINIPYPKAGAKNPVVRVFIVDTTY |
| | | PHHVGPMEVPVPEMIASSDYYFSWLTWVSSERVCL |
| | | QWLKRVQNVSVLSICDFREDWHAWECPKNQEHVEE |
| | | SRTGWAGGFFVSTPAFSQDATSYYKIFSDKDGYKH |
| | | IHYIKDTVENAIQITSGKWEAIYIFRVTQDSLFYS |
| | | SNEFEGYPGRRNIYRISIGNSPPSKKCVTCHLRKE |
| | | RCQYYTASFSYKAKYYALVCYGPGLPISTLHDGRT |
| | | DQEIQVLEENKELENSLRNIQLPKVEIKKLKDGGL |
| | | TFWYKMILPPQFDRSKKYPLLIQVYGGPCSQSVKS |
| | | VFAVNWITYLASKEGIVIALVDGRGTAFQGDKFLH |
| | | AVYRKLGVYEVEDQLTAVRKFIEMGFIDEERIAIW |
| | | GWSYGGYVSSLALASGTGLFKCGIAVAPVSSWEYY |
| | | ASIYSERFMGLPTKDDNLEHYKNSTVMARAEYFRN |
| | | VDYLLIHGTADDNVHFQNSAQIAKALVNAQVDFQA |
| | | MWYSDQNHGILSGRSQNHLYTHMTHFLKQCFSLSD |
| | | GKKKKKKGHHHHHH |
| 58 | nucleotide sequence of | CGTCCCTCAAGAGTTTACAAACCTGAAGGAAACAC |
| | murine FAP | AAAGAGAGCTCTTACCTTGAAGGATATTTTAAATG |
| | ectodomain + poly-lys- | GAACATTCTCATATAAAACATATTTTCCCAACTGG |
| | tag + his₆-tag | ATTTCAGAACAAGAATATCTTCATCAATCTGAGGA |
| | | TGATAACATAGTATTTTATAATATTGAAACAAGAG |
| | | AATCATATATCATTTTGAGTAATAGCACCATGAAA |
| | | AGTGTGAATGCTACAGATTATGGTTTGTCACCTGA |
| | | TCGGCAATTTGTGTATCTAGAAAGTGATTATTCAA |
| | | AGCTCTGGCGATATTCATACACAGCGACATACTAC |
| | | ATCTACGACCTTCAGAATGGGGAATTTGTAAGAGG |
| | | ATACGAGCTCCCTCGTCCAATTCAGTATCTATGCT |
| | | GGTCGCCTGTTGGGAGTAAATTAGCATATGTATAT |
| | | CAAAACAATATTTATTTGAAACAAAGACCAGGAGA |
| | | TCCACCTTTTCAAATAACTTATACTGGAAGAGAAA |
| | | ATAGAATATTTAATGGAATACCAGACTGGGTTTAT |
| | | GAAGAGGAAATGCTTGCCACAAAATATGCTCTTTG |
| | | GTGGTCTCCAGATGGAAAATTTTTGGCATATGTAG |
| | | AATTTAATGATTCAGATATACCAATTATTGCCTAT |
| | | TCTTATTATGGTGATGGACAGTATCCTAGAACTAT |
| | | AAATATTCCATATCCAAAGGCTGGGGCTAAGAATC |
| | | CGGTTGTTCGTGTTTTTATTGTTGACACCACCTAC |
| | | CCTCACCACGTGGGCCCAATGGAAGTGCCAGTTCC |
| | | AGAAATGATAGCCTCAAGTGACTATTATTTCAGCT |
| | | GGCTCACATGGGTGTCCAGTGAACGAGTATGCTTG |
| | | CAGTGGCTAAAAAGAGTGCAGAATGTCTCAGTCCT |
| | | GTCTATATGTGATTTCAGGGAAGACTGGCATGCAT |
| | | GGGAATGTCCAAAGAACCAGGAGCATGTAGAAGAA |
| | | AGCAGAACAGGATGGGCTGGTGGATTCTTTGTTTC |
| | | GACACCAGCTTTTAGCCAGGATGCCACTTCTTACT |
| | | ACAAAATATTTAGCGACAAGGATGGTTACAAACAT |
| | | ATTCACTACATCAAAGACACTGTGGAAAATGCTAT |
| | | TCAAATTACAAGTGGCAAGTGGGAGGCCATATATA |
| | | TATTCCGCGTAACACAGGATTCACTGTTTTATTCT |
| | | AGCAATGAATTTGAAGGTTACCCTGGAAGAAGAAA |
| | | CATCTACAGAATTAGCATTGGAAACTCTCCTCCGA |
| | | GCAAGAAGTGTGTTACTTGCCATCTAAGGAAAGAA |
| | | AGGTGCCAATATTACACAGCAAGTTTCAGCTACAA |
| | | AGCCAAGTACTATGCACTCGTCTGCTATGGCCCTG |
| | | GCCTCCCCATTTCCACCCTCCATGATGGCCGCACA |
| | | GACCAAGAAATACAAGTATTAGAAGAAAACAAAGA |
| | | ACTGGAAAATTCTCTGAGAAATATCCAGCTGCCTA |
| | | AAGTGGAGATTAAGAAGCTCAAAGACGGGGGACTG |
| | | ACTTTCTGGTACAAGATGATTCTGCCTCCTCAGTT |
| | | TGACAGATCAAAGAAGTACCCTTTGCTAATTCAAG |
| | | TGTATGGTGGTCCTTGTAGCCAGAGTGTTAAGTCT |
| | | GTGTTTGCTGTTAATTGGATAACTTATCTCGCAAG |
| | | TAAGGAGGGGATAGTCATTGCCCTGGTAGATGGTC |
| | | GGGGCACTGCTTTCCAAGGTGACAAATTCCTGCAT |
| | | GCCGTGTATCGAAAACTGGGTGTATATGAAGTTGA |
| | | GGACCAGCTCACAGCTGTCAGAAAATTCATAGAAA |
| | | TGGGTTTCATTGATGAAGAAAGAATAGCCATATGG |
| | | GGCTGGTCCTACGGAGGTTATGTTTCATCCCTGGC |
| | | CCTTGCATCTGGAACTGGTCTTTTCAAATGTGGCA |
| | | TAGCAGTGGCTCCAGTCTCCAGCTGGGAATATTAC |
| | | GCATCTATCTACTCAGAGAGATTCATGGGCCTCCC |
| | | AACAAAGGACGACAATCTCGAACACTATAAAAATT |
| | | CAACTGTGATGGCAAGAGCAGAATATTTCAGAAAT |
| | | GTAGACTATCTTCTCATCCACGGAACAGCAGATGA |
| | | TAATGTGCACTTTCAGAACTCAGCACAGATTGCTA |
| | | AAGCTTTGGTTAATGCACAAGTGGATTTCCAGGCG |
| | | ATGTGGTACTCTGACCAGAACCATGGTATATTATC |
| | | TGGGCGCTCCCAGAATCATTTATATACCCACATGA |
| | | CGCACTTCCTCAAGCAATGCTTTTCTTTATCAGAC |
| | | GGCAAAAAGAAAAAGAAAAAGGGCCACCACCATCA |
| | | CCATCAC |
| 59 | Cynomolgus FAP | RPPRVHNSEENTMRALTLKDILNGTFSYKTFFPNW |
| | ectodomain + poly-lys- | ISGQEYLHQSADNNIVLYNIETGQSYTILSNRTMK |
| | tag + his₆-tag | SVNASNYGLSPDRQFVYLESDYSKLWRYSYTATYY |
| | | IYDLSNGEFVRGNELPRPIQYLCWSPVGSKLAYVY |
| | | QNNIYLKQRPGDPPFQITFNGRENKIFNGIPDWVY |
| | | EEEMLATKYALWWSPNGKFLAYAEFNDTDIPVIAY |
| | | SYYGDEQYPRTINIPYPKAGAKNPFVRIFIIDTTY |
| | | PAYVGPQEVPVPAMIASSDYYFSWLTWVTDERVCL |
| | | QWLKRVQNVSVLSICDFREDWQTWDCPKTQEHIEE |
| | | SRTGWAGGFFVSTPVFSYDAISYYKIFSDKDGYKH |
| | | IHYIKDTVENAIQITSGKWEAINIFRVTQDSLFYS |
| | | SNEFEDYPGRRNIYRISIGSYPPSKKCVTCHLRKE |
| | | RCQYYTASFSDYAKYYALVCYGPGIPISTLHDGRT |
| | | DQEIKILEENKELENALKNIQLPKEEIKKLEVDEI |
| | | TLWYKMILPPQFDRSKKYPLLIQVYGGPCSQSVRS |
| | | VFAVNWISYLASKEGMVIALVDGRGTAFQGDKLLY |
| | | AVYRKLGVYEVEDQITAVRKFIEMGFIDEKRIAIW |
| | | GWSYGGYVSSLALASGTGLFKCGIAVAPVSSWEYY |
| | | ASVYTERFMGLPTKDDNLEHYKNSTVMARAEYFRN |
| | | VDYLLIHGTADDNVHFQNSAQIAKALVNAQVDFQA |
| | | MWYSDQNHGLSGLSTNHLYTHMTHFLKQCFSLSDG |
| | | KKKKKKGHHHHHH |
| 60 | nucleotide sequence of | CGCCCTCCAAGAGTTCATAACTCTGAAGAAAATAC |
| | cynomolgus FAP | AATGAGAGCACTCACACTGAAGGATATTTTAAATG |
| | ectodomain + poly-lys- | GGACATTTTCTTATAAAACATTTTTTCCAAACTGG |
| | tag + his₆-tag | ATTTCAGGACAAGAATATCTTCATCAATCTGCAGA |
| | | TAACAATATAGTACTTTATAATATTGAAACAGGAC |
| | | AATCATATACCATTTTGAGTAACAGAACCATGAAA |
| | | AGTGTGAATGCTTCAAATTATGGCTTATCACCTGA |
| | | TCGGCAATTTGTATATCTAGAAAGTGATTATTCAA |
| | | AGCTTTGGAGATACTCTTACACAGCAACATATTAC |
| | | ATCTATGACCTTAGCAATGGAGAATTTGTAAGAGG |
| | | AAATGAGCTTCCTCGTCCAATTCAGTATTTATGCT |
| | | GGTCGCCTGTTGGGAGTAAATTAGCATATGTCTAT |
| | | CAAAACAATATCTATTTGAAACAAAGACCAGGAGA |
| | | TCCACCTTTTCAAATAACATTTAATGGAAGAGAAA |
| | | ATAAAATATTTAATGGAATCCCAGACTGGGTTTAT |
| | | GAAGAGGAAATGCTTGCTACAAAATATGCTCTCTG |
| | | GTGGTCTCCTAATGGAAAATTTTTGGCATATGCGG |
| | | AATTTAATGATACAGATATACCAGTTATTGCCTAT |
| | | TCCTATTATGGCGATGAACAATATCCCAGAACAAT |
| | | AAATATTCCATACCCAAAGGCCGGAGCTAAGAATC |
| | | CTTTTGTTCGGATATTTATTATCGATACCACTTAC |
| | | CCTGCGTATGTAGGTCCCCAGGAAGTGCCTGTTCC |
| | | AGCAATGATAGCCTCAAGTGATTATTATTTCAGTT |
| | | GGCTCACGTGGGTTACTGATGAACGAGTATGTTTG |
| | | CAGTGGCTAAAAAGAGTCCAGAATGTTTCGGTCTT |
| | | GTCTATATGTGATTTCAGGGAAGACTGGCAGACAT |
| | | GGGATTGTCCAAAGACCCAGGAGCATATAGAAGAA |
| | | AGCAGAACTGGATGGGCTGGTGGATTCTTTGTTTC |
| | | AACACCAGTTTTCAGCTATGATGCCATTTCATACT |
| | | ACAAAATATTTAGTGACAAGGATGGCTACAAACAT |
| | | ATTCACTATATCAAAGACACTGTGGAAAATGCTAT |
| | | TCAAATTACAAGTGGCAAGTGGGAGGCCATAAATA |
| | | TATTCAGAGTAACACAGGATTCACTGTTTTATTCT |
| | | AGCAATGAATTTGAAGATTACCCTGGAAGAAGAAA |
| | | CATCTACAGAATTAGCATTGGAAGCTATCCTCCAA |
| | | GCAAGAAGTGTGTTACTTGCCATCTAAGGAAAGAA |
| | | AGGTGCCAATATTACACAGCAAGTTTCAGCGACTA |
| | | CGCCAAGTACTATGCACTTGTCTGCTATGGCCCAG |
| | | GCATCCCCATTTCCACCCTTCATGACGGACGCACT |
| | | GATCAAGAAATTAAAATCCTGGAAGAAAACAAGGA |
| | | ATTGGAAAATGCTTTGAAAAATATCCAGCTGCCTA |
| | | AAGAGGAAATTAAGAAACTTGAAGTAGATGAAATT |
| | | ACTTTATGGTACAAGATGATTCTTCCTCCTCAATT |
| | | TGACAGATCAAAGAAGTATCCCTTGCTAATTCAAG |
| | | TGTATGGTGGTCCCTGCAGTCAGAGTGTAAGGTCT |
| | | GTATTTGCTGTTAATTGGATATCTTATCTTGCAAG |
| | | TAAGGAAGGGATGGTCATTGCCTTGGTGGATGGTC |
| | | GGGGAACAGCTTTCCAAGGTGACAAACTCCTGTAT |
| | | GCAGTGTATCGAAAGCTGGGTGTTTATGAAGTTGA |
| | | AGACCAGATTACAGCTGTCAGAAAATTCATAGAAA |
| | | TGGGTTTCATTGATGAAAAAAGAATAGCCATATGG |
| | | GGCTGGTCCTATGGAGGATATGTTTCATCACTGGC |
| | | CCTTGCATCTGGAACTGGTCTTTTCAAATGTGGGA |
| | | TAGCAGTGGCTCCAGTCTCCAGCTGGGAATATTAC |
| | | GCGTCTGTCTACACAGAGAGATTCATGGGTCTCCC |
| | | AACAAAGGATGATAATCTTGAGCACTATAAGAATT |
| | | CAACTGTGATGGCAAGAGCAGAATATTTCAGAAAT |
| | | GTAGACTATCTTCTCATCCACGGAACAGCAGATGA |
| | | TAATGTGCACTTTCAAAACTCAGCACAGATTGCTA |
| | | AAGCTCTGGTTAATGCACAAGTGGATTTCCAGGCA |
| | | ATGTGGTACTCTGACCAGAACCACGGCTTATCCGG |
| | | CCTGTCCACGAACCACTTATACACCCACATGACCC |
| | | ACTTCCTAAAGCAGTGTTTCTCTTTGTCAGACGGC |
| | | AAAAAGAAAAAGAAAAAGGGCCACCACCATCACCA |
| | | TCAC |
| 61 | human CEA | UniProt no. P06731 |
| 62 | human MCSP | UniProt no. Q6UVK1 |
| 63 | human EGFR | UniProt no. P00533 |
| 64 | human CD19 | UniProt no. P15391 |
| 65 | human CD20 | Uniprot no. P11836 |
| 66 | human CD33 | UniProt no. P20138 |
| 67 | human Lymphotoxin α | UniProt no. P01374 |
| 68 | human TNF | UniProt no. P01375 |
| 69 | human Lymphotoxin β | UniProt no. Q06643 |
| 70 | human OX40L | UniProt no. P23510 |
| 71 | human CD40L | UniProt no. P29965 |
| 72 | human FasL | UniProt no. P48023 |
| 73 | human CD27L | UniProt no. P32970 |
| 74 | human CD30L | UniProt no. P32971 |
| 75 | human 4-1BBL | UniProt no. P41273 |
| 76 | human TRAIL | UniProt no. P50591 |
| 77 | human RANKL | UniProt no. O14788 |
| 78 | human TWEAK | UniProt no. O43508 |
| 79 | human APRIL | UniProt no. O75888 |
| 80 | human BAFF | UniProt no. Q9Y275 |
| 81 | human LIGHT | UniProt no. O43557 |
| 82 | human TL1A | UniProt no. O95150 |
| 83 | human GITRL | UniProt no. Q9UNG2 |
| 84 | human ectodysplasin A | UniProt no. Q92838 |
| 85 | hu 4-1BBL (50-254) | ACPWAVSGARASPGSAASPRLREGPELSPDDPAGL |
| | | LDLRQGMFAQLVAQNVLLIDGPLSWYSDPGLAGVS |
| | | LTGGLSYKEDTKELVVAKAGVYYVFFQLELRRVVA |
| | | GEGSGSVSLALHLQPLRSAAGAAALALTVDLPPAS |
| | | SEARNSAFGFQGRLLHLSAGQRLGVHLHTEARARH |
| | | AWQLTQGATVLGLFRVTPEIPAGLPSPRSE |
| 86 | Peptide linker G4S | GGGGS |
| 87 | Peptide linker (G4S)₂ | GGGGSGGGGS |
| 88 | Peptide linker (SG4)₂ | SGGGGSGGGG |
| 89 | Peptide linker (G₄S)₃ | GGGGSGGGGSGGGGS |
| 90 | Peptide linker G4(SG4)₂ | GGGGSGGGGSGGGG |
| 91 | Peptide linker (G₄S)₄ | GGGGSGGGGSGGGGSGGGGS |
| 92 | Peptide linker | GSPGSSSSGS |
| 93 | Peptide linker | GSGSGSGS |
| 94 | Peptide linker | GSGSGNGS |
| 95 | Peptide linker | GGSGSGSG |
| 96 | Peptide linker | GGSGSG |
| 97 | Peptide linker | GGSG |
| 98 | Peptide linker | GGSGNGSG |
| 99 | Peptide linker | GGNGSGSG |
| 100 | Peptide linker | GGNGSG |
| 101 | nucleotide sequence of Fc | see Table 1 |
| | hole dimeric 4-1BBL | |
| | (71-248) chain | |
| 102 | nucleotide sequence of anti- | see Table 1 |
| | FAP(4B9) Fc knob | |
| | monomeric 4-1BBL (71-248) | |
| 103 | nucleotide sequence of anti- | see Table 1 |
| | FAP(4B9) light chain | |
| 104 | Fc hole dimeric 4-1BB | see Table 1 |
| | (71-248) ligand chain | |
| 105 | anti-FAP(4B9) Fc knob | see Table 1 |
| | monomeric 41-BBL | (71-248) |
| 106 | anti-FAP(4B9) light chain | see Table 1 |
| 107 | nucleotide sequence of Fc | see Table 2 |
| | hole monomeric 4-1BBL | |
| | (71-248) chain | |
| 108 | nucleotide sequence of anti- | see Table 2 |
| | FAP(4B9) Fc knob dimeric | |
| | 4-1BBL (71-248) | |
| 109 | Fc hole monomeric 4-1BB | see Table 2 |
| | ligand (71-248) chain | |
| 110 | anti-FAP(4B9) Fc knob | see Table 2 |
| | dimeric 4-1BBL (71-248) | |
| 111 | nucleotide sequence of anti- | see Table 3 |
| | FAP(28H1) Fc knob | |
| | monomeric 4-1BBL (71-248) | |
| 112 | nucleotide sequence of anti- | see Table 3 |
| | FAP(28H1) light chain | |
| 113 | anti-FAP(28H1) Fc knob | see Table 3 |
| | monomeric 4-1BBL (71-248) | |
| 114 | anti-FAP(28H1) light chain | see Table 3 |
| 115 | nucleotide sequence of anti- | see Table 4 |
| | FAP(28H1) Fc knob dimeric | |
| | 4-1BBL (71-248) | |
| 116 | anti-FAP(28H1) Fc knob | see Table 4 |
| | dimeric 4-1BBL (71-248) | |
| 117 | nucleotide sequence of anti- | see Table 5 |
| | CD19(8B8-018) Fc knob | |
| | monomeric 4-1BBL (71-248) | |
| 118 | nucleotide sequence of anti- | see Table 5 |
| | CD19(8B8-018) light chain | |
| 119 | anti-CD19(8B8-018) Fc knob | see Table 5 |
| | monomeric 4-1-BBL | |
| | (71-248) | |
| 120 | anti-CD19(8B8-018) light | see Table 5 |
| | chain | |
| 121 | nucleotide sequence of anti- | see Table 6 |
| | CD19(8B8-018) Fc knob | |
| | dimeric 4-1BBL (71-248) | |
| 122 | anti-CD19(8B8-018) Fc knob | see Table 6 |
| | dimeric 4-1BB (71-248) | |
| 123 | nucleotide sequence of anti- | see Table 7 |
| | CD19(8B8-2B11) Fc knob | |
| | monomeric 4-1BBL (71-248) | |
| 124 | nucleotide sequence of anti- | see Table 7 |
| | CD19(8B8-2B11) light chain | |
| 125 | anti-CD19(8B8-2B11) Fc | see Table 7 |
| | knob monomeric 4-1BBL | |
| | (71-248) | |
| 126 | anti-CD19(8B8-2B11) light | see Table 7 |
| | chain | |
| 127 | nucleotide sequence of anti- | see Table 8 |
| | CD19(8B8-2B11) Fc knob | |
| | dimeric 4-1BBL (71-248) | |
| 128 | anti-CD19(8B8-2B11) Fc | see Table 8 |
| | knob dimeric 4-1BB ligand | |
| | (71-248) | |
| 129 | HVR-L1 of anti-CD19 8B8 | NSNGNT |
| 130 | HVR-L2 of anti-CD19 8B8 | KFNG |
| 131 | Nucleotide sequence of Fc | see Table 11 |
| | hole chain with HYRF | |
| | mutation | |
| 132 | Nucleotide sequence of | see Table 11 |
| | human CD19 antigen Fc knob | |
| | chain avi tag | |
| 133 | Fc hole chain with HYRF | see Table 11 |
| | mutation | |
| 134 | human CD19 antigen Fc knob | see Table 11 |
| | chain avi tag | |
| 135 | Nucleotide sequence of | see Table 11 |
| | cynomolgus CD19 antigen Fc | |
| | knob chain avi tag | |
| 136 | cynomolgus CD19 antigen Fc | see Table 11 |
| | knob chain avi tag | |
| 137 | Nucleotide sequence CD19 | CAAGTTCAATTGGTTCAATCTGGTGCTGAAGTAAA |
| | (8B8) VH Parental clone | AAAACCGGGCGCTTCCGTTAAAGTGAGCTGCAAAG |
| | | CATCTGGTTACACCTTCACTGACTATATCATGCAC |
| | | TGGGTTCGTCAGGCCCCGGGCCAGGGTCTGGAGTG |
| | | GATGGGCTACATTAACCCATACAACGACGGTTCCA |
| | | AATATACCGAGAAATTCCAGGGCCGCGTCACGATG |
| | | ACCAGCGACACTTCTATCTCCACCGCGTACATGGA |
| | | ACTGTCTAGACTGCGTTCTGACGACACCGCTGTTT |
| | | ACTATTGTGCACGCGGTACTTACTACTACGGTTCC |
| | | GCCCTCTTTGATTACTGGGGCCAAGGTACCACGGT |
| | | GACCGTAAGCTCT |
| 138 | Nucleotide sequence CD19 | GATATTGTTATGACTCAAACTCCACTGTCTCTGTC |
| | (8B8) VL Parental clone | CGTGACCCCGGGTCAGCCAGCGAGCATTTCTTGCA |
| | | AATCCAGCCAATCTCTGGAAAACTCCAACGGCAAC |
| | | ACGTACCTGAACTGGTATCTCCAGAAACCGGGTCA |
| | | GAGCCCGCAGCTGCTGATCTACCGTGTATCTAAGC |
| | | GCTTCTCCGGCGTTCCTGATCGTTTCAGCGGTTCT |
| | | GGATCCGGCACCGACTTTACTCTGAAAATCAGCCG |
| | | TGTGGAAGCTGAAGACGTTGGCGTCTACTATTGTC |
| | | TGCAGTTGACCCACGTTCCGTACACCTTCGGTCAA |
| | | GGAACTAAACTGGAAATTAAA |
| 139 | CD19 L1 reverse random | see Table 13 |
| 140 | CD19 L2 forward random | see Table 13 |
| 141 | CD19 H1 reverse random | see Table 13 |
| 142 | CD19 H2 forward random | see Table 13 |
| 143 | CD19 H3 reverse constant | see Table 13 |
| 144 | LMB3 | see Table 13 |
| 145 | CD19 L1 forward constant | see Table 14 |
| 146 | CD19 L3 reverse random | see Table 14 |
| 147 | CD19 L3 forward constant | see Table 14 |
| 148 | CD19 H3 reverse random | see Table 14 |
| 149 | Nucleotide sequence SNAP | GGCCGCCGCTAGCGGCATCGACTACAAGGACGACG |
| | tag human CD19 ECD-PDGFR | ATGACAAGGCCGGCATCGATGCCATCATGGACAAA |
| | | GACTGCGAAATGAAGCGCACCACCCTGGATAGCCC |
| | | TCTGGGCAAGCTGGAACTGTCTGGGTGCGAACAGG |
| | | GCCTGCACGAGATCAAGCTGCTGGGCAAAGGAACA |
| | | TCTGCCGCCGACGCCGTGGAAGTGCCTGCCCCAGC |
| | | CGCCGTGCTGGGCGGACCAGAGCCACTGATGCAGG |
| | | CCACCGCCTGGCTCAACGCCTACTTTCACCAGCCT |
| | | GAGGCCATCGAGGAGTTCCCTGTGCCAGCCCTGCA |
| | | CCACCCAGTGTTCCAGCAGGAGAGCTTTACCCGCC |
| | | AGGTGCTGTGGAAACTGCTGAAAGTGGTGAAGTTC |
| | | GGAGAGGTCATCAGCTACCAGCAGCTGGCCGCCCT |
| | | GGCCGGCAATCCCGCCGCCACCGCCGCCGTGAAAA |
| | | CCGCCCTGAGCGGAAATCCCGTGCCCATTCTGATC |
| | | CCCTGCCACCGGGTGGTGTCTAGCTCTGGCGCCGT |
| | | GGGGGGCTACGAGGGCGGGCTCGCCGTGAAAGAGT |
| | | GGCTGCTGGCCCACGAGGGCCACAGACTGGGCAAG |
| | | CCTGGGCTGGGTGATATCCCCGAGGAACCCCTGGT |
| | | CGTGAAGGTGGAAGAGGGCGACAATGCCGTGCTGC |
| | | AGTGCCTGAAGGGCACCTCCGATGGCCCTACCCAG |
| | | CAGCTGACCTGGTCCAGAGAGAGCCCCCTGAAGCC |
| | | CTTCCTGAAGCTGTCTCTGGGCCTGCCTGGCCTGG |
| | | GCATCCATATGAGGCCTCTGGCCATCTGGCTGTTC |
| | | ATCTTCAACGTGTCCCAGCAGATGGGCGGCTTCTA |
| | | CCTGTGTCAGCCTGGCCCCCCATCTGAGAAGGCTT |
| | | GGCAGCCTGGCTGGACCGTGAACGTGGAAGGATCC |
| | | GGCGAGCTGTTCCGGTGGAACGTGTCCGATCTGGG |
| | | CGGCCTGGGATGCGGCCTGAAGAACAGATCTAGCG |
| | | AGGGCCCCAGCAGCCCCAGCGGCAAACTGATGAGC |
| | | CCCAAGCTGTACGTGTGGGCCAAGGACAGACCCGA |
| | | GATCTGGGAGGGCGAGCCTCCTTGCCTGCCCCCTA |
| | | GAGACAGCCTGAACCAGAGCCTGAGCCAGGACCTG |
| | | ACAATGGCCCCTGGCAGCACACTGTGGCTGAGCTG |
| | | TGGCGTGCCACCCGACTCTGTGTCTAGAGGCCCTC |
| | | TGAGCTGGACCCACGTGCACCCTAAGGGCCCTAAG |
| | | AGCCTGCTGAGCCTGGAACTGAAGGACGACAGGCC |
| | | CGCCAGAGATATGTGGGTCATGGAAACCGGCCTGC |
| | | TGCTGCCTAGAGCCACAGCCCAGGATGCCGGCAAG |
| | | TACTACTGCCACAGAGGCAACCTGACCATGAGCTT |
| | | CCACCTGGAAATCACCGCCAGACCCGTGCTGTGGC |
| | | ACTGGCTGCTGAGAACAGGCGGCTGGAAGGTCGAC |
| | | GAACAAAAACTCATCTCAGAAGAGGATCTGAATGC |
| | | TGTGGGCCAGGACACGCAGGAGGTCATCGTGGTGC |
| | | CACACTCCTTGCCCTTTAAGGTGGTGGTGATCTCA |
| | | GCCATCCTGGCCCTGGTGGTGCTCACCATCATCTC |
| | | CCTTATCATCCTCATCATGCTTTGGCAGAAGAAGC |
| | | CACGT |
| 150 | Nucleotide sequence SNAP | CCGGCCGCCGCTAGCGGCATCGACTACAAGGACGA |
| | tag cynomolgus CD19 | CGATGACAAGGCCGGCATCGATGCCATCATGGACA |
| | ECD-PDGFR | AAGACTGCGAAATGAAGCGCACCACCCTGGATAGC |
| | | CCTCTGGGCAAGCTGGAACTGTCTGGGTGCGAACA |
| | | GGGCCTGCACGAGATCAAGCTGCTGGGCAAAGGAA |
| | | CATCTGCCGCCGACGCCGTGGAAGTGCCTGCCCCA |
| | | GCCGCCGTGCTGGGCGGACCAGAGCCACTGATGCA |
| | | GGCCACCGCCTGGCTCAACGCCTACTTTCACCAGC |
| | | CTGAGGCCATCGAGGAGTTCCCTGTGCCAGCCCTG |
| | | CACCACCCAGTGTTCCAGCAGGAGAGCTTTACCCG |
| | | CCAGGTGCTGTGGAAACTGCTGAAAGTGGTGAAGT |
| | | TCGGAGAGGTCATCAGCTACCAGCAGCTGGCCGCC |
| | | CTGGCCGGCAATCCCGCCGCCACCGCCGCCGTGAA |
| | | AACCGCCCTGAGCGGAAATCCCGTGCCCATTCTGA |
| | | TCCCCTGCCACCGGGTGGTGTCTAGCTCTGGCGCC |
| | | GTGGGGGGCTACGAGGGCGGGCTCGCCGTGAAAGA |
| | | GTGGCTGCTGGCCCACGAGGGCCACAGACTGGGCA |
| | | AGCCTGGGCTGGGTGATATCCCCCAGGAACCCCTG |
| | | GTCGTGAAGGTGGAAGAGGGCGACAATGCCGTGCT |
| | | CCAGTGTCTCGAGGGCACCTCCGATGGCCCTACAC |
| | | AGCAGCTCGTGTGGTGCAGAGACAGCCCCTTCGAG |
| | | CCCTTCCTGAACCTGTCTCTGGGCCTGCCTGGCAT |
| | | GGGCATCAGAATGGGCCCTCTGGGCATCTGGCTGC |
| | | TGATCTTCAACGTGTCCAACCAGACCGGCGGCTTC |
| | | TACCTGTGTCAGCCTGGCCTGCCAAGCGAGAAGGC |
| | | TTGGCAGCCTGGATGGACCGTGTCCGTGGAAGGAT |
| | | CTGGCGAGCTGTTCCGGTGGAACGTGTCCGATCTG |
| | | GGCGGCCTGGGATGCGGCCTGAAGAACAGAAGCAG |
| | | CGAGGGCCCTAGCAGCCCCAGCGGCAAGCTGAATA |
| | | GCAGCCAGCTGTACGTGTGGGCCAAGGACAGACCC |
| | | GAGATGTGGGAGGGCGAGCCTGTGTGTGGCCCCCC |
| | | TAGAGATAGCCTGAACCAGAGCCTGAGCCAGGACC |
| | | TGACAATGGCCCCTGGCAGCACACTGTGGCTGAGC |
| | | TGTGGCGTGCCACCCGACTCTGTGTCCAGAGGCCC |
| | | TCTGAGCTGGACACACGTGCGGCCTAAGGGCCCTA |
| | | AGAGCAGCCTGCTGAGCCTGGAACTGAAGGACGAC |
| | | CGGCCCGACCGGGATATGTGGGTGGTGGATACAGG |
| | | CCTGCTGCTGACCAGAGCCACAGCCCAGGATGCCG |
| | | GCAAGTACTACTGCCACAGAGGCAACTGGACCAAG |
| | | AGCTTTTACCTGGAAATCACCGCCAGACCCGCCCT |
| | | GTGGCACTGGCTGCTGAGAATCGGAGGCTGGAAGG |
| | | TCGACGAGCAGAAGCTGATCTCCGAAGAGGACCTG |
| | | AACGCCGTGGGCCAGGATACCCAGGAAGTGATCGT |
| | | GGTGCCCCACAGCCTGCCCTTCAAGGTGGTCGTGA |
| | | TCAGCGCCATTCTGGCCCTGGTGGTGCTGACCATC |
| | | ATCAGCCTGATCATCCTGATTATGCTGTGGCAGAA |
| | | AAAGCCCCGC |
| 151 | Polypeptide sequence SNAP | PAAASGIDYKDDDDKAGIDAIMDKDCEMKRTTLDS |
| | | PLGKLELSGCEQGLHEIKLLGKGTSAADAVEVPAP |
| | | AAVLGGPEPLMQATAWLNAYFHQPEAIEEFPVPAL |
| | | HHPVFQQESFTRQVLWKLLKVVKFGEVISYQQLAA |
| | | LAGNPAATAAVKTALSGNPVPILIPCHRVVSSSGA |
| | | VGGYEGGLAVKEWLLAHEGHRLGKPGLGDIPEEPL |
| | | VVKVEEGDNAVLQCLKGTSDGPTQQLTWSRESPLK |
| | | PFLKLSLGLPGLGIHMRPLAIWLFIFNVSQQMGGF |
| | | YLCQPGPPSEKAWQPGWTVNVEGSGELFRWNVSDL |
| | | GGLGCGLKNRSSEGPSSPSGKLMSPKLYVWAKDRP |
| | | EIWEGEPPCLPPRDSLNQSLSQDLTMAPGSTLWLS |
| | | CGVPPDSVSRGPLSWTHVHPKGPKSLLSLELKDDR |
| | | PARDMWVMETGLLLPRATAQDAGKYYCHRGNLTMS |
| | | FHLEITARPVLWHWLLRTGGWKVDEQKLISEEDLN |
| | | AVGQDTQEVIVVPHSLPFKVVVISAILALVVLTII |
| | | SLIILIMLWQKKPR |
| 152 | Polypeptide sequence SNAP | PAAASGIDYKDDDDKAGIDAIMDKDCEMKRTTLDS |
| | | PLGKLELSGCEQGLHEIKLLGKGTSAADAVEVPAP |
| | | AAVLGGPEPLMQATAWLNAYFHQPEAIEEFPVPAL |
| | | HHPVFQQESFTRQVLWKLLKVVKFGEVISYQQLAA |
| | | LAGNPAATAAVKTALSGNPVPILIPCHRVVSSSGA |
| | | VGGYEGGLAVKEWLLAHEGHRLGKPGLGDIPQEPL |
| | | VVKVEEGDNAVLQCLEGTSDGPTQQLVWCRDSPFE |
| | | PFLNLSLGLPGMGIRMGPLGIWLLIFNVSNQTGGF |
| | | YLCQPGLPSEKAWQPGWTVSVEGSGELFRWNVSDL |
| | | GGLGCGLKNRSSEGPSSPSGKLNSSQLYVWAKDRP |
| | | EMWEGEPVCGPPRDSLNQSLSQDLTMAPGSTLWLS |
| | | CGVPPDSVSRGPLSWTHVRPKGPKSSLLSLELKDD |
| | | RPDRDMWVVDTGLLLTRATAQDAGKYYCHRGNWTK |
| | | SFYLEITARPALWHWLLRIGGWKVDEQKLISEEDL |
| | | NAVGQDTQEVIVVPHSLPFKVVVISAILALVVLTI |
| | | ISLIILIMLWQKKPR |
| 153 | nucleotide sequence of anti- | see Table 17 |
| | CEA(T84.66-LCHA) Fc knob | |
| | monomeric 4-1BBL (71-248) | |
| 154 | nucleotide sequence of anti- | see Table 17 |
| | CEA(T84.66-LCHA) light | |
| | chain | |
| 155 | anti-CEA(T84.66-LCHA) Fc | see Table 17 |
| | knob monomeric 4-1 BBL | |
| | (71-248) | |
| 156 | anti-CEA(T84.66-LCHA) | see Table 17 |
| | light chain | |
| 157 | nucleotide sequence of anti- | see Table 18 |
| | CENT84.66-LCHA) Fc knob | |
| | dimeric 4-1BBL (71-248) | |
| 158 | anti-CEA(T84.66-LCHA) Fc | see Table 18 |
| | knob dimeric 4-1BBL | |
| | (71-248) | |
| 159 | anti-mu CEA T84.66 | MKCSWVIFFL MAVVTGVNSE VQLQQSGAEL |
| | VH | VEPGASVKLS CTASGFNIKD TYMHWVKQRP |
| | | EQGLEWIGRI DPANGNSKYV PKFQGKATIT |
| | | ADTSSNTAYL QLTSLTSEDT AVYYCAPFGY |
| | | YVSDYAMAYW GQGTSVTVSS |
| 160 | anti-mu CEA T84.66 | METDTLLLWV LLLWVPGSTG DIVLTQSPAS |
| | VL | LAVSLGQRAT MSCRAGESVD IFGVGFLHWY |
| | | QQKPGQPPKL LIYRASNLES GIPVRFSGTG |
| | | SRTDFTLIID PVEADDVATY YCQQTNEDPY |
| | | TFGGGTKLEI K |
| 161 | IGHV1-69*08 | TAAGGGGCTT CCTAGTCCTA AGGCTGAGGA |
| | IMGT Acc No. Z14309 | AGGGATCCTG GTTTAGTTAA AGAGGATTTT |
| | | ATTCACCCCT GTGTCCTCTC CACAGGTGTC |
| | | CAGTCCCAGG TCCAGCTGGT GCAATCTGGG |
| | | GCTGAGGTGA AGAAGCCTGG GTCCTCGGTG |
| | | AAGGTCTCCT GCAAGGCTTC TGGAGGCACC |
| | | TTCAGCAGCT ATACTATCAG CTGGGTGCGA |
| | | CAGGCCCCTG GACAAGGGCT TGAGTGGATG |
| | | GGAAGGATCA TCCCTATCCT TGGTACAGCA |
| | | AACTACGCAC AGAAGTTCCA GGGCAGAGTC |
| | | ACGATTACCG CGGACAAATC CACGAGCACA |
| | | GCCTACATGG AGCTGAGCAG CCTGAGATCT |
| | | GAGGACACGG CCGTGTATTA CTGTGCGAGA |
| | | GA |
| 162 | IGKV3-11*01 | CTGCAGCTGG AAGCTCAGCT CCCACCCAGC |
| | IMGT Acc No. | TGCTTTGCAT GTCCCTCCCA GCTGCCCTAC |
| | | CTTCCAGAGC CCATATCAAT GCCTGTGTCA |
| | | GAGCCCTGGG GAGGAACTGC TCAGTTAGGA |
| | | CCCAGAGGGA ACCATGGAAG CCCCAGCTCA |
| | | GCTTCTCTTC CTCCTGCTAC TCTGGCTCCC |
| | | AGGTGAGGGG AACATGAGGT GGTTTTGCAC |
| | | ATTAGTGAAA ACTCTTGCCA CCTCTGCTCA |
| | | GCAAGAAATA TAATTAAAAT TCAAAGTATA |
| | | TCAACAATTT TGGCTCTACT CAAAGACAGT |
| | | TGGTTTGATC TTGATTACAT GAGTGCATTT |
| | | CTGTTTTATT TCCAATTTCA GATACCACCG |
| | | GAGAAATTGT GTTGACACAG TCTCCAGCCA |
| | | CCCTGTCTTT GTCTCCAGGG GAAAGAGCCA |
| | | CCCTCTCCTG CAGGGCCAGT CAGAGTGTTA |
| | | GCAGCTACTT AGCCTGGTAC CAACAGAAAC |
| | | CTGGCCAGGC TCCCAGGCTC CTCATCTATG |
| | | ATGCATCCAA CAGGGCCACT GGCATCCCAG |
| | | CCAGGTTCAG TGGCAGTGGG TCTGGGACAG |
| | | ACTTCACTCT CACCATCAGC AGCCTAGAGC |
| | | CTGAAGATTT TGCAGTTTAT TACTGTCAGC |
| | | AGCGTAGCAA CTGGCCTCCC ACAGTGATTC |
| | | CACATGAAAC AAAAACCCCA ACAAGACCAT |
| | | CAGTGTTTAC TAGATTATTA TACCAGCTGC |
| | | TTCCTTTACA GACAGCTAGT GGGGTGGCCA |
| | | CTCAGTGTTA GCATCTCAGC TCTATTTGGC |
| | | CATTTTGGAG TTCAAGT |
| 163 | Parental CEA binder VH | see Table 20 |
| 164 | Parental CEA binder VL | see Table 20 |
| 165 | nucleotide sequence of DP47 | see Table 21 |
| | Fc knob monomeric 4-1BBL | |
| | (71-248) | |
| 166 | nucleotide sequence of DP47 | see Table 21 |
| | light chain | |
| 167 | DP47 Fc knob monomeric | see Table 21 |
| | 4-1BBL (71-248) | |
| 168 | DP47 light chain | see Table 21 |
| 169 | nucleotide sequence of DP47 | see Table 22 |
| | Fc knob dimeric 4-1BBL | |
| | (71-248) | |
| 170 | DP47 Fc knob dimeric | see Table 22 |
| | 4-1BBL (71-248) | |
| 171 | nucleotide sequence of anti- | see Table 23 |
| | FAP(4B9) Fc hole dimeric | |
| | 4-1BB ligand (71-248) | |
| 172 | nucleotide sequence of Fc | see Table 23 |
| | knob monomeric 4-1BBL | |
| | (71-248) | |
| 173 | anti-FAP(4B9) Fc hole | see Table 23 |
| | dimeric 4-1BB ligand | |
| | (71-248) | |
| 174 | Fc knob monomeric 4-1BBL | see Table 23 |
| | (71-248) | |
| 175 | nucleotide sequence of anti- | see Table 24 |
| | FAP(4B9) Fc hole | |
| | monomeric 4-1BBL (71-248) | |
| 176 | nucleotide sequence of Fc | see Table 24 |
| | knob dimeric 4-1BB ligand | |
| | (71-248) | |
| 177 | anti-FAP(4B9) Fc hole | see Table 24 |
| | monomeric 4-1BBL (71-248) | |
| 178 | Fc knob dimeric 4-1BB | see Table 24 |
| | ligand (71-248) | |
| 179 | nucleotide sequence of anti- | see Table 25 |
| | FAP(28H1) Fc hole dimeric | |
| | 4-1BB ligand (71-248) | |
| 180 | anti-FAP(28H1) Fc hole | see Table 25 |
| | dimeric 4-1BB ligand | |
| | (71-248) | |
| 181 | nucleotide sequence of anti- | see Table 26 |
| | FAP(28H1) Fc hole | |
| | monomeric 4-1BBL (71-248) | |
| 182 | anti-FAP(28H1) Fc hole | see Table 26 |
| | monomeric 4-1BBL (71-248) | |
| 183 | nucleotide sequence of anti- | see Table 27 |
| | CD19(8B8-018) Fc hole | |
| | dimeric 4-1BB ligand | |
| | (71-248) chain | |
| 184 | anti-CD19(8B8-018) Fc hole | see Table 27 |
| | dimeric 4-1BB (71-248) | |
| | ligand chain | |
| 185 | nucleotide sequence of anti- | see Table 28 |
| | CD19(8B8-018) Fc hole | |
| | monomeric 4-1BBL (71-248) | |
| | chain | |
| 186 | anti-CD19(8B8-018) Fc hole | see Table 28 |
| | monomeric 4-1BBL (71-248) | |
| | chain | |
| 187 | nucleotide sequence of anti- | see Table 29 |
| | CD19(8B8-2B11) Fc hole | |
| | dimeric 4-1BB ligand | |
| | (71-248) chain | |
| 188 | anti-CD19(8B8-2B11) Fc | see Table 29 |
| | hole dimeric 4-1BB ligand | |
| | (71-248) chain | |
| 189 | nucleotide sequence of anti- | see Table 30 |
| | CD19(8B8-2B11) Fc hole | |
| | monomeric 4-1BB ligand | |
| | (71-248) chain | |
| 190 | anti-CD19(8B8-2B11) Fc | see Table 30 |
| | hole monomeric 4-1BB | |
| | ligand (71-248) chain | |
| 191 | nucleotide sequence of anti- | see Table 31 |
| | CENT84.66-LCHA) Fc hole | |
| | dimeric 4-1BB ligand- | |
| | (71-248) chain | |
| 192 | anti-CEA(T84.66-LCHA) Fc | see Table 31 |
| | hole dimeric 4-1BB ligand | |
| | (71-248) chain | |
| 193 | nucleotide sequence of anti- | see Table 32 |
| | CEA(T84.66-LCHA) Fc hole | |
| | monomeric 4-1BBL (71-248) | |
| | chain | |
| 194 | anti-CEA(T84.66-LCHA) Fc | see Table 32 |
| | hole monomeric 4-1BBL | |
| | (71-248) chain | |
| 195 | nucleotide sequence of DP47 | see Table 33 |
| | Fc hole dimeric 4-1BB ligand | |
| | (71-248) chain | |
| 196 | DP47 Fc hole dimeric 4-1BB | see Table 33 |
| | ligand (71-248) chain | |
| 197 | nucleotide sequence of DP47 | see Table 34 |
| | Fc hole monomeric 4-1BBL | |
| | (71-248) chain | |
| 198 | DP47 Fc hole monomeric | see Table 34 |
| | 4-1BBL (71-248) chain | |
| 199 | nucleotide sequence of | see Table 35 |
| | dimeric 4-1BB ligand | |
| | (71-248)-CL* Fc knob chain | |
| 200 | nucleotide sequence of | see Table 35 |
| | monomeric 4-1BBL | |
| | (71-248)-CH1* | |
| 201 | nucleotide sequence of DP47 | see Table 35 |
| | Fc hole chain | |
| 202 | dimeric 4-1BB ligand | see Table 35 |
| | (71-248)-CL* Fc knob chain | |
| 203 | monomeric 4-1BBL | see Table 35 |
| | (71-248)-CH1* | |
| 204 | DP47 Fc hole chain | see Table 35 |
| 205 | nucleotide sequence of DP47 | see Table 38 |
| | heavy chain (huIgG1 | |
| | PGLALA) | |
| 206 | DP47 heavy chain (huIgG1 | see Table 38 |
| | PGLALA) | |
| 207 | nucleotide sequence of | see Table 40 |
| | human 4-1BB His | |
| 208 | human 4-1BB His | see Table 40 |
| 209 | nucleotide sequence of | see Table 44 |
| | FAP(4B9) Fc hole dimeric | |
| | 4-1BB ligand (71-254) chain | |
| 210 | nucleotide sequence of DP47 | see Table 44 |
| | Fc knob monomeric 4-1BB | |
| | (71-254) ligand | |
| 211 | nucleotide sequence of DP47 | see Table 44 |
| | (VL-CH1) light chain | |
| 212 | anti-FAP(4B9) Fc hole | see Table 44 |
| | dimeric 4-1BB ligand | |
| | (71-254) chain | |
| 213 | DP47 Fc knob monomeric | see Table 44 |
| | 4-1BB (71-254) ligand | |
| 214 | DP47 (VL-CH1) light chain | see Table 44 |
| 215 | nucleotide sequence of anti- | see Table 45 |
| | FAP(4B9) Fc hole monomeric | |
| | 4-1BBL (71-254) chain | |
| 216 | nucleotide sequence of DP47 | see Table 45 |
| | (VH-CL) Fc knob dimeric | |
| | 4-1BB ligand (71-254) chain | |
| 217 | anti-FAP(4B9) Fc hole | see Table 45 |
| | monomeric 4-1BBL (71-254) | |
| | chain | |
| 218 | DP47 (VH-CL) Fc knob dimeric | see Table 45 |
| | 4-1BB ligand (71-254) chain | |
| 219 | nucleotide sequence of | see Table 45 |
| | DP47(VH-CL) Fc hole dimeric | |
| | 4-1BB ligand (71-254) chain | |
| 220 | nucleotide sequence of FAP | see Table 46 |
| | (4B9) Fc knob monomeric | |
| | 4-1BB (71-254) ligand | |
| 221 | DP47(VH-CL) Fc hole dimeric | see Table 46 |
| | 4-1BB ligand (71-254) chain | |
| 222 | FAP (4B9) Fc knob monomeric | see Table 46 |
| | 4-1BB (71-254) ligand | |
| 223 | nucleotide sequence of DP47 | see Table 47 |
| | (VH-CL) Fc hole monomeric | |
| | 4-1BBL (71-254) chain | |
| 224 | nucleotide sequence of FAP | see Table 47 |
| | (4B9) Fc knob dimeric 4-1BB | |
| | ligand (71-254) chain | |
| 225 | DP47 (VH-CL) Fc hole | see Table 47 |
| | monomeric 4-1BBL (71-254) | |
| | chain | |
| 226 | FAP (4B9) Fc knob dimeric | see Table 47 |
| | 4-1BB ligand (71-254) chain | |
| 227 | nucleotide sequence of DP47 | see Table 48 |
| | (VHCL) Fc knob monomeric | |
| | 4-1BB (71-248) ligand | |
| 228 | DP47 (VH-CL) Fc knob | see Table 48 |
| | monomeric 4-1BB (71-248) | |
| | ligand | |
| 229 | nucleotide sequence of DP47 | see Table 49 |
| | (VH-CL) Fc knob dimeric 4- | |
| | 1BB ligand (71-248) chain | |
| 230 | DP47 (VH-CL) Fc knob dimeric | see Table 49 |
| | 4-1BB ligand (71-248) chain | |
| 231 | nucleotide sequence of | see Table 50 |
| | DP47(VH-CL) Fc hole dimeric | |
| | 4-1BB ligand (71-248) chain | |
| 232 | DP47(VH-CL) Fc hole dimeric | see Table 50 |
| | 4-1BB ligand (71-248) chain | |
| 233 | nucleotide sequence of DP47 | see Table 51 |
| | (VH-CL) Fc hole monomeric | |
| | 4-1BBL (71-248) chain | |
| 234 | DP47 (VH-CL) Fc hole | see Table 51 |
| | monomeric 4-1BBL (71-248) | |
| | chain | |
| 235 | nucleotide sequence of anti- | see Table 36 |
| | FAP (4B9) Fc hole chain | |
| 236 | anti-FAP (4B9) Fc hole chain | see Table 36 |
| 237 | Nucleotide sequence of anti- | see Table 37a |
| | CD19(8B8-018) Fc hole chain | |
| 238 | anti-CD19(8B8-018) Fc hole | see Table 37a |
| | chain | |
| 239 | nucleotide sequence of | see Table 37b |
| | dimeric hu 4-1BBL | |
| | (71-254)-CL* Fc knob chain | |
| 240 | nucleotide sequence of | see Table 37b |
| | monomeric hu 4-1BBL | |
| | (71-254)-CH1* | |
| 241 | nucleotide sequence of anti- | see Table 37b |
| | CD19(8B8-2B11) Fc hole | |
| | chain | |
| 242 | Dimeric hu 4-1BBL | see Table 37b |
| | (71-254)-CL* Fc knob chain | |
| 243 | Monomeric hu 4-1BBL | see Table 37b |
| | (71-254)-CH1* | |
| 244 | CD19(8B8-2B11) Fc hole chain | see Table 37b |
| 245 | avi tag | GLNDIFEAQKIEWHE |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the EU numbering systems according to Kabat (Kabat, E. A., et al., Sequences of Proteins of Immunological Interest, 5th ed., Public Health Service, National Institutes of Health, Bethesda, Md. (1991)) as defined above.

The following numbered paragraphs (paras) describe aspects of the present invention:
1. A TNF family ligand trimer-containing antigen binding molecule comprising
   (a) a Fab domain capable of specific binding to a target cell antigen,
   (b) a Fc domain composed of a first and a second subunit capable of stable association, and
   (c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain.
2. The TNF family ligand trimer-containing antigen binding molecule of para 1, wherein the TNF ligand family member costimulates human T-cell activation.
3. The TNF family ligand trimer-containing antigen binding molecule of paras 1 or 2, wherein the TNF ligand family member is selected from 4-1BBL and OX40L.
4. The TNF family ligand trimer-containing antigen binding molecule of any one of paras 1 to 3, wherein the TNF ligand family member is 4-1BBL.
5. The TNF family ligand trimer-containing antigen binding molecule of any one of paras 1 to 4, wherein the ectodomain of a TNF ligand family member comprises the amino acid sequence selected from the group consisting of SEQ ID NO:1, SEQ ID NO: 2, SEQ ID NO:3, SEQ ID NO:4, SEQ ID NO:5, SEQ ID NO:6, SEQ ID NO:7 and SEQ ID NO:8, particularly the amino acid sequence of SEQ ID NO:1 or SEQ ID NO:5.
6. The TNF family ligand trimer-containing antigen binding molecule of any one of paras 1 to 5, wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5.
7. The TNF family ligand trimer-containing antigen binding molecule of any one of paras 1 to 3, wherein the TNF ligand family member is OX40L.
8. The TNF family ligand trimer-containing antigen binding molecule of any one of paras 1 to 3 or 7, wherein the ectodomain of a TNF ligand family member comprises the amino acid sequence selected from the group consisting of SEQ ID NO:11 and SEQ ID NO:12.
9. The TNF family ligand trimer-containing antigen binding molecule of any one of paras 1 to 8, wherein TNF family ligand trimer-containing antigen binding molecule is monovalent for the binding to the target cell antigen.
10. The TNF family ligand trimer-containing antigen binding molecule of any one of paras 1 to 9, wherein the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.
11. The TNF family ligand trimer-containing antigen binding molecule of any one of paras 1 to 10, wherein the target cell antigen is Fibroblast Activation Protein (FAP).
12. The TNF family ligand trimer-containing antigen binding molecule of any one of paras 1 to 11, wherein the Fab domain capable of specific binding to FAP comprises
   (a) a VH domain comprising (i) CDR-H1 comprising the amino acid sequence of SEQ ID NO:13, (ii) CDR-H2 comprising the amino acid sequence of SEQ ID NO:14 and (iii) CDR-H3 comprising the amino acid sequence of SEQ ID NO:15, and a VL domain comprising (iv) CDR-L1 comprising the amino acid sequence of SEQ ID NO:16, (v) CDR-L2 comprising the amino acid sequence of SEQ ID NO:17 and (vi) CDR-L3 comprising the amino acid sequence of SEQ ID NO:18 or
   (b) a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.
13. The TNF family ligand trimer-containing antigen binding molecule of any one of paras 1 to 12, wherein the Fab domain capable of specific binding to FAP comprises a variable heavy chain comprising an amino acid sequence of SEQ ID NO:25 and a variable light chain comprising an amino acid sequence of SEQ ID NO:26 or wherein the Fab domain capable of specific binding to FAP comprises a variable heavy chain comprising an amino acid sequence of SEQ ID NO:27 and a variable light chain comprising an amino acid sequence of SEQ ID NO:28.
14. The TNF family ligand trimer-containing antigen binding molecule of any one of paras 1 to 10, wherein the target cell antigen is CD19.
15. The TNF family ligand trimer-containing antigen binding molecule of any one of paras 1 to 10 or 14, wherein Fab domain capable of specific binding to CD19 comprises
   (a) a VH domain comprising (i) CDR-H1 comprising the amino acid sequence of SEQ ID NO:29, (ii) CDR-H2 comprising the amino acid sequence of SEQ ID NO:30 and (iii) CDR-H3 comprising the amino acid sequence of SEQ ID NO:31, and a VL domain comprising (iv) CDR-L1 comprising the amino acid sequence of SEQ ID NO:32, (v) CDR-L2 comprising the amino acid sequence of SEQ ID NO:33 and (vi) CDR-L3 comprising the amino acid sequence of SEQ ID NO:34 or
   (b) a VH domain comprising (i) CDR-H1 comprising the amino acid sequence of SEQ ID NO:35, (ii) CDR-H2 comprising the amino acid sequence of SEQ ID NO:36 and (iii) CDR-H3 comprising the amino acid sequence of SEQ ID NO:37, and a VL domain comprising (iv) CDR-L1 comprising the amino acid sequence of SEQ ID NO:38, (v) CDR-L2 comprising the amino acid sequence of SEQ ID NO:39 and (vi) CDR-L3 comprising the amino acid sequence of SEQ ID NO:40.
16. The TNF family ligand trimer-containing antigen binding molecule of any one of paras 1 to 10, 14 or 15, wherein the Fab domain capable of specific binding to CD19 comprises a variable heavy chain comprising an amino acid sequence of SEQ ID NO:41 and a variable light chain comprising an amino acid sequence of SEQ ID NO:42 or wherein the Fab domain capable of specific binding to FAP comprises a variable heavy chain comprising an amino acid sequence of SEQ ID NO:43 and a variable light chain comprising an amino acid sequence of SEQ ID NO:44.
17. The TNF family ligand trimer-containing antigen binding molecule of any one of paras 1 to 10, wherein the target cell antigen is CEA.
18. The TNF family ligand trimer-containing antigen binding molecule of any one of paras 1 to 10 or 17, wherein Fab domain capable of specific binding to CEA comprises a VH domain comprising (i) CDR-H1 comprising the amino acid sequence of SEQ ID NO:45, (ii) CDR-H2 comprising the amino acid sequence of SEQ ID NO:46 and (iii) CDR-H3 comprising the amino acid sequence of SEQ ID NO:47, and a VL domain comprising (iv) CDR-L1 comprising the amino acid sequence of SEQ ID NO:48, (v) CDR-L2 comprising the amino acid sequence of SEQ ID NO:49 and (vi) CDR-L3 comprising the amino acid sequence of SEQ ID NO:50.
19. The TNF family ligand trimer-containing antigen binding molecule of any one of paras 1 to 10, 17 or 18, wherein the Fab domain capable of specific binding to CEA comprises a variable heavy chain comprising an amino acid sequence of SEQ ID NO:51 and a variable light chain comprising an amino acid sequence of SEQ ID NO:52.
20. The TNF family ligand trimer-containing antigen binding molecule of any one of paras 1 to 19, wherein the Fc domain is an IgG, particularly an IgG1 Fc domain or an IgG4 Fc domain.
21. The TNF family ligand trimer-containing antigen binding molecule of any one of paras 1 to 20, wherein the Fc domain is an IgG1 Fc domain comprising the amino acid substitutions at positions 234 and 235 (EU numbering) and/or 329 (EU numbering).
22. The TNF family ligand trimer-containing antigen binding molecule of any one of paras 1 to 21, wherein the first subunit of the Fc domain comprises the amino acid substitutions S354C and T366W (numbering according to Kabat EU index) and the second subunit of the Fc domain comprises the amino acid substitutions Y349C, T366S, L368A and Y407V (numbering according to Kabat EU index).
23. The TNF family ligand trimer-containing antigen binding molecule of any one of paras 1 to 22, wherein the first polypeptide comprising two ectodomains of a TNF ligand family member or fragments thereof that are connected to each other by a peptide linker is fused at its N-terminus to the C-terminus of the second subunit of the Fc domain and the second polypeptide comprising one ectodomain of said TNF ligand family member or a fragment thereof is fused at its N-terminus to the C-terminus of the first subunit of the Fc domain.
24. The TNF family ligand trimer-containing antigen binding molecule of any one of paras 1 to 22, wherein the first polypeptide comprising two ectodomains of a TNF ligand family member or fragments thereof that are connected to each other by a peptide linker is fused at its N-terminus to the C-terminus of the first subunit of the Fc domain and the second polypeptide comprising one ectodomain of said TNF ligand family member or a fragment thereof is fused at its N-terminus to the C-terminus of the second subunit of the Fc domain.
25. The TNF family ligand trimer-containing antigen binding molecule of any one of paras 1 to 24, wherein Fab domain capable of specific binding to a target cell antigen is is fused at the C-terminus to the N-terminus of the first subunit of the Fc domain.
26. The TNF family ligand trimer-containing antigen binding molecule of any one of paras 1 to 26 further comprising
   (d) a Fab domain that is not capable of specific binding to a target cell antigen.
27. An isolated polynucleotide encoding the TNF family ligand trimer-containing antigen binding molecule of any one of paras 1 to 16.
28. A vector, particularly an expression vector, comprising the isolated polynucleotide of para 27.
29. A host cell comprising the isolated polynucleotide of para 27 or the vector of para 28.
30. A method for producing the TNF family ligand trimer-containing antigen binding molecule of any one of paras 1 to 26, comprising the steps of
   (i) culturing the host cell of para 29 under conditions suitable for expression of the antigen binding molecule, and
   (ii) recovering the antigen binding molecule.
31. A pharmaceutical composition comprising the TNF family ligand trimer-containing antigen binding molecule of any one of paras 1 to 26 and at least one pharmaceutically acceptable excipient.
32. The TNF family ligand trimer-containing antigen binding molecule of any one of paras 1 to 26, or the pharmaceutical composition of para 31, for use as a medicament.
33. The TNF family ligand trimer-containing antigen binding molecule of any one of paras 1 to 26, or the pharmaceutical composition of para 31, for use in the treatment of cancer.
34. Use of the TNF family ligand trimer-containing antigen binding molecule of any one of paras 1 to 26 for the manufacture of a medicament for the treatment of cancer.
35. A method of treating a disease in an individual, comprising administering to said individual a therapeutically effective amount of a composition comprising a TNF family ligand trimer-containing antigen binding molecule of any one of paras 1 to 26 in a pharmaceutically acceptable form.
36. The method of para 35, wherein said disease is cancer.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min. respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Preparation of Monovalent Targeted Human 4-1BB Ligand Trimer-Containing Fc Fusion Antigen Binding Molecules

### 1.1 Preparation of Monovalent FAP-Targeted 4-1BB Ligand Trimer-Containing Fc (Kih) Fusion Antigen Binding Molecule, Wherein the 4-1BB Ligands are C-Terminally Fused (Constructs 1.11, 2.11, 1.12 and 2.12) (FAP on the Knob Chain)

The DNA sequence encoding part of the ectodomain (amino acid 71-248) of human 4-1BB ligand was synthetized according to the P41273 sequence of Uniprot database (SEQ ID NO:75).

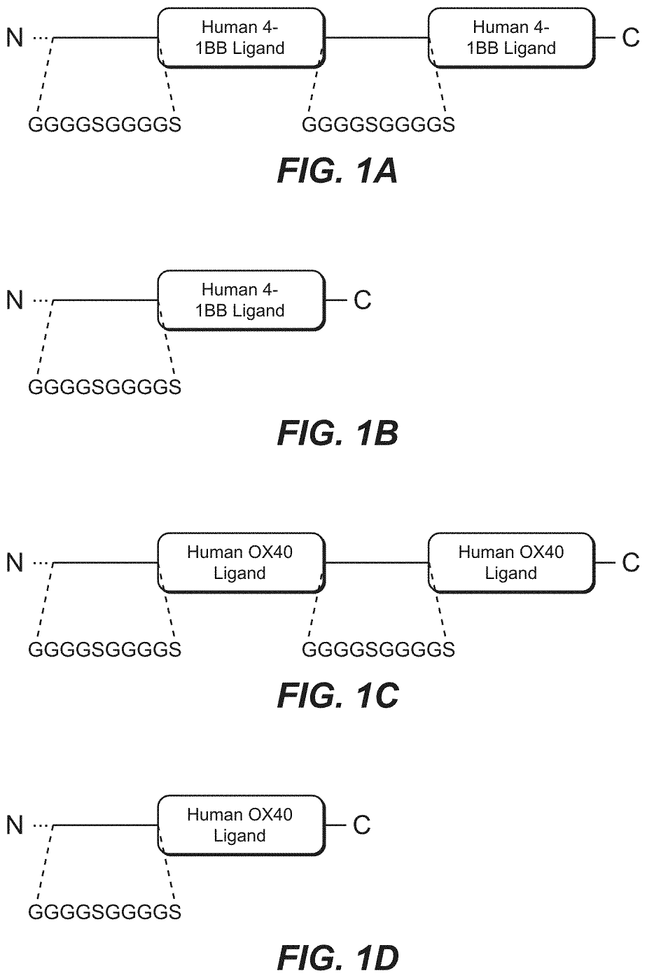

A polypeptide containing two ectodomains of 4-1BB ligand, separated by (G4S)2 linkers was subcloned in frame to the C-terminus of human IgG1 Fc hole or knob chain (Merchant, Zhu 1998), as depicted in FIG. 1a: human IgG Fc, (G4S)2 connector, human 4-1BB ligand, (G4S)2 connector, human 4-1BB ligand. A polypeptide containing one ectodomain of 4-1BB ligand was subcloned in frame to the C-terminus of human IgG1 Fc knob or hole chain as described in FIG. 1b: human IgG Fc, (G4S)2 connector, human 4-1BB ligand.

The variable region of heavy and light chain DNA sequences encoding a binder specific for fibroblast activation protein (FAP), clone 4B9 or clone 28H1, were subcloned in frame with either the constant heavy chain of the knob or the constant light chain of human IgG1. The generation and preparation of the FAP binders is described in WO 2012/020006 A2, which is incorporated herein by reference.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

For all constructs the knobs into holes heterodimerization technology was used as described in Carter, J Immunol Methods 248, 7-15 (2001). Combination of the huIgG1 Fc hole chain containing the Y349C/T366S/L368A/Y407V mutations in the CH3 domain, the anti-FAP huIgG1 knob chain containing the S354C/T366W mutations in the CH3 domain and the anti-FAP light chain allows generation of a heterodimer, which includes an assembled trimeric 4-1BB ligand and one FAP binding Fab (FIGS. 2A and 2B).

Table 1 shows the cDNA and amino acid sequences of the monovalent FAP(4B9)-targeted 4-1BB ligand (71-248) trimer-containing Fc (kih) fusion antigen binding molecule (Construct 2.11).

TABLE 1
Sequences of FAP(4B9)-targeted human 4-1BB ligand trimer
containing Fc (kih) fusion molecule Construct 2.11

| SEQ ID NO: | Description | Sequence |
|---|---|---|
| 101 | nucleotide sequence | GACAAAACTCACACATGCCCACCGTGCCCAGCACCTGA |
| | of Fc hole dimeric | AGCTGCAGGGGGACCGTCAGTCTTCCTCTTCCCCCCAA |
| | 4-1BB ligand | AACCCAAGGACACCCTCATGATCTCCCGGACCCCTGAG |
| | (71-248) chain | GTCACATGCGTGGTGGTGGACGTGAGCCACGAAGACCC |
| | | TGAGGTCAAGTTCAACTGGTACGTGGACGGCGTGGAGG |
| | | TGCATAATGCCAAGACAAAGCCGCGGGAGGAGCAGTAC |
| | | AACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCGGCGCCCCCATCGAGAAA |
| | | ACCATCTCCAAAGCCAAAGGGCAGCCCCGAGAACCACA |
| | | GGTGTGCACCCTGCCCCCATCCCGGGATGAGCTGACCA |
| | | AGAACCAGGTCAGCCTCTCGTGCGCAGTCAAAGGCTTC |
| | | TATCCCAGCGACATCGCCGTGGAGTGGGAGAGCAATGG |
| | | GCAGCCGGAGAACAACTACAAGACCACGCCTCCCGTGC |
| | | TGGACTCCGACGGCTCCTTCTTCCTCGTGAGCAAGCTC |
| | | ACCGTGGACAAGAGCAGGTGGCAGCAGGGGAACGTCTT |
| | | CTCATGCTCCGTGATGCATGAGGCTCTGCACAACCACT |
| | | ACACGCAGAAGAGCCTCTCCCTGTCTCCGGGTGGAGGC |
| | | GGCGGAAGCGGAGGAGGAGGATCCAGAGAGGGCCCTGA |
| | | GCTGAGCCCTGATGATCCTGCCGGACTGCTGGACCTGC |
| | | GGCAGGGAATGTTTGCCCAGCTGGTGGCCCAGAACGTG |
| | | CTGCTGATCGATGGCCCCCTGTCCTGGTACAGCGATCC |
| | | TGGACTGGCTGGCGTGTCACTGACAGGCGGCCTGAGCT |
| | | ACAAAGAGGACACCAAAGAACTGGTGGTGGCCAAGGCC |
| | | GGCGTGTACTACGTGTTCTTTCAGCTGGAACTGCGGAG |
| | | AGTGGTGGCCGGCGAAGGATCTGGCTCTGTGTCTCTGG |
| | | CCCTGCATCTGCAGCCTCTGAGATCTGCTGCTGGCGCC |
| | | GCTGCTCTGGCACTGACAGTGGATCTGCCTCCTGCCAG |
| | | CAGCGAGGCCCGGAATAGCGCATTTGGGTTTCAAGGCA |
| | | GGCTGCTGCACCTGTCTGCCGGCCAGAGGCTGGGAGTG |
| | | CATCTGCACACAGAGGCCAGGGCTAGACACGCCTGGCA |
| | | GCTGACACAGGGCGCTACAGTGCTGGGCCTGTTCAGAG |
| | | TGACCCCCGAGATTCCAGCAGGCCTGGGAGGCGGCGGA |
| | | TCTGGCGGCGGAGGATCTAGAGAAGGACCCGAGCTGTC |
| | | CCCCGACGATCCCGCTGGGCTGCTGGATCTGAGACAGG |
| | | GCATGTTCGCTCAGCTGGTGGCTCAGAATGTGCTGCTG |
| | | ATTGACGGACCTCTGAGCTGGTACTCCGACCCAGGGCT |
| | | GGCAGGGGTGTCCCTGACTGGGGGACTGTCCTACAAAG |
| | | AAGATACAAAAGAACTGGTGGTGGCTAAAGCTGGGGTG |
| | | TACTATGTGTTTTTTCAGCTGGAACTGAGGCGGGTGGT |
| | | GGCTGGGGAGGGCTCAGGATCTGTGTCCCTGGCTCTGC |
| | | ATCTGCAGCCACTGCGCTCTGCAGCAGGGGCTGCAGCA |
| | | CTGGCCCTGACTGTGGACCTGCCCCCAGCTTCTTCCGA |
| | | GGCCAGAAACAGCGCCTTCGGGTTCCAAGGACGCCTGC |
| | | TGCATCTGAGCGCCGGACAGCGCCTGGGAGTGCATCTG |
| | | CATACTGAAGCCAGAGCCCGGCATGCTTGGCAGCTGAC |
| | | TCAGGGGGCAACTGTGCTGGGACTGTTTCGCGTGACAC |
| | | CTGAGATCCCAGCCGGGCTC |
| 102 | nucleotide sequence | GAGGTGCAGCTGCTCGAAAGCGGCGGAGGACTGGTGCA |
| | of anti-FAP(4B9) Fc | GCCTGGCGGCAGCCTGAGACTGTCTTGCGCCGCCAGCG |
| | knob monomeric | GCTTCACCTTCAGCAGCTACGCCATGAGCTGGGTCCGC |
| | 4-1BB (71-248) | CAGGCCCCTGGCAAGGGACTGGAATGGGTGTCCGCCAT |
| | ligand | CATCGGCTCTGGCGCCAGCACCTACTACGCCGACAGCG |
| | | TGAAGGGCCGGTTCACCATCAGCCGGGACAACAGCAAG |
| | | AACACCCTGTACCTGCAGATGAACAGCCTGCGGGCCGA |
| | | GGACACCGCCGTGTACTACTGCGCCAAGGGATGGTTCG |
| | | GCGGCTTCAACTACTGGGGACAGGGCACCCTGGTCACA |
| | | GTGTCCAGCGCTAGCACCAAGGGCCCATCGGTCTTCCC |
| | | CCTGGCACCCTCCTCCAAGAGCACCTCTGGGGGCACAG |
| | | CGGCCCTGGGCTGCCTGGTCAAGGACTACTTCCCCGAA |
| | | CCGGTGACGGTGTCGTGGAACTCAGGCGCCCTGACCAG |
| | | CGGCGTGCACACCTTCCCGGCTGTCCTACAGTCCTCAG |
| | | GACTCTACTCCCTCAGCAGCGTGGTGACCGTGCCCTCC |
| | | AGCAGCTTGGGCACCCAGACCTACATCTGCAACGTGAA |
| | | TCACAAGCCCAGCAACACCAAGGTGGACAAGAAAGTTG |
| | | AGCCCAAATCTTGTGACAAAACTCACACATGCCCACCG |
| | | TGCCCAGCACCTGAAGCTGCAGGGGGACCGTCAGTCTT |
| | | CCTCTTCCCCCCAAAACCCAAGGACACCCTCATGATCT |
| | | CCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAA |
| | | GGAGTACAAGTGCAAGGTCTCCAACAAAGCCCTCGGCG |
| | | CCCCCATCGAGAAAACCATCTCCAAAGCCAAAGGGCAG |
| | | CCCCGAGAACCACAGGTGTACACCCTGCCCCCCTGCAG |
| | | AGATGAGCTGACCAAGAACCAGGTGTCCCTGTGGTGTC |
| | | TGGTCAAGGGCTTCTACCCCAGCGATATCGCCGTGGAG |
| | | TGGGAGAGCAACGGCCAGCCTGAGAACAACTACAAGAC |
| | | CACCCCCCCTGTGCTGGACAGCGACGGCAGCTTCTTCC |
| | | TGTACTCCAAACTGACCGTGGACAAGAGCCGGTGGCAG |
| | | CAGGGCAACGTGTTCAGCTGCAGCGTGATGCACGAGGC |
| | | CCTGCACAACCACTACACCCAGAAGTCCCTGAGCCTGA |
| | | GCCCCGGCGGAGGCGGCGGAAGCGGAGGAGGAGGATCC |
| | | AGAGAGGGCCCTGAGCTGAGCCCTGATGATCCTGCCGG |
| | | ACTGCTGGACCTGCGGCAGGGAATGTTTGCCCAGCTGG |
| | | TGGCCCAGAACGTGCTGCTGATCGATGGCCCCCTGTCC |
| | | TGGTACAGCGATCCTGGACTGGCTGGCGTGTCACTGAC |
| | | AGGCGGCCTGAGCTACAAAGAGGACACCAAAGAACTGG |
| | | TGGTGGCCAAGGCCGGCGTGTACTACGTGTTCTTTCAG |
| | | CTGGAACTGCGGAGAGTGGTGGCCGGCGAAGGATCTGG |
| | | CTCTGTGTCTCTGGCCCTGCATCTGCAGCCTCTGAGAT |
| | | CTGCTGCTGGCGCCGCTGCTCTGGCACTGACAGTGGAT |
| | | CTGCCTCCTGCCAGCAGCGAGGCCCGGAATAGCGCATT |
| | | TGGGTTTCAAGGCAGGCTGCTGCACCTGTCTGCCGGCC |
| | | AGAGGCTGGGAGTGCATCTGCACACAGAGGCCAGGGCT |
| | | AGACACGCCTGGCAGCTGACACAGGGCGCTACAGTGCT |
| | | GGGCCTGTTCAGAGTGACCCCCGAGATTCCTGCCGGGC |
| | | TC |
| 103 | nucleotidesequence | GAGATCGTGCTGACCCAGTCCCCCGGCACCCTGTCTCT |
| | of anti-FAP(4B9) | GAGCCCTGGCGAGAGAGCCACCCTGTCCTGCAGAGCCT |
| | light chain | CCCAGTCCGTGACCTCCTCCTACCTCGCCTGGTATCAG |
| | | CAGAAGCCCGGCCAGGCCCCTCGGCTGCTGATCAACGT |
| | | GGGCAGTCGGAGAGCCACCGGCATCCCTGACCGGTTCT |
| | | CCGGCTCTGGCTCCGGCACCGACTTCACCCTGACCATC |
| | | TCCCGGCTGGAACCCGAGGACTTCGCCGTGTACTACTG |
| | | CCAGCAGGGCATCATGCTGCCCCCCACCTTTGGCCAGG |
| | | GCACCAAGGTGGAAATCAAGCGTACGGTGGCTGCACCA |
| | | TCTGTCTTCATCTTCCCGCCATCTGATGAGCAGTTGAA |
| | | ATCTGGAACTGCCTCTGTTGTGTGCCTGCTGAATAACT |
| | | TCTATCCCAGAGAGGCCAAAGTACAGTGGAAGGTGGAT |
| | | AACGCCCTCCAATCGGGTAACTCCCAGGAGAGTGTCAC |
| | | AGAGCAGGACAGCAAGGACAGCACCTACAGCCTCAGCA |
| | | GCACCCTGACGCTGAGCAAAGCAGACTACGAGAAACAC |
| | | AAAGTCTACGCCTGCGAAGTCACCCATCAGGGCCTGAG |
| | | CTCGCCCGTCACAAAGAGCTTCAACAGGGGAGAGTGT |
| 104 | Fc hole dimeric | DKTHTCPPCPAPEAAGGPSVFLFPPKPKDTLMISRTPE |
| | 4-1BB (71-248) | VTCVVVDVSHEDPEVKFNWYVDGVEVHNAKTKPREEQY |
| | ligand chain | NSTYRVVSVLTVLHQDWLNGKEYKCKVSNKALGAPIEK |
| | | TISKAKGQPREPQVCTLPPSRDELTKNQVSLSCAVKGF |
| | | YPSDIAVEWESNGQPENNYKTTPPVLDSDGSFFLVSKL |
| | | TVDKSRWQQGNVFSCSVMHEALHNHYTQKSLSLSPGGG |
| | | GGSGGGGSREGPELSPDDPAGLLDLRQGMFAQLVAQNV |
| | | LLIDGPLSWYSDPGLAGVSLTGGLSYKEDTKELVVAKA |
| | | GVYYVFFQLELRRVVAGEGSGSVSLALHLQPLRSAAGA |
| | | AALALTVDLPPASSEARNSAFGFQGRLLHLSAGQRLGV |
| | | HLHTEARARHAWQLTQGATVLGLFRVTPEIPAGLGGGG |
| | | SGGGGSREGPELSPDDPAGLLDLRQGMFAQLVAQNVLL |
| | | IDGPLSWYSDPGLAGVSLTGGLSYKEDTKELVVAKAGV |
| | | YYVFFQLELRRVVAGEGSGSVSLALHLQPLRSAAGAAA |
| | | LALTVDLPPASSEARNSAFGFQGRLLHLSAGQRLGVHL |
| | | HTEARARHAWQLTQGATVLGLFRVTPEIPAGL |
| 105 | anti-FAP(4B9) Fc | EVQLLESGGGLVQPGGSLRLSCAASGFTFSSYAMSWVR |
| | | QAPGKGLEWVSAIIGSGASTYYADSVKGRFTISRDNSK |
| | | NTLYLQMNSLRAEDTAVYYCAKGWFGGFNYWGQGTLVT |
| | | VSSASTKGPSVFPLAPSSKSTSGGTAALGCLVKDYFPE |
| | | PVTVSWNSGALTSGVHTFPAVLQSSGLYSLSSVVTVPS |
| | | SSLGTQTYICNVNHKPSNTKVDKKVEPKSCDKTHTCPP |
| | | CPAPEAAGGPSVFLFPPKPKDTLMISRTPEVTCVVVDV |
| | | SHEDPEVKFNWYVDGVEVHNAKTKPREEQYNSTYRVVS |
| | | VLTVLHQDWLNGKEYKCKVSNKALGAPIEKTISKAKGQ |
| | | PREPQVYTLPPCRDELTKNQVSLWCLVKGFYPSDIAVE |
| | | WESNGQPENNYKTTPPVLDSDGSFFLYSKLTVDKSRWQ |
| | | QGNVFSCSVMHEALHNHYTQKSLSLSPGGGGGSGGGGS |
| | | REGPELSPDDPAGLLDLRQGMFAQLVAQNVLLIDGPLS |
| | | WYSDPGLAGVSLTGGLSYKEDTKELVVAKAGVYYVFFQ |
| | | LELRRVVAGEGSGSVSLALHLQPLRSAAGAAALALTVD |
| | | LPPASSEARNSAFGFQGRLLHLSAGQRLGVHLHTEARA |
| | | RHAWQLTQGATVLGLFRVTPEIPAGL |
| 106 | anti-FAP(4B9) | EIVLTQSPGTLSLSPGERATLSCRASQSVTSSYLAWYQ |
| | | QKPGQAPRLLINVGSRRATGIPDRFSGSGSGTDFTLTI |
| | | SRLEPEDFAVYYCQQGIMLPPTFGQGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |

Table 2 shows the cDNA and amino acid sequences of the monovalent FAP(4B9)-targeted 4-1BB ligand (71-248) trimer-containing Fc (kih) fusion antigen binding molecule (Construct 2.12).

TABLE 2
Sequences of FAP(4B9)-targeted human
4-1BB ligand trimer containing
Fc (kih) fusion molecule Construct 2.12

| SEQ ID | | |
|---|---|---|
| NO: | Description | Sequence |
| 107 | nucleotide | GACAAAACTCACACATGCCCACCGTGCCCAGCACCTGA |
| | sequence of Fc | AGCTGCAGGGGGACCGTCAGTCTTCCTCTTCCCCCCAAA |
| | hole monomeric | ACCCAAGGACACCCTCATGATCTCCCGGACCCCTGAGGT |
| | 4-1BBL (71-248) | CACATGCGTGGTGGTGGACGTGAGCCACGAAGACCCTG |
| | chain | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAGAGAGGGCCCT |
| | | GAGCTGAGCCCTGATGATCCTGCCGGACTGCTGGACCTG |
| | | CGGCAGGGAATGTTTGCCCAGCTGGTGGCCCAGAACGT |
| | | GCTGCTGATCGATGGCCCCCTGTCCTGGTACAGCGATCC |
| | | TGGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGATCTGCTGCTGGCGCCGCTG |
| | | CTCTGGCACTGACAGTGGATCTGCCTCCTGCCAGCAGCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCTGCCGGGCTC |
| 108 | nucleotide | GAGGTGCAGCTGCTCGAAAGCGGCGGAGGACTGGTGCA |
| | sequence of anti- | GCCTGGCGGCAGCCTGAGACTGTCTTGCGCCGCCAGCG |
| | FAP(4B9) Fc | GCTTCACCTTCAGCAGCTACGCCATGAGCTGGGTCCGCC |
| | knob dimeric 4- | AGGCCCCTGGCAAGGGACTGGAATGGGTGTCCGCCATC |
| | 1BB ligand (71- | ATCGGCTCTGGCGCCAGCACCTACTACGCCGACAGCGTG |
| | 248) | AAGGGCCGGTTCACCATCAGCCGGGACAACAGCAAGAA |
| | | CACCCTGTACCTGCAGATGAACAGCCTGCGGGCCGAGG |
| | | ACACCGCCGTGTACTACTGCGCCAAGGGATGGTTCGGC |
| | | GGCTTCAACTACTGGGGACAGGGCACCCTGGTCACAGT |
| | | GTCCAGCGCTAGCACCAAGGGCCCATCGGTCTTCCCCCT |
| | | GGCACCCTCCTCCAAGAGCACCTCTGGGGGCACAGCGG |
| | | CCCTGGGCTGCCTGGTCAAGGACTACTTCCCCGAACCGG |
| | | TGACGGTGTCGTGGAACTCAGGCGCCCTGACCAGCGGC |
| | | GTGCACACCTTCCCGGCTGTCCTACAGTCCTCAGGACTC |
| | | TACTCCCTCAGCAGCGTGGTGACCGTGCCCTCCAGCAGC |
| | | TTGGGCACCCAGACCTACATCTGCAACGTGAATCACAA |
| | | GCCCAGCAACACCAAGGTGGACAAGAAAGTTGAGCCCA |
| | | AATCTTGTGACAAAACTCACACATGCCCACCGTGCCCAG |
| | | CACCTGAAGCTGCAGGGGGACCGTCAGTCTTCCTCTTCC |
| | | CCCCAAAACCCAAGGACACCCTCATGATCTCCCGGACCC |
| | | CTGAGGTCACATGCGTGGTGGTGGACGTGAGCCACGAA |
| | | GACCCTGAGGTCAAGTTCAACTGGTACGTGGACGGCGT |
| | | GGAGGTGCATAATGCCAAGACAAAGCCGCGGGAGGAGC |
| | | AGTACAACAGCACGTACCGTGTGGTCAGCGTCCTCACCG |
| | | TCCTGCACCAGGACTGGCTGAATGGCAAGGAGTACAAG |
| | | TGCAAGGTCTCCAACAAAGCCCTCGGCGCCCCCATCGA |
| | | GAAAACCATCTCCAAAGCCAAAGGGCAGCCCCGAGAAC |
| | | CACAGGTGTACACCCTGCCCCCCTGCAGAGATGAGCTG |
| | | ACCAAGAACCAGGTGTCCCTGTGGTGTCTGGTCAAGGGC |
| | | TTCTACCCCAGCGATATCGCCGTGGAGTGGGAGAGCAA |
| | | CGGCCAGCCTGAGAACAACTACAAGACCACCCCCCCTG |
| | | TGCTGGACAGCGACGGCAGCTTCTTCCTGTACTCCAAAC |
| | | TGACCGTGGACAAGAGCCGGTGGCAGCAGGGCAACGTG |
| | | TTCAGCTGCAGCGTGATGCACGAGGCCCTGCACAACCA |
| | | CTACACCCAGAAGTCCCTGAGCCTGAGCCCCGGCAGAG |
| | | AGGGCCCTGAGCTGAGCCCTGATGATCCTGCCGGACTGC |
| | | TGGACCTGCGGCAGGGAATGTTTGCCCAGCTGGTGGCCC |
| | | AGAACGTGCTGCTGATCGATGGCCCCCTGTCCTGGTACA |
| | | GCGATCCTGGACTGGCTGGCGTGTCACTGACAGGCGGC |
| | | CTGAGCTACAAAGAGGACACCAAAGAACTGGTGGTGGC |
| | | CAAGGCCGGCGTGTACTACGTGTTCTTTCAGCTGGAACT |
| | | GCGGAGAGTGGTGGCCGGCGAAGGATCTGGCTCTGTGT |
| | | CTCTGGCCCTGCATCTGCAGCCTCTGAGATCTGCTGCTG |
| | | GCGCCGCTGCTCTGGCACTGACAGTGGATCTGCCTCCTG |
| | | CCAGCAGCGAGGCCCGGAATAGCGCATTTGGGTTTCAA |
| | | GGCAGGCTGCTGCACCTGTCTGCCGGCCAGAGGCTGGG |
| | | AGTGCATCTGCACACAGAGGCCAGGGCTAGACACGCCT |
| | | GGCAGCTGACACAGGGCGCTACAGTGCTGGGCCTGTTC |
| | | AGAGTGACCCCCGAGATTCCAGCAGGCCTGGGAGGCGG |
| | | CGGATCTGGCGGCGGAGGATCTAGAGAAGGACCCGAGC |
| | | TGTCCCCCGACGATCCCGCTGGGCTGCTGGATCTGAGAC |
| | | AGGGCATGTTCGCTCAGCTGGTGGCTCAGAATGTGCTGC |
| | | TGATTGACGGACCTCTGAGCTGGTACTCCGACCCAGGGC |
| | | TGGCAGGGGTGTCCCTGACTGGGGGACTGTCCTACAAA |
| | | GAAGATACAAAAGAACTGGTGGTGGCTAAAGCTGGGGT |
| | | GTACTATGTGTTTTTTCAGCTGGAACTGAGGCGGGTGGT |
| | | GGCTGGGGAGGGCTCAGGATCTGTGTCCCTGGCTCTGCA |
| | | TCTGCAGCCACTGCGCTCTGCAGCAGGGGCTGCAGCACT |
| | | GGCCCTGACTGTGGACCTGCCCCCAGCTTCTTCCGAGGC |
| | | CAGAAACAGCGCCTTCGGGTTCCAAGGACGCCTGCTGC |
| | | ATCTGAGCGCCGGACAGCGCCTGGGAGTGCATCTGCAT |
| | | ACTGAAGCCAGAGCCCGGCATGCTTGGCAGCTGACTCA |
| | | GGGGGCAACTGTGCTGGGACTGTTTCGCGTGACACCTGA |
| | | GATCCCAGCCGGGCTC |
| 103 | nucleotide | See Table 1 |
| | sequence of anti- | |
| | FAP(4B9) light | |
| | chain | |
| 109 | Fc hole | DKTHTCPPCPAPEAAGGPSVFLFPPKPKDTLMISRTPEVTC |
| | monomeric 4- | VVVDVSHEDPEVKFNWYVDGVEVHNAKTKPREEQYNSTY |
| | 1BB ligand (71- | RVVSVLTVLHQDWLNGKEYKCKVSNKALGAPIEKTISKAK |
| | 248) chain | GQPREPQVCTLPPSRDELTKNQVSLSCAVKGFYPSDIAVEW |
| | | ESNGQPENNYKTTPPVLDSDGSFFLVSKLTVDKSRWQQGN |
| | | VFSCSVMHEALHNHYTQKSLSLSPGREGPELSPDDPAGLL |
| | | DLRQGMFAQLVAQNVLLIDGPLSWYSDPGLAGVSLTGGLS |
| | | YKEDTKELVVAKAGVYYVFFQLELRRVVAGEGSGSVSLA |
| | | LHLQPLRSAAGAAALALTVDLPPASSEARNSAFGFQGRLL |
| | | HLSAGQRLGVHLHTEARARHAWQLTQGATVLGLFRVTPEI |
| | | PAGL |
| 110 | anti-FAP(4B9) Fc | EVQLLESGGGLVQPGGSLRLSCAASGFTFSSYAMSWVRQA |
| | knob dimeric 4- | PGKGLEWVSAIIGSGASTYYADSVKGRFTISRDNSKNTLYL |
| | 1BB ligand (71- | QMNSLRAEDTAVYYCAKGWFGGFNYWGQGTLVTVSSAS |
| | 248) | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGREGPELSPDDPAGLLDLRQGMFAQLVAQNVLLID |
| | | GPLSWYSDPGLAGVSLTGGLSYKEDTKELVVAKAGVYYV |
| | | FFQLELRRVVAGEGSGSVSLALHLQPLRSAAGAAALALTV |
| | | DLPPASSEARNSAFGFQGRLLHLSAGQRLGVHLHTEARAR |
| | | HAWQLTQGATVLGLFRVTPEIPAGLGGGGSGGGGSREGPE |
| | | LSPDDPAGLLDLRQGMFAQLVAQNVLLIDGPLSWYSDPGL |
| | | AGVSLTGGLSYKEDTKELVVAKAGVYYVFFQLELRRVVA |
| | | GEGSGSVSLALHLQPLRSAAGAAALALTVDLPPASSEARN |
| | | SAFGFQGRLLHLSAGQRLGVHLHTEARARHAWQLTQGAT |
| | | VLGLFRVTPEIPAGL |
| 106 | anti-FAP(4B9) | See Table 1 |
| | light chain | |

Table 3 shows the cDNA and amino acid sequences of the monovalent FAP(28H1)-targeted 4-1BB ligand trimer-containing Fc (kih) fusion antigen binding molecule (Construct 1.11).

TABLE 3
Sequences of FAP(28H1)-targeted human
4-1BB ligand trimer containing Fc (kih)
fusion molecule Construct 1.11

| SEQ ID | | |
|---|---|---|
| NO: | Description | Sequence |
| 101 | nucleotide | See Table 1 |
| | sequence of Fc | |
| | hole dimeric 4- | |
| | 1BB ligand (71- | |
| | 248) chain | |
| 111 | nucleotide | GAAGTGCAGCTGCTGGAATCCGGCGGAGGCCTGGTGCA |
| | sequence of anti- | GCCTGGCGGATCTCTGAGACTGTCCTGCGCCGCCTCCGG |
| | FAP(28H1) Fc | CTTCACCTTCTCCTCCCACGCCATGTCCTGGGTCCGACA |
| | knob monomeric | GGCTCCTGGCAAAGGCCTGGAATGGGTGTCCGCCATCTG |
| | 4-1BB ligand (71- | GGCCTCCGGCGAGCAGTACTACGCCGACTCTGTGAAGG |
| | 248) | GCCGGTTCACCATCTCCCGGGACAACTCCAAGAACACCC |
| | | TGTACCTGCAGATGAACTCCCTGCGGGCCGAGGACACC |
| | | GCCGTGTACTACTGTGCCAAGGGCTGGCTGGGCAACTTC |
| | | GACTACTGGGGACAGGGCACCCTGGTCACCGTGTCCAG |
| | | CGCTAGCACCAAGGGCCCATCGGTCTTCCCCCTGGCACC |
| | | CTCCTCCAAGAGCACCTCTGGGGGCACAGCGGCCCTGG |
| | | GCTGCCTGGTCAAGGACTACTTCCCCGAACCGGTGACGG |
| | | TGTCGTGGAACTCAGGCGCCCTGACCAGCGGCGTGCAC |
| | | ACCTTCCCGGCTGTCCTACAGTCCTCAGGACTCTACTCC |
| | | CTCAGCAGCGTGGTGACCGTGCCCTCCAGCAGCTTGGGC |
| | | ACCCAGACCTACATCTGCAACGTGAATCACAAGCCCAG |
| | | CAACACCAAGGTGGACAAGAAAGTTGAGCCCAAATCTT |
| | | GTGACAAAACTCACACATGCCCACCGTGCCCAGCACCT |
| | | GAAGCTGCAGGGGGACCGTCAGTCTTCCTCTTCCCCCCA |
| | | AAACCCAAGGACACCCTCATGATCTCCCGGACCCCTGA |
| | | GGTCACATGCGTGGTGGTGGACGTGAGCCACGAAGACC |
| | | CTGAGGTCAAGTTCAACTGGTACGTGGACGGCGTGGAG |
| | | GTGCATAATGCCAAGACAAAGCCGCGGGAGGAGCAGTA |
| | | CAACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCGGCGCCCCCATCGAGAAA |
| | | ACCATCTCCAAAGCCAAAGGGCAGCCCCGAGAACCACA |
| | | GGTGTACACCCTGCCCCCCTGCAGAGATGAGCTGACCA |
| | | AGAACCAGGTGTCCCTGTGGTGTCTGGTCAAGGGCTTCT |
| | | ACCCCAGCGATATCGCCGTGGAGTGGGAGAGCAACGGC |
| | | CAGCCTGAGAACAACTACAAGACCACCCCCCCTGTGCT |
| | | GGACAGCGACGGCAGCTTCTTCCTGTACTCCAAACTGAC |
| | | CGTGGACAAGAGCCGGTGGCAGCAGGGCAACGTGTTCA |
| | | GCTGCAGCGTGATGCACGAGGCCCTGCACAACCACTAC |
| | | ACCCAGAAGTCCCTGAGCCTGAGCCCCGGCGGAGGCGG |
| | | CGGAAGCGGAGGAGGAGGATCCAGAGAGGGCCCTGAG |
| | | CTGAGCCCTGATGATCCTGCCGGACTGCTGGACCTGCGG |
| | | CAGGGAATGTTTGCCCAGCTGGTGGCCCAGAACGTGCTG |
| | | CTGATCGATGGCCCCCTGTCCTGGTACAGCGATCCTGGA |
| | | CTGGCTGGCGTGTCACTGACAGGCGGCCTGAGCTACAA |
| | | AGAGGACACCAAAGAACTGGTGGTGGCCAAGGCCGGCG |
| | | TGTACTACGTGTTCTTTCAGCTGGAACTGCGGAGAGTGG |
| | | TGGCCGGCGAAGGATCTGGCTCTGTGTCTCTGGCCCTGC |
| | | ATCTGCAGCCTCTGAGATCTGCTGCTGGCGCCGCTGCTC |
| | | TGGCACTGACAGTGGATCTGCCTCCTGCCAGCAGCGAG |
| | | GCCCGGAATAGCGCATTTGGGTTTCAAGGCAGGCTGCTG |
| | | CACCTGTCTGCCGGCCAGAGGCTGGGAGTGCATCTGCAC |
| | | ACAGAGGCCAGGGCTAGACACGCCTGGCAGCTGACACA |
| | | GGGCGCTACAGTGCTGGGCCTGTTCAGAGTGACCCCCG |
| | | AGATTCCTGCCGGGCTC |
| 112 | nucleotide | GAGATCGTGCTGACCCAGTCCCCCGGCACCCTGTCTCTG |
| | sequence of anti- | AGCCCTGGCGAGAGAGCCACCCTGTCCTGCAGAGCCTC |
| | FAP(28H1) light | CCAGTCCGTGTCCCGGTCCTACCTCGCCTGGTATCAGCA |
| | chain | GAAGCCCGGCCAGGCCCCTCGGCTGCTGATCATCGGCG |
| | | CCTCTACCAGAGCCACCGGCATCCCTGACCGGTTCTCCG |
| | | GCTCTGGCTCCGGCACCGACTTCACCCTGACCATCTCCC |
| | | GGCTGGAACCCGAGGACTTCGCCGTGTACTACTGCCAGC |
| | | AGGGCCAGGTCATCCCTCCCACCTTTGGCCAGGGCACCA |
| | | AGGTGGAAATCAAGCGTACGGTGGCTGCACCATCTGTCT |
| | | TCATCTTCCCGCCATCTGATGAGCAGTTGAAATCTGGAA |
| | | CTGCCTCTGTTGTGTGCCTGCTGAATAACTTCTATCCCAG |
| | | AGAGGCCAAAGTACAGTGGAAGGTGGATAACGCCCTCC |
| | | AATCGGGTAACTCCCAGGAGAGTGTCACAGAGCAGGAC |
| | | AGCAAGGACAGCACCTACAGCCTCAGCAGCACCCTGAC |
| | | GCTGAGCAAAGCAGACTACGAGAAACACAAAGTCTACG |
| | | CCTGCGAAGTCACCCATCAGGGCCTGAGCTCGCCCGTCA |
| | | CAAAGAGCTTCAACAGGGGAGAGTGT |
| 104 | Fc hole dimeric 4- | See Table 1 |
| | 1BB ligand (71- | |
| | 248) chain | |
| 113 | anti-FAP(28H1) | EVQLLESGGGLVQPGGSLRLSCAASGFTFSSHAMSWVRQA |
| | Fc knob | PGKGLEWVSAIWASGEQYYADSVKGRFTISRDNSKNTLYL |
| | monomeric 4- | QMNSLRAEDTAVYYCAKGWLGNFDYWGQGTLVTVSSAS |
| | 1BB ligand (71- | TKGPSVFPLAPSSKSTSGGTAALGCLVKDYFPEPVTVSWNS |
| | 248) | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGGGGGSGGGGSREGPELSPDDPAGLLDLRQGMFAQ |
| | | LVAQNVLLIDGPLSWYSDPGLAGVSLTGGLSYKEDTKELV |
| | | VAKAGVYYVFFQLELRRVVAGEGSGSVSLALHLQPLRSAA |
| | | GAAALALTVDLPPASSEARNSAFGFQGRLLHLSAGQRLGV |
| | | HLHTEARARHAWQLTQGATVLGLFRVTPEIPAGL |
| 114 | anti-FAP(28H1) | EIVLTQSPGTLSLSPGERATLSCRASQSVSRSYLAWYQQKP |
| | light chain | GQAPRLLIIGASTRATGIPDRFSGSGSGTDFTLTISRLEPEDF |
| | | AVYYCQQGQVIPPTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |

Table 4 shows the cDNA and amino acid sequences of the monovalent FAP(28H1)-targeted 4-1BB ligand trimer-containing Fc (kih) fusion antigen binding molecule (Construct 1.12).

TABLE 4
Sequences of FAP(28H1)-targeted
human 4-1BB ligand trimer containing Fc (kih)
fusion molecule Construct 1.12

| SEQ ID | | |
|---|---|---|
| NO: | Description | Sequence |
| 107 | nucleotide | See Table 2 |
| | sequence of Fc | |
| | hole monomeric | |
| | 4-1BB ligand (71- | |
| | 248) chain | |
| 115 | nucleotide | GAAGTGCAGCTGCTGGAATCCGGCGGAGGCCTGGTGCA |
| | sequence of anti- | GCCTGGCGGATCTCTGAGACTGTCCTGCGCCGCCTCCGG |
| | FAP(28H1) Fc | CTTCACCTTCTCCTCCCACGCCATGTCCTGGGTCCGACA |
| | knob dimeric 4- | GGCTCCTGGCAAAGGCCTGGAATGGGTGTCCGCCATCTG |
| | 1BB ligand (71- | GGCCTCCGGCGAGCAGTACTACGCCGACTCTGTGAAGG |
| | 248) | GCCGGTTCACCATCTCCCGGGACAACTCCAAGAACACCC |
| | | TGTACCTGCAGATGAACTCCCTGCGGGCCGAGGACACC |
| | | GCCGTGTACTACTGTGCCAAGGGCTGGCTGGGCAACTTC |
| | | GACTACTGGGGACAGGGCACCCTGGTCACCGTGTCCAG |
| | | CGCTAGCACCAAGGGCCCATCGGTCTTCCCCCTGGCACC |
| | | CTCCTCCAAGAGCACCTCTGGGGGCACAGCGGCCCTGG |
| | | GCTGCCTGGTCAAGGACTACTTCCCCGAACCGGTGACGG |
| | | TGTCGTGGAACTCAGGCGCCCTGACCAGCGGCGTGCAC |
| | | ACCTTCCCGGCTGTCCTACAGTCCTCAGGACTCTACTCC |
| | | CTCAGCAGCGTGGTGACCGTGCCCTCCAGCAGCTTGGGC |
| | | ACCCAGACCTACATCTGCAACGTGAATCACAAGCCCAG |
| | | CAACACCAAGGTGGACAAGAAAGTTGAGCCCAAATCTT |
| | | GTGACAAAACTCACACATGCCCACCGTGCCCAGCACCT |
| | | GAAGCTGCAGGGGGACCGTCAGTCTTCCTCTTCCCCCCA |
| | | AAACCCAAGGACACCCTCATGATCTCCCGGACCCCTGA |
| | | GGTCACATGCGTGGTGGTGGACGTGAGCCACGAAGACC |
| | | CTGAGGTCAAGTTCAACTGGTACGTGGACGGCGTGGAG |
| | | GTGCATAATGCCAAGACAAAGCCGCGGGAGGAGCAGTA |
| | | CAACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCGGCGCCCCCATCGAGAAA |
| | | ACCATCTCCAAAGCCAAAGGGCAGCCCCGAGAACCACA |
| | | GGTGTACACCCTGCCCCCCTGCAGAGATGAGCTGACCA |
| | | AGAACCAGGTGTCCCTGTGGTGTCTGGTCAAGGGCTTCT |
| | | ACCCCAGCGATATCGCCGTGGAGTGGGAGAGCAACGGC |
| | | CAGCCTGAGAACAACTACAAGACCACCCCCCCTGTGCT |
| | | GGACAGCGACGGCAGCTTCTTCCTGTACTCCAAACTGAC |
| | | CGTGGACAAGAGCCGGTGGCAGCAGGGCAACGTGTTCA |
| | | GCTGCAGCGTGATGCACGAGGCCCTGCACAACCACTAC |
| | | ACCCAGAAGTCCCTGAGCCTGAGCCCCGGCAGAGAGGG |
| | | CCCTGAGCTGAGCCCTGATGATCCTGCCGGACTGCTGGA |
| | | CCTGCGGCAGGGAATGTTTGCCCAGCTGGTGGCCCAGA |
| | | ACGTGCTGCTGATCGATGGCCCCCTGTCCTGGTACAGCG |
| | | ATCCTGGACTGGCTGGCGTGTCACTGACAGGCGGCCTGA |
| | | GCTACAAAGAGGACACCAAAGAACTGGTGGTGGCCAAG |
| | | GCCGGCGTGTACTACGTGTTCTTTCAGCTGGAACTGCGG |
| | | AGAGTGGTGGCCGGCGAAGGATCTGGCTCTGTGTCTCTG |
| | | GCCCTGCATCTGCAGCCTCTGAGATCTGCTGCTGGCGCC |
| | | GCTGCTCTGGCACTGACAGTGGATCTGCCTCCTGCCAGC |
| | | AGCGAGGCCCGGAATAGCGCATTTGGGTTTCAAGGCAG |
| | | GCTGCTGCACCTGTCTGCCGGCCAGAGGCTGGGAGTGC |
| | | ATCTGCACACAGAGGCCAGGGCTAGACACGCCTGGCAG |
| | | CTGACACAGGGCGCTACAGTGCTGGGCCTGTTCAGAGTG |
| | | ACCCCCGAGATTCCAGCAGGCCTGGGAGGCGGCGGATC |
| | | TGGCGGCGGAGGATCTAGAGAAGGACCCGAGCTGTCCC |
| | | CCGACGATCCCGCTGGGCTGCTGGATCTGAGACAGGGC |
| | | ATGTTCGCTCAGCTGGTGGCTCAGAATGTGCTGCTGATT |
| | | GACGGACCTCTGAGCTGGTACTCCGACCCAGGGCTGGC |
| | | AGGGGTGTCCCTGACTGGGGGACTGTCCTACAAAGAAG |
| | | ATACAAAAGAACTGGTGGTGGCTAAAGCTGGGGTGTAC |
| | | TATGTGTTTTTTCAGCTGGAACTGAGGCGGGTGGTGGCT |
| | | GGGGAGGGCTCAGGATCTGTGTCCCTGGCTCTGCATCTG |
| | | CAGCCACTGCGCTCTGCAGCAGGGGCTGCAGCACTGGC |
| | | CCTGACTGTGGACCTGCCCCCAGCTTCTTCCGAGGCCAG |
| | | AAACAGCGCCTTCGGGTTCCAAGGACGCCTGCTGCATCT |
| | | GAGCGCCGGACAGCGCCTGGGAGTGCATCTGCATACTG |
| | | AAGCCAGAGCCCGGCATGCTTGGCAGCTGACTCAGGGG |
| | | GCAACTGTGCTGGGACTGTTTCGCGTGACACCTGAGATC |
| | | CCAGCCGGGCTC |
| 112 | nucleotide | See Table 3 |
| | sequence of anti- | |
| | FAP(28H1) light | |
| | chain | |
| 109 | Fc hole | See Table 2 |
| | monomeric 4- | |
| | 1BB ligand (71- | |
| | 248) chain | |
| 116 | anti-FAP(28H1) | EVQLLESGGGLVQPGGSLRLSCAASGFTFSSHAMSWVRQA |
| | Fc knob dimeric | PGKGLEWVSAIWASGEQYYADSVKGRFTISRDNSKNTLYL |
| | 4-1BB ligand (71- | QMNSLRAEDTAVYYCAKGWLGNFDYWGQGTLVTVSSAS |
| | 248) | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGREGPELSPDDPAGLLDLRQGMFAQLVAQNVLLID |
| | | GPLSWYSDPGLAGVSLTGGLSYKEDTKELVVAKAGVYYV |
| | | FFQLELRRVVAGEGSGSVSLALHLQPLRSAAGAAALALTV |
| | | DLPPASSEARNSAFGFQGRLLHLSAGQRLGVHLHTEARAR |
| | | HAWQLTQGATVLGLFRVTPEIPAGLGGGGSGGGGSREGPE |
| | | LSPDDPAGLLDLRQGMFAQLVAQNVLLIDGPLSWYSDPGL |
| | | AGVSLTGGLSYKEDTKELVVAKAGVYYVFFQLELRRVVA |
| | | GEGSGSVSLALHLQPLRSAAGAAALALTVDLPPASSEARN |
| | | SAFGFQGRLLHLSAGQRLGVHLHTEARARHAWQLTQGAT |
| | | VLGLFRVTPEIPAGL |
| 114 | anti-FAP(28H1) | See Table 3 |
| | light chain | |

1.2 Preparation of Monovalent CD19-Targeted 4-1BB Ligand Trimer-Containing Fc (Kih) Fusion Antigen Binding Molecule, Wherein the 4-1BB Ligands are C-Terminally Fused (Constructs 3.11, 4.11, 3.12 and 4.12) (CD19 on the Knob Chain)

The molecule was prepared as described in Example 1.1 for the monovalent FAP targeted construct, with the only difference that the anti-FAP binder was replaced by an anti-CD19 binder.

The variable region of heavy and light chain DNA sequences encoding a binder specific for CD19, clone 8B8-018 or clone 8B8-2B11, were subcloned in frame with either the constant heavy chain of the knob or the constant light chain of human IgG1. The generation of the CD19 clones is described in Example 1.3.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

For all constructs the knobs into holes heterodimerization technology was used as described in Carter, J Immunol Methods 248, 7-15 (2001). Combination of the huIgG1 Fc hole chain containing the Y349C/T366S/L368A/Y407V mutations in the CH3 domain, the anti-FAP huIgG1 knob chain containing the S354C/T366W mutations in the CH3 domain and the anti-FAP light chain allows generation of a heterodimer, which includes an assembled trimeric 4-1BB ligand and one CD19 binding Fab (FIGS. 2A and 2B).

Table 5 shows the cDNA and amino acid sequences of the monovalent CD19(8B8-018)-targeted 4-1BB ligand (71-248) trimer-containing Fc (kih) fusion antigen binding molecule (Construct 3.11).

TABLE 5
Sequences of CD19(8B8-018)-targeted
human 4-1BB ligand trimer containing Fc
(kih) fusion molecule Construct 3.11

| SEQ ID | | |
|---|---|---|
| NO: | Description | Sequence |
| 101 | nucleotide | see Table 1 |
| | sequence of Fc | |
| | hole dimeric 4- | |
| | 1BB ligand (71- | |
| | 248) chain | |
| 117 | nucleotide | CAGGTCCAGCTGGTGCAGTCCGGCGCCGAGGTCAAGAA |
| | sequence of anti- | ACCCGGGGCTTCTGTGAAGGTTTCATGCAAGGCAAGCG |
| | CD19(8B8-018) | GATACACCTTCACCGACTATATCATGCATTGGGTCAGGC |
| | Fc knob | AGGCCCCTGGCCAAGGTCTCGAATGGATGGGCTACATTA |
| | monomeric 4- | ACCCATATAATGATGGCTCCAAATACACCGAGAAGTTTC |
| | 1BB (71-248) | AGGGAAGAGTCACTATGACATCTGACACCAGTATCAGC |
| | ligand | ACTGCTTACATGGAGCTGTCCCGCCTTCGGTCTGATGAC |
| | | ACCGCAGTGTATTACTGTGCCAGGGGCACATATTACTAC |
| | | GGCTCAGCTCTGTTCGACTATTGGGGGCAGGGAACCACA |
| | | GTAACCGTGAGCTCCGCTAGCACCAAGGGCCCATCGGT |
| | | CTTCCCCCTGGCACCCTCCTCCAAGAGCACCTCTGGGGG |
| | | CACAGCGGCCCTGGGCTGCCTGGTCAAGGACTACTTCCC |
| | | CGAACCGGTGACGGTGTCGTGGAACTCAGGCGCCCTGA |
| | | CCAGCGGCGTGCACACCTTCCCGGCTGTCCTACAGTCCT |
| | | CAGGACTCTACTCCCTCAGCAGCGTGGTGACCGTGCCCT |
| | | CCAGCAGCTTGGGCACCCAGACCTACATCTGCAACGTG |
| | | AATCACAAGCCCAGCAACACCAAGGTGGACAAGAAAGT |
| | | TGAGCCCAAATCTTGTGACAAAACTCACACATGCCCACC |
| | | GTGCCCAGCACCTGAAGCTGCAGGGGGACCGTCAGTCT |
| | | TCCTCTTCCCCCCAAAACCCAAGGACACCCTCATGATCT |
| | | CCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTACACCCTGCCCCCCTGCAGA |
| | | GATGAGCTGACCAAGAACCAGGTGTCCCTGTGGTGTCTG |
| | | GTCAAGGGCTTCTACCCCAGCGATATCGCCGTGGAGTGG |
| | | GAGAGCAACGGCCAGCCTGAGAACAACTACAAGACCAC |
| | | CCCCCCTGTGCTGGACAGCGACGGCAGCTTCTTCCTGTA |
| | | CTCCAAACTGACCGTGGACAAGAGCCGGTGGCAGCAGG |
| | | GCAACGTGTTCAGCTGCAGCGTGATGCACGAGGCCCTG |
| | | CACAACCACTACACCCAGAAGTCCCTGAGCCTGAGCCC |
| | | CGGCGGAGGCGGCGGAAGCGGAGGAGGAGGATCCAGA |
| | | GAGGGCCCTGAGCTGAGCCCTGATGATCCTGCCGGACT |
| | | GCTGGACCTGCGGCAGGGAATGTTTGCCCAGCTGGTGGC |
| | | CCAGAACGTGCTGCTGATCGATGGCCCCCTGTCCTGGTA |
| | | CAGCGATCCTGGACTGGCTGGCGTGTCACTGACAGGCG |
| | | GCCTGAGCTACAAAGAGGACACCAAAGAACTGGTGGTG |
| | | GCCAAGGCCGGCGTGTACTACGTGTTCTTTCAGCTGGAA |
| | | CTGCGGAGAGTGGTGGCCGGCGAAGGATCTGGCTCTGT |
| | | GTCTCTGGCCCTGCATCTGCAGCCTCTGAGATCTGCTGC |
| | | TGGCGCCGCTGCTCTGGCACTGACAGTGGATCTGCCTCC |
| | | TGCCAGCAGCGAGGCCCGGAATAGCGCATTTGGGTTTCA |
| | | AGGCAGGCTGCTGCACCTGTCTGCCGGCCAGAGGCTGG |
| | | GAGTGCATCTGCACACAGAGGCCAGGGCTAGACACGCC |
| | | TGGCAGCTGACACAGGGCGCTACAGTGCTGGGCCTGTTC |
| | | AGAGTGACCCCCGAGATTCCTGCCGGGCTC |
| 118 | nucleotide | GACATCGTCATGACCCAGACACCCCTGTCCCTCTCTGTG |
| | sequence of anti- | ACCCCTGGCCAGCCAGCCTCAATTAGCTGCAAGTCCTCT |
| | CD19(8B8-018) | CAAAGTCTGGAGAACCCCAATGGGAACACTTACCTTAAT |
| | light chain | TGGTATCTGCAGAAACCCGGACAATCCCCTCAACTCCTG |
| | | ATCTACAGGGTCTCTAAGAGATTCTCAGGCGTGCCAGAT |
| | | CGCTTTAGCGGTTCCGGGTCTGGCACAGACTTCACCTTG |
| | | AAGATTAGTCGGGTTGAAGCTGAGGATGTGGGAGTCTA |
| | | TTACTGTCTGCAGCTCACTCATGTGCCCTACACCTTTGGT |
| | | CAGGGCACAAAACTGGAGATCAAGCGGACCGTGGCCGC |
| | | TCCCTCCGTGTTCATCTTCCCACCCTCCGACGAGCAGCT |
| | | GAAGTCCGGCACCGCCAGCGTGGTGTGCCTGCTGAACA |
| | | ACTTCTACCCCCGCGAGGCCAAGGTGCAGTGGAAGGTG |
| | | GACAACGCCCTGCAGTCCGGCAACTCCCAGGAATCCGT |
| | | GACCGAGCAGGACTCCAAGGACAGCACCTACTCCCTGT |
| | | CCTCCACCCTGACCCTGTCCAAGGCCGACTACGAGAAGC |
| | | ACAAGGTGTACGCCTGCGAAGTGACCCACCAGGGCCTG |
| | | TCCAGCCCCGTGACCAAGTCCTTCAACCGGGGCGAGTGC |
| 104 | Fc hole dimeric 4- | see Table 1 |
| | 1BB (71-248) | |
| | ligand chain | |
| 119 | anti-CD19(8B8- | QVQLVQSGAEVKKPGASVKVSCKASGYTFTDYIMHWVRQ |
| | 018) Fc knob | APGQGLEWMGYINPYNDGSKYTEKFQGRVTMTSDTSISTA |
| | monomeric 4-1- | YMELSRLRSDDTAVYYCARGTYYYGSALFDYWGQGTTVT |
| | BB (71-248) | VSSASTKGPSVFPLAPSSKSTSGGTAALGCLVKDYFPEPVT |
| | ligand | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGGGGGSGGGGSREGPELSPDDPAGLLDLRQ |
| | | GMFAQLVAQNVLLIDGPLSWYSDPGLAGVSLTGGLSYKED |
| | | TKELVVAKAGVYYVFFQLELRRVVAGEGSGSVSLALHLQP |
| | | LRSAAGAAALALTVDLPPASSEARNSAFGFQGRLLHLSAG |
| | | QRLGVHLHTEARARHAWQLTQGATVLGLFRVTPEIPAGL |
| 120 | anti-CD19(8B8- | DIVMTQTPLSLSVTPGQPASISCKSSQSLENPNGNTYLNWY |
| | 018) light chain | LQKPGQSPQLLIYRVSKRFSGVPDRFSGSGSGTDFTLKISRV |
| | | EAEDVGVYYCLQLTHVPYTFGQGTKLEIKRTVAAPSVFIFP |
| | | PSDEQLKSGTASVVCLLNNFYPREAKVQWKVDNALQSGN |
| | | SQESVTEQDSKDSTYSLSSTLTLSKADYEKHKVYACEVTH |
| | | QGLSSPVTKSFNRGEC |

Table 6 shows theecDNA and amino aid sequenes ofthe monovalent CD19(8B8-018)-targeted 4-1BB ligand (71-248) timer-eontaining Fe(kih) fusion antigen binding moleule (Construet 3.12).

TABLE 2
Sequences of CD19 (B8-018) targeted
human 4-1BB ligand trimer containing Fc
(kih) fusion molecule Construct 3.12

| SEQ ID | | |
|---|---|---|
| NO: | Description | Sequence |
| 107 | nucleotide | see Table 2 |
| | sequence of Fc | |
| | hole monomeric | |
| | 4-1BBL (71-248) | |
| | chain | |
| 121 | nucleotide | CAGGTCCAGCTGGTGCAGTCCGGCGCCGAGGTCAAGAA |
| | sequence of anti- | ACCCGGGGCTTCTGTGAAGGTTTCATGCAAGGCAAGCG |
| | CD19(8B8-018) | GATACACCTTCACCGACTATATCATGCATTGGGTCAGGC |
| | Fc knob dimeric | AGGCCCCTGGCCAAGGTCTCGAATGGATGGGCTACATTA |
| | 4-1BB (71-248) | ACCCATATAATGATGGCTCCAAATACACCGAGAAGTTTC |
| | ligand | AGGGAAGAGTCACTATGACATCTGACACCAGTATCAGC |
| | | ACTGCTTACATGGAGCTGTCCCGCCTTCGGTCTGATGAC |
| | | ACCGCAGTGTATTACTGTGCCAGGGGCACATATTACTAC |
| | | GGCTCAGCTCTGTTCGACTATTGGGGGCAGGGAACCACA |
| | | GTAACCGTGAGCTCCGCTAGCACCAAGGGCCCATCGGT |
| | | CTTCCCCCTGGCACCCTCCTCCAAGAGCACCTCTGGGGG |
| | | CACAGCGGCCCTGGGCTGCCTGGTCAAGGACTACTTCCC |
| | | CGAACCGGTGACGGTGTCGTGGAACTCAGGCGCCCTGA |
| | | CCAGCGGCGTGCACACCTTCCCGGCTGTCCTACAGTCCT |
| | | CAGGACTCTACTCCCTCAGCAGCGTGGTGACCGTGCCCT |
| | | CCAGCAGCTTGGGCACCCAGACCTACATCTGCAACGTG |
| | | AATCACAAGCCCAGCAACACCAAGGTGGACAAGAAAGT |
| | | TGAGCCCAAATCTTGTGACAAAACTCACACATGCCCACC |
| | | GTGCCCAGCACCTGAAGCTGCAGGGGGACCGTCAGTCT |
| | | TCCTCTTCCCCCCAAAACCCAAGGACACCCTCATGATCT |
| | | CCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTACACCCTGCCCCCCTGCAGA |
| | | GATGAGCTGACCAAGAACCAGGTGTCCCTGTGGTGTCTG |
| | | GTCAAGGGCTTCTACCCCAGCGATATCGCCGTGGAGTGG |
| | | GAGAGCAACGGCCAGCCTGAGAACAACTACAAGACCAC |
| | | CCCCCCTGTGCTGGACAGCGACGGCAGCTTCTTCCTGTA |
| | | CTCCAAACTGACCGTGGACAAGAGCCGGTGGCAGCAGG |
| | | GCAACGTGTTCAGCTGCAGCGTGATGCACGAGGCCCTG |
| | | CACAACCACTACACCCAGAAGTCCCTGAGCCTGAGCCC |
| | | CGGCAGAGAGGGCCCTGAGCTGAGCCCTGATGATCCTG |
| | | CCGGACTGCTGGACCTGCGGCAGGGAATGTTTGCCCAG |
| | | CTGGTGGCCCAGAACGTGCTGCTGATCGATGGCCCCCTG |
| | | TCCTGGTACAGCGATCCTGGACTGGCTGGCGTGTCACTG |
| | | ACAGGCGGCCTGAGCTACAAAGAGGACACCAAAGAACT |
| | | GGTGGTGGCCAAGGCCGGCGTGTACTACGTGTTCTTTCA |
| | | GCTGGAACTGCGGAGAGTGGTGGCCGGCGAAGGATCTG |
| | | GCTCTGTGTCTCTGGCCCTGCATCTGCAGCCTCTGAGAT |
| | | CTGCTGCTGGCGCCGCTGCTCTGGCACTGACAGTGGATC |
| | | TGCCTCCTGCCAGCAGCGAGGCCCGGAATAGCGCATTTG |
| | | GGTTTCAAGGCAGGCTGCTGCACCTGTCTGCCGGCCAGA |
| | | GGCTGGGAGTGCATCTGCACACAGAGGCCAGGGCTAGA |
| | | CACGCCTGGCAGCTGACACAGGGCGCTACAGTGCTGGG |
| | | CCTGTTCAGAGTGACCCCCGAGATTCCAGCAGGCCTGGG |
| | | AGGCGGCGGATCTGGCGGCGGAGGATCTAGAGAAGGAC |
| | | CCGAGCTGTCCCCCGACGATCCCGCTGGGCTGCTGGATC |
| | | TGAGACAGGGCATGTTCGCTCAGCTGGTGGCTCAGAATG |
| | | TGCTGCTGATTGACGGACCTCTGAGCTGGTACTCCGACC |
| | | CAGGGCTGGCAGGGGTGTCCCTGACTGGGGGACTGTCCT |
| | | ACAAAGAAGATACAAAAGAACTGGTGGTGGCTAAAGCT |
| | | GGGGTGTACTATGTGTTTTTTCAGCTGGAACTGAGGCGG |
| | | GTGGTGGCTGGGGAGGGCTCAGGATCTGTGTCCCTGGCT |
| | | CTGCATCTGCAGCCACTGCGCTCTGCAGCAGGGGCTGCA |
| | | GCACTGGCCCTGACTGTGGACCTGCCCCCAGCTTCTTCC |
| | | GAGGCCAGAAACAGCGCCTTCGGGTTCCAAGGACGCCT |
| | | GCTGCATCTGAGCGCCGGACAGCGCCTGGGAGTGCATC |
| | | TGCATACTGAAGCCAGAGCCCGGCATGCTTGGCAGCTG |
| | | ACTCAGGGGGCAACTGTGCTGGGACTGTTTCGCGTGACA |
| | | CCTGAGATCCCAGCCGGGCTC |
| 118 | nucleotide | See Table 5 |
| | sequence of anti- | |
| | CD19 (8B8-018) | |
| | light chain | |
| 109 | Fc hole | see Table 2 |
| | monomeric 4- | |
| | 1BB ligand (71- | |
| | 248) chain | |
| 122 | anti-CD19(8B8- | QVQLVQSGAEVKKPGASVKVSCKASGYTFTDYIMHWVRQ |
| | 018) Fc knob | APGQGLEWMGYINPYNDGSKYTEKFQGRVTMTSDTSISTA |
| | dimeric 4-1BB | YMELSRLRSDDTAVYYCARGTYYYGSALFDYWGQGTTVT |
| | (71-248) ligand | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGREGPELSPDDPAGLLDLRQGMFAQLVAQN |
| | | VLLIDGPLSWYSDPGLAGVSLTGGLSYKEDTKELVVAKAG |
| | | VYYVFFQLELRRVVAGEGSGSVSLALHLQPLRSAAGAAAL |
| | | ALTVDLPPASSEARNSAFGFQGRLLHLSAGQRLGVHLHTE |
| | | ARARHAWQLTQGATVLGLFRVTPEIPAGLGGGGSGGGGS |
| | | REGPELSPDDPAGLLDLRQGMFAQLVAQNVLLIDGPLSWY |
| | | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGL |
| 120 | anti-CD19(8B8- | See Table 5 |
| | 018) light chain | |

Table 7 shows the cDNA and amino acid sequences of the monovalent CD19 (8B8-2B11)-targeted 4-1BB ligand trimer-containing Fc (kih) fusion antigen binding molecule (Construct 4.11).

TABLE 7
Sequences of CD19(2B11)-targeted human
4-1BB ligand trimer containing Fc (kih)
fusion molecule Construct 4.11

| SEQ ID | | |
|---|---|---|
| NO: | Description | Sequence |
| 101 | nucleotide | See Table 1 |
| | sequence of Fc | |
| | hole dimeric 4- | |
| | 1BB ligand (71- | |
| | 248) chain | |
| 123 | nucleotide | CAGGTGCAATTGGTTCAATCTGGTGCTGAAGTAAAAAA |
| | sequence of anti- | ACCGGGCGCTTCCGTTAAAGTGAGCTGCAAAGCATCTGG |
| | CD19(8B8-2B11) | TTACACCTTCACTGACTATATCATGCACTGGGTTCGTCA |
| | Fc knob | GGCCCCGGGCCAGGGTCTGGAGTGGATGGGCTACATTA |
| | monomeric 4- | ACCCATACAACGACGGTTCCAAATATACCGAGAAATTC |
| | 1BB (71-248) | CAGGGCCGCGTCACGATGACCAGCGACACTTCTATCTCC |
| | ligand | ACCGCGTACATGGAACTGTCTAGACTGCGTTCTGACGAC |
| | | ACCGCTGTTTACTATTGTGCACGCGGTACCTACTACTAC |
| | | GGTCCACAGCTGTTTGATTACTGGGGCCAAGGTACCACG |
| | | GTGACCGTAAGCTCTGCTAGCACCAAGGGCCCATCGGTC |
| | | TTCCCCCTGGCACCCTCCTCCAAGAGCACCTCTGGGGGC |
| | | ACAGCGGCCCTGGGCTGCCTGGTCAAGGACTACTTCCCC |
| | | GAACCGGTGACGGTGTCGTGGAACTCAGGCGCCCTGAC |
| | | CAGCGGCGTGCACACCTTCCCGGCTGTCCTACAGTCCTC |
| | | AGGACTCTACTCCCTCAGCAGCGTGGTGACCGTGCCCTC |
| | | CAGCAGCTTGGGCACCCAGACCTACATCTGCAACGTGA |
| | | ATCACAAGCCCAGCAACACCAAGGTGGACAAGAAAGTT |
| | | GAGCCCAAATCTTGTGACAAAACTCACACATGCCCACC |
| | | GTGCCCAGCACCTGAAGCTGCAGGGGGACCGTCAGTCT |
| | | TCCTCTTCCCCCCAAAACCCAAGGACACCCTCATGATCT |
| | | CCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTACACCCTGCCCCCCTGCAGA |
| | | GATGAGCTGACCAAGAACCAGGTGTCCCTGTGGTGTCTG |
| | | GTCAAGGGCTTCTACCCCAGCGATATCGCCGTGGAGTGG |
| | | GAGAGCAACGGCCAGCCTGAGAACAACTACAAGACCAC |
| | | CCCCCCTGTGCTGGACAGCGACGGCAGCTTCTTCCTGTA |
| | | CTCCAAACTGACCGTGGACAAGAGCCGGTGGCAGCAGG |
| | | GCAACGTGTTCAGCTGCAGCGTGATGCACGAGGCCCTG |
| | | CACAACCACTACACCCAGAAGTCCCTGAGCCTGAGCCC |
| | | CGGCGGAGGCGGCGGAAGCGGAGGAGGAGGATCCAGA |
| | | GAGGGCCCTGAGCTGAGCCCTGATGATCCTGCCGGACT |
| | | GCTGGACCTGCGGCAGGGAATGTTTGCCCAGCTGGTGGC |
| | | CCAGAACGTGCTGCTGATCGATGGCCCCCTGTCCTGGTA |
| | | CAGCGATCCTGGACTGGCTGGCGTGTCACTGACAGGCG |
| | | GCCTGAGCTACAAAGAGGACACCAAAGAACTGGTGGTG |
| | | GCCAAGGCCGGCGTGTACTACGTGTTCTTTCAGCTGGAA |
| | | CTGCGGAGAGTGGTGGCCGGCGAAGGATCTGGCTCTGT |
| | | GTCTCTGGCCCTGCATCTGCAGCCTCTGAGATCTGCTGC |
| | | TGGCGCCGCTGCTCTGGCACTGACAGTGGATCTGCCTCC |
| | | TGCCAGCAGCGAGGCCCGGAATAGCGCATTTGGGTTTCA |
| | | AGGCAGGCTGCTGCACCTGTCTGCCGGCCAGAGGCTGG |
| | | GAGTGCATCTGCACACAGAGGCCAGGGCTAGACACGCC |
| | | TGGCAGCTGACACAGGGCGCTACAGTGCTGGGCCTGTTC |
| | | AGAGTGACCCCCGAGATTCCTGCCGGGCTC |
| 124 | nucleotide | GATATTGTCATGACTCAAACTCCACTGTCTCTGTCCGTG |
| | sequence of anti- | ACCCCGGGTCAGCCAGCGAGCATTTCTTGCAAATCCAGC |
| | CD19(8B8-2B11) | CAATCTCTGGAAACCTCCACCGGCACCACGTACCTGAAC |
| | light chain | TGGTATCTCCAGAAACCGGGTCAGAGCCCGCAGCTGCT |
| | | GATCTACCGTGTATCTAAGCGCTTCTCCGGCGTTCCTGA |
| | | TCGTTTCAGCGGTTCTGGATCCGGCACCGACTTTACTCT |
| | | GAAAATCAGCCGTGTGGAAGCTGAAGACGTTGGCGTCT |
| | | ACTATTGTCTGCAGCTGCTGGAAGATCCATACACCTTCG |
| | | GTCAAGGAACTAAACTGGAAATTAAACGTACGGTGGCT |
| | | GCACCATCTGTCTTCATCTTCCCGCCATCTGATGAGCAG |
| | | TTGAAATCTGGAACTGCCTCTGTTGTGTGCCTGCTGAAT |
| | | AACTTCTATCCCAGAGAGGCCAAAGTACAGTGGAAGGT |
| | | GGATAACGCCCTCCAATCGGGTAACTCCCAGGAGAGTG |
| | | TCACAGAGCAGGACAGCAAGGACAGCACCTACAGCCTC |
| | | AGCAGCACCCTGACGCTGAGCAAAGCAGACTACGAGAA |
| | | ACACAAAGTCTACGCCTGCGAAGTCACCCATCAGGGCC |
| | | TGAGCTCGCCCGTCACAAAGAGCTTCAACAGGGGAGAG |
| | | TGTGATATTGTCATGACTCAAACTCCACTGTCTCTGTCCG |
| | | TGACCCCGGGTCAGCCAGCGAGCATTTCTTGCAAATCCA |
| | | GCCAATCTCTGGAAACCTCCACCGGCACCACGTACCTGA |
| | | ACTGGTATCTCCAGAAACCGGGTCAGAGCCCGCAGCTG |
| | | CTGATCTACCGTGTATCTAAGCGCTTCTCCGGCGTTCCTG |
| | | ATCGTTTCAGCGGTTCTGGATCCGGCACCGACTTTACTC |
| | | TGAAAATCAGCCGTGTGGAAGCTGAAGACGTTGGCGTC |
| | | TACTATTGTCTGCAGCTGCTGGAAGATCCATACACCTTC |
| | | GGTCAAGGAACGAAACTGGAAATTAAACGTACGGTGGC |
| | | TGCACCATCTGTCTTCATCTTCCCGCCATCTGATGAGCA |
| | | GTTGAAATCTGGAACTGCCTCTGTTGTGTGCCTGCTGAA |
| | | TAACTTCTATCCCAGAGAGGCCAAAGTACAGTGGAAGG |
| | | TGGATAACGCCCTCCAATCGGGTAACTCCCAGGAGAGT |
| | | GTCACAGAGCAGGACAGCAAGGACAGCACCTACAGCCT |
| | | CAGCAGCACCCTGACGCTGAGCAAAGCAGACTACGAGA |
| | | AACACAAAGTCTACGCCTGCGAAGTCACCCATCAGGGC |
| | | CTGAGCTCGCCCGTCACAAAGAGCTTCAACAGGGGAGA |
| | | GTGT |
| 104 | Fc hole dimeric 4- | See Table 1 |
| | 1BB ligand (71- | |
| | 248) chain | |
| 125 | anti-CD19(8B8- | QVQLVQSGAEVKKPGASVKVSCKASGYTFTDYIMHWVRQ |
| | 2B11) Fc knob | APGQGLEWMGYINPYNDGSKYTEKFQGRVTMTSDTSISTA |
| | monomeric 4- | YMELSRLRSDDTAVYYCARGTYYYGPQLFDYWGQGTTVT |
| | 1BB ligand (71- | VSSASTKGPSVFPLAPSSKSTSGGTAALGCLVKDYFPEPVT |
| | 248) | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGGGGGSGGGGSREGPELSPDDPAGLLDLRQ |
| | | GMFAQLVAQNVLLIDGPLSWYSDPGLAGVSLTGGLSYKED |
| | | TKELVVAKAGVYYVFFQLELRRVVAGEGSGSVSLALHLQP |
| | | LRSAAGAAALALTVDLPPASSEARNSAFGFQGRLLHLSAG |
| | | QRLGVHLHTEARARHAWQLTQGATVLGLFRVTPEIPAGL |
| 126 | anti-CD19(8B8- | DIVMTQTPLSLSVTPGQPASISCKSSQSLETSTGTTYLNWYL |
| | 2B11) light chain | QKPGQSPQLLIYRVSKRFSGVPDRFSGSGSGTDFTLKISRVE |
| | | AEDVGVYYCLQLLEDPYTFGQGTKLEIKRTVAAPSVFIFPP |
| | | SDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |

Table 8 shows the cDNA and amino acid sequences of the monovalent CD19(8B8-2B11) targeted 4-1BB ligand trimer-containing Fc (kih) fusion antigen binding molecule (Construct 4.12).

TABLE 8
Sequences of CD19(8B8-2B11)-targeted
human 4-1BB ligand trimer containing Fc
(kih) fusion molecule Construct 4.12

| SEQ ID | | |
|---|---|---|
| NO: | Description | Sequence |
| 107 | nucleotide | See Table 2 |
| | sequence of Fc | |
| | hole monomeric | |
| | 4-1BB ligand (71- | |
| | 248) chain | |
| 127 | nucleotide | CAGGTGCAATTGGTTCAATCTGGTGCTGAAGTAAAAAA |
| | sequence of anti- | ACCGGGCGCTTCCGTTAAAGTGAGCTGCAAAGCATCTGG |
| | CD19(8B8-2B11) | TTACACCTTCACTGACTATATCATGCACTGGGTTCGTCA |
| | Fc knob dimeric | GGCCCCGGGCCAGGGTCTGGAGTGGATGGGCTACATTA |
| | 4-1BB (71-248) | ACCCATACAACGACGGTTCCAAATATACCGAGAAATTC |
| | ligand | CAGGGCCGCGTCACGATGACCAGCGACACTTCTATCTCC |
| | | ACCGCGTACATGGAACTGTCTAGACTGCGTTCTGACGAC |
| | | ACCGCTGTTTACTATTGTGCACGCGGTACCTACTACTAC |
| | | GGTCCACAGCTGTTTGATTACTGGGGCCAAGGTACCACG |
| | | GTGACCGTAAGCTCTGCTAGCACCAAGGGCCCATCGGTC |
| | | TTCCCCCTGGCACCCTCCTCCAAGAGCACCTCTGGGGGC |
| | | ACAGCGGCCCTGGGCTGCCTGGTCAAGGACTACTTCCCC |
| | | GAACCGGTGACGGTGTCGTGGAACTCAGGCGCCCTGAC |
| | | CAGCGGCGTGCACACCTTCCCGGCTGTCCTACAGTCCTC |
| | | AGGACTCTACTCCCTCAGCAGCGTGGTGACCGTGCCCTC |
| | | CAGCAGCTTGGGCACCCAGACCTACATCTGCAACGTGA |
| | | ATCACAAGCCCAGCAACACCAAGGTGGACAAGAAAGTT |
| | | GAGCCCAAATCTTGTGACAAAACTCACACATGCCCACC |
| | | GTGCCCAGCACCTGAAGCTGCAGGGGGACCGTCAGTCT |
| | | TCCTCTTCCCCCCAAAACCCAAGGACACCCTCATGATCT |
| | | CCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTACACCCTGCCCCCCTGCAGA |
| | | GATGAGCTGACCAAGAACCAGGTGTCCCTGTGGTGTCTG |
| | | GTCAAGGGCTTCTACCCCAGCGATATCGCCGTGGAGTGG |
| | | GAGAGCAACGGCCAGCCTGAGAACAACTACAAGACCAC |
| | | CCCCCCTGTGCTGGACAGCGACGGCAGCTTCTTCCTGTA |
| | | CTCCAAACTGACCGTGGACAAGAGCCGGTGGCAGCAGG |
| | | GCAACGTGTTCAGCTGCAGCGTGATGCACGAGGCCCTG |
| | | CACAACCACTACACCCAGAAGTCCCTGAGCCTGAGCCC |
| | | CGGCAGAGAGGGCCCTGAGCTGAGCCCTGATGATCCTG |
| | | CCGGACTGCTGGACCTGCGGCAGGGAATGTTTGCCCAG |
| | | CTGGTGGCCCAGAACGTGCTGCTGATCGATGGCCCCCTG |
| | | TCCTGGTACAGCGATCCTGGACTGGCTGGCGTGTCACTG |
| | | ACAGGCGGCCTGAGCTACAAAGAGGACACCAAAGAACT |
| | | GGTGGTGGCCAAGGCCGGCGTGTACTACGTGTTCTTTCA |
| | | GCTGGAACTGCGGAGAGTGGTGGCCGGCGAAGGATCTG |
| | | GCTCTGTGTCTCTGGCCCTGCATCTGCAGCCTCTGAGAT |
| | | CTGCTGCTGGCGCCGCTGCTCTGGCACTGACAGTGGATC |
| | | TGCCTCCTGCCAGCAGCGAGGCCCGGAATAGCGCATTTG |
| | | GGTTTCAAGGCAGGCTGCTGCACCTGTCTGCCGGCCAGA |
| | | GGCTGGGAGTGCATCTGCACACAGAGGCCAGGGCTAGA |
| | | CACGCCTGGCAGCTGACACAGGGCGCTACAGTGCTGGG |
| | | CCTGTTCAGAGTGACCCCCGAGATTCCAGCAGGCCTGGG |
| | | AGGCGGCGGATCTGGCGGCGGAGGATCTAGAGAAGGAC |
| | | CCGAGCTGTCCCCCGACGATCCCGCTGGGCTGCTGGATC |
| | | TGAGACAGGGCATGTTCGCTCAGCTGGTGGCTCAGAATG |
| | | TGCTGCTGATTGACGGACCTCTGAGCTGGTACTCCGACC |
| | | CAGGGCTGGCAGGGGTGTCCCTGACTGGGGGACTGTCCT |
| | | ACAAAGAAGATACAAAAGAACTGGTGGTGGCTAAAGCT |
| | | GGGGTGTACTATGTGTTTTTTCAGCTGGAACTGAGGCGG |
| | | GTGGTGGCTGGGGAGGGCTCAGGATCTGTGTCCCTGGCT |
| | | CTGCATCTGCAGCCACTGCGCTCTGCAGCAGGGGCTGCA |
| | | GCACTGGCCCTGACTGTGGACCTGCCCCCAGCTTCTTCC |
| | | GAGGCCAGAAACAGCGCCTTCGGGTTCCAAGGACGCCT |
| | | GCTGCATCTGAGCGCCGGACAGCGCCTGGGAGTGCATC |
| | | TGCATACTGAAGCCAGAGCCCGGCATGCTTGGCAGCTG |
| | | ACTCAGGGGGCAACTGTGCTGGGACTGTTTCGCGTGACA |
| | | CCTGAGATCCCAGCCGGGCTC |
| 124 | nucleotide | See Table 7 |
| | sequence of anti- | |
| | CD19(8B8-2B11) | |
| | light chain | |
| 109 | Fc hole | See Table 2 |
| | monomeric 4- | |
| | 1BB ligand (71- | |
| | 248) chain | |
| 128 | anti-CD19(8B8- | QVQLVQSGAEVKKPGASVKVSCKASGYTFTDYIMHWVRQ |
| | 2B11) Fc knob | APGQGLEWMGYINPYNDGSKYTEKFQGRVTMTSDTSISTA |
| | dimeric 4-1BB | YMELSRLRSDDTAVYYCARGTYYYGPQLFDYWGQGTTVT |
| | ligand (71-248) | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALGAPIEKTISKAKGQPREPQVYTLPPCRD |
| | | ELTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSPGREGPELSPDDPAGLLDLRQGMFAQLVAQN |
| | | VLLIDGPLSWYSDPGLAGVSLTGGLSYKEDTKELVVAKAG |
| | | VYYVFFQLELRRVVAGEGSGSVSLALHLQPLRSAAGAAAL |
| | | ALTVDLPPASSEARNSAFGFQGRLLHLSAGQRLGVHLHTE |
| | | ARARHAWQLTQGATVLGLFRVTPEIPAGLGGGGSGGGGS |
| | | REGPELSPDDPAGLLDLRQGMFAQLVAQNVLLIDGPLSWY |
| | | SDPGLAGVSLTGGLSYKEDTKELVVAKAGVYYVFFQLELR |
| | | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGL |
| 126 | anti-CD19(8B8- | See Table 7 |
| | 2B11) light chain | |

1.3. Generation of Anti-CD19 Binders 8B8-018 and 8B8-2B11

1.3.1 Generation of Anti-CD19 Clone 8B8-018

a) Immunization and Generation of Mouse Anti-Human CD19 Antibodies (Hybridomas)

Balb/c mice were immunized six times and boosted with CD19-transfected HEK293 cells (mean receptor density 35,000 per cell). The immune response was monitored by testing serum samples with a CD19-cell-ELISA on human CD19-transfected NIH-3T3 cells. Spleen cells from mice with sufficient titers of anti-human CD19 antibody were used for immortalization by fusion with mouse myeloma cell line P3X63 Ag8.653. Three fusions were carried out and hybridoma supernatants screened by cell-ELISA on human CD19-transfected NIH-3T3 cells and FACS binding assay using Daudi (CD19+) and CD19-cells for anti-human CD19 specific antibodies (see Example 1 of WO 2011/147834).

b) Hybridoma Screening and Cell Biological Functional Evaluation of Anti-CD19 Antibodies

A cell ELISA was applied for screening of hybridomas, and to identify those hybridomas that secrete antibodies against human-CD19. NIH3T3 cells transfected with human-CD19 were used as positive cells; non-transfected NIH3T3 cells were used as negative control cells. For the assessment of the positive hybridomas the OD ratio between transfected and non-transfected NIH3T3 cells was quantified.
Culture Medium: DMEM high glucose (4.5 mg/ml), 10% FCS, Na-Pyruvate, NEAA, Glutamine
Antibodies positive control: anti CD19 monoclonal antibody (IgG1) Pharmingen Cat #555409 c=1 mg/ml
Detection antibody: Goat anti-Mouse IgG (H+L) HRP Conjugate Bio-Rad Cat #170-06516
Dilution 1: 2000 in 1×ELISA Blocking Reagent
Other reagents: Fibronectin Roche Cat #838039 c=1 mg/ml
Glutardialdehyde: 25% stock solution//Grade Agar Scientific #R102 final concentration: 0.05% in PBS
ELISA Blocking Reagent: 1× stock solution//Roche Cat#1112589
TMB substrate: Roche Cat#11432559
Stop Solution: 1 M H2SO4
BioRad Cat#170-6516 Dilution 1: 2000 in 1×ELISA Blocking Reagent

Day 1:
Fibronectin coating: 5 μg/cm² in PBS; 96well plate=32 cm²; 160 μg/plate in 6 ml
PBS, 50 μl/well
incubate 45 min at RT, aspirate coating solution
Seed 1.25×104 cells/well in 50 μl culture medium in a 96well plate
incubate 40 hours at 37° C.
add to upper half of the plate: NIH3T3 cells expressing CD19
add to lower half of the plate: non-transfected NIH3T3 cells

Day 3:
Addition of positive control antibody or samples (supernatant or mouse serum) in 50 μl culture medium
incubate for 2 h at 4° C.
Remove medium, fix cells with 100 μl Glutardialdehyde (0.05% in PBS)
Wash two times with 200 μl PBS
Addition of detection antibody 1:2000, 50 μl/well
incubate 2 h at RT
wash three times with 200 μl PBS
add 50 μl TMB, incubate for 30 min. at RT,
stop by addition of 25 μl 1 M H2SO4; read extinction at 450 nm/620 nm
Calculation of results: ratio OD NIH3T3 CD19: OD NIH3T3 non-transfected

The selected antibody demonstrated specific binding to CD19 transfected NIH3T3 cells as compared to untransfected NIH3T3 cells (see Example 2 of WO 2011/147834).

c) Humanization of anti-CD19 antibody

The CD19 binding specificity of the murine antibody was transferred onto a human acceptor framework to eliminate potential immunogenicity issues arising from sequence stretches that the human body will recognize as foreign. This was done by engrafting the entire complementary determining regions (CDR) of the murine (donor) antibody onto a human (acceptor) antibody framework, and is called CDR-grafting or antibody humanization.

The murine amino acid sequence was aligned with a collection of human germ-line antibody V genes, and sorted according to sequence identity and homology. Before selecting one particular acceptor sequence, the so-called canonical loop structures of the donor antibody have to be determined (Morea, V., et al., Methods, Vol 20, Issue 3 (2000) 267-279). These canonical loop structures are determined by the type of residues present at the so-called canonical positions. These positions lie (partially) outside of the CDR regions, and have to be kept functionally equivalent in the final construct in order to retain the CDR conformation of the parental (donor) antibody. The human germ-line sequence VBASE_VH1_1 was chosen as the acceptor for the heavy chain and sequence VBASE_VK2_5 was chosen for the light chain.

It was found that the wild-type humanized anti-human CD19 antibody 8B8 has three deamidation hotspots in the HVR-L: NSNGNT (SEQ ID NO: 129). Additionally it was found that in the HVR-H2 a further deamidation hotspot is present: KFNG (SEQ ID NO: 130). To address the deamidation hotspot in the HVR-H2 an N (Asn) to Q (Gln) point mutation at position 64 (numbering according to Kabat) has been introduced. To address the deamidation hotspots in the light chain and to obtain a humanized anti-human CD19 antibody with improved deamidation stability a single mutation at position 27e from S (serine) to P (proline) (numbering according to Kabat) was introduced. Thus, clone 8B8-018 with the CDRs as shown in Table 16 below was generated.

TABLE 9
Comparison of 8B8-018 with humanized wild-type CD19 antibody 8B8

| | variant | |
|---|---|---|
| parameter | wt 8B8 | 8B8-018 |
| K_{D} (BIAcore) [nM] | 5 | 6 |
| t_{1/2} [min] | — | 43.6 |
| human CD19 binding after | 46 | 95 |
| pH 7.4 incubation [%] | | |
| human CD19 binding after | 90 | 99 |
| pH 6.0 incubation [%] | | |
| SEC main peak after | >95 | >95 |
| incubation [%] | | |

Additionally, 8B8-018 maintains the cross-reactivity to cynomolgus CD19 as shown in the following Table 10.

| EC50 [μg/ml] | wt 8B8 | 8B8-018 |
|---|---|---|
| huCD19 ECD | 0.087 | 0.084 |
| cyCD19 ECD | 0.313 | 0.255 |

1.3.2 Preparation, Purification and Characterization of CD19 Antigen Fc Fusion for Phage Display Campaign

In order to express and purify the human and cynomolgus CD19 ectodomain in a monomeric state (human CD19 see SEQ ID NO:64), the respective DNA fragment was fused to a human IgG1 Fc gene segment containing the “knob” mutations (human: SEQ ID NO: 132; cynomolgus: SEQ ID NO: 135) and was transfected with an “Fc-hole” (SEQ ID NO: 131) counterpart (Merchant et al., 1998). An IgA cleavage site (PTPPTP) was introduced between the antigen ectodomain and the Fc knob chain. An Avi tag for directed biotinylation was introduced at the C-terminus of the antigen-Fc knob chain and mutations H435R and Y436F were introduced in the Fc hole for purification purposes (Jendeberg L. et al, J. Immunological methods, 1997). Combination of the antigen-Fc knob chain containing the S354C/T366W mutations (human: SEQ ID NO: 134; cynomolgus: SEQ ID NO: 136), with a Fc hole chain containing the Y349C/T366S/L368A/Y407V mutations (SEQ ID NO: 133) allows generation of a heterodimeric Fc fusion fragment which includes a single copy of the CD19 ectodomain (in analogy to the 4-1BB construct in FIG. 3C). Table 13 lists the cDNA and amino acid sequences of the antigen Fc-fusion construct.

TABLE 11
cDNA and Amino acid sequences
of monomeric human and cynomolgus
CD19 antigen Fc(kih) fusion molecule

| SEQ ID | | |
|---|---|---|
| NO: | Antigen | Sequence |
| 131 | Nucleotide | GACAAAACTCACACATGCCCACCGTGCCCAGCACCTGAAG |
| | sequence of | CTGCAGGGGGACCGTCAGTCTTCCTCTTCCCCCCAAAACCC |
| | Fc hole chain | AAGGACACCCTCATGATCTCCCGGACCCCTGAGGTCACATG |
| | with HYRF | CGTGGTGGTGGACGTGAGCCACGAAGACCCTGAGGTCAAG |
| | mutation | TTCAACTGGTACGTGGACGGCGTGGAGGTGCATAATGCCA |
| | | AGACAAAGCCGCGGGAGGAGCAGTACAACAGCACGTACC |
| | | GTGTGGTCAGCGTCCTCACCGTCCTGCACCAGGACTGGCTG |
| | | AATGGCAAGGAGTACAAGTGCAAGGTCTCCAACAAAGCCC |
| | | TCGGCGCCCCCATCGAGAAAACCATCTCCAAAGCCAAAGG |
| | | GCAGCCCCGAGAACCACAGGTGTGCACCCTGCCCCCATCC |
| | | CGGGATGAGCTGACCAAGAACCAGGTCAGCCTCTCGTGCG |
| | | CAGTCAAAGGCTTCTATCCCAGCGACATCGCCGTGGAGTG |
| | | GGAGAGCAATGGGCAGCCGGAGAACAACTACAAGACCAC |
| | | GCCTCCCGTGCTGGACTCCGACGGCTCCTTCTTCCTCGTGA |
| | | GCAAGCTCACCGTGGACAAGAGCAGGTGGCAGCAGGGGA |
| | | ACGTCTTCTCATGCTCCGTGATGCATGAGGCTCTGCACAAC |
| | | CGCTTCACGCAGAAGAGCCTCTCCCTGTCTCCGGGTAAA |
| 132 | Nucleotide | CCCGAGGAACCCCTGGTCGTGAAGGTGGAAGAGGGCGACA |
| | sequence of | ATGCCGTGCTGCAGTGCCTGAAGGGCACCTCCGATGGCCCT |
| | human CD19 | ACCCAGCAGCTGACCTGGTCCAGAGAGAGCCCCCTGAAGC |
| | antigen Fc | CCTTCCTGAAGCTGTCTCTGGGCCTGCCTGGCCTGGGCATC |
| | knob chain | CATATGAGGCCTCTGGCCATCTGGCTGTTCATCTTCAACGT |
| | avi tag | GTCCCAGCAGATGGGCGGCTTCTACCTGTGTCAGCCTGGCC |
| | | CCCCATCTGAGAAGGCTTGGCAGCCTGGCTGGACCGTGAA |
| | | CGTGGAAGGATCCGGCGAGCTGTTCCGGTGGAACGTGTCC |
| | | GATCTGGGCGGCCTGGGATGCGGCCTGAAGAACAGATCTA |
| | | GCGAGGGCCCCAGCAGCCCCAGCGGCAAACTGATGAGCCC |
| | | CAAGCTGTACGTGTGGGCCAAGGACAGACCCGAGATCTGG |
| | | GAGGGCGAGCCTCCTTGCCTGCCCCCTAGAGACAGCCTGA |
| | | ACCAGAGCCTGAGCCAGGACCTGACAATGGCCCCTGGCAG |
| | | CACACTGTGGCTGAGCTGTGGCGTGCCACCCGACTCTGTGT |
| | | CTAGAGGCCCTCTGAGCTGGACCCACGTGCACCCTAAGGG |
| | | CCCTAAGAGCCTGCTGAGCCTGGAACTGAAGGACGACAGG |
| | | CCCGCCAGAGATATGTGGGTCATGGAAACCGGCCTGCTGC |
| | | TGCCTAGAGCCACAGCCCAGGATGCCGGCAAGTACTACTG |
| | | CCACAGAGGCAACCTGACCATGAGCTTCCACCTGGAAATC |
| | | ACCGCCAGACCCGTGCTGTGGCACTGGCTGCTGAGAACAG |
| | | GCGGCTGGAAGGTCGACGCTAGCGGTGGTAGTCCGACACC |
| | | TCCGACACCCGGGGGTGGTTCTGCAGACAAAACTCACACA |
| | | TGCCCACCGTGCCCAGCACCTGAAGCCGCAGGGGGACCGT |
| | | CAGTCTTCCTCTTCCCCCCAAAACCCAAGGACACCCTCATG |
| | | ATCTCCCGGACCCCTGAGGTCACATGCGTGGTGGTGGACGT |
| | | GAGCCACGAAGACCCTGAGGTCAAGTTCAACTGGTACGTG |
| | | GACGGCGTGGAGGTGCATAATGCCAAGACAAAGCCGCGGG |
| | | AGGAGCAGTACAACAGCACGTACCGTGTGGTCAGCGTCCT |
| | | CACCGTCCTGCACCAGGACTGGCTGAATGGCAAGGAGTAC |
| | | AAGTGCAAGGTCTCCAACAAAGCCCTCGGAGCCCCCATCG |
| | | AGAAAACCATCTCCAAAGCCAAAGGGCAGCCCCGAGAACC |
| | | ACAGGTGTACACCCTGCCCCCATGCCGGGATGAGCTGACC |
| | | AAGAACCAGGTCAGCCTGTGGTGCCTGGTCAAAGGCTTCT |
| | | ATCCCAGCGACATCGCCGTGGAGTGGGAGAGCAATGGGCA |
| | | GCCGGAGAACAACTACAAGACCACGCCTCCCGTGCTGGAC |
| | | TCCGACGGCTCCTTCTTCCTCTACAGCAAGCTCACCGTGGA |
| | | CAAGAGCAGGTGGCAGCAGGGGAACGTCTTCTCATGCTCC |
| | | GTGATGCATGAGGCTCTGCACAACCACTACACGCAGAAGA |
| | | GCCTCTCCCTGTCTCCGGGTAAATCCGGAGGCCTGAACGAC |
| | | ATCTTCGAGGCCCAGAAGATTGAATGGCACGAG |
| 133 | Fc hole chain | DKTHTCPPCPAPEAAGGPSVFLFPPKPKDTLMISRTPEVTCVV |
| | with HYRF | VDVSHEDPEVKFNWYVDGVEVHNAKTKPREEQYNSTYRVVS |
| | mutation | VLTVLHQDWLNGKEYKCKVSNKALGAPIEKTISKAKGQPREP |
| | | QVCTLPPSRDELTKNQVSLSCAVKGFYPSDIAVEWESNGQPE |
| | | NNYKTTPPVLDSDGSFFLVSKLTVDKSRWQQGNVFSCSVMHE |
| | | ALHNRFTQKSLSLSPGK |
| 134 | human CD19 | PEEPLVVKVEEGDNAVLQCLKGTSDGPTQQLTWSRESPLKPF |
| | antigen Fc | LKLSLGLPGLGIHMRPLAIWLFIFNVSQQMGGFYLCQPGPPSE |
| | knob chain | KAWQPGWTVNVEGSGELFRWNVSDLGGLGCGLKNRSSEGPS |
| | avi tag | SPSGKLMSPKLYVWAKDRPEIWEGEPPCLPPRDSLNQSLSQDL |
| | | TMAPGSTLWLSCGVPPDSVSRGPLSWTHVHPKGPKSLLSLEL |
| | | KDDRPARDMWVMETGLLLPRATAQDAGKYYCHRGNLTMSF |
| | | HLEITARPVLWHWLLRTGGWKVDASGGSPTPPTPGGGSADK |
| | | THTCPPCPAPEAAGGPSVFLFPPKPKDTLMISRTPEVTCVVVD |
| | | VSHEDPEVKFNWYVDGVEVHNAKTKPREEQYNSTYRVVSVL |
| | | TVLHQDWLNGKEYKCKVSNKALGAPIEKTISKAKGQPREPQV |
| | | YTLPPCRDELTKNQVSLWCLVKGFYPSDIAVEWESNGQPENN |
| | | YKTTPPVLDSDGSFFLYSKLTVDKSRWQQGNVFSCSVMHEAL |
| | | HNHYTQKSLSLSPGKSGGLNDIFEAQKIEWHE |
| 135 | Nucleotide | CCCCAGGAACCCCTGGTCGTGAAGGTGGAAGAGGGCGACA |
| | sequence of | ATGCCGTGCTCCAGTGCCTGGAAGGCACCTCCGATGGCCCT |
| | cynomolgus | ACACAGCAGCTCGTGTGGTGCAGAGACAGCCCCTTCGAGC |
| | CD19 antigen | CCTTCCTGAACCTGTCTCTGGGCCTGCCTGGCATGGGCATC |
| | Fc knob | AGAATGGGCCCTCTGGGCATCTGGCTGCTGATCTTCAACGT |
| | chain avi tag | GTCCAACCAGACCGGCGGCTTCTACCTGTGTCAGCCTGGCC |
| | | TGCCAAGCGAGAAGGCTTGGCAGCCTGGATGGACCGTGTC |
| | | CGTGGAAGGATCTGGCGAGCTGTTCCGGTGGAACGTGTCC |
| | | GATCTGGGCGGCCTGGGATGCGGCCTGAAGAACAGAAGCA |
| | | GCGAGGGCCCTAGCAGCCCCAGCGGCAAGCTGAATAGCAG |
| | | CCAGCTGTACGTGTGGGCCAAGGACAGACCCGAGATGTGG |
| | | GAGGGCGAGCCTGTGTGTGGCCCCCCTAGAGATAGCCTGA |
| | | ACCAGAGCCTGAGCCAGGACCTGACAATGGCCCCTGGCAG |
| | | CACACTGTGGCTGAGCTGTGGCGTGCCACCCGACTCTGTGT |
| | | CCAGAGGCCCTCTGAGCTGGACACACGTGCGGCCAAAGGG |
| | | CCCTAAGAGCAGCCTGCTGAGCCTGGAACTGAAGGACGAC |
| | | CGGCCCGACCGGGATATGTGGGTGGTGGATACAGGCCTGC |
| | | TGCTGACCAGAGCCACAGCCCAGGATGCCGGCAAGTACTA |
| | | CTGCCACAGAGGCAACTGGACCAAGAGCTTTTACCTGGAA |
| | | ATCACCGCCAGACCCGCCCTGTGGCACTGGCTGCTGAGAAT |
| | | CGGAGGCTGGAAGGTCGACGCTAGCGGTGGTAGTCCGACA |
| | | CCTCCGACACCCGGGGGTGGTTCTGCAGACAAAACTCACA |
| | | CATGCCCACCGTGCCCAGCACCTGAAGCCGCAGGGGGACC |
| | | GTCAGTCTTCCTCTTCCCCCCAAAACCCAAGGACACCCTCA |
| | | TGATCTCCCGGACCCCTGAGGTCACATGCGTGGTGGTGGAC |
| | | GTGAGCCACGAAGACCCTGAGGTCAAGTTCAACTGGTACG |
| | | TGGACGGCGTGGAGGTGCATAATGCCAAGACAAAGCCGCG |
| | | GGAGGAGCAGTACAACAGCACGTACCGTGTGGTCAGCGTC |
| | | CTCACCGTCCTGCACCAGGACTGGCTGAATGGCAAGGAGT |
| | | ACAAGTGCAAGGTCTCCAACAAAGCCCTCGGAGCCCCCAT |
| | | CGAGAAAACCATCTCCAAAGCCAAAGGGCAGCCCCGAGAA |
| | | CCACAGGTGTACACCCTGCCCCCATGCCGGGATGAGCTGA |
| | | CCAAGAACCAGGTCAGCCTGTGGTGCCTGGTCAAAGGCTT |
| | | CTATCCCAGCGACATCGCCGTGGAGTGGGAGAGCAATGGG |
| | | CAGCCGGAGAACAACTACAAGACCACGCCTCCCGTGCTGG |
| | | ACTCCGACGGCTCCTTCTTCCTCTACAGCAAGCTCACCGTG |
| | | GACAAGAGCAGGTGGCAGCAGGGGAACGTCTTCTCATGCT |
| | | CCGTGATGCATGAGGCTCTGCACAACCACTACACGCAGAA |
| | | GAGCCTCTCCCTGTCTCCGGGTAAATCCGGAGGCCTGAACG |
| | | ACATCTTCGAGGCCCAGAAGATTGAATGGCACGAG |
| 136 | cynomolgus | PQEPLVVKVEEGDNAVLQCLEGTSDGPTQQLVWCRDSPFEPF |
| | CD19 antigen | LNLSLGLPGMGIRMGPLGIWLLIFNVSNQTGGFYLCQPGLPSE |
| | Fc knob | KAWQPGWTVSVEGSGELFRWNVSDLGGLGCGLKNRSSEGPS |
| | chain avi tag | SPSGKLNSSQLYVWAKDRPEMWEGEPVCGPPRDSLNQSLSQD |
| | | LTMAPGSTLWLSCGVPPDSVSRGPLSWTHVRPKGPKSSLLSLE |
| | | LKDDRPDRDMWVVDTGLLLTRATAQDAGKYYCHRGNWTKS |
| | | FYLEITARPALWHWLLRIGGWKVDASGGSPTPPTPGGGSADK |
| | | THTCPPCPAPEAAGGPSVFLFPPKPKDTLMISRTPEVTCVVVD |
| | | VSHEDPEVKFNWYVDGVEVHNAKTKPREEQYNSTYRVVSVL |
| | | TVLHQDWLNGKEYKCKVSNKALGAPIEKTISKAKGQPREPQV |
| | | YTLPPCRDELTKNQVSLWCLVKGFYPSDIAVEWESNGQPENN |
| | | YKTTPPVLDSDGSFFLYSKLTVDKSRWQQGNVFSCSVMHEAL |
| | | HNHYTQKSLSLSPGKSGGLNDIFEAQKIEWHE |

For the production of the monomeric antigen/Fc fusion molecules, exponentially growing suspension CHO cells were co-transfected with two plasmids encoding the two components of fusion protein (knob and hole chains) using standard methods.

Secreted protein was purified from cell culture supernatant by affinity chromatography using Protein A, followed by size exclusion chromatography. For affinity chromatography, the supernatant was loaded on a MabSelect Sure column volume (CV)=5-15 mL, resin from GE Healthcare) equilibrated with Sodium Phosphate (20 mM), Sodium Citrate (20 mM), 0.5M sodium chloride buffer (pH 7.5). Unbound protein was removed by washing with at least 6 column volumes of the same buffer. The bound protein was eluted using a linear gradient; step 1, 10 CV from 0 to 60% elution buffer (20 mM sodium citrate, 500 mM Sodium chloride buffer (pH 2.5)); step 2, 2 CV from 60 to 100% elution buffer. For the linear gradient an additional 2 column volumes step elution with 100% elution buffer was applied.

The pH of collected fractions was adjusted by adding 1/40 (v/v) of 2M Tris, pH8.0. The protein was concentrated and filtered prior to loading on a HiLoad Superdex 200 column (GE Healthcare) equilibrated with 2 mM MOPS, 150 mM sodium chloride, 0.02% (w/v) sodium azide solution of pH 7.4.

Table 12 summarizes the yield and final monomer content of monomeric human and cynomolgus CD19 antigen Fc(kih) fusion protein.

TABLE 12
Biochemical analysis of monomeric human and cynomolgus CD19
antigen Fc(kih) fusion protein

| | Monomer [%] | Yield |
|---|---|---|
| Construct | (SEC) | [mg/l] |
| monomeric human CD19 Fc(kih) fusion protein | 91 | 0.2 |
| monomeric cynomolgus CD19 Fc(kih) fusion | 95 | 3.56 |
| protein | | |

Part of the purified antigen was in vitro biotinylated using the BirA biotin-protein ligase standard reaction kit (Avidity, Cat. # BirA500) according to the manufacturer's instructions. The biotinylation degree for the human CD19-containing fusion was 94%, for the respective cynomolgus CD19 construct 100%. The biotinylated protein was then used for selection, screening and characterization of affinity-matured 8B8-derived clones devoid of the de-amidation hotspots N27d and N28. Two phage display libraries were generated in which a) both asparagine residues at positions 27d and 28 were eliminated and b) additional CDRs of heavy and light chain were randomized in order to select for 8B8 variants with an improved affinity.

1.3.3 Generation of 8B8 Affinity Maturation Libraries Devoid of CDR-L1 Hotspots

Generation of affinity-matured 8B8-derived antibodies without the de-amidation sites N27d and N28, located in CDR-L1, was carried out by phage display using standard protocols (Silacci et al, 2005). In a first step, the VL and VH DNA sequences of the humanized parental clone 8B8 (SEQ ID NO: 137 and SEQ ID NO: 138) were cloned into a phagemid which was then used as a template for randomization. In a next step, two libraries were generated for the selection of favourable clones by phage display. In order to eliminate the above-mentioned hotspot positions, a LCDR1 randomization primer (SEQ ID NO: 139) that only allowed amino acids S T Q E at positions 27d and 28 was used for both libraries. Maturation library 1 was randomized in CDR1 and 2 of both the light and the heavy chain, while maturation library 2 was randomized in CDR1 and 3 of the light chain and in CDR3 of the heavy chain. For the generation of the maturation library 1, randomized in CDR1 and 2 of both the light and the heavy chain, three fragments were assembled by “splicing by overlapping extension” (SOE) PCR and cloned into the phage vector. The following primer combinations were used to generate the library fragments: fragment 1 (LMB3 (SEQ ID NO: 144) and CD19 L1 reverse random (SEQ ID NO: 139), fragment 2 (CD19 L2 forward random (SEQ ID NO: 140) and CD19 H1 reverse random (SEQ ID NO: 141), and fragment 3 (CD19 H2 forward random (SEQ ID NO: 142) and CD19 H3 reverse constant (SEQ ID NO: 143) (Table 13). After assembly of sufficient amounts of full length randomized fragment, it was digested with NcoI/NheI alongside with identically treated acceptor phagemid vector. A 3-fold molar excess of library insert was ligated with 10 μg of phagemid vector. Purified ligations were used for 20 transformations resulting in 2×10 exp9 transformants. Phagemid particles displaying the 8B8 affinity maturation library were rescued and purified by PEG/NaCl purification to be used for selections.

The generation of the second library, randomized in CDR1 and 3 of the light chain and in CDR3 of the heavy chain, was done similarly. The following primer combinations were used to generate the library fragments: fragment 1 (LMB3 (SEQ ID NO: 144) and CD19 L1 reverse random (SEQ ID NO: 139), fragment 2 (CD19 L1 forward constant (SEQ ID NO 145) and CD19 L3 reverse random (SEQ ID NO: 146, and fragment 3 (CD19 L3 forward constant (SEQ ID NO: 147) and CD19 H3 reverse random (SEQ ID NO: 148) (Table 14). After assembly of sufficient amounts of full length randomized fragment, it was digested with NcoI/KpnI alongside with identically treated acceptor phagemid vector. A 3-fold molar excess of library insert was ligated with 20 ug of phagemid vector. Purified ligations were used for 40 transformations resulting in 2 ×10 exp9 transformants. Phagemid particles displaying the 8B8 affinity maturation library were rescued and purified by PEG/NaClp urification to be used for selections.

TABLE 13
Primers for 8B8 affinity maturation and hotspot
removal library L1_L2/H1_H2

| SEQ | | |
|---|---|---|
| ID | Name | Sequence |
| 139 | CD19 L1 | CAG CTG CGG GCT CTG ACC CGG TTT |
| | reverse | CTG GAG ATA CCA GTT CAG 1 CGT |
| | random | 2 GCC 3 GGA 4 TTC CAG AGA TTG |
| | | GCT GGA TTT GCA AGA AAT G |
| | | 1: 40% Y, 6% A/S/T/G/P/D/N/E/Q/V, |
| | | 2: 40% N, 6% A/S/T/Y/G/P/D/E/Q/V, |
| | | 3: 25% S/T/Q/E, 4: 25% S/T/Q/E |
| 140 | CD19 L2 | CTC CAG AAA CCG GGT CAG AGC CCG |
| | forward | CAG CTG CTG ATC TAC 5 GTA TCT 6 |
| | random | CGC 7 8 GGC GTT 9 GAT CGT TTC |
| | | AGC GGT TCT GGA TCC GGC ACC |
| | | 5: 30% R, 20% E, 5% A/S/T/Y/G/P/ |
| | | D/N/Q/V, 6: 30% K, 20% S, 5% A/N/ |
| | | T/Y/G/P/D/E/Q/V, 7: 40% F, 5% A/ |
| | | S/T/Y/G/P/D/E/Q/V/I/L, 8: 40% S, |
| | | 6.6% A/T/Y/G/P/D/E/Q/V, 9: 50% P, |
| | | 50% L |
| 141 | CD19 H1 | CAT CCA CTC CAG ACC CTG GCC CGG |
| | reverse | GGC CTG ACG AAC CCA 10 CAT 11 12 |
| | random | 13 14 GAA 15 GTA ACC AGA TGC TTT |
| | | GCA GCT CAC TTT AAC GGA AGC |
| | | 10: 52% H, 4% G/A/S/P/T/N/Y/D/ |
| | | E/Q/V/I, 11: 30% I, 15% Y, 5% |
| | | G/A/S/T/P/N/H/D/E/Q/V, 12: 52% |
| | | Y, 4% G/A/S/P/T/N/H/D/E/Q/V/I, |
| | | 13: 30% D, 15% G, 5% A/S/P/Y/ |
| | | N/H/D/E/Q/V/I, 14: 52% T, 4% G/ |
| | | A/S/P/Y/N/H/D/E/Q/V/I, 15: 52% |
| | | T, 4% G/A/S/P/Y/N/H/D/E/Q/V/I |
| 142 | CD19 H2 | CAG GCC CCG GGC CAG GGT CTG GAG |
| | forward | TGG ATG GGC 16 ATT 17 CCA 18 19 |
| | random | 20 21 TCC 22 TAT ACC 23 AAA TTC |
| | | CAG GGC CGC GTC ACG ATG ACC |
| | | 16: 45% Y, 5% A/S/P/T/N/H/D/E/Q/ |
| | | V/I, 17: 52% N, 4% G/A/S/P/Y/T/ |
| | | H/D/E/Q/V/I, 18: 40% Y, 5% G/A/ |
| | | S/P/T/N/H/D/E/Q/V/I, 19: 30% N, |
| | | 15% S, 5% G/A/T/P/Y/H/D/E/Q/V/I, |
| | | 20: 30% D, 15% G, 5% A/S/T/P/Y/ |
| | | N/H/E/Q/V/I, 21: 52% G, 4% N/A/ |
| | | S/P/Y/T/H/D/E/Q/V/I, 22: 30% K, |
| | | 15% N, 4% G/A/S/P/Y/T/H/D/E/Q/ |
| | | V/I, 23: 30% E, 15% Q, 5% G/A/ |
| | | S/T/P/Y/N/H/D/V/I |
| 143 | CD19 H3 | CGTCACCGGTTCGGGGAAGTAGTCCTTGACCAG |
| | reverse | |
| | constant | |
| 144 | LMB3 | CAGGAAACAGCTATGACCATGATTAC |

TABLE 14
Primers for 8B8 affinity maturation and
hotspot removal library L1_L3/H3

| SEQ | | |
|---|---|---|
| ID | Name | Sequence |
| 145 | CD19 L1 | TGGTATCTCCAGAAACCGGGTCAGAGCCCGCAG |
| | forward | |
| | constant | |
| 139 | CD19 L1 | See Table 15 |
| | reverse | |
| | random | |
| 146 | CD19 L3 | TTT AAT TTC CAG TTT AGT TCC TTG |
| | reverse | ACC GAA GGT 24 25 26 27 28 29 CTG |
| | random | CAG ACA ATA GTA GAC GCC AAC |
| | | GTC TTC AGC |
| | | 24: 52% Y, 4% G/A/S/T/N/P/D/E/ |
| | | Q/V/L/I, 25: 52% P, 4% G/A/S/T/ |
| | | Y/N/H/D/E/Q/V/I, 26: 42% V, |
| | | 10% L, 4% G/A/S/T/Y/N/P/D/E/Q/ |
| | | V/I, 27: 52% H, 4% G/A/S/T/Y/ |
| | | N/P/D/E/Q/V/I, 28: 42% T, 10% I, |
| | | 4% G/A/S/T/Y/N/P/D/E/Q/V/L, 29: |
| | | 45% L, 11% G, 4% A/S/T/Y/N/P/D/ |
| | | E/Q/V/I |
| 147 | CD19 L3 | ACCTTCGGTCAAGGAACTAAACTGGAAATTAAACG |
| | forward | |
| | constant | |
| 148 | CD19 H3 | TT GGT GCT AGC AGA GCT TAC GGT |
| | reverse | CAC CGT GGT ACC TTG GCC CCA GTA |
| | random | ATC AAA 30 31 32 33 34 35 36 37 |
| | | 38 GCG TGC ACA ATA GTA AAC AGC |
| | | GGT GTC |
| | | 30: 50% L, 3.8% G/A/S/T/P/H/Y/N/ |
| | | D/E/Q/V/I, 31: 50% A, 4.2% G/S/ |
| | | T/P/H/Y/N/D/E/Q/V/I, 32: 50% S, |
| | | 4.2% |
| | | G/A/T/P/H/Y/N/D/E/Q/V/I, 33: |
| | | 50% G, 4.2% S/A/T/P/H/Y/N/D/ |
| | | E/Q/V/I, 34: 50% Y, 4.2% G/ |
| | | A/T/P/H/S/N/D/E/Q/V/I, 35: |
| | | 50% Y, 4.2% G/A/T/P/H/S/N/D/E/ |
| | | Q/V/I, 36: 50% Y, 4.2% G/A/T/ |
| | | P/H/S/N/D/E/Q/V/I, 37: 50% T, |
| | | 4.2% G/A/Y/P/H/S/N/D/E/Q/V/I, |
| | | 38: 50% G, 4.2% Y/A/T/P/H/S/N/ |
| | | D/E/Q/V/I |
| 144 | LMB3 | See Table 15 |

1.3.4 Selection of Affinity Matured 818-Derived Clones Devoid of CDR-L1 Hotspots N27d and N28

For the selection of affinity-matured clones devoid of the CDR-L1 hotspots N27d and N28, two selection approaches by phage display were performed:

In the first approach, the selection was executed on human CD19-Fc fusion protein using both phage display libraries. Panning rounds were performed in solution according to the following pattern: 1. binding of ˜10¹² phagemid particles to 30 nM biotinylated CD19-Fc protein for 0.5 h in a total volume of 1 ml, 2. capture of biotinylated CD19-Fc protein and specifically bound phage particles by addition of 5.4×10⁷ streptavidin-coated magnetic beads for 10 min, 3. washing of beads using 5×1 ml PBS/Tween20 and 5×1 ml PBS, 4. elution of phage particles by addition of 1 ml 100 mM TEA for 10 min and neutralization by adding 500 ul 1M Tris/HCl pH 7.4, 5. re-infection of exponentially growing E. coli TG1 bacteria, and 6.infection with helperphage VCSM13 and subsequent PEG/NaCi precipitation of phagemid particles to be used in subsequent selection rounds. Selections were carried out over 3 rounds using decreasing antigen concentrations (30×10⁻⁹M, 10×10⁻⁹M, and 3×10⁻⁹M). In round 2 and 3, capture of antigen:phage complexes was performed using neutravidin plates instead of streptavidin beads. Neutravidin plates were washed with 5×PBS/Tween20 and 5×PBS. In round 3, the neutravidin plate was incubated overnight in 2 liters PBS for an “off-rate” selection before phage was eluted from the plate. Furthermore, cynomolgus CD19-Fc protein was used in round 2 in order to enrich cross-reactive binders.

In the second selection approach, the phage panning was executed on cells transiently expressing either the human or cynomolgus CD19 ECD on the cell surface. For the transient transfection of HEK cells, expression plasmids were generated that harbor the DNA sequences (from 5′ to 3′) for the following protein segments: A Flag tag, a SNAP tag, the CD19 ECD of either human or cynomolgus origin, and the transmembrane region of the Platelet-derived growth factor receptor (PDGFR) (SEQ ID NOs: 149 and 150). The expression of the respective proteins (SEQ ID NOs: 151 and 152) on the cell surface was confirmed by flow cytometry using an anti-Flag antibody for detection. Both libraries were exposed in the first selection round to cells either expressing the human or cynomolgus CD19 ECD-containing protein fusion. For the subsequent panning rounds, the species of the CD19 ECD was alternated accordingly. Cells transiently transfected with an irrelevant membrane protein were used for pre-clearing.

Panning rounds were performed according to the following pattern:

1. Transfection of HEK cells with constructs expressing either CD19 ECD or an irrelevant transmembrane protein according to the standard procedure described before,
2. Incubation of the cells for total 48 h at 37° C. in an incubator with a 5% CO₂ atmosphere,
3. Isolation of cells by centrifugation (3 min at 250×g) and re-suspension of 1×10E7 CD19 ECD-positive cells and 1×10E7 negative cells in PBS/5% BSA, respectively,
4. Pre-clearing of unspecific phage by incubating the phage library with 1×107 CD19-negative cells for 60 min at 4° C. using a gently rotating tube rotator,
5. Centrifugation of cells at 250×g for 3 min and transfer of supernatant into a fresh tube and addition of 1×10E7 CD19-positive cells and incubation for 60 min at 4° C. by gentle rotation on a tube rotator,
6. Washing of cells by centrifugation for 1 min at 250×g, aspiration of the supernatant, and re-suspension in 1 ml PBS (8 times),
7. Phage elution with 1 ml 100 mM TEA, incubation for 5 min at RT, and neutralization of the eluate with 500 ul 1M Tris-HCl, pH7.6,
8. re-infection of exponentially growing E. coli TG1 bacteria, and
9. infection with helperphage VCSM13 and subsequent PEG/NaCi precipitation of phagemid particles to be used in subsequent selection rounds. Selections were carried out over 3 rounds.

For both selection approaches, specific binders were identified by ELISA as follows: 100 ul of 30 nM biotinylated CD19-Fc protein per well were coated on neutravidin plates. Fab-containing bacterial supernatants were added and binding Fabs were detected via their Flag-tags using an anti-Flag/HRP secondary antibody.

Clones that were ELISA-positive on recombinant human CD19 were further tested in a cell-based ELISA using cells that were transiently transfected with the human CD19 ECD-containing expression plasmid (SEQ ID NO: 149). This analysis was performed as follows: 48 h after transfection, HEK cells were harvested and centrifuged at 250×g for 5 min. Cells were then re suspended in ice-cold PBS BSA 2% to 4×10⁶ cells/ml and incubated for 20 min on ice to block unspecific binding sites. 4×10⁵ cells in 100 ul were distributed to each well of a 96 well plate and centrifuged at 250×g and 4° C. for 3 min. Supernatant was aspirated off and 50 ul bacterial supernatant containing soluble Fab fragments was diluted with 50 ul ice-cold PBS/BSA 2%, added to the plate, mixed with the cells and incubated for 1 h at 4C. Afterwards, cells were washed 3 times with ice cold PBS before 100 ul PBS BSA 2% per well containing a 1:2000 dilution of anti-Fab-HRP antibody were added. After an incubation time of 1 h, cells were washed again 3 times with ice-cold PBS. For the development, 100 ul “1-step ultra TMB-ELISA” substrate was added per well. After an incubation time of 10 minutes, supernatant was transferred to a new 96-well plate containing 40 ul H₂SO₄ 1M per well and absorbance was measured 450 nM. Clones exhibiting significant signals over background were subjected to a kinetic screening experiment by SPR-analysis using ProteOn XPR36.

1.3.5 Identification of Affinity-Matured 8B8-Derived Variants by SPR

In order to further characterize the ELISA-positive clones, the off-rate was measured by surface plasmon resonance and compared with the parental humanized clone 8B8.

For this experiment, 7000 RU of polyclonal anti-human Fab antibody were immobilized on all 6 channels of a GLM chip by Amine coupling (NaAcetate pH4.5, 25 l/min, 240s) (vertical orientation). Each antibody-containing bacterial supernatant was filtered and 2-fold diluted with PBS, and then injected for 360s at 25 μl/minute to achieve immobilization levels of between 100 and 400 response units (RU) in vertical orientation. Injection of monomeric CD19-Fc: For one-shot kinetics measurements, injection direction was changed to horizontal orientation, three-fold dilution series of purified monomeric CD19-Fc (varying concentration ranges between 150 and 6 nM) were injected simultaneously at 50 μl/min along separate channels 1-4, with association times of 180 s, and dissociation times of 300 s. A human IgG Fc fragment (150 nM) was injected in channel 5 as a negative control for specific binding to the affinity matured CD19 variants along with a PBS injection in the 6th channel to provide an “in-line” blank for referencing. Regeneration was performed by two pulses of 10 mM glycine pH 1.5 and 50 mM NaOH for 30s at 90 ul/min(horizontal orientation). Dissociation rate constants (k_{off}) were calculated using a simple one-to-one Langmuir binding model in ProteOn Manager v3.1 software by simultaneously fitting the sensorgrams. Clones expressing Fabs with the slowest dissociation rate constants were identified. The variable domains of the corresponding phagemids were sequenced. Importantly, both asparagine residue in CDR-L1 (position 27d and 28) were replaced by a serine or a threonine, demonstrating that both de-amidation sites were removed.

TABLE 15
Dissociation constants of parental 8B8 and selected clone 2B11
obtained in screening analysis with bacterial supernatant

| clone | Dissociation constant kd (1/s) |
|---|---|
| Parental 8B8 | 3.01E−4 |
| 2B11 | 4.37E−6 |

Table 16 shows the amino acid sequences of the CDRs and variable regions VH and VLof clones 818-018 and 818-21B11, respectively.

TABLE 16
Sequences of of the CDRs and variable regions
VH and VL of clones 8B8-018 and 8B8-2B11

| SEQ | De- | |
|---|---|---|
| ID | scrip- | |
| NO: | tion | Sequence |
| 29 | CD19 | DYIMH |
| | (8B8- | |
| | 018) | |
| | CDR-H1 | |
| 30 | CD19 | YINPYNDGSKYTEKFQG |
| | (8B8- | |
| | 018) | |
| | CDR-H2 | |
| 31 | CD19 | GTYYYGSALFDY |
| | (8B8- | |
| | 018) | |
| | CDR-H3 | |
| 32 | CD19 | KSSQSLENPNGNTYLN |
| | (8B8- | |
| | 018) | |
| | CDR-L1 | |
| 33 | CD19 | RVSKRFS |
| | (8B8- | |
| | 018) | |
| | CDR-L2 | |
| 34 | CD19 | LQLTHVPYT |
| | (8B8- | |
| | 018) | |
| | CDR-L3 | |
| 41 | CD19 | QVQLVQSGAEVKKPGASVKVSCKASGYTFTDYIMH |
| | (8B8- | WVRQAPGQGLEWMGYINPYNDGSKYTEKFQGRVT |
| | 018) | MTSDTSISTAYMELSRLRSDDTAVYYCARGTYYYGS |
| | VH | ALFDYWGQGTTVTVSS |
| 42 | CD19 | DIVMTQTPLSLSVTPGQPASISCKSSQSLENPNGNTYL |
| | (8B8- | NWYLQKPGQSPQLLIYRVSKRFSGVPDRFSGSGSGTD |
| | 018) | FTLKISRVEAEDVGVYYCLQLTHVPYTFGQGTKLEIK |
| | VL | |
| 35 | CD19 | DYIMH |
| | (8B8- | |
| | 2B11) | |
| | CDR-H1 | |
| 36 | CD19 | YINPYNDGSKYTEKFQG |
| | (8B8- | |
| | 2B11) | |
| | CDR-H2 | |
| 37 | CD19 | GTYYYGPQLFDY |
| | (8B8- | |
| | 2B11) | |
| | CDR-H3 | |
| 38 | CD19 | KSSQSLETSTGTTYLN |
| | (8B8- | |
| | 2B11) | |
| | CDR-L1 | |
| 39 | CD19 | RVSKRFS |
| | (8B8- | |
| | 2B11) | |
| | CDR-L2 | |
| 40 | CD19 | LQLLEDPYT |
| | (8B8- | |
| | 2B11) | |
| | CDR-L3 | |
| 43 | CD19 | QVQLVQSGAEVKKPGASVKVSCKASGYTFTDYIMH |
| | (8B8- | WVRQAPGQGLEWMGYINPYNDGSKYTEKFQGRVT |
| | 2B11) | MTSDTSISTAYMELSRLRSDDTAVYYCARGTYYYGP |
| | VH | QLFDYWGQGTTVTVSS |
| 44 | CD19 | DIVMTQTPLSLSVTPGQPASISCKSSQSLETSTGTTYL |
| | (8B8- | NWYLQKPGQSPQLLIYRVSKRFSGVPDRFSGSGSGTD |
| | 2B11) | FTLKISRVEAEDVGVYYCLQLLEDPYTFGQGTKLEIK |
| | VL | |

1.4 Preparation of Monovalent CEA-Targeted 4-1BB Ligand Trimer-Containing Fc (Kih) Fusion Antigen Binding Molecule, Wherein the 4-1BB Ligands are C-Terminally Fused (Constructs 5.11 and 5.12) (CEA on the Knob Chain)

The molecule was prepared as described in Example 1.1 for the monovalent FAP targeted construct, with the only difference that the anti-FAP binder was replaced by an anti-CEA binder.

The variable region of heavy and light chain DNA sequences encoding binder specific for carcinoembryonic antigen (CEA), clone T84.66-LCHA, were subcloned in frame with either the constant heavy chain of the knob or the constant light chain of human IgG1. The generation of the CEA clone is described in Example 1.5.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

For all constructs the knobs into holes heterodimerization technology was used as described in Carter, J Immunol Methods 248, 7-15 (2001). Combination of the huIgG1 Fc hole chain containing the Y349C/T366S/L368A/Y407V mutations in the CH3 domain, the anti-FAP huIgG1 knob chain containing the S354C/T366W mutations in the CH3 domain and the anti-FAP light chain allows generation of a heterodimer, which includes an assembled trimeric 4-1BB ligand and one CEA binding Fab (FIGS. 2A and 2B).

Table 17 shows the cDNA and amino acid sequences of the monovalent CEA(T84.66-LCHA)-targeted 4-1BB ligand (71-248) trimer-containing Fc (kih) fusion antigen binding molecule (Construct 5.11).

TABLE 17
Sequences of CEA(T84.66-LCHA)-targeted human
4-1BB ligand trimer containing Fc (kih)
fusion molecule Construct 5.11

| SEQ | De- | |
|---|---|---|
| ID | scrip- | |
| NO: | tion | Sequence |
| 101 | nucleo- | see Table 1 |
| | tide | |
| | se- | |
| | quence | |
| | of | |
| | Fc hole | |
| | dimeric | |
| | 4-1BB | |
| | ligand | |
| | (71- | |
| | 248) | |
| | chain | |
| 153 | nucleo- | CAGGTGCAGCTGGTGCAGTCTGGCGCCGAAGTGAAGAA |
| | tide | ACCCGGCAGCAGCGTGAAGGTGTCCTGCAAGGCCAGCG |
| | se- | GCTTCAACATCAAGGACACCTACATGCACTGGGTGCGCC |
| | quence | AGGCCCCTGGACAGGGACTGGAATGGATGGGCAGAATC |
| | of | GACCCCGCCAACGGCAACAGCAAATACGTGCCCAAGTT |
| | anti- | CCAGGGCAGAGTGACCATCACCGCCGACACCAGCACCT |
| | CEA | CCACCGCCTACATGGAACTGAGCAGCCTGCGGAGCGAG |
| | (T84. | GACACCGCCGTGTACTACTGTGCCCCCTTCGGCTACTAC |
| | 66- | GTGTCCGACTACGCCATGGCCTATTGGGGCCAGGGCAC |
| | LCHA) | ACTCGTGACCGTGTCCTCTGCTAGCACCAAGGGCCCATC |
| | Fc knob | GGTCTTCCCCCTGGCACCCTCCTCCAAGAGCACCTCTGG |
| | mono- | GGGCACAGCGGCCCTGGGCTGCCTGGTCAAGGACTACT |
| | meric | TCCCCGAACCGGTGACGGTGTCGTGGAACTCAGGCGCC |
| | 4-1BB | CTGACCAGCGGCGTGCACACCTTCCCGGCTGTCCTACAG |
| | (71- | TCCTCAGGACTCTACTCCCTCAGCAGCGTGGTGACCGTG |
| | 248) | CCCTCCAGCAGCTTGGGCACCCAGACCTACATCTGCAAC |
| | ligand | GTGAATCACAAGCCCAGCAACACCAAGGTGGACAAGAA |
| | | AGTTGAGCCCAAATCTTGTGACAAAACTCACACATGCCC |
| | | ACCGTGCCCAGCACCTGAAGCTGCAGGGGGACCGTCAG |
| | | TCTTCCTCTTCCCCCCAAAACCCAAGGACACCCTCATGA |
| | | TCTCCCGGACCCCTGAGGTCACATGCGTGGTGGTGGACG |
| | | TGAGCCACGAAGACCCTGAGGTCAAGTTCAACTGGTAC |
| | | GTGGACGGCGTGGAGGTGCATAATGCCAAGACAAAGCC |
| | | GCGGGAGGAGCAGTACAACAGCACGTACCGTGTGGTCA |
| | | GCGTCCTCACCGTCCTGCACCAGGACTGGCTGAATGGCA |
| | | AGGAGTACAAGTGCAAGGTCTCCAACAAAGCCCTCGGC |
| | | GCCCCCATCGAGAAAACCATCTCCAAAGCCAAAGGGCA |
| | | GCCCCGAGAACCACAGGTGTACACCCTGCCCCCCTGCA |
| | | GAGATGAGCTGACCAAGAACCAGGTGTCCCTGTGGTGT |
| | | CTGGTCAAGGGCTTCTACCCCAGCGATATCGCCGTGGAG |
| | | TGGGAGAGCAACGGCCAGCCTGAGAACAACTACAAGAC |
| | | CACCCCCCCTGTGCTGGACAGCGACGGCAGCTTCTTCCT |
| | | GTACTCCAAACTGACCGTGGACAAGAGCCGGTGGCAGC |
| | | AGGGCAACGTGTTCAGCTGCAGCGTGATGCACGAGGCC |
| | | CTGCACAACCACTACACCCAGAAGTCCCTGAGCCTGAG |
| | | CCCCGGCGGAGGCGGCGGAAGCGGAGGAGGAGGATCC |
| | | AGAGAGGGCCCTGAGCTGAGCCCTGATGATCCTGCCGG |
| | | ACTGCTGGACCTGCGGCAGGGAATGTTTGCCCAGCTGGT |
| | | GGCCCAGAACGTGCTGCTGATCGATGGCCCCCTGTCCTG |
| | | GTACAGCGATCCTGGACTGGCTGGCGTGTCACTGACAGG |
| | | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGATCTGCT |
| | | GCTGGCGCCGCTGCTCTGGCACTGACAGTGGATCTGCCT |
| | | CCTGCCAGCAGCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCTGCCGGGCTC |
| 154 | nucleo- | GAGATCGTGCTGACCCAGAGCCCTGCCACCCTGTCACTG |
| | tide | TCTCCAGGCGAGAGAGCCACCCTGAGCTGTAGAGCCGG |
| | se- | CGAGAGCGTGGACATCTTCGGCGTGGGATTTCTGCACTG |
| | quence | GTATCAGCAGAAGCCCGGCCAGGCCCCCAGACTGCTGA |
| | of | TCTACAGAGCCAGCAACCGGGCCACAGGCATCCCCGCC |
| | anti- | AGATTTTCTGGCTCTGGCAGCGGCACCGACTTCACCCTG |
| | CEA | ACAATCAGCAGCCTGGAACCCGAGGACTTCGCCGTGTA |
| | (T84. | CTACTGCCAGCAGACCAACGAGGACCCCTACACCTTTGG |
| | 66- | CCAGGGCACCAAGCTGGAAATCAAGCGTACGGTGGCTG |
| | LCHA) | CACCATCTGTCTTCATCTTCCCGCCATCTGATGAGCAGT |
| | light | TGAAATCTGGAACTGCCTCTGTTGTGTGCCTGCTGAATA |
| | chain | ACTTCTATCCCAGAGAGGCCAAAGTACAGTGGAAGGTGG |
| | | ATAACGCCCTCCAATCGGGTAACTCCCAGGAGAGTGTC |
| | | ACAGAGCAGGACAGCAAGGACAGCACCTACAGCCTCAG |
| | | CAGCACCCTGACGCTGAGCAAAGCAGACTACGAGAAAC |
| | | ACAAAGTCTACGCCTGCGAAGTCACCCATCAGGGCCTG |
| | | AGCTCGCCCGTCACAAAGAGCTTCAACAGGGGAGAGTG |
| | | T |
| 104 | Fc hole | see Table 1 |
| | dimeric | |
| | 4-1BB | |
| | (71- | |
| | 248) | |
| | ligand | |
| | chain | |
| 155 | anti- | QVQLVQSGAEVKKPGSSVKVSCKASGFNIKDTYMHWVRQ |
| | CEA | APGQGLEWMGRIDPANGNSKYVPKFQGRVTITADTSTST |
| | (T84. | AYMELSSLRSEDTAVYYCAPFGYYVSDYAMAYWGQGTLV |
| | 66- | TVSSASTKGPSVFPLAPSSKSTSGGTAALGCLVKDYFPE |
| | LCHA) | PVTVSWNSGALTSGVHTFPAVLQSSGLYSLSSVVTVPSS |
| | Fc knob | SLGTQTYICNVNHKPSNTKVDKKVEPKSCDKTHTCPPCP |
| | mono- | APEAAGGPSVFLFPPKPKDTLMISRTPEVTCVVVDVSHE |
| | meric | DPEVKFNWYVDGVEVHNAKTKPREEQYNSTYRVVSVLTV |
| | 4-1-BB | LHQDWLNGKEYKCKVSNKALGAPIEKTISKAKGQPREPQ |
| | (71- | VYTLPPCRDELTKNQVSLWCLVKGFYPSDIAVEWESNGQ |
| | 248) | PENNYKTTPPVLDSDGSFFLYSKLTVDKSRWQQGNVFSC |
| | ligand | SVMHEALHNHYTQKSLSLSPGGGGGSGGGGSREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGL |
| 156 | anti- | EIVLTQSPATLSLSPGERATLSCRAGESVDIFGVGFLHW |
| | CEA | YQQKPGQAPRLLIYRASNRATGIPARFSGSGSGTDFTLT |
| | (T84. | ISSLEPEDFAVYYCQQTNEDPYTFGQGTKLEIKRTVAAP |
| | 66- | SVFIFPPSDEQLKSGTASVVCLLNNFYPREAKVQWKVDN |
| | LCHA) | ALQSGNSQESVTEQDSKDSTYSLSSTLTLSKADYEKHKV |
| | light | YACEVTHQGLSSPVTKSFNRGEC |
| | chain | |

Table 18 shows the cDNA and amino acid sequences of the monovalent CEA(T84.66-LCHA)-targeted 4-1BB ligand (71-248) trimer-containing Fc (kih) fusion antigen binding molecule (Construct 5.12).

TABLE 18
Sequences of CEA(T84.66-LCHA)-targeted human
4-1BB ligand trimer containing Fc (kih)
fusion molecule Construct 5.12

| SEQ | De- | |
|---|---|---|
| ID | scrip- | |
| NO: | tion | Sequence |
| 107 | nucleo- | see Table 2 |
| | tide | |
| | se- | |
| | quence | |
| | of | |
| | Fc hole | |
| | mono- | |
| | meric | |
| | 4-1BBL | |
| | (71- | |
| | 248) | |
| | chain | |
| 157 | nucleo- | CAGGTGCAGCTGGTGCAGTCTGGCGCCGAAGTGAAGAA |
| | tide | ACCCGGCAGCAGCGTGAAGGTGTCCTGCAAGGCCAGCG |
| | se- | GCTTCAACATCAAGGACACCTACATGCACTGGGTGCGCC |
| | quence | AGGCCCCTGGACAGGGACTGGAATGGATGGGCAGAATC |
| | of | GACCCCGCCAACGGCAACAGCAAATACGTGCCCAAGTT |
| | anti- | CCAGGGCAGAGTGACCATCACCGCCGACACCAGCACCT |
| | CEA | CCACCGCCTACATGGAACTGAGCAGCCTGCGGAGCGAG |
| | (T84. | GACACCGCCGTGTACTACTGTGCCCCCTTCGGCTACTAC |
| | 66- | GTGTCCGACTACGCCATGGCCTATTGGGGCCAGGGCAC |
| | LCHA) | ACTCGTGACCGTGTCCTCTGCTAGCACCAAGGGCCCATC |
| | Fc knob | GGTCTTCCCCCTGGCACCCTCCTCCAAGAGCACCTCTGG |
| | dimeric | GGGCACAGCGGCCCTGGGCTGCCTGGTCAAGGACTACT |
| | 4-1BB | TCCCCGAACCGGTGACGGTGTCGTGGAACTCAGGCGCC |
| | (71- | CTGACCAGCGGCGTGCACACCTTCCCGGCTGTCCTACAG |
| | 248 | TCCTCAGGACTCTACTCCCTCAGCAGCGTGGTGACCGTG |
| | ligand | CCCTCCAGCAGCTTGGGCACCCAGACCTACATCTGCAAC |
| | | GTGAATCACAAGCCCAGCAACACCAAGGTGGACAAGAA |
| | | AGTTGAGCCCAAATCTTGTGACAAAACTCACACATGCCC |
| | | ACCGTGCCCAGCACCTGAAGCTGCAGGGGGACCGTCAG |
| | | TCTTCCTCTTCCCCCCAAAACCCAAGGACACCCTCATGA |
| | | TCTCCCGGACCCCTGAGGTCACATGCGTGGTGGTGGACG |
| | | TGAGCCACGAAGACCCTGAGGTCAAGTTCAACTGGTAC |
| | | GTGGACGGCGTGGAGGTGCATAATGCCAAGACAAAGCC |
| | | GCGGGAGGAGCAGTACAACAGCACGTACCGTGTGGTCA |
| | | GCGTCCTCACCGTCCTGCACCAGGACTGGCTGAATGGCA |
| | | AGGAGTACAAGTGCAAGGTCTCCAACAAAGCCCTCGGC |
| | | GCCCCCATCGAGAAAACCATCTCCAAAGCCAAAGGGCA |
| | | GCCCCGAGAACCACAGGTGTACACCCTGCCCCCCTGCA |
| | | GAGATGAGCTGACCAAGAACCAGGTGTCCCTGTGGTGT |
| | | CTGGTCAAGGGCTTCTACCCCAGCGATATCGCCGTGGAG |
| | | TGGGAGAGCAACGGCCAGCCTGAGAACAACTACAAGAC |
| | | CACCCCCCCTGTGCTGGACAGCGACGGCAGCTTCTTCCT |
| | | GTACTCCAAACTGACCGTGGACAAGAGCCGGTGGCAGC |
| | | AGGGCAACGTGTTCAGCTGCAGCGTGATGCACGAGGCC |
| | | CTGCACAACCACTACACCCAGAAGTCCCTGAGCCTGAG |
| | | CCCCGGCAGAGAGGGCCCTGAGCTGAGCCCTGATGATC |
| | | CTGCCGGACTGCTGGACCTGCGGCAGGGAATGTTTGCCC |
| | | AGCTGGTGGCCCAGAACGTGCTGCTGATCGATGGCCCCC |
| | | TGTCCTGGTACAGCGATCCTGGACTGGCTGGCGTGTCAC |
| | | TGACAGGCGGCCTGAGCTACAAAGAGGACACCAAAGAA |
| | | CTGGTGGTGGCCAAGGCCGGCGTGTACTACGTGTTCTTT |
| | | CAGCTGGAACTGCGGAGAGTGGTGGCCGGCGAAGGATC |
| | | TGGCTCTGTGTCTCTGGCCCTGCATCTGCAGCCTCTGAG |
| | | ATCTGCTGCTGGCGCCGCTGCTCTGGCACTGACAGTGGA |
| | | TCTGCCTCCTGCCAGCAGCGAGGCCCGGAATAGCGCATT |
| | | TGGGTTTCAAGGCAGGCTGCTGCACCTGTCTGCCGGCCA |
| | | GAGGCTGGGAGTGCATCTGCACACAGAGGCCAGGGCTA |
| | | GACACGCCTGGCAGCTGACACAGGGCGCTACAGTGCTG |
| | | GGCCTGTTCAGAGTGACCCCCGAGATTCCAGCAGGCCTG |
| | | GGAGGCGGCGGATCTGGCGGCGGAGGATCTAGAGAAGG |
| | | ACCCGAGCTGTCCCCCGACGATCCCGCTGGGCTGCTGGA |
| | | TCTGAGACAGGGCATGTTCGCTCAGCTGGTGGCTCAGAA |
| | | TGTGCTGCTGATTGACGGACCTCTGAGCTGGTACTCCGA |
| | | CCCAGGGCTGGCAGGGGTGTCCCTGACTGGGGGACTGT |
| | | CCTACAAAGAAGATACAAAAGAACTGGTGGTGGCTAAA |
| | | GCTGGGGTGTACTATGTGTTTTTTCAGCTGGAACTGAGG |
| | | CGGGTGGTGGCTGGGGAGGGCTCAGGATCTGTGTCCCTG |
| | | GCTCTGCATCTGCAGCCACTGCGCTCTGCAGCAGGGGCT |
| | | GCAGCACTGGCCCTGACTGTGGACCTGCCCCCAGCTTCT |
| | | TCCGAGGCCAGAAACAGCGCCTTCGGGTTCCAAGGACG |
| | | CCTGCTGCATCTGAGCGCCGGACAGCGCCTGGGAGTGC |
| | | ATCTGCATACTGAAGCCAGAGCCCGGCATGCTTGGCAG |
| | | CTGACTCAGGGGGCAACTGTGCTGGGACTGTTTCGCGTG |
| | | ACACCTGAGATCCCAGCCGGGCTC |
| 154 | nucleo- | See Table 17 |
| | tide | |
| | se- | |
| | quence | |
| | of | |
| | anti- | |
| | CEA | |
| | (T84. | |
| | 66- | |
| | LCHA) | |
| | light | |
| | chain | |
| 109 | Fc hole | see Table 2 |
| | mono- | |
| | meric | |
| | 4-1BB | |
| | ligand | |
| | (71- | |
| | 248) | |
| | chain | |
| 158 | anti- | QVQLVQSGAEVKKPGSSVKVSCKASGFNIKDTYMHWVRQ |
| | CEA | APGQGLEWMGRIDPANGNSKYVPKFQGRVTITADTSTST |
| | (T84. | AYMELSSLRSEDTAVYYCAPFGYYVSDYAMAYWGQGTLV |
| | 66- | TVSSASTKGPSVFPLAPSSKSTSGGTAALGCLVKDYFPE |
| | LCHA) | PVTVSWNSGALTSGVHTFPAVLQSSGLYSLSSVVTVPSS |
| | Fc knob | SLGTQTYICNVNHKPSNTKVDKKVEPKSCDKTHTCPPCP |
| | dimeric | APEAAGGPSVFLFPPKPKDTLMISRTPEVTCVVVDVSHE |
| | 4-1BB | DPEVKFNWYVDGVEVHNAKTKPREEQYNSTYRVVSVLTV |
| | (71- | LHQDWLNGKEYKCKVSNKALGAPIEKTISKAKGQPREPQ |
| | 248) | VYTLPPCRDELTKNQVSLWCLVKGFYPSDIAVEWESNGQ |
| | ligand | PENNYKTTPPVLDSDGSFFLYSKLTVDKSRWQQGNVFSC |
| | | SVMHEALHNHYTQKSLSLSPGREGPELSPDDPAGLLDLR |
| | | QGMFAQLVAQNVLLIDGPLSWYSDPGLAGVSLTGGLSYK |
| | | EDTKELVVAKAGVYYVFFQLELRRVVAGEGSGSVSLALH |
| | | LQPLRSAAGAAALALTVDLPPASSEARNSAFGFQGRLLH |
| | | LSAGQRLGVHLHTEARARHAWQLTQGATVLGLFRVTPEI |
| | | PAGLGGGGSGGGGSREGPELSPDDPAGLLDLRQGMFAQL |
| | | VAQNVLLIDGPLSWYSDPGLAGVSLTGGLSYKEDTKELV |
| | | VAKAGVYYVFFQLELRRVVAGEGSGSVSLALHLQPLRSA |
| | | AGAAALALTVDLPPASSEARNSAFGFQGRLLHLSAGQRL |
| | | GVHLHTEARARHAWQLTQGATVLGLFRVTPEIPAGL |
| 156 | anti- | See Table 17 |
| | CEA | |
| | (T84. | |
| | 66- | |
| | LCHA) | |
| | light | |
| | chain | |

1.5 Generation of Anti-CEA Clone T84.66-LCHA

1.5.1 Humanization of Anti-CEA Clone T84.66

Novel humanized variants of the murine antibody T84.66 (Wagener et al., J Immunol 130, 2308 (1983), Neumaier et al., J Immunol 135, 3604 (1985)) were developed by grafting of the CDRs onto human germline framework acceptor sequences.

Humanization of an antibody from non-human origin consists essentially of transplanting the CDR residues from the non-human antibody (donor) onto the framework of a human (acceptor) antibody. Normally the acceptor framework is selected by aligning the sequence of the donor to a collection of potential acceptor sequences and choosing one that has either reasonable homology to the donor, or shows similar amino acids at some positions critical for structure and activity. In the present case, the search for the antibody acceptor framework was performed by aligning the mouse T84.66 protein (NCBI Acc No: CAA36980 for the heavy chain (SEQ ID NO:159), and CAA36979 (SEQ ID NO:160) for the light chain) sequence to a collection of human germ-line sequences and picking that human sequence that showed high sequence identity. Here, the sequence IGHV1-69*08 from the IMGT database was chosen as the heavy chain framework acceptor sequence (IMGT Acc No. Z14309, SEQ ID NO:161), and the IGKV3-11*01 sequence (IMGT Acc No. X01668, SEQ ID NO:162) was chosen to be the framework acceptor for the light chain. Onto these two acceptor frameworks, the three complementary determining regions (CDRs) of the mouse heavy and light variable domains were grafted. Since the framework 4 (FR4) region is not part of the variable region of the germ line V gene, the alignment for that position was done individually. The JH4 sequence was chosen for the heavy chain, and the JK2 sequence was chosen for the light chain.

11.1.2 Binding of Different Humanized Variants of T84.66 IgG to Cells

The binding of different humanized variants of T84.66 IgG was tested on CEA-expressing human gastric adenocarcinoma cells (MKN45, DSMZ ACC 409).

Cells were harvested, counted, checked for viability and re-suspended at 2×10⁶ cells/ml in FACS buffer (100 l PBS 0.1% BSA). 100 μl of cell suspension (containing 0.2×10⁶ cells) were incubated in round-bottom 96-well plate for 30 min at 4° C. with increasing concentrations of the CEA IgG (4 ng/ml-60 μg/ml), washed twice with cold PBS 0.1% BSA, re-incubated for further 30 min at 4° C. with the PE-conjugated AffiniPure F(ab′)2 Fragment goat anti-human IgG Fcg Fragment Specific secondary antibody (Jackson Immuno Research Lab PE #109-116-170), washed twice with cold PBS 0.1% BSA and immediately analyzed by FACS using a FACS Cantoll (Software FACS Diva). Binding curves and EC50 values were obtained and calculated using GraphPadPrism5.

Table 19 shows the different binding data of selected humanized variants of the T84.66 IgG to human CEA, expressed on MKN45 cells. Based on the calculated EC50 binding values the humanized variant 1 was selected for further evaluation.

TABLE 19
Binding of different humanized variants of T84.66 IgGs to cells

| | EC50 (μg/ml) |
|---|---|
| Parental chimeric T84.66 | 0.99 |
| Humanized variant 1 | 1.5 |
| Humanized variant 2 | 8.6 |
| Humanized variant 3 | 1.4 |
| Humanized variant 4 | 3.1 |

Humanized variant 1 is termed in the following T84.66-LCHA. The amino acid sequences of its CDRs and of the VH and VL as well as the amino acid sequences of the VH and VL domain of the parental chimeric T84.66 clone are shown in Table 20.

TABLE 20
Amino acid sequences of the variable domains
of CEA clone T84.66-LCHA and its parental
antibody T84.66

| De- | | SEQ |
|---|---|---|
| scrip- | | ID |
| tion | Sequence | NO: |
| CEA | DTYMH | 45 |
| CDR-H1 | | |
| CEA | RIDPANGNSKYVPKFQG | 46 |
| CDR-H2 | | |
| CEA | FGYYVSDYAMAY | 47 |
| CDR-H3 | | |
| CEA | RAGESVDIFGVGFLH | 48 |
| CDR-L1 | | |
| CEA | RASNRAT | 49 |
| CDR-L2 | | |
| CEA | QQTNEDPYT | 50 |
| CDR-L3 | | |
| Parental | EVQLQQSGAELVEPGASVKLSCTASGFNIKD | 163 |
| CEA | TYMHWVKQRPEQGLEWIGRIDPANGNSKYVP | |
| binder | KFQGKATITADTSSNTAYLQLTSLTSEDTAV | |
| VH | YYCAPFGYYVSDYAMAYWGQGTSVTVSS | |
| Parental | DIVLTQSPASLAVSLGQRATMSCRAGESVDI | 164 |
| CEA | FGVGFLHWYQQKPGQPPKLLIYRASNLESGI | |
| binder VL | PVRFSGTGSRTDFTLIIDPVEADDVATYYCQ | |
| | QTNEDPYTFGGGTKLEIK | |
| Humanized | QVQLVQSGAEVKKPGSSVKVSCKASGFNIKD | 51 |
| CEA | TYMHWVRQAPGQGLEWMGRIDPANGNSKYVP | |
| binder | KFQGRVTITADTSTSTAYMELSSLRSEDTAV | |
| CEA | YYCAPFGYYVSDYAMAYWGQGTLVTVSS | |
| (T84.66- | | |
| LCHA) VH | | |
| Humanized | EIVLTQSPATLSLSPGERATLSCRAGESVDI | 52 |
| CEA binder | FGVGFLHWYQQKPGQAPRLLIYRASNRATGI | |
| CEA | PARFSGSGSGTDFTLTISSLEPEDFAVYYCQ | |
| (T84.66- | QTNEDPYTFGQGTKLEIK | |
| LCHA) VL | | |

1.6 Preparation of monovalent untargeted 4-1BB ligand trimer-containing F(kih) fusion antigen binding molecules, wherein the 4-1BB ligands are C-terminally fused (Controls H and I)(DP47 on the knob chain)

The molecule was prepared as described in Example 1.1 for the monovalent FAP targeted construct, with the only difference that the anti-FAP binder (VH-VL) was replaced by a germline control, termed DP47, not binding to the antigen.

Table 21 shows the cDNA and amino acid sequences of the monovalent untargeted 4-1BB ligand (71-248)trimer-containing Fc (kih) fusion antigen binding molecule (Control H).

TABLE 21
Sequences of monovalent DP47-untargeted human
4-1BB ligand trimer containing Fc (kih)
fusion molecule Control H

| SEQ | De- | |
|---|---|---|
| ID | scrip- | |
| NO: | tion | Sequence |
| 101 | nucleo- | see Table 1 |
| | tide | |
| | se- | |
| | quence | |
| | of | |
| | Fc hole | |
| | dimeric | |
| | 4-1BB | |
| | ligand | |
| | (71- | |
| | 248) | |
| | chain | |
| 165 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of DP47 | GTGGTAGTGGTGGTAGCACATACTACGCAGACTCCGTGA |
| | Fc knob | AGGGCCGGTTCACCATCTCCAGAGACAATTCCAAGAAC |
| | mono- | ACGCTGTATCTGCAGATGAACAGCCTGAGAGCCGAGGA |
| | meric | CACGGCCGTATATTACTGTGCGAAAGGCAGCGGATTTGA |
| | 4-1BB | CTACTGGGGCCAAGGAACCCTGGTCACCGTCTCGAGT |
| | (71- | GCTAGCACCAAGGGCCCATCGGTCTTCCCCCTGGCACCC |
| | 248) | TCCTCCAAGAGCACCTCTGGGGGCACAGCGGCCCTGGG |
| | ligand | CTGCCTGGTCAAGGACTACTTCCCCGAACCGGTGACGGT |
| | | GTCGTGGAACTCAGGCGCCCTGACCAGCGGCGTGCACA |
| | | CCTTCCCGGCTGTCCTACAGTCCTCAGGACTCTACTCCC |
| | | TCAGCAGCGTGGTGACCGTGCCCTCCAGCAGCTTGGGCA |
| | | CCCAGACCTACATCTGCAACGTGAATCACAAGCCCAGC |
| | | AACACCAAGGTGGACAAGAAAGTTGAGCCCAAATCTTG |
| | | TGACAAAACTCACACATGCCCACCGTGCCCAGCACCTG |
| | | AAGCTGCAGGGGGACCGTCAGTCTTCCTCTTCCCCCCAA |
| | | AACCCAAGGACACCCTCATGATCTCCCGGACCCCTGAG |
| | | GTCACATGCGTGGTGGTGGACGTGAGCCACGAAGACCC |
| | | TGAGGTCAAGTTCAACTGGTACGTGGACGGCGTGGAGG |
| | | TGCATAATGCCAAGACAAAGCCGCGGGAGGAGCAGTAC |
| | | AACAGCACGTACCGTGTGGTCAGCGTCCTCACCGTCCTG |
| | | CACCAGGACTGGCTGAATGGCAAGGAGTACAAGTGCAA |
| | | GGTCTCCAACAAAGCCCTCGGCGCCCCCATCGAGAAAA |
| | | CCATCTCCAAAGCCAAAGGGCAGCCCCGAGAACCACAG |
| | | GTGTACACCCTGCCCCCCTGCAGAGATGAGCTGACCAA |
| | | GAACCAGGTGTCCCTGTGGTGTCTGGTCAAGGGCTTCTA |
| | | CCCCAGCGATATCGCCGTGGAGTGGGAGAGCAACGGCC |
| | | AGCCTGAGAACAACTACAAGACCACCCCCCCTGTGCTG |
| | | GACAGCGACGGCAGCTTCTTCCTGTACTCCAAACTGACC |
| | | GTGGACAAGAGCCGGTGGCAGCAGGGCAACGTGTTCAG |
| | | CTGCAGCGTGATGCACGAGGCCCTGCACAACCACTACA |
| | | CCCAGAAGTCCCTGAGCCTGAGCCCCGGCGGAGGCGGC |
| | | GGAAGCGGAGGAGGAGGATCCAGAGAGGGCCCTGAGCT |
| | | GAGCCCTGATGATCCTGCCGGACTGCTGGACCTGCGGCA |
| | | GGGAATGTTTGCCCAGCTGGTGGCCCAGAACGTGCTGCT |
| | | GATCGATGGCCCCCTGTCCTGGTACAGCGATCCTGGACT |
| | | GGCTGGCGTGTCACTGACAGGCGGCCTGAGCTACAAAG |
| | | AGGACACCAAAGAACTGGTGGTGGCCAAGGCCGGCGTG |
| | | TACTACGTGTTCTTTCAGCTGGAACTGCGGAGAGTGGTG |
| | | GCCGGCGAAGGATCTGGCTCTGTGTCTCTGGCCCTGCAT |
| | | CTGCAGCCTCTGAGATCTGCTGCTGGCGCCGCTGCTCTG |
| | | GCACTGACAGTGGATCTGCCTCCTGCCAGCAGCGAGGC |
| | | CCGGAATAGCGCATTTGGGTTTCAAGGCAGGCTGCTGCA |
| | | CCTGTCTGCCGGCCAGAGGCTGGGAGTGCATCTGCACAC |
| | | AGAGGCCAGGGCTAGACACGCCTGGCAGCTGACACAGG |
| | | GCGCTACAGTGCTGGGCCTGTTCAGAGTGACCCCCGAG |
| | | ATTCCTGCCGGGCTC |
| 166 | nucleo- | GAAATCGTGTTAACGCAGTCTCCAGGCACCCTGTCTTTG |
| | tide | TCTCCAGGGGAAAGAGCCACCCTCTCTTGCAGGGCCAGT |
| | se- | CAGAGTGTTAGCAGCAGCTACTTAGCCTGGTACCAGCAG |
| | quence | AAACCTGGCCAGGCTCCCAGGCTCCTCATCTATGGAGCA |
| | of DP47 | TCCAGCAGGGCCACTGGCATCCCAGACAGGTTCAGTGG |
| | light | CAGTGGATCCGGGACAGACTTCACTCTCACCATCAGCAG |
| | chain | ACTGGAGCCTGAAGATTTTGCAGTGTATTACTGTCAGCA |
| | | GTATGGTAGCTCACCGCTGACGTTCGGCCAGGGGACCA |
| | | AAGTGGAAATCAAACGTACGGTGGCTGCACCATCTGTCT |
| | | TCATCTTCCCGCCATCTGATGAGCAGTTGAAATCTGGAA |
| | | CTGCCTCTGTTGTGTGCCTGCTGAATAACTTCTATCCCA |
| | | GAGAGGCCAAAGTACAGTGGAAGGTGGATAACGCCCTCC |
| | | AATCGGGTAACTCCCAGGAGAGTGTCACAGAGCAGGAC |
| | | AGCAAGGACAGCACCTACAGCCTCAGCAGCACCCTGAC |
| | | GCTGAGCAAAGCAGACTACGAGAAACACAAAGTCTACG |
| | | CCTGCGAAGTCACCCATCAGGGCCTGAGCTCGCCCGTCA |
| | | CAAAGAGCTTCAACAGGGGAGAGTGT |
| 104 | Fc hole | see Table 1 |
| | dimeric | |
| | 4-1BB | |
| | (71- | |
| | 248) | |
| | ligand | |
| | chain | |
| 167 | DP47 | EVQLLESGGGLVQPGGSLRLSCAASGFTFSSYAMSWVRQ |
| | Fc knob | APGKGLEWVSAISGSGGSTYYADSVKGRFTISRDNSKNT |
| | mono- | LYLQMNSLRAEDTAVYYCAKGSGFDYWGQGTLVTVSSAS |
| | meric | TKGPSVFPLAPSSKSTSGGTAALGCLVKDYFPEPVTVSW |
| | 4-1-BB | NSGALTSGVHTFPAVLQSSGLYSLSSVVTVPSSSLGTQT |
| | (71- | YICNVNHKPSNTKVDKKVEPKSCDKTHTCPPCPAPEAAG |
| | 248) | GPSVFLFPPKPKDTLMISRTPEVTCVVVDVSHEDPEVKF |
| | ligand | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSPGGGGGSGGGGSREGPELSPDDPAGL |
| | | LDLRQGMFAQLVAQNVLLIDGPLSWYSDPGLAGVSLTGG |
| | | LSYKEDTKELVVAKAGVYYVFFQLELRRVVAGEGSGSVS |
| | | LALHLQPLRSAAGAAALALTVDLPPASSEARNSAFGFQG |
| | | RLLHLSAGQRLGVHLHTEARARHAWQLTQGATVLGLFRV |
| | | TPEIPAGL |
| 168 | DP47 | EIVLTQSPGTLSLSPGERATLSCRASQSVSSSYLAWYQQ |
| | light | KPGQAPRLLIYGASSRATGIPDRFSGSGSGTDFTLTISR |
| | chain | LEPEDFAVYYCQQYGSSPLTFGQGTKVEIKRTVAAPSVF |
| | | IFPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |

Table 22 shows the cDNA and amino aid sequences of the monovalent untargeted 4-1BB ligand (71-248)trimer-containing Fe(kih) fusion antigen binding molecule (Control I).

TABLE 22
Sequences of monovalent DP47-untargeted human
4-1BB ligand trimer containing Fc (kih)
fusion molecule Control I

| SEQ | De- | |
|---|---|---|
| ID | scrip- | |
| NO: | tion | Sequence |
| 107 | nucleo- | see Table 2 |
| | tide | |
| | se- | |
| | quence | |
| | of | |
| | Fc hole | |
| | mono- | |
| | meric | |
| | 4-1BBL | |
| | (71- | |
| | 248) | |
| | chain | |
| 169 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of DP47 | GTGGTAGTGGTGGTAGCACATACTACGCAGACTCCGTGA |
| | Fc knob | AGGGCCGGTTCACCATCTCCAGAGACAATTCCAAGAAC |
| | dimeric | ACGCTGTATCTGCAGATGAACAGCCTGAGAGCCGAGGA |
| | 4-1BBL | CACGGCCGTATATTACTGTGCGAAAGGCAGCGGATTTGA |
| | (71- | CTACTGGGGCCAAGGAACCCTGGTCACCGTCTCGAGTGC |
| | 248) | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCCTGCAGAGATGAGCTGACCAAGA |
| | | ACCAGGTGTCCCTGTGGTGTCTGGTCAAGGGCTTCTACC |
| | | CCAGCGATATCGCCGTGGAGTGGGAGAGCAACGGCCAG |
| | | CCTGAGAACAACTACAAGACCACCCCCCCTGTGCTGGA |
| | | CAGCGACGGCAGCTTCTTCCTGTACTCCAAACTGACCGT |
| | | GGACAAGAGCCGGTGGCAGCAGGGCAACGTGTTCAGCT |
| | | GCAGCGTGATGCACGAGGCCCTGCACAACCACTACACC |
| | | CAGAAGTCCCTGAGCCTGAGCCCCGGCAGAGAGGGCCC |
| | | TGAGCTGAGCCCTGATGATCCTGCCGGACTGCTGGACCT |
| | | GCGGCAGGGAATGTTTGCCCAGCTGGTGGCCCAGAACG |
| | | TGCTGCTGATCGATGGCCCCCTGTCCTGGTACAGCGATC |
| | | CTGGACTGGCTGGCGTGTCACTGACAGGCGGCCTGAGCT |
| | | ACAAAGAGGACACCAAAGAACTGGTGGTGGCCAAGGCC |
| | | GGCGTGTACTACGTGTTCTTTCAGCTGGAACTGCGGAGA |
| | | GTGGTGGCCGGCGAAGGATCTGGCTCTGTGTCTCTGGCC |
| | | CTGCATCTGCAGCCTCTGAGATCTGCTGCTGGCGCCGCT |
| | | GCTCTGGCACTGACAGTGGATCTGCCTCCTGCCAGCAGC |
| | | GAGGCCCGGAATAGCGCATTTGGGTTTCAAGGCAGGCT |
| | | GCTGCACCTGTCTGCCGGCCAGAGGCTGGGAGTGCATCT |
| | | GCACACAGAGGCCAGGGCTAGACACGCCTGGCAGCTGA |
| | | CACAGGGCGCTACAGTGCTGGGCCTGTTCAGAGTGACC |
| | | CCCGAGATTCCAGCAGGCCTGGGAGGCGGCGGATCTGG |
| | | CGGCGGAGGATCTAGAGAAGGACCCGAGCTGTCCCCCG |
| | | ACGATCCCGCTGGGCTGCTGGATCTGAGACAGGGCATGT |
| | | TCGCTCAGCTGGTGGCTCAGAATGTGCTGCTGATTGACG |
| | | GACCTCTGAGCTGGTACTCCGACCCAGGGCTGGCAGGG |
| | | GTGTCCCTGACTGGGGGACTGTCCTACAAAGAAGATAC |
| | | AAAAGAACTGGTGGTGGCTAAAGCTGGGGTGTACTATG |
| | | TGTTTTTTCAGCTGGAACTGAGGCGGGTGGTGGCTGGGG |
| | | AGGGCTCAGGATCTGTGTCCCTGGCTCTGCATCTGCAGC |
| | | CACTGCGCTCTGCAGCAGGGGCTGCAGCACTGGCCCTG |
| | | ACTGTGGACCTGCCCCCAGCTTCTTCCGAGGCCAGAAAC |
| | | AGCGCCTTCGGGTTCCAAGGACGCCTGCTGCATCTGAGC |
| | | GCCGGACAGCGCCTGGGAGTGCATCTGCATACTGAAGC |
| | | CAGAGCCCGGCATGCTTGGCAGCTGACTCAGGGGGCAA |
| | | CTGTGCTGGGACTGTTTCGCGTGACACCTGAGATCCCAG |
| | | CCGGGCTC |
| 166 | nucleo- | See Table 21 |
| | tide | |
| | se- | |
| | quence | |
| | of DP47 | |
| | light | |
| | chain | |
| 109 | Fc hole | see Table 2 |
| | mono- | |
| | meric | |
| | 4-1BB | |
| | ligand | |
| | (71- | |
| | 248) | |
| | chain | |
| 170 | DP47 | EVQLLESGGGLVQPGGSLRLSCAASGFTFSSYAMSWVRQ |
| | Fc knob | APGKGLEWVSAISGSGGSTYYADSVKGRFTISRDNSKNT |
| | dimeric | LYLQMNSLRAEDTAVYYCAKGSGFDYWGQGTLVTVSSAS |
| | 4-1BBL | TKGPSVFPLAPSSKSTSGGTAALGCLVKDYFPEPVTVSW |
| | (71- | NSGALTSGVHTFPAVLQSSGLYSLSSVVTVPSSSLGTQT |
| | 248) | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSPGREGPELSPDDPAGLLDLRQGMFAQ |
| | | LVAQNVLLIDGPLSWYSDPGLAGVSLTGGLSYKEDTKEL |
| | | VVAKAGVYYVFFQLELRRVVAGEGSGSVSLALHLQPLRS |
| | | AAGAAALALTVDLPPASSEARNSAFGFQGRLLHLSAGQR |
| | | LGVHLHTEARARHAWQLTQGATVLGLFRVTPEIPAGLGG |
| | | GGSGGGGSREGPELSPDDPAGLLDLRQGMFAQLVAQNVL |
| | | LIDGPLSWYSDPGLAGVSLTGGLSYKEDTKELVVAKAGV |
| | | YYVFFQLELRRVVAGEGSGSVSLALHLQPLRSAAGAAAL |
| | | ALTVDLPPASSEARNSAFGFQGRLLHLSAGQRLGVHLHT |
| | | EARARHAWQLTQGATVLGLFRVTPEIPAGL |
| 168 | DP47 | See Table 21 |
| | light | |
| | chain | |

1.7 Preparation of Monovalent FAP-Targeted 4-1BB Ligand Trimer-Containing Fc (Kih) Fusion Antigen Binding Molecule, Wherein the 4-1BB Ligands are C-Terminally Fused (Constructs 1.13, 2.13, 1.14 and 2.14) (FAP on the Hole Chain)

A polypeptide containing two ectodomains of 4-1BB ligand, separated by (G4S)2 linkers was subcloned in frame to the C-terminus of human IgG1 Fc hole or knob chain (Merchant, Zhu 1998), as depicted in FIG. 1a: human IgG Fc, (G4S)2 connector, human 4-1BB ligand, (G4S)2 connector, human 4-1BB ligand. A polypeptide containing one ectodomain of 4-1BB ligand was subcloned in frame to the C-terminus of human IgG1 Fc knob or hole chain as described in FIG. 1b: human IgG Fc, (G4S)2 connector, human 4-1BB ligand.

The variable region of heavy and light chain DNA sequences encoding a binder specific for fibroblast activation protein (FAP), clone 4B9 or clone 28H1, were subcloned in frame with either the constant heavy chain of the hole or the constant light chain of human IgG1. The generation and preparation of the FAP binders is described in WO 2012/020006 A2, which is incorporated herein by reference.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

For all constructs the knobs into holes heterodimerization technology was used as described in Carter, J Immunol Methods 248, 7-15 (2001). Combination of the huIgG1 Fc hole chain containing the Y349C/T366S/L368A/Y407V mutations in the CH3 domain, the anti-FAP huIgG1 knob chain containing the S354C/T366W mutations in the CH3 domain and the anti-FAP light chain allows generation of a heterodimer, which includes an assembled trimeric 4-1BB ligand and one FAP binding Fab (FIGS. 2C and 2D).

Table 23 shows the cDNA and amino acid sequences of the monovalent FAP(4B9)-targeted 4-1BB ligand (71-248) trimer-containing Fc (kih) fusion antigen binding molecule (Construct 2.13).

TABLE 23
Sequences of FAP(4B9)-targeted human 4-1BB
ligand trimer containing Fc (kih) fusion
molecule Construct 2.13

| SEQ | De- | |
|---|---|---|
| ID | scrip- | |
| NO: | tion | Sequence |
| 171 | nucleo- | GAGGTGCAGCTGCTCGAAAGCGGCGGAGGACTGGTGCA |
| | tide | GCCTGGCGGCAGCCTGAGACTGTCTTGCGCCGCCAGCG |
| | se- | GCTTCACCTTCAGCAGCTACGCCATGAGCTGGGTCCGCC |
| | quence | AGGCCCCTGGCAAGGGACTGGAATGGGTGTCCGCCATC |
| | of | ATCGGCTCTGGCGCCAGCACCTACTACGCCGACAGCGTG |
| | anti- | AAGGGCCGGTTCACCATCAGCCGGGACAACAGCAAGAA |
| | FAP | CACCCTGTACCTGCAGATGAACAGCCTGCGGGCCGAGG |
| | (4B9) | ACACCGCCGTGTACTACTGCGCCAAGGGATGGTTCGGC |
| | Fc hole | GGCTTCAACTACTGGGGACAGGGCACCCTGGTCACAGT |
| | dimeric | GTCCAGCGCTAGCACCAAGGGCCCCTCCGTGTTCCCCCT |
| | 4-1BB | GGCCCCCAGCAGCAAGAGCACCAGCGGCGGCACAGCCG |
| | ligand | CTCTGGGCTGCCTGGTCAAGGACTACTTCCCCGAGCCCG |
| | (71- | TGACCGTGTCCTGGAACAGCGGAGCCCTGACCTCCGGC |
| | 248) | GTGCACACCTTCCCCGCCGTGCTGCAGAGTTCTGGCCTG |
| | chain | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAAACTCACACATGCCCACCGTGCCCA |
| | | GCACCTGAAGCTGCAGGGGGACCGTCAGTCTTCCTCTTC |
| | | CCCCCAAAACCCAAGGACACCCTCATGATCTCCCGGACC |
| | | CCTGAGGTCACATGCGTGGTGGTGGACGTGAGCCACGA |
| | | AGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTGCACCCTGCCCCCATCCCGGGATGAGCTG |
| | | ACCAAGAACCAGGTCAGCCTCTCGTGCGCAGTCAAAGG |
| | | CTTCTATCCCAGCGACATCGCCGTGGAGTGGGAGAGCA |
| | | ATGGGCAGCCGGAGAACAACTACAAGACCACGCCTCCC |
| | | GTGCTGGACTCCGACGGCTCCTTCTTCCTCGTGAGCAAG |
| | | CTCACCGTGGACAAGAGCAGGTGGCAGCAGGGGAACGT |
| | | CTTCTCATGCTCCGTGATGCATGAGGCTCTGCACAACCA |
| | | CTACACGCAGAAGAGCCTCTCCCTGTCTCCGGGTGGAGG |
| | | CGGCGGAAGCGGAGGAGGAGGATCCAGAGAGGGCCCT |
| | | GAGCTGAGCCCTGATGATCCTGCCGGACTGCTGGACCTG |
| | | CGGCAGGGAATGTTTGCCCAGCTGGTGGCCCAGAACGT |
| | | GCTGCTGATCGATGGCCCCCTGTCCTGGTACAGCGATCC |
| | | TGGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGATCTGCTGCTGGCGCCGCTG |
| | | CTCTGGCACTGACAGTGGATCTGCCTCCTGCCAGCAGCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCAGCAGGCCTGGGAGGCGGCGGATCTGGC |
| | | GGCGGAGGATCTAGAGAAGGACCCGAGCTGTCCCCCGA |
| | | CGATCCCGCTGGGCTGCTGGATCTGAGACAGGGCATGTT |
| | | CGCTCAGCTGGTGGCTCAGAATGTGCTGCTGATTGACGG |
| | | ACCTCTGAGCTGGTACTCCGACCCAGGGCTGGCAGGGG |
| | | TGTCCCTGACTGGGGGACTGTCCTACAAAGAAGATACA |
| | | AAAGAACTGGTGGTGGCTAAAGCTGGGGTGTACTATGT |
| | | GTTTTTTCAGCTGGAACTGAGGCGGGTGGTGGCTGGGGA |
| | | GGGCTCAGGATCTGTGTCCCTGGCTCTGCATCTGCAGCC |
| | | ACTGCGCTCTGCAGCAGGGGCTGCAGCACTGGCCCTGA |
| | | CTGTGGACCTGCCCCCAGCTTCTTCCGAGGCCAGAAACA |
| | | GCGCCTTCGGGTTCCAAGGACGCCTGCTGCATCTGAGCG |
| | | CCGGACAGCGCCTGGGAGTGCATCTGCATACTGAAGCC |
| | | AGAGCCCGGCATGCTTGGCAGCTGACTCAGGGGGCAAC |
| | | TGTGCTGGGACTGTTTCGCGTGACACCTGAGATCCCAGC |
| | | CGGGCTC |
| 172 | nucleo- | GACAAAACTCACACATGCCCACCGTGCCCAGCACCTGA |
| | tide | AGCTGCAGGGGGACCGTCAGTCTTCCTCTTCCCCCCAAA |
| | se- | ACCCAAGGACACCCTCATGATCTCCCGGACCCCTGAGGT |
| | quence | CACATGCGTGGTGGTGGACGTGAGCCACGAAGACCCTG |
| | of | AGGTCAAGTTCAACTGGTACGTGGACGGCGTGGAGGTG |
| | Fc knob | CATAATGCCAAGACAAAGCCGCGGGAGGAGCAGTACAA |
| | mono- | CAGCACGTACCGTGTGGTCAGCGTCCTCACCGTCCTGCA |
| | meric | CCAGGACTGGCTGAATGGCAAGGAGTACAAGTGCAAGG |
| | 4-1BB | TCTCCAACAAAGCCCTCGGCGCCCCCATCGAGAAAACC |
| | (71- | ATCTCCAAAGCCAAAGGGCAGCCCCGAGAACCACAGGT |
| | 248) | GTACACCCTGCCCCCCTGCAGAGATGAGCTGACCAAGA |
| | ligand | ACCAGGTGTCCCTGTGGTGTCTGGTCAAGGGCTTCTACC |
| | | CCAGCGATATCGCCGTGGAGTGGGAGAGCAACGGCCAG |
| | | CCTGAGAACAACTACAAGACCACCCCCCCTGTGCTGGA |
| | | CAGCGACGGCAGCTTCTTCCTGTACTCCAAACTGACCGT |
| | | GGACAAGAGCCGGTGGCAGCAGGGCAACGTGTTCAGCT |
| | | GCAGCGTGATGCACGAGGCCCTGCACAACCACTACACC |
| | | CAGAAGTCCCTGAGCCTGAGCCCCGGCGGAGGCGGCGG |
| | | AAGCGGAGGAGGAGGATCCAGAGAGGGCCCTGAGCTGA |
| | | GCCCTGATGATCCTGCCGGACTGCTGGACCTGCGGCAGG |
| | | GAATGTTTGCCCAGCTGGTGGCCCAGAACGTGCTGCTGA |
| | | TCGATGGCCCCCTGTCCTGGTACAGCGATCCTGGACTGG |
| | | CTGGCGTGTCACTGACAGGCGGCCTGAGCTACAAAGAG |
| | | GACACCAAAGAACTGGTGGTGGCCAAGGCCGGCGTGTA |
| | | CTACGTGTTCTTTCAGCTGGAACTGCGGAGAGTGGTGGC |
| | | CGGCGAAGGATCTGGCTCTGTGTCTCTGGCCCTGCATCT |
| | | GCAGCCTCTGAGATCTGCTGCTGGCGCCGCTGCTCTGGC |
| | | ACTGACAGTGGATCTGCCTCCTGCCAGCAGCGAGGCCC |
| | | GGAATAGCGCATTTGGGTTTCAAGGCAGGCTGCTGCACC |
| | | TGTCTGCCGGCCAGAGGCTGGGAGTGCATCTGCACACA |
| | | GAGGCCAGGGCTAGACACGCCTGGCAGCTGACACAGGG |
| | | CGCTACAGTGCTGGGCCTGTTCAGAGTGACCCCCGAGAT |
| | | TCCTGCCGGGCTC |
| 103 | nucleo- | see Table 1 |
| | tide | |
| | se- | |
| | quence | |
| | of | |
| | anti- | |
| | FAP | |
| | (4B9) | |
| | light | |
| | chain | |
| 173 | anti- | EVQLLESGGGLVQPGGSLRLSCAASGFTFSSYAMSWVRQ |
| | FAP | APGKGLEWVSAIIGSGASTYYADSVKGRFTISRDNSKNT |
| | (4B9) | LYLQMNSLRAEDTAVYYCAKGWFGGFNYWGQGTLVTVSS |
| | Fc hole | ASTKGPSVFPLAPSSKSTSGGTAALGCLVKDYFPEPVTV |
| | dimeric | SWNSGALTSGVHTFPAVLQSSGLYSLSSVVTVPSSSLGT |
| | 4-1BB | QTYICNVNHKPSNTKVDKKVEPKSCDKTHTCPPCPAPEA |
| | (71- | AGGPSVFLFPPKPKDTLMISRTPEVTCVVVDVSHEDPEV |
| | 248) | KFNWYVDGVEVHNAKTKPREEQYNSTYRVVSVLTVLHQD |
| | ligand | WLNGKEYKCKVSNKALGAPIEKTISKAKGQPREPQVCTL |
| | chain | PPSRDELTKNQVSLSCAVKGFYPSDIAVEWESNGQPENN |
| | | YKTTPPVLDSDGSFFLVSKLTVDKSRWQQGNVFSCSVMH |
| | | EALHNHYTQKSLSLSPGGGGGSGGGGSREGPELSPDDPA |
| | | GLLDLRQGMFAQLVAQNVLLIDGPLSWYSDPGLAGVSLT |
| | | GGLSYKEDTKELVVAKAGVYYVFFQLELRRVVAGEGSGS |
| | | VSLALHLQPLRSAAGAAALALTVDLPPASSEARNSAFGF |
| | | QGRLLHLSAGQRLGVHLHTEARARHAWQLTQGATVLGLF |
| | | RVTPEIPAGLGGGGSGGGGSREGPELSPDDPAGLLDLRQ |
| | | GMFAQLVAQNVLLIDGPLSWYSDPGLAGVSLTGGLSYKE |
| | | DTKELVVAKAGVYYVFFQLELRRVVAGEGSGSVSLALHL |
| | | QPLRSAAGAAALALTVDLPPASSEARNSAFGFQGRLLHL |
| | | SAGQRLGVHLHTEARARHAWQLTQGATVLGLFRVTPEIP |
| | | AGL |
| 174 | Fc knob | DKTHTCPPCPAPEAAGGPSVFLFPPKPKDTLMISRTPEV |
| | mono- | TCVVVDVSHEDPEVKFNWYVDGVEVHNAKTKPREEQYNS |
| | meric | TYRVVSVLTVLHQDWLNGKEYKCKVSNKALGAPIEKTIS |
| | 41-BBL | KAKGQPREPQVYTLPPCRDELTKNQVSLWCLVKGFYPSD |
| | (71- | IAVEWESNGQPENNYKTTPPVLDSDGSFFLYSKLTVDKS |
| | 248) | RWQQGNVFSCSVMHEALHNHYTQKSLSLSPGGGGGSGGG |
| | | GSREGPELSPDDPAGLLDLRQGMFAQLVAQNVLLIDGPL |
| | | SWYSDPGLAGVSLTGGLSYKEDTKELVVAKAGVYYVFFQ |
| | | LELRRVVAGEGSGSVSLALHLQPLRSAAGAAALALTVDL |
| | | PPASSEARNSAFGFQGRLLHLSAGQRLGVHLHTEARARH |
| | | AWQLTQGATVLGLFRVTPEIPAGL |
| 106 | anti- | see Table 1 |
| | FAP | |
| | (4B9) | |
| | light | |
| | chain | |

Table 24 shows the cDNA and amino acid sequences of the monovalent FAP(4B9)-targeted 4-1BB ligand (71-248)trimer-containing Fc (kih) fusion antigen binding molecule (Construct 2.14).

TABLE 24
Sequences of FAP(4B9)-targeted human 4-1BB
ligand trimer containing Fc (kih) fusion
molecule Construct 2.14

| SEQ | De- | |
|---|---|---|
| ID | scrip- | |
| NO: | tion | Sequence |
| 175 | nucleo- | GAGGTGCAGCTGCTCGAAAGCGGCGGAGGACTGGTGCA |
| | tide | GCCTGGCGGCAGCCTGAGACTGTCTTGCGCCGCCAGCG |
| | se- | GCTTCACCTTCAGCAGCTACGCCATGAGCTGGGTCCGCC |
| | quence | AGGCCCCTGGCAAGGGACTGGAATGGGTGTCCGCCATC |
| | of | ATCGGCTCTGGCGCCAGCACCTACTACGCCGACAGCGTG |
| | anti- | AAGGGCCGGTTCACCATCAGCCGGGACAACAGCAAGAA |
| | FAP | CACCCTGTACCTGCAGATGAACAGCCTGCGGGCCGAGG |
| | (4B9) | ACACCGCCGTGTACTACTGCGCCAAGGGATGGTTCGGC |
| | Fc hole | GGCTTCAACTACTGGGGACAGGGCACCCTGGTCACAGT |
| | mono- | GTCCAGCGCTAGCACCAAGGGCCCCTCCGTGTTCCCCCT |
| | meric | GGCCCCCAGCAGCAAGAGCACCAGCGGCGGCACAGCCG |
| | 4-1BBL | CTCTGGGCTGCCTGGTCAAGGACTACTTCCCCGAGCCCG |
| | (71- | TGACCGTGTCCTGGAACAGCGGAGCCCTGACCTCCGGC |
| | 248) | GTGCACACCTTCCCCGCCGTGCTGCAGAGTTCTGGCCTG |
| | chain | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAAACTCACACATGCCCACCGTGCCCA |
| | | GCACCTGAAGCTGCAGGGGGACCGTCAGTCTTCCTCTTC |
| | | CCCCCAAAACCCAAGGACACCCTCATGATCTCCCGGACC |
| | | CCTGAGGTCACATGCGTGGTGGTGGACGTGAGCCACGA |
| | | AGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTGCACCCTGCCCCCATCCCGGGATGAGCTG |
| | | ACCAAGAACCAGGTCAGCCTCTCGTGCGCAGTCAAAGG |
| | | CTTCTATCCCAGCGACATCGCCGTGGAGTGGGAGAGCA |
| | | ATGGGCAGCCGGAGAACAACTACAAGACCACGCCTCCC |
| | | GTGCTGGACTCCGACGGCTCCTTCTTCCTCGTGAGCAAG |
| | | CTCACCGTGGACAAGAGCAGGTGGCAGCAGGGGAACGT |
| | | CTTCTCATGCTCCGTGATGCATGAGGCTCTGCACAACCA |
| | | CTACACGCAGAAGAGCCTCTCCCTGTCTCCGGGTGGAGG |
| | | CGGCGGAAGCGGAGGAGGAGGATCCAGAGAGGGCCCT |
| | | GAGCTGAGCCCTGATGATCCTGCCGGACTGCTGGACCTG |
| | | CGGCAGGGAATGTTTGCCCAGCTGGTGGCCCAGAACGT |
| | | GCTGCTGATCGATGGCCCCCTGTCCTGGTACAGCGATCC |
| | | TGGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGATCTGCTGCTGGCGCCGCTG |
| | | CTCTGGCACTGACAGTGGATCTGCCTCCTGCCAGCAGCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCTGCCGGGCTC |
| 176 | nucleo- | GACAAAACTCACACATGCCCACCGTGCCCAGCACCTGA |
| | tide | AGCTGCAGGGGGACCGTCAGTCTTCCTCTTCCCCCCAAA |
| | se- | ACCCAAGGACACCCTCATGATCTCCCGGACCCCTGAGGT |
| | quence | CACATGCGTGGTGGTGGACGTGAGCCACGAAGACCCTG |
| | of | AGGTCAAGTTCAACTGGTACGTGGACGGCGTGGAGGTG |
| | Fc knob | CATAATGCCAAGACAAAGCCGCGGGAGGAGCAGTACAA |
| | dimeric | CAGCACGTACCGTGTGGTCAGCGTCCTCACCGTCCTGCA |
| | 4-1BB | CCAGGACTGGCTGAATGGCAAGGAGTACAAGTGCAAGG |
| | ligand | TCTCCAACAAAGCCCTCGGCGCCCCCATCGAGAAAACC |
| | (71- | ATCTCCAAAGCCAAAGGGCAGCCCCGAGAACCACAGGT |
| | 248) | GTACACCCTGCCCCCCTGCAGAGATGAGCTGACCAAGA |
| | | ACCAGGTGTCCCTGTGGTGTCTGGTCAAGGGCTTCTACC |
| | | CCAGCGATATCGCCGTGGAGTGGGAGAGCAACGGCCAG |
| | | CCTGAGAACAACTACAAGACCACCCCCCCTGTGCTGGA |
| | | CAGCGACGGCAGCTTCTTCCTGTACTCCAAACTGACCGT |
| | | GGACAAGAGCCGGTGGCAGCAGGGCAACGTGTTCAGCT |
| | | GCAGCGTGATGCACGAGGCCCTGCACAACCACTACACC |
| | | CAGAAGTCCCTGAGCCTGAGCCCCGGCGGAGGCGGCGG |
| | | AAGCGGAGGAGGAGGATCCAGAGAGGGCCCTGAGCTGA |
| | | GCCCTGATGATCCTGCCGGACTGCTGGACCTGCGGCAGG |
| | | GAATGTTTGCCCAGCTGGTGGCCCAGAACGTGCTGCTGA |
| | | TCGATGGCCCCCTGTCCTGGTACAGCGATCCTGGACTGG |
| | | CTGGCGTGTCACTGACAGGCGGCCTGAGCTACAAAGAG |
| | | GACACCAAAGAACTGGTGGTGGCCAAGGCCGGCGTGTA |
| | | CTACGTGTTCTTTCAGCTGGAACTGCGGAGAGTGGTGGC |
| | | CGGCGAAGGATCTGGCTCTGTGTCTCTGGCCCTGCATCT |
| | | GCAGCCTCTGAGATCTGCTGCTGGCGCCGCTGCTCTGGC |
| | | ACTGACAGTGGATCTGCCTCCTGCCAGCAGCGAGGCCC |
| | | GGAATAGCGCATTTGGGTTTCAAGGCAGGCTGCTGCACC |
| | | TGTCTGCCGGCCAGAGGCTGGGAGTGCATCTGCACACA |
| | | GAGGCCAGGGCTAGACACGCCTGGCAGCTGACACAGGG |
| | | CGCTACAGTGCTGGGCCTGTTCAGAGTGACCCCCGAGAT |
| | | TCCAGCAGGCCTGGGAGGCGGCGGATCTGGCGGCGGAG |
| | | GATCTAGAGAAGGACCCGAGCTGTCCCCCGACGATCCC |
| | | GCTGGGCTGCTGGATCTGAGACAGGGCATGTTCGCTCAG |
| | | CTGGTGGCTCAGAATGTGCTGCTGATTGACGGACCTCTG |
| | | AGCTGGTACTCCGACCCAGGGCTGGCAGGGGTGTCCCT |
| | | GACTGGGGGACTGTCCTACAAAGAAGATACAAAAGAAC |
| | | TGGTGGTGGCTAAAGCTGGGGTGTACTATGTGTTTTTTC |
| | | AGCTGGAACTGAGGCGGGTGGTGGCTGGGGAGGGCTCA |
| | | GGATCTGTGTCCCTGGCTCTGCATCTGCAGCCACTGCGC |
| | | TCTGCAGCAGGGGCTGCAGCACTGGCCCTGACTGTGGA |
| | | CCTGCCCCCAGCTTCTTCCGAGGCCAGAAACAGCGCCTT |
| | | CGGGTTCCAAGGACGCCTGCTGCATCTGAGCGCCGGAC |
| | | AGCGCCTGGGAGTGCATCTGCATACTGAAGCCAGAGCC |
| | | CGGCATGCTTGGCAGCTGACTCAGGGGGCAACTGTGCTG |
| | | GGACTGTTTCGCGTGACACCTGAGATCCCAGCCGGGCTC |
| 103 | nucleo- | See Table 1 |
| | tide | |
| | se- | |
| | quence | |
| | of | |
| | anti- | |
| | FAP | |
| | (4B9) | |
| | light | |
| | chain | |
| 177 | anti- | EVQLLESGGGLVQPGGSLRLSCAASGFTFSSYAMSWVRQ |
| | FAP | APGKGLEWVSAIIGSGASTYYADSVKGRFTISRDNSKNT |
| | (4B9) | LYLQMNSLRAEDTAVYYCAKGWFGGFNYWGQGTLVTVSS |
| | Fc hole | ASTKGPSVFPLAPSSKSTSGGTAALGCLVKDYFPEPVTV |
| | mono- | SWNSGALTSGVHTFPAVLQSSGLYSLSSVVTVPSSSLGT |
| | meric | QTYICNVNHKPSNTKVDKKVEPKSCDKTHTCPPCPAPEA |
| | 4-1BB | AGGPSVFLFPPKPKDTLMISRTPEVTCVVVDVSHEDPEV |
| | ligand | KFNWYVDGVEVHNAKTKPREEQYNSTYRVVSVLTVLHQD |
| | (71- | WLNGKEYKCKVSNKALGAPIEKTISKAKGQPREPQVCTL |
| | 248) | PPSRDELTKNQVSLSCAVKGFYPSDIAVEWESNGQPENN |
| | chain | YKTTPPVLDSDGSFFLVSKLTVDKSRWQQGNVFSCSVMH |
| | | EALHNHYTQKSLSLSPGGGGGSGGGGSREGPELSPDDPA |
| | | GLLDLRQGMFAQLVAQNVLLIDGPLSWYSDPGLAGVSLT |
| | | GGLSYKEDTKELVVAKAGVYYVFFQLELRRVVAGEGSGS |
| | | VSLALHLQPLRSAAGAAALALTVDLPPASSEARNSAFGF |
| | | QGRLLHLSAGQRLGVHLHTEARARHAWQLTQGATVLGLF |
| | | RVTPEIPAGL |
| 178 | Fc knob | DKTHTCPPCPAPEAAGGPSVFLFPPKPKDTLMISRTPEV |
| | dimeric | TCVVVDVSHEDPEVKFNWYVDGVEVHNAKTKPREEQYNS |
| | 4-1BB | TYRVVSVLTVLHQDWLNGKEYKCKVSNKALGAPIEKTIS |
| | ligand | KAKGQPREPQVYTLPPCRDELTKNQVSLWCLVKGFYPSD |
| | (71- | IAVEWESNGQPENNYKTTPPVLDSDGSFFLYSKLTVDKS |
| | 248) | RWQQGNVFSCSVMHEALHNHYTQKSLSLSPGGGGGSGGG |
| | | GSREGPELSPDDPAGLLDLRQGMFAQLVAQNVLLIDGPL |
| | | SWYSDPGLAGVSLTGGLSYKEDTKELVVAKAGVYYVFFQ |
| | | LELRRVVAGEGSGSVSLALHLQPLRSAAGAAALALTVDL |
| | | PPASSEARNSAFGFQGRLLHLSAGQRLGVHLHTEARARH |
| | | AWQLTQGATVLGLFRVTPEIPAGLGGGGSGGGGSREGPE |
| | | LSPDDPAGLLDLRQGMFAQLVAQNVLLIDGPLSWYSDPG |
| | | LAGVSLTGGLSYKEDTKELVVAKAGVYYVFFQLELRRVV |
| | | AGEGSGSVSLALHLQPLRSAAGAAALALTVDLPPASSEA |
| | | RNSAFGFQGRLLHLSAGQRLGVHLHTEARARHAWQLTQG |
| | | ATVLGLFRVTPEIPAGL |
| 106 | anti- | See Table 1 |
| | FAP | |
| | (4B9) | |
| | light | |
| | chain | |

Table 25 shows the cDNA and amino acid sequences of the monovalent FAP(28H1)-targeted 4-1BB ligand trimer-containing Fc (kih) fusion antigen binding molecule (Construct 1.13).

TABLE 25
Sequences of FAP(28H1)-targeted human 4-1BB
ligand trimer containing Fc (kih) fusion
molecule Construct 1.13

| SEQ | De- | |
|---|---|---|
| ID | scrip- | |
| NO: | tion | Sequence |
| 179 | nucleo- | GAAGTGCAGCTGCTGGAATCCGGCGGAGGCCTGGTGCA |
| | tide | GCCTGGCGGATCTCTGAGACTGTCCTGCGCCGCCTCCGG |
| | se- | CTTCACCTTCTCCTCCCACGCCATGTCCTGGGTCCGACA |
| | quence | GGCTCCTGGCAAAGGCCTGGAATGGGTGTCCGCCATCTG |
| | of | GGCCTCCGGCGAGCAGTACTACGCCGACTCTGTGAAGG |
| | anti- | GCCGGTTCACCATCTCCCGGGACAACTCCAAGAACACCC |
| | FAP | TGTACCTGCAGATGAACTCCCTGCGGGCCGAGGACACC |
| | (28H1) | GCCGTGTACTACTGTGCCAAGGGCTGGCTGGGCAACTTC |
| | Fc hole | GACTACTGGGGACAGGGCACCCTGGTCACCGTGTCCAG |
| | dimeric | CGCTAGCACCAAGGGCCCCTCCGTGTTCCCCCTGGCCCC |
| | 4-1BB | CAGCAGCAAGAGCACCAGCGGCGGCACAGCCGCTCTGG |
| | ligand | GCTGCCTGGTCAAGGACTACTTCCCCGAGCCCGTGACCG |
| | (71- | TGTCCTGGAACAGCGGAGCCCTGACCTCCGGCGTGCAC |
| | 248) | ACCTTCCCCGCCGTGCTGCAGAGTTCTGGCCTGTATAGC |
| | chain | CTGAGCAGCGTGGTCACCGTGCCTTCTAGCAGCCTGGGC |
| | | ACCCAGACCTACATCTGCAACGTGAACCACAAGCCCAG |
| | | CAACACCAAGGTGGACAAGAAGGTGGAGCCCAAGAGCT |
| | | GCGACAAAACTCACACATGCCCACCGTGCCCAGCACCT |
| | | GAAGCTGCAGGGGGACCGTCAGTCTTCCTCTTCCCCCCA |
| | | AAACCCAAGGACACCCTCATGATCTCCCGGACCCCTGA |
| | | GGTCACATGCGTGGTGGTGGACGTGAGCCACGAAGACC |
| | | CTGAGGTCAAGTTCAACTGGTACGTGGACGGCGTGGAG |
| | | GTGCATAATGCCAAGACAAAGCCGCGGGAGGAGCAGTA |
| | | CAACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCGGCGCCCCCATCGAGAAA |
| | | ACCATCTCCAAAGCCAAAGGGCAGCCCCGAGAACCACA |
| | | GGTGTGCACCCTGCCCCCATCCCGGGATGAGCTGACCAA |
| | | GAACCAGGTCAGCCTCTCGTGCGCAGTCAAAGGCTTCTA |
| | | TCCCAGCGACATCGCCGTGGAGTGGGAGAGCAATGGGC |
| | | AGCCGGAGAACAACTACAAGACCACGCCTCCCGTGCTG |
| | | GACTCCGACGGCTCCTTCTTCCTCGTGAGCAAGCTCACC |
| | | GTGGACAAGAGCAGGTGGCAGCAGGGGAACGTCTTCTC |
| | | ATGCTCCGTGATGCATGAGGCTCTGCACAACCACTACAC |
| | | GCAGAAGAGCCTCTCCCTGTCTCCGGGTGGAGGCGGCG |
| | | GAAGCGGAGGAGGAGGATCCAGAGAGGGCCCTGAGCTG |
| | | AGCCCTGATGATCCTGCCGGACTGCTGGACCTGCGGCAG |
| | | GGAATGTTTGCCCAGCTGGTGGCCCAGAACGTGCTGCTG |
| | | ATCGATGGCCCCCTGTCCTGGTACAGCGATCCTGGACTG |
| | | GCTGGCGTGTCACTGACAGGCGGCCTGAGCTACAAAGA |
| | | GGACACCAAAGAACTGGTGGTGGCCAAGGCCGGCGTGT |
| | | ACTACGTGTTCTTTCAGCTGGAACTGCGGAGAGTGGTGG |
| | | CCGGCGAAGGATCTGGCTCTGTGTCTCTGGCCCTGCATC |
| | | TGCAGCCTCTGAGATCTGCTGCTGGCGCCGCTGCTCTGG |
| | | CACTGACAGTGGATCTGCCTCCTGCCAGCAGCGAGGCCC |
| | | GGAATAGCGCATTTGGGTTTCAAGGCAGGCTGCTGCACC |
| | | TGTCTGCCGGCCAGAGGCTGGGAGTGCATCTGCACACA |
| | | GAGGCCAGGGCTAGACACGCCTGGCAGCTGACACAGGG |
| | | CGCTACAGTGCTGGGCCTGTTCAGAGTGACCCCCGAGAT |
| | | TCCAGCAGGCCTGGGAGGCGGCGGATCTGGCGGCGGAG |
| | | GATCTAGAGAAGGACCCGAGCTGTCCCCCGACGATCCC |
| | | GCTGGGCTGCTGGATCTGAGACAGGGCATGTTCGCTCAG |
| | | CTGGTGGCTCAGAATGTGCTGCTGATTGACGGACCTCTG |
| | | AGCTGGTACTCCGACCCAGGGCTGGCAGGGGTGTCCCT |
| | | GACTGGGGGACTGTCCTACAAAGAAGATACAAAAGAAC |
| | | TGGTGGTGGCTAAAGCTGGGGTGTACTATGTGTTTTTTC |
| | | AGCTGGAACTGAGGCGGGTGGTGGCTGGGGAGGGCTCA |
| | | GGATCTGTGTCCCTGGCTCTGCATCTGCAGCCACTGCGC |
| | | TCTGCAGCAGGGGCTGCAGCACTGGCCCTGACTGTGGA |
| | | CCTGCCCCCAGCTTCTTCCGAGGCCAGAAACAGCGCCTT |
| | | CGGGTTCCAAGGACGCCTGCTGCATCTGAGCGCCGGAC |
| | | AGCGCCTGGGAGTGCATCTGCATACTGAAGCCAGAGCC |
| | | CGGCATGCTTGGCAGCTGACTCAGGGGGCAACTGTGCTG |
| | | GGACTGTTTCGCGTGACACCTGAGATCCCAGCCGGGCTC |
| 172 | nucleo- | see Table 23 |
| | tide | |
| | se- | |
| | quence | |
| | of Fc | |
| | knob | |
| | mono- | |
| | meric | |
| | 4-1BB | |
| | ligand | |
| | (71- | |
| | 248) | |
| 112 | nucleo- | see Table 3 |
| | tide | |
| | se- | |
| | quence | |
| | of | |
| | anti- | |
| | FAP | |
| | (28H1) | |
| | light | |
| | chain | |
| 180 | anti- | EVQLLESGGGLVQPGGSLRLSCAASGFTFSSHAMSWVRQ |
| | FAP | APGKGLEWVSAIWASGEQYYADSVKGRFTISRDNSKNTL |
| | (28H1) | YLQMNSLRAEDTAVYYCAKGWLGNFDYWGQGTLVTVSSA |
| | Fc hole | STKGPSVFPLAPSSKSTSGGTAALGCLVKDYFPEPVTVS |
| | dimeric | WNSGALTSGVHTFPAVLQSSGLYSLSSVVTVPSSSLGTQ |
| | 4-1BB | TYICNVNHKPSNTKVDKKVEPKSCDKTHTCPPCPAPEAA |
| | ligand | GGPSVFLFPPKPKDTLMISRTPEVTCVVVDVSHEDPEVK |
| | (71- | FNWYVDGVEVHNAKTKPREEQYNSTYRVVSVLTVLHQDW |
| | 248) | LNGKEYKCKVSNKALGAPIEKTISKAKGQPREPQVCTLP |
| | chain | PSRDELTKNQVSLSCAVKGFYPSDIAVEWESNGQPENNY |
| | | KTTPPVLDSDGSFFLVSKLTVDKSRWQQGNVFSCSVMHE |
| | | ALHNHYTQKSLSLSPGGGGGSGGGGSREGPELSPDDPAG |
| | | LLDLRQGMFAQLVAQNVLLIDGPLSWYSDPGLAGVSLTG |
| | | GLSYKEDTKELVVAKAGVYYVFFQLELRRVVAGEGSGSV |
| | | SLALHLQPLRSAAGAAALALTVDLPPASSEARNSAFGFQ |
| | | GRLLHLSAGQRLGVHLHTEARARHAWQLTQGATVLGLFR |
| | | VTPEIPAGLGGGGSGGGGSREGPELSPDDPAGLLDLRQG |
| | | MFAQLVAQNVLLIDGPLSWYSDPGLAGVSLTGGLSYKED |
| | | TKELVVAKAGVYYVFFQLELRRVVAGEGSGSVSLALHLQ |
| | | PLRSAAGAAALALTVDLPPASSEARNSAFGFQGRLLHLS |
| | | AGQRLGVHLHTEARARHAWQLTQGATVLGLFRVTPEIPA |
| | | GL |
| 174 | Fc knob | see Table 23 |
| | mono- | |
| | meric | |
| | 4-1BB | |
| | ligand | |
| | (71- | |
| | 248) | |
| 114 | anti- | see Table 3 |
| | FAP | |
| | (28H1) | |
| | light | |
| | chain | |

Table 26 shows the cDNA and amino acid sequences of the monovalent FAP(28H1)-targeted 4-1BB ligand trimer-containing Fc (kih) fusion antigen binding molecule (Construct 1.14).

TABLE 26
Sequences of FAP(28H1)-targeted human 4-1BB
ligand trimer containing Fc (kih) fusion
molecule Construct 1.14

| SEQ | De- | |
|---|---|---|
| ID | scrip- | |
| NO: | tion | Sequence |
| 181 | nucleo- | GAAGTGCAGCTGCTGGAATCCGGCGGAGGCCTGGTGCA |
| | tide | GCCTGGCGGATCTCTGAGACTGTCCTGCGCCGCCTCCGG |
| | se- | CTTCACCTTCTCCTCCCACGCCATGTCCTGGGTCCGACA |
| | quence | GGCTCCTGGCAAAGGCCTGGAATGGGTGTCCGCCATCTG |
| | of | GGCCTCCGGCGAGCAGTACTACGCCGACTCTGTGAAGG |
| | anti- | GCCGGTTCACCATCTCCCGGGACAACTCCAAGAACACCC |
| | FAP | TGTACCTGCAGATGAACTCCCTGCGGGCCGAGGACACC |
| | (28H1) | GCCGTGTACTACTGTGCCAAGGGCTGGCTGGGCAACTTC |
| | Fc hole | GACTACTGGGGACAGGGCACCCTGGTCACCGTGTCCAG |
| | mono- | CGCTAGCACCAAGGGCCCCTCCGTGTTCCCCCTGGCCCC |
| | meric | CAGCAGCAAGAGCACCAGCGGCGGCACAGCCGCTCTGG |
| | 4-1BB | GCTGCCTGGTCAAGGACTACTTCCCCGAGCCCGTGACCG |
| | ligand | TGTCCTGGAACAGCGGAGCCCTGACCTCCGGCGTGCAC |
| | (71- | ACCTTCCCCGCCGTGCTGCAGAGTTCTGGCCTGTATAGC |
| | 248) | CTGAGCAGCGTGGTCACCGTGCCTTCTAGCAGCCTGGGC |
| | chain | ACCCAGACCTACATCTGCAACGTGAACCACAAGCCCAG |
| | | CAACACCAAGGTGGACAAGAAGGTGGAGCCCAAGAGCT |
| | | GCGACAAAACTCACACATGCCCACCGTGCCCAGCACCT |
| | | GAAGCTGCAGGGGGACCGTCAGTCTTCCTCTTCCCCCCA |
| | | AAACCCAAGGACACCCTCATGATCTCCCGGACCCCTGA |
| | | GGTCACATGCGTGGTGGTGGACGTGAGCCACGAAGACC |
| | | CTGAGGTCAAGTTCAACTGGTACGTGGACGGCGTGGAG |
| | | GTGCATAATGCCAAGACAAAGCCGCGGGAGGAGCAGTA |
| | | CAACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCGGCGCCCCCATCGAGAAA |
| | | ACCATCTCCAAAGCCAAAGGGCAGCCCCGAGAACCACA |
| | | GGTGTGCACCCTGCCCCCATCCCGGGATGAGCTGACCAA |
| | | GAACCAGGTCAGCCTCTCGTGCGCAGTCAAAGGCTTCTA |
| | | TCCCAGCGACATCGCCGTGGAGTGGGAGAGCAATGGGC |
| | | AGCCGGAGAACAACTACAAGACCACGCCTCCCGTGCTG |
| | | GACTCCGACGGCTCCTTCTTCCTCGTGAGCAAGCTCACC |
| | | GTGGACAAGAGCAGGTGGCAGCAGGGGAACGTCTTCTC |
| | | ATGCTCCGTGATGCATGAGGCTCTGCACAACCACTACAC |
| | | GCAGAAGAGCCTCTCCCTGTCTCCGGGTGGAGGCGGCG |
| | | GAAGCGGAGGAGGAGGATCCAGAGAGGGCCCTGAGCTG |
| | | AGCCCTGATGATCCTGCCGGACTGCTGGACCTGCGGCAG |
| | | GGAATGTTTGCCCAGCTGGTGGCCCAGAACGTGCTGCTG |
| | | ATCGATGGCCCCCTGTCCTGGTACAGCGATCCTGGACTG |
| | | GCTGGCGTGTCACTGACAGGCGGCCTGAGCTACAAAGA |
| | | GGACACCAAAGAACTGGTGGTGGCCAAGGCCGGCGTGT |
| | | ACTACGTGTTCTTTCAGCTGGAACTGCGGAGAGTGGTGG |
| | | CCGGCGAAGGATCTGGCTCTGTGTCTCTGGCCCTGCATC |
| | | TGCAGCCTCTGAGATCTGCTGCTGGCGCCGCTGCTCTGG |
| | | CACTGACAGTGGATCTGCCTCCTGCCAGCAGCGAGGCCC |
| | | GGAATAGCGCATTTGGGTTTCAAGGCAGGCTGCTGCACC |
| | | TGTCTGCCGGCCAGAGGCTGGGAGTGCATCTGCACACA |
| | | GAGGCCAGGGCTAGACACGCCTGGCAGCTGACACAGGG |
| | | CGCTACAGTGCTGGGCCTGTTCAGAGTGACCCCCGAGAT |
| | | TCCTGCCGGGCTC |
| 176 | nucleo- | see Table 24 |
| | tide | |
| | se | |
| | quence | |
| | of Fc | |
| | knob | |
| | dimeric | |
| | 4-1BBL | |
| | (71- | |
| | 248) | |
| 112 | nucleo- | See Table 3 |
| | tide | |
| | se- | |
| | quence | |
| | of | |
| | anti- | |
| | FAP | |
| | (28H1) | |
| | light | |
| | chain | |
| 182 | anti- | EVQLLESGGGLVQPGGSLRLSCAASGFTFSSHAMSWVRQ |
| | FAP | APGKGLEWVSAIWASGEQYYADSVKGRFTISRDNSKNTL |
| | (28H1) | YLQMNSLRAEDTAVYYCAKGWLGNFDYWGQGTLVTVSSA |
| | Fc hole | STKGPSVFPLAPSSKSTSGGTAALGCLVKDYFPEPVTVS |
| | mono- | WNSGALTSGVHTFPAVLQSSGLYSLSSVVTVPSSSLGTQ |
| | meric | TYICNVNHKPSNTKVDKKVEPKSCDKTHTCPPCPAPEAA |
| | 4-1BBL | GGPSVFLFPPKPKDTLMISRTPEVTCVVVDVSHEDPEVK |
| | (71- | FNWYVDGVEVHNAKTKPREEQYNSTYRVVSVLTVLHQDW |
| | 248) | LNGKEYKCKVSNKALGAPIEKTISKAKGQPREPQVCTLP |
| | chain | PSRDELTKNQVSLSCAVKGFYPSDIAVEWESNGQPENNY |
| | | KTTPPVLDSDGSFFLVSKLTVDKSRWQQGNVFSCSVMHE |
| | | ALHNHYTQKSLSLSPGGGGGSGGGGSREGPELSPDDPAG |
| | | LLDLRQGMFAQLVAQNVLLIDGPLSWYSDPGLAGVSLTG |
| | | GLSYKEDTKELVVAKAGVYYVFFQLELRRVVAGEGSGSV |
| | | SLALHLQPLRSAAGAAALALTVDLPPASSEARNSAFGFQ |
| | | GRLLHLSAGQRLGVHLHTEARARHAWQLTQGATVLGLFR |
| | | VTPEIPAGL |
| 178 | Fc knob | see Table 24 |
| | dimeric | |
| | 4-1BB | |
| | ligand | |
| | (71- | |
| | 248) | |
| 114 | anti- | See Table 3 |
| | FAP | |
| | (28H1) | |
| | light | |
| | chain | |

1.8 Preparation of Monovalent CD19-Targeted 4-1BB Ligand Trimer-Containing Fc (Kih) Fusion Antigen Binding Molecule, Wherein the 4-1BB Ligands are C-Terminally Fused (Constructs 3.13, 4.13, 3.14 and 4.14) (CD19 on the Hole Chain)

The molecules are prepared as described in Example 1.7 for the monovalent FAP-targeted 4-1BB ligand trimer-containing Fc (kih) fusion antigen binding molecules, with the only difference that the anti-FAP binder was replaced by an anti-CD19 binder.

The variable region of heavy and light chain DNA sequences encoding a binder specific for CD19, clone 8B8-018 or 8B8-2B11, are subcloned in frame with either the constant heavy chain of the hole or the constant light chain of human IgG1.

Combination of the anti-CD19huIgG1 Fc hole chain containing the Y349C/T366S/L368A/Y407V mutations, the huIgG1 knob chain containing the S354C/T366W mutations and the anti-CD19 light chain allows generation of a heterodimer, which includes an assembled trimeric 4-1BB ligand and one CD19binding Fab (FIG. 2C or 2D).

Table 27 shows, respectively, the DNA and amino acid sequences of the monovalent targeted CD19(8B8-018) split trimeric 4-1BB ligand(71-248) C-terminal Fc(kih) fusion antigen binding molecule (construct 3.13).

TABLE 27
Sequences of CD19(8B8-018)-targeted human
4-1BB ligand trimer containing Fc (kih)
fusion molecule Construct 3.13

| SEQ | De- | |
|---|---|---|
| ID | scrip- | |
| NO: | tion | Sequence |
| 183 | nucleo- | CAGGTCCAGCTGGTGCAGTCCGGCGCCGAGGTCAAGAA |
| | tide | ACCCGGGGCTTCTGTGAAGGTTTCATGCAAGGCAAGCG |
| | se- | GATACACCTTCACCGACTATATCATGCATTGGGTCAGGC |
| | quence | AGGCCCCTGGCCAAGGTCTCGAATGGATGGGCTACATTA |
| | of | ACCCATATAATGATGGCTCCAAATACACCGAGAAGTTTC |
| | anti- | AGGGAAGAGTCACTATGACATCTGACACCAGTATCAGC |
| | CD19 | ACTGCTTACATGGAGCTGTCCCGCCTTCGGTCTGATGAC |
| | (8B8- | ACCGCAGTGTATTACTGTGCCAGGGGCACATATTACTAC |
| | 018) | GGCTCAGCTCTGTTCGACTATTGGGGGCAGGGAACCACA |
| | Fc hole | GTAACCGTGAGCTCCGCTAGCACCAAGGGCCCCTCCGTG |
| | dimeric | TTCCCCCTGGCCCCCAGCAGCAAGAGCACCAGCGGCGG |
| | 4-1BB | CACAGCCGCTCTGGGCTGCCTGGTCAAGGACTACTTCCC |
| | ligand | CGAGCCCGTGACCGTGTCCTGGAACAGCGGAGCCCTGA |
| | (71- | CCTCCGGCGTGCACACCTTCCCCGCCGTGCTGCAGAGTT |
| | 248) | CTGGCCTGTATAGCCTGAGCAGCGTGGTCACCGTGCCTT |
| | chain | CTAGCAGCCTGGGCACCCAGACCTACATCTGCAACGTG |
| | | AACCACAAGCCCAGCAACACCAAGGTGGACAAGAAGGT |
| | | GGAGCCCAAGAGCTGCGACAAAACTCACACATGCCCAC |
| | | CGTGCCCAGCACCTGAAGCTGCAGGGGGACCGTCAGTC |
| | | TTCCTCTTCCCCCCAAAACCCAAGGACACCCTCATGATC |
| | | TCCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTGCACCCTGCCCCCATCCCGG |
| | | GATGAGCTGACCAAGAACCAGGTCAGCCTCTCGTGCGC |
| | | AGTCAAAGGCTTCTATCCCAGCGACATCGCCGTGGAGTG |
| | | GGAGAGCAATGGGCAGCCGGAGAACAACTACAAGACC |
| | | ACGCCTCCCGTGCTGGACTCCGACGGCTCCTTCTTCCTC |
| | | GTGAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTGGAGGCGGCGGAAGCGGAGGAGGAGGATCCAGA |
| | | GAGGGCCCTGAGCTGAGCCCTGATGATCCTGCCGGACT |
| | | GCTGGACCTGCGGCAGGGAATGTTTGCCCAGCTGGTGGC |
| | | CCAGAACGTGCTGCTGATCGATGGCCCCCTGTCCTGGTA |
| | | CAGCGATCCTGGACTGGCTGGCGTGTCACTGACAGGCG |
| | | GCCTGAGCTACAAAGAGGACACCAAAGAACTGGTGGTG |
| | | GCCAAGGCCGGCGTGTACTACGTGTTCTTTCAGCTGGAA |
| | | CTGCGGAGAGTGGTGGCCGGCGAAGGATCTGGCTCTGT |
| | | GTCTCTGGCCCTGCATCTGCAGCCTCTGAGATCTGCTGC |
| | | TGGCGCCGCTGCTCTGGCACTGACAGTGGATCTGCCTCC |
| | | TGCCAGCAGCGAGGCCCGGAATAGCGCATTTGGGTTTCA |
| | | AGGCAGGCTGCTGCACCTGTCTGCCGGCCAGAGGCTGG |
| | | GAGTGCATCTGCACACAGAGGCCAGGGCTAGACACGCC |
| | | TGGCAGCTGACACAGGGCGCTACAGTGCTGGGCCTGTTC |
| | | AGAGTGACCCCCGAGATTCCAGCAGGCCTGGGAGGCGG |
| | | CGGATCTGGCGGCGGAGGATCTAGAGAAGGACCCGAGC |
| | | TGTCCCCCGACGATCCCGCTGGGCTGCTGGATCTGAGAC |
| | | AGGGCATGTTCGCTCAGCTGGTGGCTCAGAATGTGCTGC |
| | | TGATTGACGGACCTCTGAGCTGGTACTCCGACCCAGGGC |
| | | TGGCAGGGGTGTCCCTGACTGGGGGACTGTCCTACAAA |
| | | GAAGATACAAAAGAACTGGTGGTGGCTAAAGCTGGGGT |
| | | GTACTATGTGTTTTTTCAGCTGGAACTGAGGCGGGTGGT |
| | | GGCTGGGGAGGGCTCAGGATCTGTGTCCCTGGCTCTGCA |
| | | TCTGCAGCCACTGCGCTCTGCAGCAGGGGCTGCAGCACT |
| | | GGCCCTGACTGTGGACCTGCCCCCAGCTTCTTCCGAGGC |
| | | CAGAAACAGCGCCTTCGGGTTCCAAGGACGCCTGCTGC |
| | | ATCTGAGCGCCGGACAGCGCCTGGGAGTGCATCTGCAT |
| | | ACTGAAGCCAGAGCCCGGCATGCTTGGCAGCTGACTCA |
| | | GGGGGCAACTGTGCTGGGACTGTTTCGCGTGACACCTGA |
| | | GATCCCAGCCGGGCTC |
| 172 | nucleo- | see Table 23 |
| | tide | |
| | se- | |
| | quence | |
| | of | |
| | Fc knob | |
| | mono- | |
| | meric | |
| | 4-1BB | |
| | (71- | |
| | 248) | |
| | ligand | |
| 118 | nucleo- | see Table 5 |
| | tide | |
| | se- | |
| | quence | |
| | of | |
| | anti- | |
| | CD19 | |
| | (8B8- | |
| | 018) | |
| | light | |
| | chain | |
| 184 | anti- | QVQLVQSGAEVKKPGASVKVSCKASGYTFTDYIMHWVRQ |
| | CD19 | APGQGLEWMGYINPYNDGSKYTEKFQGRVTMTSDTSIST |
| | (8B8- | AYMELSRLRSDDTAVYYCARGTYYYGSALFDYWGQGTTV |
| | 018) | TVSSASTKGPSVFPLAPSSKSTSGGTAALGCLVKDYFPE |
| | Fc hole | PVTVSWNSGALTSGVHTFPAVLQSSGLYSLSSVVTVPSS |
| | dimeric | SLGTQTYICNVNHKPSNTKVDKKVEPKSCDKTHTCPPCP |
| | 4-1BB | APEAAGGPSVFLFPPKPKDTLMISRTPEVTCVVVDVSHE |
| | (71- | DPEVKFNWYVDGVEVHNAKTKPREEQYNSTYRVVSVLTV |
| | 248) | LHQDWLNGKEYKCKVSNKALGAPIEKTISKAKGQPREPQ |
| | ligand | VCTLPPSRDELTKNQVSLSCAVKGFYPSDIAVEWESNGQ |
| | chain | PENNYKTTPPVLDSDGSFFLVSKLTVDKSRWQQGNVFSC |
| | | SVMHEALHNHYTQKSLSLSPGGGGGSGGGGSREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGLGGGGSGGGGSREGPELSPDDPAGLL |
| | | DLRQGMFAQLVAQNVLLIDGPLSWYSDPGLAGVSLTGGL |
| | | SYKEDTKELVVAKAGVYYVFFQLELRRVVAGEGSGSVSL |
| | | ALHLQPLRSAAGAAALALTVDLPPASSEARNSAFGFQGR |
| | | LLHLSAGQRLGVHLHTEARARHAWQLTQGATVLGLFRVT |
| | | PEIPAGL |
| 174 | Fc knob | see Table 23 |
| | mono- | |
| | meric | |
| | 41-BBL | |
| | (71- | |
| | 248) | |
| 120 | anti- | see Table 5 |
| | FAP | |
| | (4B9) | |
| | light | |
| | chain | |

Table 28 shows the DNA and amino acid sequences of the monovalent CD19(8B8-018)-targeted 4-1BB ligand (71-248) timer-containing Fc (kih) fusion antigen binding molecule (Construct 3.14).

TABLE 28
Sequences of CD19(8B8-018)-targeted human
4-1BB ligand trimer containing Fc (kih)
fusion molecule Construct 3.14

| SEQ | De- | |
|---|---|---|
| ID | scrip- | |
| NO: | tion | Sequence |
| 185 | nucleo- | CAGGTCCAGCTGGTGCAGTCCGGCGCCGAGGTCAAGAA |
| | tide | ACCCGGGGCTTCTGTGAAGGTTTCATGCAAGGCAAGCG |
| | se- | GATACACCTTCACCGACTATATCATGCATTGGGTCAGGC |
| | quence | AGGCCCCTGGCCAAGGTCTCGAATGGATGGGCTACATTA |
| | of | ACCCATATAATGATGGCTCCAAATACACCGAGAAGTTTC |
| | anti- | AGGGAAGAGTCACTATGACATCTGACACCAGTATCAGC |
| | CD19 | ACTGCTTACATGGAGCTGTCCCGCCTTCGGTCTGATGAC |
| | (8B8- | ACCGCAGTGTATTACTGTGCCAGGGGCACATATTACTAC |
| | 018) | GGCTCAGCTCTGTTCGACTATTGGGGGCAGGGAACCACA |
| | Fc hole | GTAACCGTGAGCTCCGCTAGCACCAAGGGCCCCTCCGTG |
| | mono- | TTCCCCCTGGCCCCCAGCAGCAAGAGCACCAGCGGCGG |
| | meric | CACAGCCGCTCTGGGCTGCCTGGTCAAGGACTACTTCCC |
| | 4-1BBL | CGAGCCCGTGACCGTGTCCTGGAACAGCGGAGCCCTGA |
| | (71- | CCTCCGGCGTGCACACCTTCCCCGCCGTGCTGCAGAGTT |
| | 248) | CTGGCCTGTATAGCCTGAGCAGCGTGGTCACCGTGCCTT |
| | chain | CTAGCAGCCTGGGCACCCAGACCTACATCTGCAACGTG |
| | | AACCACAAGCCCAGCAACACCAAGGTGGACAAGAAGGT |
| | | GGAGCCCAAGAGCTGCGACAAAACTCACACATGCCCAC |
| | | CGTGCCCAGCACCTGAAGCTGCAGGGGGACCGTCAGTC |
| | | TTCCTCTTCCCCCCAAAACCCAAGGACACCCTCATGATC |
| | | TCCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTGCACCCTGCCCCCATCCCGG |
| | | GATGAGCTGACCAAGAACCAGGTCAGCCTCTCGTGCGC |
| | | AGTCAAAGGCTTCTATCCCAGCGACATCGCCGTGGAGTG |
| | | GGAGAGCAATGGGCAGCCGGAGAACAACTACAAGACC |
| | | ACGCCTCCCGTGCTGGACTCCGACGGCTCCTTCTTCCTC |
| | | GTGAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTGGAGGCGGCGGAAGCGGAGGAGGAGGATCCAGA |
| | | GAGGGCCCTGAGCTGAGCCCTGATGATCCTGCCGGACT |
| | | GCTGGACCTGCGGCAGGGAATGTTTGCCCAGCTGGTGGC |
| | | CCAGAACGTGCTGCTGATCGATGGCCCCCTGTCCTGGTA |
| | | CAGCGATCCTGGACTGGCTGGCGTGTCACTGACAGGCG |
| | | GCCTGAGCTACAAAGAGGACACCAAAGAACTGGTGGTG |
| | | GCCAAGGCCGGCGTGTACTACGTGTTCTTTCAGCTGGAA |
| | | CTGCGGAGAGTGGTGGCCGGCGAAGGATCTGGCTCTGT |
| | | GTCTCTGGCCCTGCATCTGCAGCCTCTGAGATCTGCTGC |
| | | TGGCGCCGCTGCTCTGGCACTGACAGTGGATCTGCCTCC |
| | | TGCCAGCAGCGAGGCCCGGAATAGCGCATTTGGGTTTCA |
| | | AGGCAGGCTGCTGCACCTGTCTGCCGGCCAGAGGCTGG |
| | | GAGTGCATCTGCACACAGAGGCCAGGGCTAGACACGCC |
| | | TGGCAGCTGACACAGGGCGCTACAGTGCTGGGCCTGTTC |
| | | AGAGTGACCCCCGAGATTCCTGCCGGGCTC |
| 176 | nucleo- | see Table 24 |
| | tide | |
| | se- | |
| | quence | |
| | of | |
| | Fc knob | |
| | dimeric | |
| | 4-1BB | |
| | ligand | |
| | (71- | |
| | 248) | |
| 118 | nucleo- | See Table 5 |
| | tide | |
| | se- | |
| | quence | |
| | of | |
| | anti- | |
| | CD19 | |
| | (8B8- | |
| | 018) | |
| | light | |
| | chain | |
| 186 | anti- | QVQLVQSGAEVKKPGASVKVSCKASGYTFTDYIMHWVRQ |
| | CD19 | APGQGLEWMGYINPYNDGSKYTEKFQGRVTMTSDTSIST |
| | (8B8- | AYMELSRLRSDDTAVYYCARGTYYYGSALFDYWGQGTTV |
| | 018) | TVSSASTKGPSVFPLAPSSKSTSGGTAALGCLVKDYFPE |
| | Fc hole | PVTVSWNSGALTSGVHTFPAVLQSSGLYSLSSVVTVPSS |
| | mono- | SLGTQTYICNVNHKPSNTKVDKKVEPKSCDKTHTCPPCP |
| | meric | APEAAGGPSVFLFPPKPKDTLMISRTPEVTCVVVDVSHE |
| | 4-1BB | DPEVKFNWYVDGVEVHNAKTKPREEQYNSTYRVVSVLTV |
| | ligand | LHQDWLNGKEYKCKVSNKALGAPIEKTISKAKGQPREPQ |
| | (71- | VCTLPPSRDELTKNQVSLSCAVKGFYPSDIAVEWESNGQ |
| | 248) | PENNYKTTPPVLDSDGSFFLVSKLTVDKSRWQQGNVFS |
| | chain | CSVMHEALHNHYTQKSLSLSPGGGGGSGGGGSREGPEL |
| | | SPDDPAGLLDLRQGMFAQLVAQNVLLIDGPLSWYSDPG |
| | | LAGVSLTGGLSYKEDTKELVVAKAGVYYVFFQLELRRV |
| | | VAGEGSGSVSLALHLQPLRSAAGAAALALTVDLPPASS |
| | | EARNSAFGFQGRLLHLSAGQRLGVHLHTEARARHAWQL |
| | | TQGATVLGLFRVTPEIPAGL |
| 178 | Fc knob | see Table 24 |
| | dimeric | |
| | 4-1BB | |
| | ligand | |
| | (71- | |
| | 248) | |
| 120 | anti- | See Table 5 |
| | CD19 | |
| | (8B8- | |
| | 018) | |
| | light | |
| | chain | |

Table 29 shows the cDNA and amino acid sequences of the monovalent CD19(8B8-2B11)-targeted 4-1BB ligand trimer-containing Fc (kih) fusion antigen binding molecule (Construct 4.13).

TABLE 29
Sequences of CD19(8B8-2B11)-targeted human
4-1BB ligand trimer containing Fc (kih)
fusion molecule Construct 4.13

| SEQ | De- | |
|---|---|---|
| ID | scrip- | |
| NO: | tion | Sequence |
| 187 | nucleo- | CAGGTGCAATTGGTTCAATCTGGTGCTGAAGTAAAAAA |
| | tide | ACCGGGCGCTTCCGTTAAAGTGAGCTGCAAAGCATCTGG |
| | se- | TTACACCTTCACTGACTATATCATGCACTGGGTTCGTCA |
| | quence | GGCCCCGGGCCAGGGTCTGGAGTGGATGGGCTACATTA |
| | of | ACCCATACAACGACGGTTCCAAATATACCGAGAAATTC |
| | anti- | CAGGGCCGCGTCACGATGACCAGCGACACTTCTATCTCC |
| | CD19 | ACCGCGTACATGGAACTGTCTAGACTGCGTTCTGACGAC |
| | (8B8- | ACCGCTGTTTACTATTGTGCACGCGGTACCTACTACTAC |
| | 2B11) | GGTCCACAGCTGTTTGATTACTGGGGCCAAGGTACCACG |
| | Fc hole | GTGACCGTAAGCTCTGCTAGCACCAAGGGCCCCTCCGTG |
| | dimenc | TTCCCCCTGGCCCCCAGCAGCAAGAGCACCAGCGGCGG |
| | 4-1BB | CACAGCCGCTCTGGGCTGCCTGGTCAAGGACTACTTCCC |
| | ligand | CGAGCCCGTGACCGTGTCCTGGAACAGCGGAGCCCTGA |
| | (71- | CCTCCGGCGTGCACACCTTCCCCGCCGTGCTGCAGAGTT |
| | 248) | CTGGCCTGTATAGCCTGAGCAGCGTGGTCACCGTGCCTT |
| | chain | CTAGCAGCCTGGGCACCCAGACCTACATCTGCAACGTG |
| | | AACCACAAGCCCAGCAACACCAAGGTGGACAAGAAGGT |
| | | GGAGCCCAAGAGCTGCGACAAAACTCACACATGCCCAC |
| | | CGTGCCCAGCACCTGAAGCTGCAGGGGGACCGTCAGTC |
| | | TTCCTCTTCCCCCCAAAACCCAAGGACACCCTCATGATC |
| | | TCCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTGCACCCTGCCCCCATCCCGG |
| | | GATGAGCTGACCAAGAACCAGGTCAGCCTCTCGTGCGC |
| | | AGTCAAAGGCTTCTATCCCAGCGACATCGCCGTGGAGTG |
| | | GGAGAGCAATGGGCAGCCGGAGAACAACTACAAGACC |
| | | ACGCCTCCCGTGCTGGACTCCGACGGCTCCTTCTTCCTC |
| | | GTGAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTGGAGGCGGCGGAAGCGGAGGAGGAGGATCCAGA |
| | | GAGGGCCCTGAGCTGAGCCCTGATGATCCTGCCGGACT |
| | | GCTGGACCTGCGGCAGGGAATGTTTGCCCAGCTGGTGGC |
| | | CCAGAACGTGCTGCTGATCGATGGCCCCCTGTCCTGGTA |
| | | CAGCGATCCTGGACTGGCTGGCGTGTCACTGACAGGCG |
| | | GCCTGAGCTACAAAGAGGACACCAAAGAACTGGTGGTG |
| | | GCCAAGGCCGGCGTGTACTACGTGTTCTTTCAGCTGGAA |
| | | CTGCGGAGAGTGGTGGCCGGCGAAGGATCTGGCTCTGT |
| | | GTCTCTGGCCCTGCATCTGCAGCCTCTGAGATCTGCTGC |
| | | TGGCGCCGCTGCTCTGGCACTGACAGTGGATCTGCCTCC |
| | | TGCCAGCAGCGAGGCCCGGAATAGCGCATTTGGGTTTCA |
| | | AGGCAGGCTGCTGCACCTGTCTGCCGGCCAGAGGCTGG |
| | | GAGTGCATCTGCACACAGAGGCCAGGGCTAGACACGCC |
| | | TGGCAGCTGACACAGGGCGCTACAGTGCTGGGCCTGTTC |
| | | AGAGTGACCCCCGAGATTCCAGCAGGCCTGGGAGGCGG |
| | | CGGATCTGGCGGCGGAGGATCTAGAGAAGGACCCGAGC |
| | | TGTCCCCCGACGATCCCGCTGGGCTGCTGGATCTGAGAC |
| | | AGGGCATGTTCGCTCAGCTGGTGGCTCAGAATGTGCTGC |
| | | TGATTGACGGACCTCTGAGCTGGTACTCCGACCCAGGGC |
| | | TGGCAGGGGTGTCCCTGACTGGGGGACTGTCCTACAAA |
| | | GAAGATACAAAAGAACTGGTGGTGGCTAAAGCTGGGGT |
| | | GTACTATGTGTTTTTTCAGCTGGAACTGAGGCGGGTGGT |
| | | GGCTGGGGAGGGCTCAGGATCTGTGTCCCTGGCTCTGCA |
| | | TCTGCAGCCACTGCGCTCTGCAGCAGGGGCTGCAGCACT |
| | | GGCCCTGACTGTGGACCTGCCCCCAGCTTCTTCCGAGGC |
| | | CAGAAACAGCGCCTTCGGGTTCCAAGGACGCCTGCTGC |
| | | ATCTGAGCGCCGGACAGCGCCTGGGAGTGCATCTGCAT |
| | | ACTGAAGCCAGAGCCCGGCATGCTTGGCAGCTGACTCA |
| | | GGGGGCAACTGTGCTGGGACTGTTTCGCGTGACACCTGA |
| | | GATCCCAGCCGGGCTC |
| 172 | nucleo- | see Table 23 |
| | tide | |
| | se- | |
| | quence | |
| | of | |
| | Fc knob | |
| | mono- | |
| | meric | |
| | 4-1BB | |
| | ligand | |
| | (71- | |
| | 248) | |
| 124 | nucleo- | see Table 7 |
| | tide | |
| | se- | |
| | quence | |
| | of | |
| | anti- | |
| | CD19 | |
| | (8B8- | |
| | 2B11) | |
| | light | |
| | chain | |
| 188 | anti- | QVQLVQSGAEVKKPGASVKVSCKASGYTFTDYIMHWVRQ |
| | CD19 | APGQGLEWMGYINPYNDGSKYTEKFQGRVTMTSDTSIST |
| | (8B8- | AYMELSRLRSDDTAVYYCARGTYYYGPQLFDYWGQGTTV |
| | 2B11) | TVSSASTKGPSVFPLAPSSKSTSGGTAALGCLVKDYFPE |
| | Fc hole | PVTVSWNSGALTSGVHTFPAVLQSSGLYSLSSVVTVPSS |
| | dimeric | SLGTQTYICNVNHKPSNTKVDKKVEPKSCDKTHTCPPCP |
| | 4-1BB | APEAAGGPSVFLFPPKPKDTLMISRTPEVTCVVVDVSHE |
| | ligand | DPEVKFNWYVDGVEVHNAKTKPREEQYNSTYRVVSVLTV |
| | (71- | LHQDWLNGKEYKCKVSNKALGAPIEKTISKAKGQPREPQ |
| | 248) | VCTLPPSRDELTKNQVSLSCAVKGFYPSDIAVEWESNGQ |
| | chain | PENNYKTTPPVLDSDGSFFLVSKLTVDKSRWQQGNVFSC |
| | | SVMHEALHNHYTQKSLSLSPGGGGGSGGGGSREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGLGGGGSGGGGSREGPELSPDDPAGLL |
| | | DLRQGMFAQLVAQNVLLIDGPLSWYSDPGLAGVSLTGGL |
| | | SYKEDTKELVVAKAGVYYVFFQLELRRVVAGEGSGSVSL |
| | | ALHLQPLRSAAGAAALALTVDLPPASSEARNSAFGFQGR |
| | | LLHLSAGQRLGVHLHTEARARHAWQLTQGATVLGLFRVT |
| | | PEIPAGL |
| 174 | Fc knob | see Table 23 |
| | mono- | |
| | meric | |
| | 4-1BB | |
| | ligand | |
| | (71- | |
| | 248) | |
| 126 | anti- | see Table 7 |
| | CD19 | |
| | (8B8- | |
| | 2B11) | |
| | light | |
| | chain | |

Table 30 shows the cDNA and amino acid sequences ofthe monovalent CD9(8B8-2B11)-targeted 4-1BB ligand trimer-containing Fc (kih) fusion antigen binding molecule (Construct

TABLE 30
Sequences of CD19(8B8-2B11)-targeted human
4-1BB ligand trimer containing Fc (kih)
fusion molecule Construct 4.14

| SEQ | De- | |
|---|---|---|
| ID | scrip- | |
| NO: | tion | Sequence |
| 189 | nucleo- | CAGGTGCAATTGGTTCAATCTGGTGCTGAAGTAAAAAA |
| | tide | ACCGGGCGCTTCCGTTAAAGTGAGCTGCAAAGCATCTGG |
| | se- | TTACACCTTCACTGACTATATCATGCACTGGGTTCGTCA |
| | quence | GGCCCCGGGCCAGGGTCTGGAGTGGATGGGCTACATTA |
| | of | ACCCATACAACGACGGTTCCAAATATACCGAGAAATTC |
| | anti- | CAGGGCCGCGTCACGATGACCAGCGACACTTCTATCTCC |
| | CD19 | ACCGCGTACATGGAACTGTCTAGACTGCGTTCTGACGAC |
| | (8B8- | ACCGCTGTTTACTATTGTGCACGCGGTACCTACTACTAC |
| | 2B11) | GGTCCACAGCTGTTTGATTACTGGGGCCAAGGTACCACG |
| | Fc hole | GTGACCGTAAGCTCTGCTAGCACCAAGGGCCCCTCCGTG |
| | mono- | TTCCCCCTGGCCCCCAGCAGCAAGAGCACCAGCGGCGG |
| | meric | CACAGCCGCTCTGGGCTGCCTGGTCAAGGACTACTTCCC |
| | 4-1BB | CGAGCCCGTGACCGTGTCCTGGAACAGCGGAGCCCTGA |
| | ligand | CCTCCGGCGTGCACACCTTCCCCGCCGTGCTGCAGAGTT |
| | (71- | CTGGCCTGTATAGCCTGAGCAGCGTGGTCACCGTGCCTT |
| | 248) | CTAGCAGCCTGGGCACCCAGACCTACATCTGCAACGTG |
| | chain | AACCACAAGCCCAGCAACACCAAGGTGGACAAGAAGGT |
| | | GGAGCCCAAGAGCTGCGACAAAACTCACACATGCCCAC |
| | | CGTGCCCAGCACCTGAAGCTGCAGGGGGACCGTCAGTC |
| | | TTCCTCTTCCCCCCAAAACCCAAGGACACCCTCATGATC |
| | | TCCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTGCACCCTGCCCCCATCCCGG |
| | | GATGAGCTGACCAAGAACCAGGTCAGCCTCTCGTGCGC |
| | | AGTCAAAGGCTTCTATCCCAGCGACATCGCCGTGGAGTG |
| | | GGAGAGCAATGGGCAGCCGGAGAACAACTACAAGACC |
| | | ACGCCTCCCGTGCTGGACTCCGACGGCTCCTTCTTCCTC |
| | | GTGAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTGGAGGCGGCGGAAGCGGAGGAGGAGGATCCAGA |
| | | GAGGGCCCTGAGCTGAGCCCTGATGATCCTGCCGGACT |
| | | GCTGGACCTGCGGCAGGGAATGTTTGCCCAGCTGGTGGC |
| | | CCAGAACGTGCTGCTGATCGATGGCCCCCTGTCCTGGTA |
| | | CAGCGATCCTGGACTGGCTGGCGTGTCACTGACAGGCG |
| | | GCCTGAGCTACAAAGAGGACACCAAAGAACTGGTGGTG |
| | | GCCAAGGCCGGCGTGTACTACGTGTTCTTTCAGCTGGAA |
| | | CTGCGGAGAGTGGTGGCCGGCGAAGGATCTGGCTCTGT |
| | | GTCTCTGGCCCTGCATCTGCAGCCTCTGAGATCTGCTGC |
| | | TGGCGCCGCTGCTCTGGCACTGACAGTGGATCTGCCTCC |
| | | TGCCAGCAGCGAGGCCCGGAATAGCGCATTTGGGTTTCA |
| | | AGGCAGGCTGCTGCACCTGTCTGCCGGCCAGAGGCTGG |
| | | GAGTGCATCTGCACACAGAGGCCAGGGCTAGACACGCC |
| | | TGGCAGCTGACACAGGGCGCTACAGTGCTGGGCCTGTTC |
| | | AGAGTGACCCCCGAGATTCCTGCCGGGCTC |
| 176 | nucleo- | see Table 24 |
| | tide | |
| | se- | |
| | quence | |
| | of | |
| | Fc knob | |
| | dimeric | |
| | 4-1BBL | |
| | (71- | |
| | 248) | |
| 124 | nucleo- | See Table 7 |
| | tide | |
| | se- | |
| | quence | |
| | of | |
| | anti- | |
| | CD19 | |
| | (8B8- | |
| | 2B11) | |
| | light | |
| | chain | |
| 190 | anti- | QVQLVQSGAEVKKPGASVKVSCKASGYTFTDYIMHWVRQ |
| | CD19 | APGQGLEWMGYINPYNDGSKYTEKFQGRVTMTSDTSIST |
| | (8B8- | AYMELSRLRSDDTAVYYCARGTYYYGPQLFDYWGQGTTV |
| | 2B11) | TVSSASTKGPSVFPLAPSSKSTSGGTAALGCLVKDYFPE |
| | Fc hole | PVTVSWNSGALTSGVHTFPAVLQSSGLYSLSSVVTVPSS |
| | mono- | SLGTQTYICNVNHKPSNTKVDKKVEPKSCDKTHTCPPCP |
| | meric | APEAAGGPSVFLFPPKPKDTLMISRTPEVTCVVVDVSHE |
| | 4-1BBL | DPEVKFNWYVDGVEVHNAKTKPREEQYNSTYRVVSVLTV |
| | (71- | LHQDWLNGKEYKCKVSNKALGAPIEKTISKAKGQPREPQ |
| | 248) | VCTLPPSRDELTKNQVSLSCAVKGFYPSDIAVEWESNGQ |
| | chain | PENNYKTTPPVLDSDGSFFLVSKLTVDKSRWQQGNVFSC |
| | | SVMHEALHNHYTQKSLSLSPGGGGGSGGGGSREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGL |
| 178 | Fc knob | see Table 24 |
| | dimeric | |
| | 4-1BB | |
| | ligand | |
| | (71- | |
| | 248) | |
| 126 | anti- | See Table 7 |
| | CD19 | |
| | (8B8- | |
| | 2B11) | |
| | light | |
| | chain | |

1.9 Preparation of Monovalent CEA-Targeted 4-1BB Ligand Trimer-Containing Fc(Kih) Fusion Antigen Binding Molecule, Wherein the 4-1BB Ligands are C-Terminally Fused (Constructs 5.13 and 5.14) (CEA on the Hole Chain)

The molecules are prepared as described in Example 1.7 for the monovalent FAP-targeted construct. with the only difference that the anti-FAP binder was replaced by an anti-CEA binder.

The variable region of heavy and light chain DNA sequences encoding binder specific for carcinoembryonic antigen (CEA), clone T84.66-LCHA, are subcloned in frame with either the constant heavy chain of the knob or the constant light chain of human IgG1. The generation of the CEA clone is described in Example 1.5.

Combination of the huIgG1 Fc hole chain containing the Y349C/T366S/L368A/Y407V mutations in the CH3 domain, the anti-CEA huIgG1 knob chain containing the S354C/T366W mutations in the CH3 domain and the anti-CEA light chain allows generation of a heterodimer, which includes an assembled trimeric 4-1BB ligand and one CEA binding Fab (FIGS. 2C and 2D).

Table 31 shows the cDNA and amino acid sequences of the monovalent CEA(T84.66-LCHA)-targeted 4-1BB ligand (71-248) trimer-containing Fc (kih) fusion antigen binding molecule (Construct 5.13).

TABLE 17
Sequences of CEA(T84.66-LCHA)-targeted human
4-1BB ligand trimer containing Fc (kih)
fusion molecule Construct 5.13

| SEQ | De- | |
|---|---|---|
| ID | scrip- | |
| NO: | tion | Sequence |
| 191 | nucleo- | CAGGTGCAGCTGGTGCAGTCTGGCGCCGAAGTGAAGAA |
| | tide | ACCCGGCAGCAGCGTGAAGGTGTCCTGCAAGGCCAGCG |
| | se- | GCTTCAACATCAAGGACACCTACATGCACTGGGTGCGCC |
| | quence | AGGCCCCTGGACAGGGACTGGAATGGATGGGCAGAATC |
| | of | GACCCCGCCAACGGCAACAGCAAATACGTGCCCAAGTT |
| | anti- | CCAGGGCAGAGTGACCATCACCGCCGACACCAGCACCT |
| | CEA | CCACCGCCTACATGGAACTGAGCAGCCTGCGGAGCGAG |
| | (T84. | GACACCGCCGTGTACTACTGTGCCCCCTTCGGCTACTAC |
| | 66- | GTGTCCGACTACGCCATGGCCTATTGGGGCCAGGGCAC |
| | LCHA) | ACTCGTGACCGTGTCCTCTGCTAGCACCAAGGGCCCCTC |
| | Fc hole | CGTGTTCCCCCTGGCCCCCAGCAGCAAGAGCACCAGCG |
| | dimeric | GCGGCACAGCCGCTCTGGGCTGCCTGGTCAAGGACTACT |
| | 4-1BB | TCCCCGAGCCCGTGACCGTGTCCTGGAACAGCGGAGCC |
| | ligand | CTGACCTCCGGCGTGCACACCTTCCCCGCCGTGCTGCAG |
| | (71- | AGTTCTGGCCTGTATAGCCTGAGCAGCGTGGTCACCGTG |
| | 248) | CCTTCTAGCAGCCTGGGCACCCAGACCTACATCTGCAAC |
| | chain | GTGAACCACAAGCCCAGCAACACCAAGGTGGACAAGAA |
| | | GGTGGAGCCCAAGAGCTGCGACAAAACTCACACATGCC |
| | | CACCGTGCCCAGCACCTGAAGCTGCAGGGGGACCGTCA |
| | | GTCTTCCTCTTCCCCCCAAAACCCAAGGACACCCTCATG |
| | | ATCTCCCGGACCCCTGAGGTCACATGCGTGGTGGTGGAC |
| | | GTGAGCCACGAAGACCCTGAGGTCAAGTTCAACTGGTA |
| | | CGTGGACGGCGTGGAGGTGCATAATGCCAAGACAAAGC |
| | | CGCGGGAGGAGCAGTACAACAGCACGTACCGTGTGGTC |
| | | AGCGTCCTCACCGTCCTGCACCAGGACTGGCTGAATGGC |
| | | AAGGAGTACAAGTGCAAGGTCTCCAACAAAGCCCTCGG |
| | | CGCCCCCATCGAGAAAACCATCTCCAAAGCCAAAGGGC |
| | | AGCCCCGAGAACCACAGGTGTGCACCCTGCCCCCATCCC |
| | | GGGATGAGCTGACCAAGAACCAGGTCAGCCTCTCGTGC |
| | | GCAGTCAAAGGCTTCTATCCCAGCGACATCGCCGTGGA |
| | | GTGGGAGAGCAATGGGCAGCCGGAGAACAACTACAAGA |
| | | CCACGCCTCCCGTGCTGGACTCCGACGGCTCCTTCTTCC |
| | | TCGTGAGCAAGCTCACCGTGGACAAGAGCAGGTGGCAG |
| | | CAGGGGAACGTCTTCTCATGCTCCGTGATGCATGAGGCT |
| | | CTGCACAACCACTACACGCAGAAGAGCCTCTCCCTGTCT |
| | | CCGGGTGGAGGCGGCGGAAGCGGAGGAGGAGGATCCA |
| | | GAGAGGGCCCTGAGCTGAGCCCTGATGATCCTGCCGGA |
| | | CTGCTGGACCTGCGGCAGGGAATGTTTGCCCAGCTGGTG |
| | | GCCCAGAACGTGCTGCTGATCGATGGCCCCCTGTCCTGG |
| | | TACAGCGATCCTGGACTGGCTGGCGTGTCACTGACAGGC |
| | | GGCCTGAGCTACAAAGAGGACACCAAAGAACTGGTGGT |
| | | GGCCAAGGCCGGCGTGTACTACGTGTTCTTTCAGCTGGA |
| | | ACTGCGGAGAGTGGTGGCCGGCGAAGGATCTGGCTCTG |
| | | TGTCTCTGGCCCTGCATCTGCAGCCTCTGAGATCTGCTG |
| | | CTGGCGCCGCTGCTCTGGCACTGACAGTGGATCTGCCTC |
| | | CTGCCAGCAGCGAGGCCCGGAATAGCGCATTTGGGTTTC |
| | | AAGGCAGGCTGCTGCACCTGTCTGCCGGCCAGAGGCTG |
| | | GGAGTGCATCTGCACACAGAGGCCAGGGCTAGACACGC |
| | | CTGGCAGCTGACACAGGGCGCTACAGTGCTGGGCCTGTT |
| | | CAGAGTGACCCCCGAGATTCCAGCAGGCCTGGGAGGCG |
| | | GCGGATCTGGCGGCGGAGGATCTAGAGAAGGACCCGAG |
| | | CTGTCCCCCGACGATCCCGCTGGGCTGCTGGATCTGAGA |
| | | CAGGGCATGTTCGCTCAGCTGGTGGCTCAGAATGTGCTG |
| | | CTGATTGACGGACCTCTGAGCTGGTACTCCGACCCAGGG |
| | | CTGGCAGGGGTGTCCCTGACTGGGGGACTGTCCTACAAA |
| | | GAAGATACAAAAGAACTGGTGGTGGCTAAAGCTGGGGT |
| | | GTACTATGTGTTTTTTCAGCTGGAACTGAGGCGGGTGGT |
| | | GGCTGGGGAGGGCTCAGGATCTGTGTCCCTGGCTCTGCA |
| | | TCTGCAGCCACTGCGCTCTGCAGCAGGGGCTGCAGCACT |
| | | GGCCCTGACTGTGGACCTGCCCCCAGCTTCTTCCGAGGC |
| | | CAGAAACAGCGCCTTCGGGTTCCAAGGACGCCTGCTGC |
| | | ATCTGAGCGCCGGACAGCGCCTGGGAGTGCATCTGCAT |
| | | ACTGAAGCCAGAGCCCGGCATGCTTGGCAGCTGACTCA |
| | | GGGGGCAACTGTGCTGGGACTGTTTCGCGTGACACCTGA |
| | | GATCCCAGCCGGGCTC |
| 172 | nucleo- | see Table 23 |
| | tide | |
| | se- | |
| | quence | |
| | of | |
| | Fc knob | |
| | mono- | |
| | meric | |
| | 4-1BBL | |
| | (71- | |
| | 248) | |
| 154 | nucleo- | see Table 17 |
| | tide | |
| | se- | |
| | quence | |
| | of | |
| | anti- | |
| | CEA | |
| | (T84. | |
| | 66- | |
| | LCHA) | |
| | light | |
| | chain | |
| 192 | anti- | QVQLVQSGAEVKKPGSSVKVSCKASGFNIKDTYMHWVRQ |
| | CEA | APGQGLEWMGRIDPANGNSKYVPKFQGRVTITADTSTST |
| | (T84. | AYMELSSLRSEDTAVYYCAPFGYYVSDYAMAYWGQGTLV |
| | 66- | TVSSASTKGPSVFPLAPSSKSTSGGTAALGCLVKDYFPE |
| | LCHA) | PVTVSWNSGALTSGVHTFPAVLQSSGLYSLSSVVTVPSS |
| | Fc hole | SLGTQTYICNVNHKPSNTKVDKKVEPKSCDKTHTCPPCP |
| | dimeric | APEAAGGPSVFLFPPKPKDTLMISRTPEVTCVVVDVSHE |
| | 4-1BB | DPEVKFNWYVDGVEVHNAKTKPREEQYNSTYRVVSVLTV |
| | (71- | LHQDWLNGKEYKCKVSNKALGAPIEKTISKAKGQPREPQ |
| | 248) | VCTLPPSRDELTKNQVSLSCAVKGFYPSDIAVEWESNGQ |
| | ligand | PENNYKTTPPVLDSDGSFFLVSKLTVDKSRWQQGNVFSC |
| | chain | SVMHEALHNHYTQKSLSLSPGGGGGSGGGGSREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGLGGGGSGGGGSREGPELSPDDPAGLL |
| | | DLRQGMFAQLVAQNVLLIDGPLSWYSDPGLAGVSLTGGL |
| | | SYKEDTKELVVAKAGVYYVFFQLELRRVVAGEGSGSVSL |
| | | ALHLQPLRSAAGAAALALTVDLPPASSEARNSAFGFQGR |
| | | LLHLSAGQRLGVHLHTEARARHAWQLTQGATVLGLFRVT |
| | | PEIPAGL |
| 174 | Fc knob | see Table 23 |
| | mono- | |
| | meric | |
| | 4-1-BB | |
| | (71- | |
| | 248) | |
| | ligand | |
| 156 | anti- | see Table 17 |
| | CEA | |
| | (T84. | |
| | 66- | |
| | LCHA) | |
| | light | |
| | chain | |

Table 32 shows the cDNA and amino acid sequences of the monovalent CEA(T84.66-LCHA)-targeted 4-1BB ligand (71-248)trimer-containing Fc (kih) fusion antigen binding molecule (Construct 5.14).

TABLE 32
Sequences of CEA(T84.66-LCHA)-targetedhuman
4-1BB ligand trimer containing Fc (kih)
fusion molecule Construct 5.14

| SEQ | De- | |
|---|---|---|
| ID | scrip- | |
| NO: | tion | Sequence |
| 193 | nucleo- | CAGGTGCAGCTGGTGCAGTCTGGCGCCGAAGTGAAGAA |
| | tide | ACCCGGCAGCAGCGTGAAGGTGTCCTGCAAGGCCAGCG |
| | se- | GCTTCAACATCAAGGACACCTACATGCACTGGGTGCGCC |
| | quence | AGGCCCCTGGACAGGGACTGGAATGGATGGGCAGAATC |
| | of | GACCCCGCCAACGGCAACAGCAAATACGTGCCCAAGTT |
| | anti- | CCAGGGCAGAGTGACCATCACCGCCGACACCAGCACCT |
| | CEA | CCACCGCCTACATGGAACTGAGCAGCCTGCGGAGCGAG |
| | (T84. | GACACCGCCGTGTACTACTGTGCCCCCTTCGGCTACTAC |
| | 66- | GTGTCCGACTACGCCATGGCCTATTGGGGCCAGGGCAC |
| | LCHA) | ACTCGTGACCGTGTCCTCTGCTAGCACCAAGGGCCCCTC |
| | Fc hole | CGTGTTCCCCCTGGCCCCCAGCAGCAAGAGCACCAGCG |
| | mono- | GCGGCACAGCCGCTCTGGGCTGCCTGGTCAAGGACTACT |
| | meric | TCCCCGAGCCCGTGACCGTGTCCTGGAACAGCGGAGCC |
| | 4-1BBL | CTGACCTCCGGCGTGCACACCTTCCCCGCCGTGCTGCAG |
| | (71- | AGTTCTGGCCTGTATAGCCTGAGCAGCGTGGTCACCGTG |
| | 248) | CCTTCTAGCAGCCTGGGCACCCAGACCTACATCTGCAAC |
| | chain | GTGAACCACAAGCCCAGCAACACCAAGGTGGACAAGAA |
| | | GGTGGAGCCCAAGAGCTGCGACAAAACTCACACATGCC |
| | | CACCGTGCCCAGCACCTGAAGCTGCAGGGGGACCGTCA |
| | | GTCTTCCTCTTCCCCCCAAAACCCAAGGACACCCTCATG |
| | | ATCTCCCGGACCCCTGAGGTCACATGCGTGGTGGTGGAC |
| | | GTGAGCCACGAAGACCCTGAGGTCAAGTTCAACTGGTA |
| | | CGTGGACGGCGTGGAGGTGCATAATGCCAAGACAAAGC |
| | | CGCGGGAGGAGCAGTACAACAGCACGTACCGTGTGGTC |
| | | AGCGTCCTCACCGTCCTGCACCAGGACTGGCTGAATGGC |
| | | AAGGAGTACAAGTGCAAGGTCTCCAACAAAGCCCTCGG |
| | | CGCCCCCATCGAGAAAACCATCTCCAAAGCCAAAGGGC |
| | | AGCCCCGAGAACCACAGGTGTGCACCCTGCCCCCATCCC |
| | | GGGATGAGCTGACCAAGAACCAGGTCAGCCTCTCGTGC |
| | | GCAGTCAAAGGCTTCTATCCCAGCGACATCGCCGTGGA |
| | | GTGGGAGAGCAATGGGCAGCCGGAGAACAACTACAAGA |
| | | CCACGCCTCCCGTGCTGGACTCCGACGGCTCCTTCTTCC |
| | | TCGTGAGCAAGCTCACCGTGGACAAGAGCAGGTGGCAG |
| | | CAGGGGAACGTCTTCTCATGCTCCGTGATGCATGAGGCT |
| | | CTGCACAACCACTACACGCAGAAGAGCCTCTCCCTGTCT |
| | | CCGGGTGGAGGCGGCGGAAGCGGAGGAGGAGGATCCA |
| | | GAGAGGGCCCTGAGCTGAGCCCTGATGATCCTGCCGGA |
| | | CTGCTGGACCTGCGGCAGGGAATGTTTGCCCAGCTGGTG |
| | | GCCCAGAACGTGCTGCTGATCGATGGCCCCCTGTCCTGG |
| | | TACAGCGATCCTGGACTGGCTGGCGTGTCACTGACAGGC |
| | | GGCCTGAGCTACAAAGAGGACACCAAAGAACTGGTGGT |
| | | GGCCAAGGCCGGCGTGTACTACGTGTTCTTTCAGCTGGA |
| | | ACTGCGGAGAGTGGTGGCCGGCGAAGGATCTGGCTCTG |
| | | TGTCTCTGGCCCTGCATCTGCAGCCTCTGAGATCTGCTG |
| | | CTGGCGCCGCTGCTCTGGCACTGACAGTGGATCTGCCTC |
| | | CTGCCAGCAGCGAGGCCCGGAATAGCGCATTTGGGTTTC |
| | | AAGGCAGGCTGCTGCACCTGTCTGCCGGCCAGAGGCTG |
| | | GGAGTGCATCTGCACACAGAGGCCAGGGCTAGACACGC |
| | | CTGGCAGCTGACACAGGGCGCTACAGTGCTGGGCCTGTT |
| | | CAGAGTGACCCCCGAGATTCCTGCCGGGCTC |
| 176 | nucleo- | see Table 24 |
| | tide | |
| | se- | |
| | quence | |
| | of | |
| | Fc knob | |
| | dimeric | |
| | 4-1BB | |
| | (71- | |
| | 248) | |
| | ligand | |
| 154 | nucleo- | See Table 17 |
| | tide | |
| | se- | |
| | quence | |
| | of | |
| | anti- | |
| | CEA | |
| | (T84. | |
| | 66- | |
| | LCHA) | |
| | light | |
| | chain | |
| 194 | anti- | QVQLVQSGAEVKKPGSSVKVSCKASGFNIKDTYMHWVRQ |
| | CEA | APGQGLEWMGRIDPANGNSKYVPKFQGRVTITADTSTST |
| | (T84. | AYMELSSLRSEDTAVYYCAPFGYYVSDYAMAYWGQGTLV |
| | 66- | TVSSASTKGPSVFPLAPSSKSTSGGTAALGCLVKDYFPE |
| | LCHA) | PVTVSWNSGALTSGVHTFPAVLQSSGLYSLSSVVTVPSS |
| | Fc hole | SLGTQTYICNVNHKPSNTKVDKKVEPKSCDKTHTCPPCP |
| | mono- | APEAAGGPSVFLFPPKPKDTLMISRTPEVTCVVVDVSHE |
| | meric | DPEVKFNWYVDGVEVHNAKTKPREEQYNSTYRVVSVLTV |
| | 4-1BB | LHQDWLNGKEYKCKVSNKALGAPIEKTISKAKGQPREPQ |
| | ligand | VCTLPPSRDELTKNQVSLSCAVKGFYPSDIAVEWESNGQ |
| | (71- | PENNYKTTPPVLDSDGSFFLVSKLTVDKSRWQQGNVFSC |
| | 248) | SVMHEALHNHYTQKSLSLSPGGGGGSGGGGSREGPELSP |
| | chain | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGL |
| 178 | Fc knob | see Table 24 |
| | dimeric | |
| | 4-1BB | |
| | (71- | |
| | 248) | |
| | ligand | |
| 156 | anti- | See Table 17 |
| | CEA | |
| | (T84. | |
| | 66- | |
| | LCHA) | |
| | light | |
| | chain | |

1.10 Preparation of Monovalent Untargeted 4-1BB Ligand Trimer-Containing Fc(Kih) Fusion Antigen Binding Molecules, Wherein the 4-1BB Ligands are C-Terminally Fused (Controls J and K) (DP47 on the Hole Chain)

The molecule was prepared as described in Example 1.7 for the monovalent FAP targeted construct, with the only difference that the anti-FAP binder (VH-VL) was replaced by a germline control, termed DP47, not binding to the antigen.

Table 33 shows the cDNA and amino acid sequences of the monovalent untargeted 4-1BB ligand (71-248)trimer-containing Fc (kih) fusion antigen binding molecule (Control J).

TABLE 33
Sequences of monovalent DP47-untargeted human
4-1BB ligand trimer containing Fc (kih) fusion
molecule Control J

| SEQ | De- | |
|---|---|---|
| ID | scrip- | |
| NO: | tion | Sequence |
| 195 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of DP47 | GTGGTAGTGGTGGTAGCACATACTACGCAGACTCCGTGA |
| | Fc hole | AGGGCCGGTTCACCATCTCCAGAGACAATTCCAAGAAC |
| | dimeric | ACGCTGTATCTGCAGATGAACAGCCTGAGAGCCGAGGA |
| | 4-1BB | CACGGCCGTATATTACTGTGCGAAAGGCAGCGGATTTGA |
| | ligand | CTACTGGGGCCAAGGAACCCTGGTCACCGTCTCGAGTGC |
| | (71- | TAGCACCAAGGGCCCCTCCGTGTTCCCCCTGGCCCCCAG |
| | 248) | CAGCAAGAGCACCAGCGGCGGCACAGCCGCTCTGGGCT |
| | chain | GCCTGGTCAAGGACTACTTCCCCGAGCCCGTGACCGTGT |
| | | CCTGGAACAGCGGAGCCCTGACCTCCGGCGTGCACACC |
| | | TTCCCCGCCGTGCTGCAGAGTTCTGGCCTGTATAGCCTG |
| | | AGCAGCGTGGTCACCGTGCCTTCTAGCAGCCTGGGCACC |
| | | CAGACCTACATCTGCAACGTGAACCACAAGCCCAGCAA |
| | | CACCAAGGTGGACAAGAAGGTGGAGCCCAAGAGCTGCG |
| | | ACAAAACTCACACATGCCCACCGTGCCCAGCACCTGAA |
| | | GCTGCAGGGGGACCGTCAGTCTTCCTCTTCCCCCCAAAA |
| | | CCCAAGGACACCCTCATGATCTCCCGGACCCCTGAGGTC |
| | | ACATGCGTGGTGGTGGACGTGAGCCACGAAGACCCTGA |
| | | GGTCAAGTTCAACTGGTACGTGGACGGCGTGGAGGTGC |
| | | ATAATGCCAAGACAAAGCCGCGGGAGGAGCAGTACAAC |
| | | AGCACGTACCGTGTGGTCAGCGTCCTCACCGTCCTGCAC |
| | | CAGGACTGGCTGAATGGCAAGGAGTACAAGTGCAAGGT |
| | | CTCCAACAAAGCCCTCGGCGCCCCCATCGAGAAAACCA |
| | | TCTCCAAAGCCAAAGGGCAGCCCCGAGAACCACAGGTG |
| | | TGCACCCTGCCCCCATCCCGGGATGAGCTGACCAAGAA |
| | | CCAGGTCAGCCTCTCGTGCGCAGTCAAAGGCTTCTATCC |
| | | CAGCGACATCGCCGTGGAGTGGGAGAGCAATGGGCAGC |
| | | CGGAGAACAACTACAAGACCACGCCTCCCGTGCTGGAC |
| | | TCCGACGGCTCCTTCTTCCTCGTGAGCAAGCTCACCGTG |
| | | GACAAGAGCAGGTGGCAGCAGGGGAACGTCTTCTCATG |
| | | CTCCGTGATGCATGAGGCTCTGCACAACCACTACACGCA |
| | | GAAGAGCCTCTCCCTGTCTCCGGGTGGAGGCGGCGGAA |
| | | GCGGAGGAGGAGGATCCAGAGAGGGCCCTGAGCTGAGC |
| | | CCTGATGATCCTGCCGGACTGCTGGACCTGCGGCAGGG |
| | | AATGTTTGCCCAGCTGGTGGCCCAGAACGTGCTGCTGAT |
| | | CGATGGCCCCCTGTCCTGGTACAGCGATCCTGGACTGGC |
| | | TGGCGTGTCACTGACAGGCGGCCTGAGCTACAAAGAGG |
| | | ACACCAAAGAACTGGTGGTGGCCAAGGCCGGCGTGTAC |
| | | TACGTGTTCTTTCAGCTGGAACTGCGGAGAGTGGTGGCC |
| | | GGCGAAGGATCTGGCTCTGTGTCTCTGGCCCTGCATCTG |
| | | CAGCCTCTGAGATCTGCTGCTGGCGCCGCTGCTCTGGCA |
| | | CTGACAGTGGATCTGCCTCCTGCCAGCAGCGAGGCCCG |
| | | GAATAGCGCATTTGGGTTTCAAGGCAGGCTGCTGCACCT |
| | | GTCTGCCGGCCAGAGGCTGGGAGTGCATCTGCACACAG |
| | | AGGCCAGGGCTAGACACGCCTGGCAGCTGACACAGGGC |
| | | GCTACAGTGCTGGGCCTGTTCAGAGTGACCCCCGAGATT |
| | | CCAGCAGGCCTGGGAGGCGGCGGATCTGGCGGCGGAGG |
| | | ATCTAGAGAAGGACCCGAGCTGTCCCCCGACGATCCCG |
| | | CTGGGCTGCTGGATCTGAGACAGGGCATGTTCGCTCAGC |
| | | TGGTGGCTCAGAATGTGCTGCTGATTGACGGACCTCTGA |
| | | GCTGGTACTCCGACCCAGGGCTGGCAGGGGTGTCCCTG |
| | | ACTGGGGGACTGTCCTACAAAGAAGATACAAAAGAACT |
| | | GGTGGTGGCTAAAGCTGGGGTGTACTATGTGTTTTTTCA |
| | | GCTGGAACTGAGGCGGGTGGTGGCTGGGGAGGGCTCAG |
| | | GATCTGTGTCCCTGGCTCTGCATCTGCAGCCACTGCGCT |
| | | CTGCAGCAGGGGCTGCAGCACTGGCCCTGACTGTGGAC |
| | | CTGCCCCCAGCTTCTTCCGAGGCCAGAAACAGCGCCTTC |
| | | GGGTTCCAAGGACGCCTGCTGCATCTGAGCGCCGGACA |
| | | GCGCCTGGGAGTGCATCTGCATACTGAAGCCAGAGCCC |
| | | GGCATGCTTGGCAGCTGACTCAGGGGGCAACTGTGCTG |
| | | GGACTGTTTCGCGTGACACCTGAGATCCCAGCCGGGCTC |
| 172 | nucleo- | see Table 23 |
| | tide | |
| | se- | |
| | quence | |
| | of Fc | |
| | knob | |
| | mono- | |
| | meric | |
| | 4-1BBL | |
| | (71- | |
| | 248) | |
| 166 | nucleo- | see Table 21 |
| | tide | |
| | se- | |
| | quence | |
| | of DP47 | |
| | light | |
| | chain | |
| 196 | DP47 Fc | EVQLLESGGGLVQPGGSLRLSCAASGFTFSSYAMSWVRQ |
| | hole | APGKGLEWVSAISGSGGSTYYADSVKGRFTISRDNSKNT |
| | dimeric | LYLQMNSLRAEDTAVYYCAKGSGFDYWGQGTLVTVSSAS |
| | 4-1BB | TKGPSVFPLAPSSKSTSGGTAALGCLVKDYFPEPVTVSW |
| | ligand | NSGALTSGVHTFPAVLQSSGLYSLSSVVTVPSSSLGTQT |
| | (71- | YICNVNHKPSNTKVDKKVEPKSCDKTHTCPPCPAPEAAG |
| | 248) | GPSVFLFPPKPKDTLMISRTPEVTCVVVDVSHEDPEVKF |
| | chain | NWYVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNYK |
| | | TTPPVLDSDGSFFLVSKLTVDKSRWQQGNVFSCSVMHEA |
| | | LHNHYTQKSLSLSPGGGGGSGGGGSREGPELSPDDPAGL |
| | | LDLRQGMFAQLVAQNVLLIDGPLSWYSDPGLAGVSLTGG |
| | | LSYKEDTKELVVAKAGVYYVFFQLELRRVVAGEGSGSVS |
| | | LALHLQPLRSAAGAAALALTVDLPPASSEARNSAFGFQG |
| | | RLLHLSAGQRLGVHLHTEARARHAWQLTQGATVLGLFRV |
| | | TPEIPAGLGGGGSGGGGSREGPELSPDDPAGLLDLRQGM |
| | | FAQLVAQNVLLIDGPLSWYSDPGLAGVSLTGGLSYKEDT |
| | | KELVVAKAGVYYVFFQLELRRVVAGEGSGSVSLALHLQP |
| | | LRSAAGAAALALTVDLPPASSEARNSAFGFQGRLLHLSA |
| | | GQRLGVHLHTEARARHAWQLTQGATVLGLFRVTPEIPAG |
| | | L |
| 174 | Fc knob | see Table 23 |
| | mono- | |
| | meric | |
| | 4-1-BB | |
| | (71- | |
| | 248) | |
| | ligand | |
| 168 | DP47 | see Table 21 |
| | light | |
| | chain | |

Table 34 shows the cDNA and amino acid sequences of the monovalent untargeted 4-1BB ligand (71-248) timer-containing Fc (kih) fusion antigen binding molecule (Control K).

TABLE 34
Sequences of monovalent DP47-untargeted human
4-1BB ligand trimer containing Fc (kih) fusion
molecule Control K

| SEQ | De- | |
|---|---|---|
| ID | scrip- | |
| NO: | tion | Sequence |
| 197 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of DP47 | GTGGTAGTGGTGGTAGCACATACTACGCAGACTCCGTGA |
| | Fc hole | AGGGCCGGTTCACCATCTCCAGAGACAATTCCAAGAAC |
| | mono- | ACGCTGTATCTGCAGATGAACAGCCTGAGAGCCGAGGA |
| | meric | CACGGCCGTATATTACTGTGCGAAAGGCAGCGGATTTGA |
| | 4-1BBL | CTACTGGGGCCAAGGAACCCTGGTCACCGTCTCGAGTGC |
| | (71- | TAGCACCAAGGGCCCCTCCGTGTTCCCCCTGGCCCCCAG |
| | 248) | CAGCAAGAGCACCAGCGGCGGCACAGCCGCTCTGGGCT |
| | chain | GCCTGGTCAAGGACTACTTCCCCGAGCCCGTGACCGTGT |
| | | CCTGGAACAGCGGAGCCCTGACCTCCGGCGTGCACACC |
| | | TTCCCCGCCGTGCTGCAGAGTTCTGGCCTGTATAGCCTG |
| | | AGCAGCGTGGTCACCGTGCCTTCTAGCAGCCTGGGCACC |
| | | CAGACCTACATCTGCAACGTGAACCACAAGCCCAGCAA |
| | | CACCAAGGTGGACAAGAAGGTGGAGCCCAAGAGCTGCG |
| | | ACAAAACTCACACATGCCCACCGTGCCCAGCACCTGAA |
| | | GCTGCAGGGGGACCGTCAGTCTTCCTCTTCCCCCCAAAA |
| | | CCCAAGGACACCCTCATGATCTCCCGGACCCCTGAGGTC |
| | | ACATGCGTGGTGGTGGACGTGAGCCACGAAGACCCTGA |
| | | GGTCAAGTTCAACTGGTACGTGGACGGCGTGGAGGTGC |
| | | ATAATGCCAAGACAAAGCCGCGGGAGGAGCAGTACAAC |
| | | AGCACGTACCGTGTGGTCAGCGTCCTCACCGTCCTGCAC |
| | | CAGGACTGGCTGAATGGCAAGGAGTACAAGTGCAAGGT |
| | | CTCCAACAAAGCCCTCGGCGCCCCCATCGAGAAAACCA |
| | | TCTCCAAAGCCAAAGGGCAGCCCCGAGAACCACAGGTG |
| | | TGCACCCTGCCCCCATCCCGGGATGAGCTGACCAAGAA |
| | | CCAGGTCAGCCTCTCGTGCGCAGTCAAAGGCTTCTATCC |
| | | CAGCGACATCGCCGTGGAGTGGGAGAGCAATGGGCAGC |
| | | CGGAGAACAACTACAAGACCACGCCTCCCGTGCTGGAC |
| | | TCCGACGGCTCCTTCTTCCTCGTGAGCAAGCTCACCGTG |
| | | GACAAGAGCAGGTGGCAGCAGGGGAACGTCTTCTCATG |
| | | CTCCGTGATGCATGAGGCTCTGCACAACCACTACACGCA |
| | | GAAGAGCCTCTCCCTGTCTCCGGGTGGAGGCGGCGGAA |
| | | GCGGAGGAGGAGGATCCAGAGAGGGCCCTGAGCTGAGC |
| | | CCTGATGATCCTGCCGGACTGCTGGACCTGCGGCAGGG |
| | | AATGTTTGCCCAGCTGGTGGCCCAGAACGTGCTGCTGAT |
| | | CGATGGCCCCCTGTCCTGGTACAGCGATCCTGGACTGGC |
| | | TGGCGTGTCACTGACAGGCGGCCTGAGCTACAAAGAGG |
| | | ACACCAAAGAACTGGTGGTGGCCAAGGCCGGCGTGTAC |
| | | TACGTGTTCTTTCAGCTGGAACTGCGGAGAGTGGTGGCC |
| | | GGCGAAGGATCTGGCTCTGTGTCTCTGGCCCTGCATCTG |
| | | CAGCCTCTGAGATCTGCTGCTGGCGCCGCTGCTCTGGCA |
| | | CTGACAGTGGATCTGCCTCCTGCCAGCAGCGAGGCCCG |
| | | GAATAGCGCATTTGGGTTTCAAGGCAGGCTGCTGCACCT |
| | | GTCTGCCGGCCAGAGGCTGGGAGTGCATCTGCACACAG |
| | | AGGCCAGGGCTAGACACGCCTGGCAGCTGACACAGGGC |
| | | GCTACAGTGCTGGGCCTGTTCAGAGTGACCCCCGAGATT |
| | | CCTGCCGGGCTC |
| 176 | nucleo- | see Table 24 |
| | tide | |
| | se- | |
| | quence | |
| | of | |
| | Fc knob | |
| | dimeric | |
| | 4-1BBL | |
| | (71- | |
| | 248) | |
| 166 | nucleo- | See Table 21 |
| | tide | |
| | se- | |
| | quence | |
| | of DP47 | |
| | light | |
| | chain | |
| 198 | DP47 | EVQLLESGGGLVQPGGSLRLSCAASGFTFSSYAMSWVRQ |
| | Fc hole | APGKGLEWVSAISGSGGSTYYADSVKGRFTISRDNSKNT |
| | mono- | LYLQMNSLRAEDTAVYYCAKGSGFDYWGQGTLVTVSSAS |
| | meric | TKGPSVFPLAPSSKSTSGGTAALGCLVKDYFPEPVTVSW |
| | 4-1BBL | NSGALTSGVHTFPAVLQSSGLYSLSSVVTVPSSSLGTQT |
| | (71- | YICNVNHKPSNTKVDKKVEPKSCDKTHTCPPCPAPEAAG |
| | 248) | GPSVFLFPPKPKDTLMISRTPEVTCVVVDVSHEDPEVKF |
| | chain | NWYVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNYK |
| | | TTPPVLDSDGSFFLVSKLTVDKSRWQQGNVFSCSVMHEA |
| | | LHNHYTQKSLSLSPGGGGGSGGGGSREGPELSPDDPAGL |
| | | LDLRQGMFAQLVAQNVLLIDGPLSWYSDPGLAGVSLTGG |
| | | LSYKEDTKELVVAKAGVYYVFFQLELRRVVAGEGSGSVS |
| | | LALHLQPLRSAAGAAALALTVDLPPASSEARNSAFGFQG |
| | | RLLHLSAGQRLGVHLHTEARARHAWQLTQGATVLGLFRV |
| | | TPEIPAGL |
| 178 | Fc knob | see Table 24 |
| | dimeric | |
| | 4-1BBL | |
| | (71- | |
| | 248) | |
| 168 | DP47 | See Table 21 |
| | light | |
| | chain | |

1.11 Preparation of Additional Control Molecules

Control D, a molecule with N-terminal fused 4-1BBL moieties as depicted In FIG. 3B was prepared in combination with the germline control, termed DP47, not binding to the antigen.

The molecule comprises the Pro329Gly, Leu234Ala and Leu235Ala mutations in the constant region of the knob and hole heavy chains to abrogate binding to Fcgamma receptors according to the method described in International Patent Appl. Publ. No. WO 2012/130831 A1.

The polypeptide encoding the dimeric 4-1BB ligand fused to human CL domain was subcloned in frame with the human IgG1 heavy chain CH2 and CH3 domains on the knob and the polypeptide encoding the monomeric 4-1BB ligand was fused to human CH1 domain. To improve correct pairing the following mutations have been introduced in the crossed CH-CL. In the dimeric 4-1BB ligand fused to human CL, E123R and Q124K. In the monomeric 4-1BB ligand fused to human CH1, K147E and K213E.

Table 35 shows the cDNA and amino acid sequences of the monovalent “untargeted” 4-1BB ligand (71-248) trimer-containing Fc (kih) fusion antigen binding molecule (Control D).

TABLE 35
Sequences of monovalent DP47-untargeted human
4-1BB ligand trimer containing Fc (kih) fusion
molecule with CH-CL cross and with charged
residues* (Control D)

| SEQ | De- | |
|---|---|---|
| ID | scrip- | |
| NO: | tion | Sequence |
| 199 | nucleo- | AGAGAGGGCCCTGAGCTGAGCCCCGATGATCCTGCTGG |
| | tide | ACTGCTGGACCTGCGGCAGGGCATGTTTGCTCAGCTGGT |
| | se- | GGCCCAGAACGTGCTGCTGATCGATGGCCCCCTGTCCTG |
| | quence | GTACAGCGATCCTGGACTGGCTGGCGTGTCACTGACAGG |
| | of | CGGCCTGAGCTACAAAGAGGACACCAAAGAACTGGTGG |
| | dimeric | TGGCCAAGGCCGGCGTGTACTACGTGTTCTTTCAGCTGG |
| | 4-1BB | AACTGCGGAGAGTGGTGGCCGGCGAAGGATCTGGCTCT |
| | ligand | GTGTCTCTGGCCCTGCATCTGCAGCCTCTGAGATCTGCT |
| | (71- | GCTGGCGCCGCTGCTCTGGCACTGACAGTGGATCTGCCT |
| | 248) | CCTGCCAGCAGCGAGGCCCGGAATAGCGCATTTGGGTTT |
| | CL* Fc | CAAGGCAGGCTGCTGCACCTGTCTGCCGGCCAGAGGCT |
| | knob | GGGAGTGCATCTGCACACAGAGGCCAGGGCTAGACACG |
| | chain | CCTGGCAGCTGACACAGGGCGCTACAGTGCTGGGCCTG |
| | | TTCAGAGTGACCCCCGAGATTCCAGCCGGACTGGGAGG |
| | | CGGCGGATCTGGCGGCGGAGGATCTAGAGAAGGACCCG |
| | | AGCTGTCCCCTGACGATCCAGCCGGGCTGCTGGATCTGA |
| | | GACAGGGAATGTTCGCCCAGCTGGTGGCTCAGAATGTG |
| | | CTGCTGATTGACGGACCTCTGAGCTGGTACTCCGACCCA |
| | | GGGCTGGCAGGGGTGTCCCTGACTGGGGGACTGTCCTAC |
| | | AAAGAAGATACAAAAGAACTGGTGGTGGCTAAAGCTGG |
| | | GGTGTACTATGTGTTTTTTCAGCTGGAACTGAGGCGGGT |
| | | GGTGGCTGGGGAGGGCTCAGGATCTGTGTCCCTGGCTCT |
| | | GCATCTGCAGCCACTGCGCTCTGCAGCAGGGGCTGCAG |
| | | CACTGGCCCTGACTGTGGACCTGCCCCCAGCTTCTTCCG |
| | | AGGCCAGAAACAGCGCCTTCGGGTTCCAAGGACGCCTG |
| | | CTGCATCTGAGCGCCGGACAGCGCCTGGGAGTGCATCT |
| | | GCATACTGAAGCCAGAGCCCGGCATGCTTGGCAGCTGA |
| | | CTCAGGGGGCAACTGTGCTGGGACTGTTTCGCGTGACAC |
| | | CTGAGATCCCCGCTGGACTGGGCGGAGGCGGTTCCGGA |
| | | GGGGGAGGATCTCGTACGGTGGCTGCACCATCTGTCTTT |
| | | ATCTTCCCACCCAGCGACCGGAAGCTGAAGTCTGGCAC |
| | | AGCCAGCGTCGTGTGCCTGCTGAATAACTTCTACCCCCG |
| | | CGAGGCCAAGGTGCAGTGGAAGGTGGACAATGCCCTGC |
| | | AGAGCGGCAACAGCCAGGAAAGCGTGACCGAGCAGGA |
| | | CAGCAAGGACTCCACCTACAGCCTGAGCAGCACCCTGA |
| | | CCCTGAGCAAGGCCGACTACGAGAAGCACAAGGTGTAC |
| | | GCCTGCGAAGTGACCCACCAGGGCCTGTCTAGCCCCGTG |
| | | ACCAAGAGCTTCAACCGGGGCGAGTGCGACAAGACCCA |
| | | CACCTGTCCTCCATGCCCTGCCCCTGAAGCTGCTGGCGG |
| | | CCCTAGCGTGTTCCTGTTCCCCCCAAAGCCCAAGGACAC |
| | | CCTGATGATCAGCCGGACCCCTGAAGTGACCTGCGTGGT |
| | | GGTGGATGTGTCCCACGAGGACCCTGAAGTGAAGTTCA |
| | | ATTGGTACGTGGACGGCGTGGAAGTGCACAATGCCAAG |
| | | ACCAAGCCGCGGGAGGAGCAGTACAACAGCACGTACCG |
| | | TGTGGTCAGCGTCCTCACCGTCCTGCACCAGGACTGGCT |
| | | GAATGGCAAGGAGTACAAGTGCAAGGTCTCCAACAAAG |
| | | CCCTCGGCGCCCCCATCGAGAAAACCATCTCCAAAGCC |
| | | AAAGGGCAGCCCCGAGAACCACAGGTGTACACCCTGCC |
| | | CCCATGCCGGGATGAGCTGACCAAGAACCAGGTCAGCC |
| | | TGTGGTGCCTGGTCAAAGGCTTCTATCCCAGCGACATCG |
| | | CCGTGGAGTGGGAGAGCAATGGGCAGCCGGAGAACAAC |
| | | TACAAGACCACGCCTCCCGTGCTGGACTCCGACGGCTCC |
| | | TTCTTCCTCTACAGCAAGCTCACCGTGGACAAGAGCAGG |
| | | TGGCAGCAGGGGAACGTCTTCTCATGCTCCGTGATGCAT |
| | | GAGGCTCTGCACAACCACTACACGCAGAAGAGCCTCTC |
| | | CCTGTCTCCGGGTAAA |
| 200 | nucleo- | AGAGAGGGCCCTGAGCTGAGCCCCGATGATCCTGCTGG |
| | tide | ACTGCTGGACCTGCGGCAGGGCATGTTTGCTCAGCTGGT |
| | se- | GGCCCAGAACGTGCTGCTGATCGATGGCCCCCTGTCCTG |
| | quence | GTACAGCGATCCTGGACTGGCTGGCGTGTCACTGACAGG |
| | of | CGGCCTGAGCTACAAAGAGGACACCAAAGAACTGGTGG |
| | mono- | TGGCCAAGGCCGGCGTGTACTACGTGTTCTTTCAGCTGG |
| | meric | AACTGCGGAGAGTGGTGGCCGGCGAAGGATCTGGCTCT |
| | 4-1BBL | GTGTCTCTGGCCCTGCATCTGCAGCCTCTGAGATCTGCT |
| | (71- | GCTGGCGCCGCTGCTCTGGCACTGACAGTGGATCTGCCT |
| | 248)- | CCTGCCAGCAGCGAGGCCCGGAATAGCGCATTTGGGTTT |
| | CH1* | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGACTGGGAGG |
| | | CGGAGGTTCCGGAGGCGGAGGATCTGCTAGCACAAAGG |
| | | GCCCCAGCGTGTTCCCTCTGGCCCCTAGCAGCAAGAGCA |
| | | CATCTGGCGGAACAGCCGCCCTGGGCTGCCTGGTGGAA |
| | | GATTACTTCCCCGAGCCCGTGACCGTGTCCTGGAATTCT |
| | | GGCGCCCTGACAAGCGGCGTGCACACCTTTCCAGCCGTG |
| | | CTGCAGAGCAGCGGCCTGTACTCTCTGAGCAGCGTCGTG |
| | | ACAGTGCCCAGCAGCTCTCTGGGCACCCAGACCTACATC |
| | | TGCAACGTGAACCACAAGCCCAGCAACACCAAGGTGGA |
| | | CGAGAAGGTGGAACCCAAGTCCTGC |
| 201 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of | GTGGTAGTGGTGGTAGCACATACTACGCAGACTCCGTGA |
| | DP47 | AGGGCCGGTTCACCATCTCCAGAGACAATTCCAAGAAC |
| | Fc hole | ACGCTGTATCTGCAGATGAACAGCCTGAGAGCCGAGGA |
| | chain | CACGGCCGTATATTACTGTGCGAAAGGCAGCGGATTTGA |
| | | CTACTGGGGCCAAGGAACCCTGGTCACCGTCTCGAGTGC |
| | | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 166 | nucleo- | see Table 21 |
| | tide | |
| | se- | |
| | quence | |
| | of DP47 | |
| | light | |
| | chain | |
| 202 | dimeric | REGPELSPDDPAGLLDLRQGMFAQLVAQNVLLIDGPLSW |
| | 4-1BB | YSDPGLAGVSLTGGLSYKEDTKELVVAKAGVYYVFFQLE |
| | ligand | LRRVVAGEGSGSVSLALHLQPLRSAAGAAALALTVDLPP |
| | (71- | ASSEARNSAFGFQGRLLHLSAGQRLGVHLHTEARARHAW |
| | 248) | QLTQGATVLGLFRVTPEIPAGLGGGGSGGGGSREGPELS |
| | CL* Fc | PDDPAGLLDLRQGMFAQLVAQNVLLIDGPLSWYSDPGLA |
| | knob | GVSLTGGLSYKEDTKELVVAKAGVYYVFFQLELRRVVAG |
| | chain | EGSGSVSLALHLQPLRSAAGAAALALTVDLPPASSEARN |
| | | SAFGFQGRLLHLSAGQRLGVHLHTEARARHAWQLTQGAT |
| | | VLGLFRVTPEIPAGLGGGGSGGGGSRTVAAPSVFIFPPS |
| | | DRKLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGECDKTHTCPPCPAPEAAGGPSVFLFP |
| | | PKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALGAPIEKTISKAKGQPREPQVYTLPPCRDELTKN |
| | | QVSLWCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 203 | mono- | REGPELSPDDPAGLLDLRQGMFAQLVAQNVLLIDGPLSW |
| | meric | YSDPGLAGVSLTGGLSYKEDTKELVVAKAGVYYVFFQLE |
| | 4-1BBL | LRRVVAGEGSGSVSLALHLQPLRSAAGAAALALTVDLPP |
| | (71- | ASSEARNSAFGFQGRLLHLSAGQRLGVHLHTEARARHAW |
| | 248) | QLTQGATVLGLFRVTPEIPAGLGGGGSGGGGSASTKGPS |
| | CH1* | VFPLAPSSKSTSGGTAALGCLVEDYFPEPVTVSWNSGAL |
| | | TSGVHTFPAVLQSSGLYSLSSVVTVPSSSLGTQTYICNV |
| | | NHKPSNTKVDEKVEPKSC |
| 204 | DP47 | EVQLLESGGGLVQPGGSLRLSCAASGFTFSSYAMSWVRQ |
| | Fc hole | APGKGLEWVSAISGSGGSTYYADSVKGRFTISRDNSKNT |
| | chain | LYLQMNSLRAEDTAVYYCAKGSGFDYWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNYK |
| | | TTPPVLDSDGSFFLVSKLTVDKSRWQQGNVFSCSVMHEA |
| | | LHNHYTQKSLSLSPGK |
| 168 | DP47 | see Table 21 |
| | light | |
| | chain | |

A FAP-targeted variant of Control D was prepared, wherein instead of DP47 the variable region of heavy and light chain DNA sequences encoding a binder specific for fibroblast activation protein (FAP), clone 4B9, were subcloned in frame with either the constant heavy chain of the hole or the constant light chain of human IgG1. Table 36 shows the cDNA and amino acid sequences of the monovalent FAP-targeted 4-1BB ligand (71-248) trimer-containing Fc (kih) fusion antigen binding molecule (Construct 2.4).

TABLE 36
Sequences of monovalent FAP(4B9)-targeted human
4-1BB ligand (71-248) containing Fc (kih) fusion
molecule Construct 2.4

| SEQ | De- | |
|---|---|---|
| ID | scrip- | |
| NO: | tion | Sequence |
| 199 | nucleo- | see Table 35 |
| | tide | |
| | se- | |
| | quence | |
| | of | |
| | dimeric | |
| | hu 4- | |
| | 1BBL | |
| | (71- | |
| | 248) | |
| | CL* | |
| | Fc knob | |
| | chain | |
| 200 | nucleo- | see Table 35 |
| | tide | |
| | se- | |
| | quence | |
| | of | |
| | mono- | |
| | meric | |
| | hu 4- | |
| | 1BBL | |
| | (71- | |
| | 248) | |
| | CH1* | |
| 235 | nucleo- | GAGGTGCAGCTGCTCGAAAGCGGCGGAGGACTGGTGCA |
| | tide | GCCTGGCGGCAGCCTGAGACTGTCTTGCGCCGCCAGCG |
| | se- | GCTTCACCTTCAGCAGCTACGCCATGAGCTGGGTCCGCC |
| | quence | AGGCCCCTGGCAAGGGACTGGAATGGGTGTCCGCCATC |
| | of | ATCGGCTCTGGCGCCAGCACCTACTACGCCGACAGCGTG |
| | anti- | AAGGGCCGGTTCACCATCAGCCGGGACAACAGCAAGAA |
| | FAP | CACCCTGTACCTGCAGATGAACAGCCTGCGGGCCGAGG |
| | (4B9) | ACACCGCCGTGTACTACTGCGCCAAGGGATGGTTCGGC |
| | Fc hole | GGCTTCAACTACTGGGGACAGGGCACCCTGGTCACAGT |
| | chain | GTCCAGCGCTAGCACCAAGGGCCCCTCCGTGTTCCCCCT |
| | | GGCCCCCAGCAGCAAGAGCACCAGCGGCGGCACAGCCG |
| | | CTCTGGGCTGCCTGGTCAAGGACTACTTCCCCGAGCCCG |
| | | TGACCGTGTCCTGGAACAGCGGAGCCCTGACCTCCGGC |
| | | GTGCACACCTTCCCCGCCGTGCTGCAGAGTTCTGGCCTG |
| | | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAAACTCACACATGCCCACCGTGCCCA |
| | | GCACCTGAAGCTGCAGGGGGACCGTCAGTCTTCCTCTTC |
| | | CCCCCAAAACCCAAGGACACCCTCATGATCTCCCGGACC |
| | | CCTGAGGTCACATGCGTGGTGGTGGACGTGAGCCACGA |
| | | AGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTGCACCCTGCCCCCATCCCGGGATGAGCTG |
| | | ACCAAGAACCAGGTCAGCCTCTCGTGCGCAGTCAAAGG |
| | | CTTCTATCCCAGCGACATCGCCGTGGAGTGGGAGAGCA |
| | | ATGGGCAGCCGGAGAACAACTACAAGACCACGCCTCCC |
| | | GTGCTGGACTCCGACGGCTCCTTCTTCCTCGTGAGCAAG |
| | | CTCACCGTGGACAAGAGCAGGTGGCAGCAGGGGAACGT |
| | | CTTCTCATGCTCCGTGATGCATGAGGCTCTGCACAACCA |
| | | CTACACGCAGAAGAGCCTCTCCCTGTCTCCGGGTAAA |
| 103 | nucleo- | see Table 1 |
| | tide | |
| | se- | |
| | quence | |
| | of | |
| | anti- | |
| | FAP | |
| | (4B9) | |
| | light | |
| | chain | |
| 202 | Dimeric | see Table 35 |
| | hu 4- | |
| | 1BBL | |
| | (71- | |
| | 248) | |
| | CL* Fc | |
| | knob | |
| | chain | |
| 203 | Mono- | see Table 35 |
| | meric | |
| | hu 4- | |
| | 1BBL | |
| | (71- | |
| | 248) | |
| | CH1* | |
| 236 | anti- | EVQLLESGGGLVQPGGSLRLSCAASGFTFSSYAMSWVRQ |
| | FAP | APGKGLEWVSAIIGSGASTYYADSVKGRFTISRDNSKNT |
| | (4B9) | LYLQMNSLRAEDTAVYYCAKGWFGGFNYWGQGTLVTVSS |
| | Fc hole | ASTKGPSVFPLAPSSKSTSGGTAALGCLVKDYFPEPVTV |
| | chain | SWNSGALTSGVHTFPAVLQSSGLYSLSSVVTVPSSSLGT |
| | | QTYICNVNHKPSNTKVDKKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTL |
| | | PPSRDELTKNQVSLSCAVKGFYPSDIAVEWESNGQPENN |
| | | YKTTPPVLDSDGSFFLVSKLTVDKSRWQQGNVFSCSVMH |
| | | EALHNHYTQKSLSLSPGK |
| 106 | anti- | see Table 1 |
| | FAP | |
| | (4B9) | |
| | light | |
| | chain | |

CD19-targeted variants were also prepared, wherein instead of DP47 the variable region of heavy and light chain DNA sequences encoding a binder specific for CD19, clones 8B8-018 and 8B8-2B11, were subcloned in frame with either the constant heavy chain of the hole or the constant light chain of human IgG1. The DNA sequence encoding part of the ectodomain (amino acids 71-254 or amino acids 71-248) of human 4-1BB ligand was synthesized according to the P41273 sequence of Uniprot database. Table 37a shows the cDNA and amino acid sequences of the monovalent CD19(8B8-018) targeted split trimeric 4-1BB ligand (71-248) Fe(kih) fusion antigen binding molecule with crossed CH-CL and charged residues(construct 3.4). Table 37b shows the cDNA and amino acid sequences of the monovalent CD19-targeted 4-1 BB ligand (71-254)trimer-containing Fe(kih) fusion antigen binding molecule (Construct 4.1).

TABLE 37a
cDNA and amino acid sequences of monovalent
CD19(8B8-018) targeted split trimeric 4-1BB
ligand (71-248) Fc (kih) fusion containing
CH-CL cross with charged residues (construct
3.4). * charged residues

| SEQ | De- | |
|---|---|---|
| ID | scrip- | |
| NO: | tion | Sequence |
| 199 | nucleo- | see Table 35 |
| | tide | |
| | se- | |
| | quence | |
| | of | |
| | dimeric | |
| | hu 4- | |
| | 1BBL | |
| | (71- | |
| | 248) | |
| | CL* | |
| | Fc knob | |
| | chain | |
| 200 | nucleo- | see Table 35 |
| | tide | |
| | se- | |
| | quence | |
| | of | |
| | mono- | |
| | meric | |
| | hu 4- | |
| | 1BBL | |
| | (71- | |
| | 248) | |
| | CH1* | |
| 237 | Nucleo- | CAGGTCCAGCTGGTGCAGTCCGGCGCCGAGGTCAAGAA |
| | tide | ACCCGGGGCTTCTGTGAAGGTTTCATGCAAGGCAAGCG |
| | se- | GATACACCTTCACCGACTATATCATGCATTGGGTCAGGC |
| | quence | AGGCCCCTGGCCAAGGTCTCGAATGGATGGGCTACATTA |
| | anti- | ACCCATATAATGATGGCTCCAAATACACCGAGAAGTTTC |
| | CD19 | AGGGAAGAGTCACTATGACATCTGACACCAGTATCAGC |
| | (8B8- | ACTGCTTACATGGAGCTGTCCCGCCTTCGGTCTGATGAC |
| | 018) | ACCGCAGTGTATTACTGTGCCAGGGGCACATATTACTAC |
| | Fc hole | GGCTCAGCTCTGTTCGACTATTGGGGGCAGGGAACCACA |
| | chain | GTAACCGTGAGCTCCGCTAGCACCAAGGGCCCCTCCGTG |
| | | TTCCCCCTGGCCCCCAGCAGCAAGAGCACCAGCGGCGG |
| | | CACAGCCGCTCTGGGCTGCCTGGTCAAGGACTACTTCCC |
| | | CGAGCCCGTGACCGTGTCCTGGAACAGCGGAGCCCTGA |
| | | CCTCCGGCGTGCACACCTTCCCCGCCGTGCTGCAGAGTT |
| | | CTGGCCTGTATAGCCTGAGCAGCGTGGTCACCGTGCCTT |
| | | CTAGCAGCCTGGGCACCCAGACCTACATCTGCAACGTG |
| | | AACCACAAGCCCAGCAACACCAAGGTGGACAAGAAGGT |
| | | GGAGCCCAAGAGCTGCGACAAAACTCACACATGCCCAC |
| | | CGTGCCCAGCACCTGAAGCTGCAGGGGGACCGTCAGTC |
| | | TTCCTCTTCCCCCCAAAACCCAAGGACACCCTCATGATC |
| | | TCCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTGCACCCTGCCCCCATCCCGG |
| | | GATGAGCTGACCAAGAACCAGGTCAGCCTCTCGTGCGC |
| | | AGTCAAAGGCTTCTATCCCAGCGACATCGCCGTGGAGTG |
| | | GGAGAGCAATGGGCAGCCGGAGAACAACTACAAGACC |
| | | ACGCCTCCCGTGCTGGACTCCGACGGCTCCTTCTTCCTC |
| | | GTGAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTAAA |
| 118 | Nucleo- | see Table 5 |
| | tide | |
| | se- | |
| | quence | |
| | anti- | |
| | CD19 | |
| | (8B8- | |
| | 018) | |
| | light | |
| | chain | |
| 202 | Dimeric | see Table 35 |
| | hu 4- | |
| | 1BBL | |
| | (71- | |
| | 248) | |
| | CL* Fc | |
| | knob | |
| | chain | |
| 203 | Mono- | see Table 35 |
| | meric | |
| | hu 4- | |
| | 1BBL | |
| | (71- | |
| | 248) | |
| | CH1* | |
| 238 | anti- | QVQLVQSGAEVKKPGASVKVSCKASGYTFTDYIMHWVRQ |
| | CD19 | APGQGLEWMGYINPYNDGSKYTEKFQGRVTMTSDTSIST |
| | (8B8- | AYMELSRLRSDDTAVYYCARGTYYYGSALFDYWGQGTTV |
| | 018) | TVSSASTKGPSVFPLAPSSKSTSGGTAALGCLVKDYFPE |
| | Fc hole | PVTVSWNSGALTSGVHTFPAVLQSSGLYSLSSVVTVPSS |
| | chain | SLGTQTYICNVNHKPSNTKVDKKVEPKSCDKTHTCPPCP |
| | | APEAAGGPSVFLFPPKPKDTLMISRTPEVTCVVVDVSHE |
| | | DPEVKFNWYVDGVEVHNAKTKPREEQYNSTYRVVSVLTV |
| | | LHQDWLNGKEYKCKVSNKALGAPIEKTISKAKGQPREPQ |
| | | VCTLPPSRDELTKNQVSLSCAVKGFYPSDIAVEWESNGQ |
| | | PENNYKTTPPVLDSDGSFFLVSKLTVDKSRWQQGNVFSC |
| | | SVMHEALHNHYTQKSLSLSPGK |
| 120 | anti- | see Table 5 |
| | CD19 | |
| | (8B8- | |
| | 018) | |
| | light | |
| | chain | |

TABLE 37b
Sequences of monovalent CD19(8B8-2B11)-targeted
human 4-1BB ligand (71-254) containing Fc (kih)
fusion molecule Construct 4.1

| SEQ | De- | |
|---|---|---|
| ID | scrip- | |
| NO: | tion | Sequence |
| 239 | nucleo- | AGAGAGGGCCCTGAGCTGAGCCCCGATGATCCTGCTGG |
| | tide | ACTGCTGGACCTGCGGCAGGGCATGTTTGCTCAGCTGGT |
| | se- | GGCCCAGAACGTGCTGCTGATCGATGGCCCCCTGTCCTG |
| | quence | GTACAGCGATCCTGGACTGGCTGGCGTGTCACTGACAGG |
| | of | CGGCCTGAGCTACAAAGAGGACACCAAAGAACTGGTGG |
| | dimeric | TGGCCAAGGCCGGCGTGTACTACGTGTTCTTTCAGCTGG |
| | hu 4- | AACTGCGGAGAGTGGTGGCCGGCGAAGGATCTGGCTCT |
| | 1BBL | GTGTCTCTGGCCCTGCATCTGCAGCCTCTGAGAAGCGCT |
| | (71- | GCTGGCGCTGCAGCTCTGGCACTGACAGTGGATCTGCCT |
| | 254)- | CCTGCCAGCTCCGAGGCCCGGAATAGCGCATTTGGGTTT |
| | CL* Fc | CAAGGCAGGCTGCTGCACCTGTCTGCCGGCCAGAGGCT |
| | knob | GGGAGTGCATCTGCACACAGAGGCCAGGGCTAGACACG |
| | chain | CCTGGCAGCTGACACAGGGCGCTACAGTGCTGGGCCTG |
| | | TTCAGAGTGACCCCCGAGATTCCAGCCGGCCTGCCTTCT |
| | | CCAAGAAGCGAAGGCGGAGGCGGATCTGGCGGCGGAG |
| | | GATCTAGAGAGGGACCCGAACTGTCCCCTGACGATCCA |
| | | GCCGGGCTGCTGGATCTGAGACAGGGAATGTTCGCCCA |
| | | GCTGGTGGCTCAGAATGTGCTGCTGATTGACGGACCTCT |
| | | GAGCTGGTACTCCGACCCAGGGCTGGCAGGGGTGTCCC |
| | | TGACTGGGGGACTGTCCTACAAAGAAGATACAAAAGAA |
| | | CTGGTGGTGGCTAAAGCTGGGGTGTACTATGTGTTTTTT |
| | | CAGCTGGAACTGAGGCGGGTGGTGGCTGGGGAGGGCTC |
| | | AGGATCTGTGTCCCTGGCTCTGCATCTGCAGCCACTGCG |
| | | CTCTGCTGCTGGCGCAGCTGCACTGGCTCTGACTGTGGA |
| | | CCTGCCACCAGCCTCTAGCGAGGCCAGAAACAGCGCCT |
| | | TCGGGTTCCAAGGACGCCTGCTGCATCTGAGCGCCGGAC |
| | | AGCGCCTGGGAGTGCATCTGCATACTGAAGCCAGAGCC |
| | | CGGCATGCTTGGCAGCTGACTCAGGGGGCAACTGTGCTG |
| | | GGACTGTTTCGCGTGACACCTGAGATCCCTGCCGGACTG |
| | | CCAAGCCCTAGATCAGAAGGGGGCGGAGGTTCCGGAGG |
| | | GGGAGGATCTCGTACGGTGGCTGCACCATCTGTCTTTAT |
| | | CTTCCCACCCAGCGACCGGAAGCTGAAGTCTGGCACAG |
| | | CCAGCGTCGTGTGCCTGCTGAATAACTTCTACCCCCGCG |
| | | AGGCCAAGGTGCAGTGGAAGGTGGACAATGCCCTGCAG |
| | | AGCGGCAACAGCCAGGAAAGCGTGACCGAGCAGGACA |
| | | GCAAGGACTCCACCTACAGCCTGAGCAGCACCCTGACC |
| | | CTGAGCAAGGCCGACTACGAGAAGCACAAGGTGTACGC |
| | | CTGCGAAGTGACCCACCAGGGCCTGTCTAGCCCCGTGAC |
| | | CAAGAGCTTCAACCGGGGCGAGTGCGACAAGACCCACA |
| | | CCTGTCCTCCATGCCCTGCCCCTGAAGCTGCTGGCGGCC |
| | | CTAGCGTGTTCCTGTTCCCCCCAAAGCCCAAGGACACCC |
| | | TGATGATCAGCCGGACCCCTGAAGTGACCTGCGTGGTGG |
| | | TGGATGTGTCCCACGAGGACCCTGAAGTGAAGTTCAATT |
| | | GGTACGTGGACGGCGTGGAAGTGCACAATGCCAAGACC |
| | | AAGCCGCGGGAGGAGCAGTACAACAGCACGTACCGTGT |
| | | GGTCAGCGTCCTCACCGTCCTGCACCAGGACTGGCTGAA |
| | | TGGCAAGGAGTACAAGTGCAAGGTCTCCAACAAAGCCC |
| | | TCGGCGCCCCCATCGAGAAAACCATCTCCAAAGCCAAA |
| | | GGGCAGCCCCGAGAACCACAGGTGTACACCCTGCCCCC |
| | | ATGCCGGGATGAGCTGACCAAGAACCAGGTCAGCCTGT |
| | | GGTGCCTGGTCAAAGGCTTCTATCCCAGCGACATCGCCG |
| | | TGGAGTGGGAGAGCAATGGGCAGCCGGAGAACAACTAC |
| | | AAGACCACGCCTCCCGTGCTGGACTCCGACGGCTCCTTC |
| | | TTCCTCTACAGCAAGCTCACCGTGGACAAGAGCAGGTG |
| | | GCAGCAGGGGAACGTCTTCTCATGCTCCGTGATGCATGA |
| | | GGCTCTGCACAACCACTACACGCAGAAGAGCCTCTCCCT |
| | | GTCTCCGGGTAAA |
| 240 | nucleo- | AGAGAGGGCCCTGAGCTGAGCCCCGATGATCCTGCTGG |
| | tide | ACTGCTGGACCTGCGGCAGGGCATGTTTGCTCAGCTGGT |
| | se- | GGCCCAGAACGTGCTGCTGATCGATGGCCCCCTGTCCTG |
| | quence | GTACAGCGATCCTGGACTGGCTGGCGTGTCACTGACAGG |
| | of | CGGCCTGAGCTACAAAGAGGACACCAAAGAACTGGTGG |
| | mono- | TGGCCAAGGCCGGCGTGTACTACGTGTTCTTTCAGCTGG |
| | meric | AACTGCGGAGAGTGGTGGCCGGCGAAGGATCTGGCTCT |
| | hu 4- | GTGTCTCTGGCCCTGCATCTGCAGCCTCTGAGAAGCGCT |
| | 1BBL | GCTGGCGCTGCAGCTCTGGCTCTGACAGTGGATCTGCCT |
| | (71- | CCTGCCAGCTCCGAGGCCCGGAATAGCGCATTTGGGTTT |
| | 254) | CAAGGCCGGCTGCTGCACCTGTCTGCCGGCCAGAGACT |
| | CH1* | GGGAGTGCATCTGCACACAGAGGCCAGAGCCAGGCACG |
| | | CCTGGCAGCTGACACAGGGCGCTACAGTGCTGGGCCTG |
| | | TTCAGAGTGACCCCCGAGATTCCTGCCGGCCTGCCTAGC |
| | | CCTAGATCTGAAGGCGGCGGAGGTTCCGGAGGCGGAGG |
| | | ATCTGCTAGCACAAAGGGCCCCAGCGTGTTCCCTCTGGC |
| | | CCCTAGCAGCAAGAGCACATCTGGCGGAACAGCCGCCC |
| | | TGGGCTGCCTGGTGGAAGATTACTTCCCCGAGCCCGTGA |
| | | CCGTGTCCTGGAATTCTGGCGCCCTGACAAGCGGCGTGC |
| | | ACACCTTTCCAGCCGTGCTGCAGAGCAGCGGCCTGTACT |
| | | CTCTGAGCAGCGTCGTGACAGTGCCCAGCAGCTCTCTGG |
| | | GCACCCAGACCTACATCTGCAACGTGAACCACAAGCCC |
| | | AGCAACACCAAGGTGGACGAGAAGGTGGAACCCAAGTC |
| | | CTGC |
| 241 | nucleo- | CAGGTGCAATTGGTTCAATCTGGTGCTGAAGTAAAAAA |
| | tide | ACCGGGCGCTTCCGTTAAAGTGAGCTGCAAAGCATCTGG |
| | se- | TTACACCTTCACTGACTATATCATGCACTGGGTTCGTCA |
| | quence | GGCCCCGGGCCAGGGTCTGGAGTGGATGGGCTACATTA |
| | of | ACCCATACAACGACGGTTCCAAATATACCGAGAAATTC |
| | anti- | CAGGGCCGCGTCACGATGACCAGCGACACTTCTATCTCC |
| | CD19 | ACCGCGTACATGGAACTGTCTAGACTGCGTTCTGACGAC |
| | (8B8- | ACCGCTGTTTACTATTGTGCACGCGGTACCTACTACTAC |
| | 2B11) | GGTCCACAGCTGTTTGATTACTGGGGCCAAGGTACCACG |
| | Fc hole | GTGACCGTAAGCTCTGCTAGCACCAAGGGCCCCTCCGTG |
| | chain | TTCCCCCTGGCCCCCAGCAGCAAGAGCACCAGCGGCGG |
| | | CACAGCCGCTCTGGGCTGCCTGGTCAAGGACTACTTCCC |
| | | CGAGCCCGTGACCGTGTCCTGGAACAGCGGAGCCCTGA |
| | | CCTCCGGCGTGCACACCTTCCCCGCCGTGCTGCAGAGTT |
| | | CTGGCCTGTATAGCCTGAGCAGCGTGGTCACCGTGCCTT |
| | | CTAGCAGCCTGGGCACCCAGACCTACATCTGCAACGTG |
| | | AACCACAAGCCCAGCAACACCAAGGTGGACAAGAAGGT |
| | | GGAGCCCAAGAGCTGCGACAAAACTCACACATGCCCAC |
| | | CGTGCCCAGCACCTGAAGCTGCAGGGGGACCGTCAGTC |
| | | TTCCTCTTCCCCCCAAAACCCAAGGACACCCTCATGATC |
| | | TCCCGGACCCCTGAGGTCACATGCGTGGTGGTGGACGTG |
| | | AGCCACGAAGACCCTGAGGTCAAGTTCAACTGGTACGT |
| | | GGACGGCGTGGAGGTGCATAATGCCAAGACAAAGCCGC |
| | | GGGAGGAGCAGTACAACAGCACGTACCGTGTGGTCAGC |
| | | GTCCTCACCGTCCTGCACCAGGACTGGCTGAATGGCAAG |
| | | GAGTACAAGTGCAAGGTCTCCAACAAAGCCCTCGGCGC |
| | | CCCCATCGAGAAAACCATCTCCAAAGCCAAAGGGCAGC |
| | | CCCGAGAACCACAGGTGTGCACCCTGCCCCCATCCCGG |
| | | GATGAGCTGACCAAGAACCAGGTCAGCCTCTCGTGCGC |
| | | AGTCAAAGGCTTCTATCCCAGCGACATCGCCGTGGAGTG |
| | | GGAGAGCAATGGGCAGCCGGAGAACAACTACAAGACC |
| | | ACGCCTCCCGTGCTGGACTCCGACGGCTCCTTCTTCCTC |
| | | GTGAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTAAA |
| 124 | nucleo- | see Table 7 |
| | tide | |
| | se- | |
| | quence | |
| | of | |
| | anti- | |
| | CD19 | |
| | (8B8- | |
| | 2B11) | |
| | light | |
| | chain | |
| 242 | Dimeric | REGPELSPDDPAGLLDLRQGMFAQLVAQNVLLIDGPLSW |
| | hu 4- | YSDPGLAGVSLTGGLSYKEDTKELVVAKAGVYYVFFQLE |
| | 1BBL | LRRVVAGEGSGSVSLALHLQPLRSAAGAAALALTVDLPP |
| | (71- | ASSEARNSAFGFQGRLLHLSAGQRLGVHLHTEARARHAW |
| | 254) | QLTQGATVLGLFRVTPEIPAGLPSPRSEGGGGSGGGGSR |
| | CL* Fc | EGPELSPDDPAGLLDLRQGMFAQLVAQNVLLIDGPLSWY |
| | knob | SDPGLAGVSLTGGLSYKEDTKELVVAKAGVYYVFFQLEL |
| | chain | RRVVAGEGSGSVSLALHLQPLRSAAGAAALALTVDLPPA |
| | | SSEARNSAFGFQGRLLHLSAGQRLGVHLHTEARARHAWQ |
| | | LTQGATVLGLFRVTPEIPAGLPSPRSEGGGGSGGGGSRT |
| | | VAAPSVFIFPPSDRKLKSGTASVVCLLNNFYPREAKVQW |
| | | KVDNALQSGNSQESVTEQDSKDSTYSLSSTLTLSKADYE |
| | | KHKVYACEVTHQGLSSPVTKSFNRGECDKTHTCPPCPAP |
| | | EAAGGPSVFLFPPKPKDTLMISRTPEVTCVVVDVSHEDP |
| | | EVKFNWYVDGVEVHNAKTKPREEQYNSTYRVVSVLTVLH |
| | | QDWLNGKEYKCKVSNKALGAPIEKTISKAKGQPREPQVY |
| | | TLPPCRDELTKNQVSLWCLVKGFYPSDIAVEWESNGQPE |
| | | NNYKTTPPVLDSDGSFFLYSKLTVDKSRWQQGNVFSCSV |
| | | MHEALHNHYTQKSLSLSPGK |
| 243 | Mono- | REGPELSPDDPAGLLDLRQGMFAQLVAQNVLLIDGPLSW |
| | meric | YSDPGLAGVSLTGGLSYKEDTKELVVAKAGVYYVFFQLE |
| | hu 4- | LRRVVAGEGSGSVSLALHLQPLRSAAGAAALALTVDLPP |
| | 1BBL | ASSEARNSAFGFQGRLLHLSAGQRLGVHLHTEARARHAW |
| | (71- | QLTQGATVLGLFRVTPEIPAGLPSPRSEGGGGSGGGGSA |
| | 254)- | STKGPSVFPLAPSSKSTSGGTAALGCLVEDYFPEPVTVS |
| | CH1* | WNSGALTSGVHTFPAVLQSSGLYSLSSVVTVPSSSLGTQ |
| | | TYICNVNHKPSNTKVDEKVEPKSC |
| 244 | CD19 | QVQLVQSGAEVKKPGASVKVSCKASGYTFTDYIMHWVRQ |
| | (8B8- | APGQGLEWMGYINPYNDGSKYTEKFQGRVTMTSDTSIST |
| | 2B11) | YMELSRLRSDDTAVYYCARGTYYYGPQLFDYWGQGTTVT |
| | Fc hole | VSSASTKGPSVFPLAPSSKSTSGGTAALGCLVKDYFPEP |
| | chain | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALGAPIEKTISKAKGQPREPQV |
| | | CTLPPSRDELTKNQVSLSCAVKGFYPSDIAVEWESNGQP |
| | | ENNYKTTPPVLDSDGSFFLVSKLTVDKSRWQQGNVFSCS |
| | | VMHEALHNHYTQKSLSLSPGK |
| 126 | anti- | see Table 7 |
| | CD19 | |
| | (8B8- | |
| | 2B11) | |
| | light | |
| | chain | |

An additional control used in the assays, termed control F, was an untargeted DP47, germline control, human IgG1, containing the Pro329Gy, Leu234Aa and Leu235Aa mutations (termed PGLALA) to abrogate binding to Fcgamma receptors according to the method described in International Patent Appl. Publ. No. WO 2012/130831 A1.

Table 38 shows the cDNA and amino acid sequences of the “untargeted” DP47 hu IgG1 PGLALA antibody (Control F).

TABLE 38
Sequences of DP47-untargeted human IgG1 PGLALA
(Control F)

| SEQ | De- | |
|---|---|---|
| ID | scrip- | |
| NO: | tion | Sequence |
| 205 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of DP47 | GTGGTAGTGGTGGTAGCACATACTACGCAGACTCCGTGA |
| | heavy | AGGGCCGGTTCACCATCTCCAGAGACAATTCCAAGAAC |
| | chain | ACGCTGTATCTGCAGATGAACAGCCTGAGAGCCGAGGA |
| | (huIgG1 | CACGGCCGTATATTACTGTGCGAAAGGCAGCGGATTTGA |
| | PGLALA) | CTACTGGGGCCAAGGAACCCTGGTCACCGTCTCGAGTGC |
| | | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCATCCCGGGATGAGCTGACCAAGA |
| | | ACCAGGTCAGCCTGACCTGCCTGGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCTACAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 166 | nucleo- | see Table 21 |
| | tide | |
| | se- | |
| | quence | |
| | of DP47 | |
| | light | |
| | chain | |
| 206 | DP47 | EVQLLESGGGLVQPGGSLRLSCAASGFTFSSYAMSWVRQ |
| | heavy | APGKGLEWVSAISGSGGSTYYADSVKGRFTISRDNSKNT |
| | chain | LYLQMNSLRAEDTAVYYCAKGSGFDYWGQGTLVTVSSAS |
| | (huIgG1 | TKGPSVFPLAPSSKSTSGGTAALGCLVKDYFPEPVTVSW |
| | PGLALA) | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 168 | DP47 | see Table 21 |
| | light | |
| | chain | |

### Example 2

### Production of Monovalent Targeted Human 4-1BB Ligand Trimer-Containing Fc Fusion Antigen Binding Molecules

The targeted and untargeted monovalent trimeric 4-1BB ligand C-terminal Fc (kih) fusion antigen binding molecule encoding sequences were cloned into a plasmid vector, which drives expression of the insert from an MPSV promoter and contains a synthetic polyA sequence located at the 3′ end of the CDS. In addition, the vector contains an EBV OriP sequence for episomal maintenance of the plasmid.

The split trimeric 4-1BB ligand Fc (kih) fusion antigen binding molecules were produced by co-transfecting HEK293-EBNA cells with the mammalian expression vectors using polyethylenimine. The cells were transfected with the corresponding expression vectors. For variants x.11, x.12, x.13, x.14 and the control molecules H, I, J and K, at a 1:1:1 ratio (“vector knob chain”: “vector hole chain”: “vector light chain”). For control D, at a ratio of 1:1:1:1 (“vector knob chain”: “vector hole chain”: “vector light chain”: “vector light chain2”) and for human IgG1, control F, at a 1:1 ratio (“vector heavy chain”: “vector light chain).

For production in 500 mL shake flasks, 300 million HEK293 EBNA cells were seeded 24 hours before transfection. For transfection cells were centrifuged for 10 minutes at 210×g, and the supernatant was replaced by 20 mL pre-warmed CD CHO medium. Expression vectors (200 μg of total DNA) were mixed in 20 mL CD CHO medium. After addition of 540 μL PEI, the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO₂ atmosphere. After the incubation, 160 mL of Excell medium supplemented with 6 mM L-Glutamine, 5 g/L PEPSOY and 1.2 mM valproic acid was added and cells were cultured for 24 hours. One day after transfection 12% Feed were added. After culturing for 7 days, the supernatant was collected by centrifugation for 30-40 minutes at least 400×g. The solution was sterile filtered (0.22 μm filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a MabSelect Sure column (CV=5-15 mL, resin from GE Healthcare) equilibrated with Sodium Phosphate (20 mM), Sodium Citrate (20 mM) buffer (pH 7.5). Unbound protein was removed by washing with at least 6 column volumes of the same buffer. The bound protein was eluted using either a linear gradient (20 CV) or a step elution (8 CV) with 20 mM sodium citrate, 100 mM Sodium chloride, 100 mM Glycine buffer (pH 3.0). For the linear gradient an additional 4 column volumes step elution was applied.

The pH of collected fractions was adjusted by adding 1/10 (v/v) of 0.5M sodium phosphate, pH 8.0. The protein was concentrated prior to loading on a HiLoad Superdex 200 column (GE Healthcare) equilibrated with 20 mM Histidine, 140 mM sodium chloride, 0.01% (v/v) Tween20 solution of pH 6.0.

The protein concentration was determined by measuring the optical density (OD) at 280 nm, using a molar extinction coefficient calculated on the basis of the amino acid sequence. Purity and molecular weight of the targeted trimeric 4-1BB ligand Fc (kih) fusion antigen binding molecules was analyzed by SDS-PAGE in the presence and absence of a reducing agent (5 mM 1,4-dithiotreitol) and staining with Coomassie SimpleBlue™ SafeStain (Invitrogen USA) or CE-SDS using Caliper LabChip GXII (Perkin Elmer). The aggregate content of samples was analyzed using a TSKgel G3000 SW XL analytical size-exclusion column (Tosoh) equilibrated in 25 mM K2HPO₄, 125 mM NaCl, 200 mM L-Arginine Monohydrocloride, 0.02% (w/v) NaN₃, pH 6.7 running buffer at 25° C.

Table 39 summarizes the yield and final monomer content of the targeted split trimeric C-terminal 4-1BB ligand Fc(kih) fusion antigen binding molecules.

TABLE 39
Biochemical analysis of exemplary targeted split trimeric C-terminal
4-1BB ligand Fc(kih) fusion antigen binding molecules

| | Monomer | |
|---|---|---|
| | [%] | Yield |
| Construct | (SEC) | [mg/l] |
| monovalent targeted FAP(4B9) split trimeric C- | 97.8 | 19 |
| terminal 4-1BB ligand (71-248) Fc (kih) fusion | | |
| antigen binding molecule | | |
| Construct 2.11 | | |
| monovalent targeted CD19(8B8-018) split | 100 | 16 |
| trimeric C-terminal 4-1BB ligand (71-248) Fc | | |
| (kih) fusion antigen binding molecule | | |
| Construct 3.11 | | |
| monovalent targeted CD19(8B8-2B11) split | 100 | 6 |
| trimeric C-terminal 4-1BB ligand (71-248) Fc | | |
| (kih) fusion antigen binding molecule | | |
| Construct 4.11 | | |
| monovalent DP47-untargeted split trimeric | 99.5 | 25.9 |
| human 4-1BB ligand (71-254) Fc (kih) fusion | | |
| antigen (Control D) | | |
| germline DP47 human IgG1 PGLALA | 100 | 50 |
| Control F | | |

### Example 3

### Affinity Determination by Surface Plasmon Resonance

3.1 Preparation, Purification and Characterization of Antigens or Surface Plasmon Resonance

Human CDT19 Fe Fusion Protein

The preparation, purification and characterization of the human CD19 ectodomain fused to a human IgG1 Fc domain is described in Example 1.3.2.

Human 4-1BB

The ectodomain of human 4-1 BB (Q07011, amino acid 24-186) was subcloned in frame with an avi (GLNDIFEAQKIEHESEQ ID NO. 245) and a hexahistidine tag (Table 40).

TABLE 40
Sequences of monomeric human 4-1BB His molecule

| SEQ | | |
|---|---|---|
| ID | anti- | |
| NO: | gen | Sequence |
| 207 | nucleo- | CTGCAGGACCCCTGCAGCAACTGCCCTGCCGGCACCT |
| | tide | TCTGCGACAACAACCGGAACCAGATCTGCAGCCCCTG |
| | se- | CCCCCCCAACAGCTTCAGCTCTGCCGGCGGACAGCGG |
| | quence | ACCTGCGACATCTGCAGACAGTGCAAGGGCGTGTTCA |
| | of | GAACCCGGAAAGAGTGCAGCAGCACCAGCAACGCCG |
| | human | AGTGCGACTGCACCCCCGGCTTCCATTGTCTGGGAGC |
| | 4-1BB | CGGCTGCAGCATGTGCGAGCAGGACTGCAAGCAGGG |
| | His | CCAGGAACTGACCAAGAAGGGCTGCAAGGACTGCTG |
| | | CTTCGGCACCTTCAACGACCAGAAGCGGGGCATCTGC |
| | | CGGCCCTGGACCAACTGTAGCCTGGACGGCAAGAGCG |
| | | TGCTGGTCAACGGCACCAAAGAACGGGACGTCGTGTG |
| | | CGGCCCCAGCCCTGCTGATCTGTCTCCTGGGGCCAGC |
| | | AGCGTGACCCCTCCTGCCCCTGCCAGAGAGCCTGGCC |
| | | ACTCTCCTCAGGTCGACGAACAGTTATATTTTCAGGG |
| | | CGGCTCAGGCCTGAACGACATCTTCGAGGCCCAGAAG |
| | | ATCGAGTGGCACGAGGCTCGAGCTCACCACCATCACC |
| | | ATCAC |
| 208 | human | LQDPCSNCPAGTFCDNNRNQICSPCPPNSFSSAGGQRTC |
| | 4-1BB | DICRQCKGVFRTRKECSSTSNAECDCTPGFHCLGAGCSM |
| | His | CEQDCKQGQELTKKGCKDCCFGTFNDQKRGICRPWTNC |
| | | SLDGKSVLVNGTKERDVVCGPSPADLSPGASSVTPPAPA |
| | | REPGHSPQVDEQLYFQGGSGLNDIFEAQKIEWHEARAH |
| | | HHHHH |

Protein production was performed as described above for the CD19 Fc fusion protein in Example 1.3.2. Secreted proteins were purified from cell culture supernatants by chelating chromatography, followed by size exclusion chromatography.

The first chromatographic step was performed on a NiNTA Superflow Cartridge (5 ml, Qiagen) equilibrated in 20 mM sodium phosphate, 500 nM sodium chloride, pH7.4. Elution was performed by applying a gradient over 12 column volume from 5% to 45% of elution buffer (20 mM sodium phosphate, 500 nM sodium chloride, 500 mM Imidazole, pH7.4).

The protein was concentrated and filtered prior to loading on a HiLoad Superdex 75 column (GE Healthcare) equilibrated with 2 mM MOPS, 150 mM sodium chloride, 0.02% (w/v) sodium azide solution of pH 7.4. Table 41 summarizes the yield and final monomer content of monomeric human avi His-tagged 4-1BB.

TABLE 41
Biochemical analysis of monomeric human 4-1BB avi His

| | Monomer | Yield |
|---|---|---|
| Construct | [%] (SEC) | [mg/l] |
| monomeric human 4-1BB avi His | 100 | 16.4 |

3.2 Affinity Determination

Binding of targeted split trimeric C-terminal 4-1BB ligand antigen binding molecules to the recombinant 4-1BB or CD19 was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany).

Affinity for Human 4-1BB

Anti-human Fc antibody (Biacore, Freiburg/Germany) was directly coupled on a CM5 chip at pH 5.0 using the standard amine coupling kit (Biacore, Freiburg/Germany). The immobilization level was approximately 4000 RU. Targeted split trimeric C-terminal 4-1BB ligand was captured for 90 seconds at concentration of 4 nM. Recombinant human 4-1BB avi his was passed at a concentration range from 2.7 to 2000 nM with a flow of 30 μL/minutes through the flow cells over 120 seconds. The dissociation was monitored for 120 seconds. Bulk refractive index differences were corrected for by subtracting the response obtained on reference flow cell. Here, the antigens were flown over a surface with immobilized anti-human Fe antibody but on which HBS-EP has been injected rather than the antibodies.

Affinity for Human CD19

Anti-human Fab antibody (Biacore, Freiburg/Germany) was directly coupled on a CM5 chip at pH 5.0 using the standard amine coupling kit (Biacore, Freiburg/Germany). The immobilization level was approximately 7000 RU. Targeted split trimeric C-terminal 4-1BB ligand was captured for 60 seconds at concentrations between 75 and 100 nM. Recombinant human CD19 Fc(kih) was passed at a concentration range from 7.8 to 500 nM with a flow of 30 μL/minutes through the flow cells over 220 seconds. The dissociation was monitored for 600/2500 seconds. Bulk refractive index differences were corrected for by subtracting the response obtained on reference flow cell. Here, the antigens were flown over a surface with immobilized anti-human Fc antibody but on which HBS-EP has been injected rather than the antibodies.

Kinetic constants were derived using the Biacore T200 Evaluation Software (vAA, Biacore AB, Uppsala/Sweden), to fit rate equations for 1:1 Langmuir binding by numerical integration and used to estimate qualitatively the avidity.

TABLE 42
Binding of targeted split trimeric C-terminal 4-1BB ligand antigen
binding molecules to recombinant human 4-1BB

| | Recombinant human 4-1 BB | | | |
|---|---|---|---|---|
| Construct | ka (1/Ms) | kd (1/s) | KD (M) | KD (M) |
| Construct 2.11 | 7.62E+04 | 2.63E−02 | 3.47E−07 | 2.079E−08 |
| Construct 4.11 | 7.37E+04 | 2.72E−02 | 3.69E−07 | 1.762E−08 |

Affinity constants for the interaction between targeted split trimeric C-terminal 4-1BB ligand antigen binding molecules and human 4-1BB avi his were determined by fitting to a 1:1 Langmuir binding. Results are the average from three experiments.

TABLE 43
Binding of targeted split trimeric C-terminal 4-1BB ligand to
recombinant human CD19

| | Recombinant human CD19 | | | |
|---|---|---|---|---|
| Construct | ka (1/Ms) | kd (1/s) | KD (M) | KD (M) |
| Construct 4.11 | 5.22E+04 | 4.57E−05 | 8.85E−10 | 1.32E−10 |

Affinity constants for the interaction between targeted split trimeric C-terminal 4-1BB ligand antigen binding molecules and human CD19 Fc(kih) were determined by fitting to a 1:1 Langmuir binding. Results are the average from three experiments.

3.3 Determination of Simultaneous Binding of Targeted C-Terminal 4-1BB Split Trimeric Ligand Fc Fusion Antigen Binding Molecules by Surface Plasmon Resonance

The capacity of binding simultaneously human 4-1BB Fc(kih) and human FAP, or human CD19, respectively was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany).

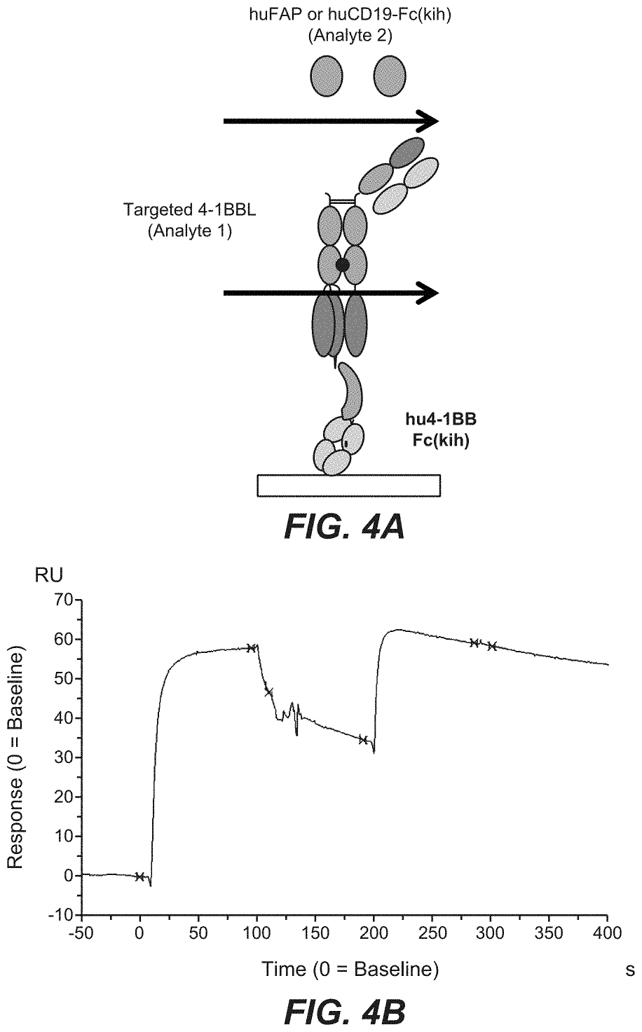

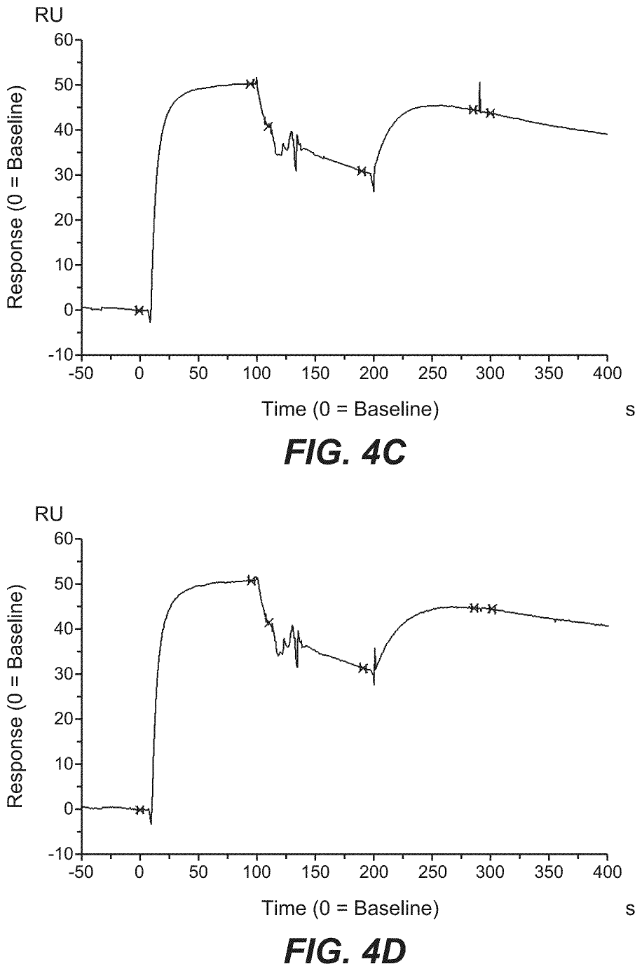

Biotinylated human 4-1BB Fc(kih) was directly coupled to a flow cell of a streptavidin (SA) sensor chip. Immobilization levels up to 200 resonance units (RU) were used. The targeted trimeric C-terminal 4-1BB ligand Fc (kih) fusion molecules were passed at a concentration range of 200 nM with a flow of 30 μL/minute through the flow cells over 90 seconds and dissociation was set to zero seconds. Human FAP or human CD19 Fc(kih) was injected as second analyte with a flow of 30 μL/minute through the flow cells over 90 seconds at a concentration of 500 nM (FIG. 4A). The dissociation was monitored for 120 sec. Bulk refractive index differences were corrected for by subtracting the response obtained in a reference flow cell, where no protein was immobilized. The bispecific constructs could bind simultaneously human 4-1BB and human FAP or human CD19, respectively (FIGS. 4B, 4C and 4D).

### Example 4

### Preparation and Purification of FAP-Targeted/DP47 Human 4-1BB Ligand Trimer-Containing Fc Fusion Antigen Binding Molecules

4.1 Preparation of Monovalent FAP(4B9)-Targeted/DP47 4-1BB Ligand Trimer-Containing Fc (Kih) Fusion Antigen Binding Molecule, Wherein the 4-1BB Ligands are C-Terminally Fused (Constructs 2.15 and 2.16) (FAP on the Hole Chain)

The DNA sequence encoding part of the ectodomain (amino acids 71-254 of human 4-1BB ligand was synthetized according to the P41273 sequence of Uniprot database.

A polypeptide containing two ectodomains of 4-1BB ligand, separated by (G4S)2 linkers was subcloned in frame to the C-terminus of human IgG1 Fc hole or knob chain, as depicted in FIG. 1A: human IgG1 Fc, (G4S)2 connector, human 4-1BB ligand, (G4S)2 connector, human 4-1BB ligand. A polypeptide containing one ectodomain of 4-1BB ligand was subcloned in frame to the C-terminus of human IgG1 Fe knob or hole chain as described in FIG. 1B: human IgG1 Fe, (G4S)2 connector, human 4-1BB ligand.

The variable region of heavy and light chain DNA sequences encoding a binder specific for fibroblast activation protein (FAP), clone 4B9, were subcloned in frame with either the constant heavy chain of the hole or the constant light chain of human IgG1. The variable region of heavy and light chain DNA sequences encoding a non-binding clone, clone DP47, were subcloned in frame with either the constant heavy chain of the knob or the constant light chain of human IgG1. The crossmab technology was applied to reduce the formation of wrongly paired light chains (International patent application No. WO 2010/145792 A1). In this example a crossed Fab unit (VHCL) of the binder DP47 was fused to the knob chain.

The Pro329Gly, Leu234Ala and Leu235Ala mutations have been introduced in the constant region of the knob and hole heavy chains to abrogate binding to Fc gamma receptors (International Patent Appl. Publ. No. WO 2012/130831 A1).

Combination of the anti-FAP huIgG1 Fc hole chain containing the Y349C/T366S/L368A/Y407V mutations, the DP47 huIgG1 Fc knob chain containing the S354C/T366W mutations and the two light chains, anti-FAP and DP47, allows generation of a heterodimer, which includes an assembled trimeric 4-1BB ligand, one FAP binding Fab and one non binding DP47 Fab (FIGS. 5A and 5B).

Table 44 shows the cDNA and amino acid sequences of the monovalent FAP(4B9)-targeted 4-1BB ligand (71-254) trimer-containing Fc (kih) fusion antigen binding molecule, wherein the dimeric 4-1BBL is fused to the hole chain (Construct 2.15).

TABLE 44
Sequences of FAP(4B9)-targeted/DP47 human 4-1BB
ligand trimer containing Fc (kih) fusion antigen
binding molecule Construct 2.15

| SEQ | De- | |
|---|---|---|
| ID | scrip- | |
| NO: | tion | Sequence |
| 209 | nucleo- | GAGGTGCAGCTGCTCGAAAGCGGCGGAGGACTGGTGCA |
| | tide | GCCTGGCGGCAGCCTGAGACTGTCTTGCGCCGCCAGCG |
| | se- | GCTTCACCTTCAGCAGCTACGCCATGAGCTGGGTCCGCC |
| | quence | AGGCCCCTGGCAAGGGACTGGAATGGGTGTCCGCCATC |
| | of | ATCGGCTCTGGCGCCAGCACCTACTACGCCGACAGCGTG |
| | anti- | AAGGGCCGGTTCACCATCAGCCGGGACAACAGCAAGAA |
| | FAP | CACCCTGTACCTGCAGATGAACAGCCTGCGGGCCGAGG |
| | (4B9) | ACACCGCCGTGTACTACTGCGCCAAGGGATGGTTCGGC |
| | Fc hole | GGCTTCAACTACTGGGGACAGGGCACCCTGGTCACAGT |
| | dimeric | GTCCAGCGCTAGCACCAAGGGCCCCTCCGTGTTCCCCCT |
| | 4-1BB | GGCCCCCAGCAGCAAGAGCACCAGCGGCGGCACAGCCG |
| | ligand | CTCTGGGCTGCCTGGTCAAGGACTACTTCCCCGAGCCCG |
| | (71- | TGACCGTGTCCTGGAACAGCGGAGCCCTGACCTCCGGC |
| | 254) | GTGCACACCTTCCCCGCCGTGCTGCAGAGTTCTGGCCTG |
| | chain | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAAACTCACACATGCCCACCGTGCCCA |
| | | GCACCTGAAGCTGCAGGGGGACCGTCAGTCTTCCTCTTC |
| | | CCCCCAAAACCCAAGGACACCCTCATGATCTCCCGGACC |
| | | CCTGAGGTCACATGCGTGGTGGTGGACGTGAGCCACGA |
| | | AGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTGCACCCTGCCCCCATCCCGGGATGAGCTG |
| | | ACCAAGAACCAGGTCAGCCTCTCGTGCGCAGTCAAAGG |
| | | CTTCTATCCCAGCGACATCGCCGTGGAGTGGGAGAGCA |
| | | ATGGGCAGCCGGAGAACAACTACAAGACCACGCCTCCC |
| | | GTGCTGGACTCCGACGGCTCCTTCTTCCTCGTGAGCAAG |
| | | CTCACCGTGGACAAGAGCAGGTGGCAGCAGGGGAACGT |
| | | CTTCTCATGCTCCGTGATGCATGAGGCTCTGCACAACCA |
| | | CTACACGCAGAAGAGCCTCTCCCTGTCTCCGGGTGGAGG |
| | | CGGCGGAAGCGGAGGAGGAGGATCCAGAGAGGGCCCT |
| | | GAGCTGAGCCCCGATGATCCTGCTGGACTGCTGGACCTG |
| | | CGGCAGGGCATGTTTGCTCAGCTGGTGGCCCAGAACGTG |
| | | CTGCTGATCGATGGCCCCCTGTCCTGGTACAGCGATCCT |
| | | GGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGAAGCGCTGCTGGCGCTGCAG |
| | | CTCTGGCACTGACAGTGGATCTGCCTCCTGCCAGCTCCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCAGCCGGCCTGCCTTCTCCAAGAAGCGAA |
| | | GGCGGAGGCGGATCTGGCGGCGGAGGATCTAGAGAGGG |
| | | ACCCGAACTGTCCCCTGACGATCCAGCCGGGCTGCTGGA |
| | | TCTGAGACAGGGAATGTTCGCCCAGCTGGTGGCTCAGA |
| | | ATGTGCTGCTGATTGACGGACCTCTGAGCTGGTACTCCG |
| | | ACCCAGGGCTGGCAGGGGTGTCCCTGACTGGGGGACTG |
| | | TCCTACAAAGAAGATACAAAAGAACTGGTGGTGGCTAA |
| | | AGCTGGGGTGTACTATGTGTTTTTTCAGCTGGAACTGAG |
| | | GCGGGTGGTGGCTGGGGAGGGCTCAGGATCTGTGTCCCT |
| | | GGCTCTGCATCTGCAGCCACTGCGCTCTGCTGCTGGCGC |
| | | AGCTGCACTGGCTCTGACTGTGGACCTGCCACCAGCCTC |
| | | TAGCGAGGCCAGAAACAGCGCCTTCGGGTTCCAAGGAC |
| | | GCCTGCTGCATCTGAGCGCCGGACAGCGCCTGGGAGTG |
| | | CATCTGCATACTGAAGCCAGAGCCCGGCATGCTTGGCA |
| | | GCTGACTCAGGGGGCAACTGTGCTGGGACTGTTTCGCGT |
| | | GACACCTGAGATCCCTGCCGGACTGCCAAGCCCTAGATC |
| | | AGAA |
| 210 | nucleo- | GAGGTGCAATTGTTGGAGTCTGGGGGAGGCTTGGTACA |
| | tide | GCCTGGGGGGTCCCTGAGACTCTCCTGTGCAGCCTCCGG |
| | se- | ATTCACCTTTAGCAGTTATGCCATGAGCTGGGTCCGCCA |
| | quence | GGCTCCAGGGAAGGGGCTGGAGTGGGTCTCAGCTATTA |
| | of DP47 | GTGGTAGTGGTGGTAGCACATACTACGCAGACTCCGTGA |
| | (VHCL) | AGGGCCGGTTCACCATCTCCAGAGACAATTCCAAGAAC |
| | Fc knob | ACGCTGTATCTGCAGATGAACAGCCTGAGAGCCGAGGA |
| | mono- | CACGGCCGTATATTACTGTGCGAAAGGCAGCGGATTTGA |
| | meric | CTACTGGGGCCAAGGAACCCTGGTCACCGTCTCGAGCG |
| | 4-1BB | CTAGTGTGGCCGCTCCCTCCGTGTTTATCTTTCCCCCATC |
| | (71- | CGATGAACAGCTGAAAAGCGGCACCGCCTCCGTCGTGT |
| | 254) | GTCTGCTGAACAATTTTTACCCTAGGGAAGCTAAAGTGC |
| | ligand | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAGACCCACACCTGTCCCCCTTG |
| | | TCCTGCCCCTGAAGCTGCTGGCGGCCCTTCTGTGTTCCT |
| | | GTTCCCCCCAAAGCCCAAGGACACCCTGATGATCAGCC |
| | | GGACCCCCGAAGTGACCTGCGTGGTGGTGGATGTGTCCC |
| | | ACGAGGACCCTGAAGTGAAGTTCAATTGGTACGTGGAC |
| | | GGCGTGGAAGTGCACAACGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTACACCCTGCCCCCCTGCAGAGAT |
| | | GAGCTGACCAAGAACCAGGTGTCCCTGTGGTGTCTGGTC |
| | | AAGGGCTTCTACCCCAGCGATATCGCCGTGGAGTGGGA |
| | | GAGCAACGGCCAGCCTGAGAACAACTACAAGACCACCC |
| | | CCCCTGTGCTGGACAGCGACGGCAGCTTCTTCCTGTACT |
| | | CCAAACTGACCGTGGACAAGAGCCGGTGGCAGCAGGGC |
| | | AACGTGTTCAGCTGCAGCGTGATGCACGAGGCCCTGCA |
| | | CAACCACTACACCCAGAAGTCCCTGAGCCTGAGCCCCG |
| | | GCGGAGGCGGCGGAAGCGGAGGAGGAGGATCCAGAGA |
| | | GGGCCCTGAGCTGAGCCCCGATGATCCTGCTGGACTGCT |
| | | GGACCTGCGGCAGGGCATGTTTGCTCAGCTGGTGGCCCA |
| | | GAACGTGCTGCTGATCGATGGCCCCCTGTCCTGGTACAG |
| | | CGATCCTGGACTGGCTGGCGTGTCACTGACAGGCGGCCT |
| | | GAGCTACAAAGAGGACACCAAAGAACTGGTGGTGGCCA |
| | | AGGCCGGCGTGTACTACGTGTTCTTTCAGCTGGAACTGC |
| | | GGAGAGTGGTGGCCGGCGAAGGATCTGGCTCTGTGTCTC |
| | | TGGCCCTGCATCTGCAGCCTCTGAGAAGCGCTGCTGGCG |
| | | CTGCAGCTCTGGCACTGACAGTGGATCTGCCTCCTGCCA |
| | | GCTCCGAGGCCCGGAATAGCGCATTTGGGTTTCAAGGCA |
| | | GGCTGCTGCACCTGTCTGCCGGCCAGAGGCTGGGAGTG |
| | | CATCTGCACACAGAGGCCAGGGCTAGACACGCCTGGCA |
| | | GCTGACACAGGGCGCTACAGTGCTGGGCCTGTTCAGAGT |
| | | GACCCCCGAGATTCCAGCCGGCCTGCCTTCTCCAAGAAG |
| | | CGAA |
| 103 | nucleo- | see Table 1 |
| | tide | |
| | se- | |
| | quence | |
| | of | |
| | anti- | |
| | FAP | |
| | (4B9) | |
| | light | |
| | chain | |
| 211 | nucleo- | GAAATCGTGTTAACGCAGTCTCCAGGCACCCTGTCTTTG |
| | tide | TCTCCAGGGGAAAGAGCCACCCTCTCTTGCAGGGCCAGT |
| | se- | CAGAGTGTTAGCAGCAGCTACTTAGCCTGGTACCAGCAG |
| | quence | AAACCTGGCCAGGCTCCCAGGCTCCTCATCTATGGAGCA |
| | of DP47 | TCCAGCAGGGCCACTGGCATCCCAGACAGGTTCAGTGG |
| | (VL- | CAGTGGATCCGGGACAGACTTCACTCTCACCATCAGCAG |
| | CH1) | ACTGGAGCCTGAAGATTTTGCAGTGTATTACTGTCAGCA |
| | light | GTATGGTAGCTCACCGCTGACGTTCGGCCAGGGGACCA |
| | chain | AAGTGGAAATCAAGAGCTCCGCTAGCACCAAAGGCCCT |
| | | TCCGTGTTTCCTCTGGCTCCTAGCTCCAAGTCCACCTCTG |
| | | GAGGCACCGCTGCTCTCGGATGCCTCGTGAAGGATTATT |
| | | TTCCTGAGCCTGTGACAGTGTCCTGGAATAGCGGAGCAC |
| | | TGACCTCTGGAGTGCATACTTTCCCCGCTGTGCTGCAGT |
| | | CCTCTGGACTGTACAGCCTGAGCAGCGTGGTGACAGTGC |
| | | CCAGCAGCAGCCTGGGCACCCAGACCTACATCTGCAAC |
| | | GTGAACCACAAGCCCAGCAACACCAAGGTGGACAAGAA |
| | | GGTGGAACCCAAGTCTTGT |
| 212 | anti- | EVQLLESGGGLVQPGGSLRLSCAASGFTFSSYAMSWVRQ |
| | FAP | APGKGLEWVSAIIGSGASTYYADSVKGRFTISRDNSKNT |
| | (4B9) | LYLQMNSLRAEDTAVYYCAKGWFGGFNYWGQGTLVTVSS |
| | Fc hole | ASTKGPSVFPLAPSSKSTSGGTAALGCLVKDYFPEPVTV |
| | dimeric | SWNSGALTSGVHTFPAVLQSSGLYSLSSVVTVPSSSLGT |
| | 4-1BB | QTYICNVNHKPSNTKVDKKVEPKSCDKTHTCPPCPAPEA |
| | ligand | AGGPSVFLFPPKPKDTLMISRTPEVTCVVVDVSHEDPEV |
| | (71- | KFNWYVDGVEVHNAKTKPREEQYNSTYRVVSVLTVLHQD |
| | 254) | WLNGKEYKCKVSNKALGAPIEKTISKAKGQPREPQVCTL |
| | chain | PPSRDELTKNQVSLSCAVKGFYPSDIAVEWESNGQPENN |
| | | YKTTPPVLDSDGSFFLVSKLTVDKSRWQQGNVFSCSVMH |
| | | EALHNHYTQKSLSLSPGGGGGSGGGGSREGPELSPDDPA |
| | | GLLDLRQGMFAQLVAQNVLLIDGPLSWYSDPGLAGVSLT |
| | | GGLSYKEDTKELVVAKAGVYYVFFQLELRRVVAGEGSGS |
| | | VSLALHLQPLRSAAGAAALALTVDLPPASSEARNSAFGF |
| | | QGRLLHLSAGQRLGVHLHTEARARHAWQLTQGATVLGLF |
| | | RVTPEIPAGLPSPRSEGGGGSGGGGSREGPELSPDDPAG |
| | | LLDLRQGMFAQLVAQNVLLIDGPLSWYSDPGLAGVSLTG |
| | | GLSYKEDTKELVVAKAGVYYVFFQLELRRVVAGEGSGSV |
| | | SLALHLQPLRSAAGAAALALTVDLPPASSEARNSAFGFQ |
| | | GRLLHLSAGQRLGVHLHTEARARHAWQLTQGATVLGLFR |
| | | VTPEIPAGLPSPRSE |
| 213 | DP47 | EVQLLESGGGLVQPGGSLRLSCAASGFTFSSYAMSWVRQ |
| | (VH-CL) | APGKGLEWVSAISGSGGSTYYADSVKGRFTISRDNSKNT |
| | Fc knob | LYLQMNSLRAEDTAVYYCAKGSGFDYWGQGTLVTVSSAS |
| | mono- | VAAPSVFIFPPSDEQLKSGTASVVCLLNNFYPREAKVQW |
| | meric | KVDNALQSGNSQESVTEQDSKDSTYSLSSTLTLSKADYE |
| | 4-1BB | KHKVYACEVTHQGLSSPVTKSFNRGECDKTHTCPPCPAP |
| | (71- | EAAGGPSVFLFPPKPKDTLMISRTPEVTCVVVDVSHEDP |
| | 254) | EVKFNWYVDGVEVHNAKTKPREEQYNSTYRVVSVLTVLH |
| | ligand | QDWLNGKEYKCKVSNKALGAPIEKTISKAKGQPREPQVY |
| | | TLPPCRDELTKNQVSLWCLVKGFYPSDIAVEWESNGQPE |
| | | NNYKTTPPVLDSDGSFFLYSKLTVDKSRWQQGNVFSCSV |
| | | MHEALHNHYTQKSLSLSPGGGGGSGGGGSREGPELSPDD |
| | | PAGLLDLRQGMFAQLVAQNVLLIDGPLSWYSDPGLAGVS |
| | | LTGGLSYKEDTKELVVAKAGVYYVFFQLELRRVVAGEGS |
| | | GSVSLALHLQPLRSAAGAAALALTVDLPPASSEARNSAF |
| | | GFQGRLLHLSAGQRLGVHLHTEARARHAWQLTQGATVLG |
| | | LFRVTPEIPAGLPSPRSE |
| 106 | anti- | see Table 1 |
| | FAP | |
| | (4B9) | |
| | light | |
| | chain | |
| 214 | DP47 | EIVLTQSPGTLSLSPGERATLSCRASQSVSSSYLAWYQQ |
| | (VL- | KPGQAPRLLIYGASSRATGIPDRFSGSGSGTDFTLTISR |
| | CH1) | LEPEDFAVYYCQQYGSSPLTFGQGTKVEIKSSASTKGPS |
| | light | VFPLAPSSKSTSGGTAALGCLVKDYFPEPVTVSWNSGAL |
| | chain | TSGVHTFPAVLQSSGLYSLSSVVTVPSSSLGTQTYICNV |
| | | NHKPSNTKVDKKVEPKSC |

Table 45 shows the cDNA and amino acid sequences of the monovalent FAP(4B9)-targeted/DP47 4-1BB ligand (71-254) trimer-containing Fc (kih) fusion antigen binding molecule, wherein the dimeric 4-1BBL is fused to the Fc knob chain (Construct 2.16).

TABLE 45
Sequences of FAP(4B9)-targeted human 4-1BB ligand trimer containing Fc (kih)
fusion molecule Construct 2.16

| SEQ ID | | |
|---|---|---|
| NO: | Description | Sequence |
| 215 | nucleotide | GAGGTGCAGCTGCTCGAAAGCGGCGGAGGACTGGTGCA |
| | sequence of anti- | GCCTGGCGGCAGCCTGAGACTGTCTTGCGCCGCCAGCG |
| | FAP(4B9) Fc | GCTTCACCTTCAGCAGCTACGCCATGAGCTGGGTCCGCC |
| | hole monomeric | AGGCCCCTGGCAAGGGACTGGAATGGGTGTCCGCCATC |
| | 4-1BBL (71-254) | ATCGGCTCTGGCGCCAGCACCTACTACGCCGACAGCGTG |
| | chain | AAGGGCCGGTTCACCATCAGCCGGGACAACAGCAAGAA |
| | | CACCCTGTACCTGCAGATGAACAGCCTGCGGGCCGAGG |
| | | ACACCGCCGTGTACTACTGCGCCAAGGGATGGTTCGGC |
| | | GGCTTCAACTACTGGGGACAGGGCACCCTGGTCACAGT |
| | | GTCCAGCGCTAGCACCAAGGGCCCCTCCGTGTTCCCCCT |
| | | GGCCCCCAGCAGCAAGAGCACCAGCGGCGGCACAGCCG |
| | | CTCTGGGCTGCCTGGTCAAGGACTACTTCCCCGAGCCCG |
| | | TGACCGTGTCCTGGAACAGCGGAGCCCTGACCTCCGGC |
| | | GTGCACACCTTCCCCGCCGTGCTGCAGAGTTCTGGCCTG |
| | | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAAACTCACACATGCCCACCGTGCCCA |
| | | GCACCTGAAGCTGCAGGGGGACCGTCAGTCTTCCTCTTC |
| | | CCCCCAAAACCCAAGGACACCCTCATGATCTCCCGGACC |
| | | CCTGAGGTCACATGCGTGGTGGTGGACGTGAGCCACGA |
| | | AGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTGCACCCTGCCCCCATCCCGGGATGAGCTG |
| | | ACCAAGAACCAGGTCAGCCTCTCGTGCGCAGTCAAAGG |
| | | CTTCTATCCCAGCGACATCGCCGTGGAGTGGGAGAGCA |
| | | ATGGGCAGCCGGAGAACAACTACAAGACCACGCCTCCC |
| | | GTGCTGGACTCCGACGGCTCCTTCTTCCTCGTGAGCAAG |
| | | CTCACCGTGGACAAGAGCAGGTGGCAGCAGGGGAACGT |
| | | CTTCTCATGCTCCGTGATGCATGAGGCTCTGCACAACCA |
| | | CTACACGCAGAAGAGCCTCTCCCTGTCTCCGGGTGGAGG |
| | | CGGCGGAAGCGGAGGAGGAGGATCCAGAGAGGGCCCT |
| | | GAGCTGAGCCCCGATGATCCTGCTGGACTGCTGGACCTG |
| | | CGGCAGGGCATGTTTGCTCAGCTGGTGGCCCAGAACGTG |
| | | CTGCTGATCGATGGCCCCCTGTCCTGGTACAGCGATCCT |
| | | GGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGAAGCGCTGCTGGCGCTGCAG |
| | | CTCTGGCACTGACAGTGGATCTGCCTCCTGCCAGCTCCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCAGCCGGCCTGCCTTCTCCAAGAAGCGAA |
| 216 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | (VH-CL) Fc knob | ATTCACCTTTAGCAGTTATGCCATGAGCTGGGTCCGCCA |
| | dimeric 4-1BB | GGCTCCAGGGAAGGGGCTGGAGTGGGTCTCAGCTATTA |
| | ligand (71-254) | GTGGTAGTGGTGGTAGCACATACTACGCAGACTCCGTGA |
| | chain | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAGACCCACACCTGTCCCCCTTG |
| | | TCCTGCCCCTGAAGCTGCTGGCGGCCCTTCTGTGTTCCT |
| | | GTTCCCCCCAAAGCCCAAGGACACCCTGATGATCAGCC |
| | | GGACCCCCGAAGTGACCTGCGTGGTGGTGGATGTGTCCC |
| | | ACGAGGACCCTGAAGTGAAGTTCAATTGGTACGTGGAC |
| | | GGCGTGGAAGTGCACAACGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTACACCCTGCCCCCCTGCAGAGAT |
| | | GAGCTGACCAAGAACCAGGTGTCCCTGTGGTGTCTGGTC |
| | | AAGGGCTTCTACCCCAGCGATATCGCCGTGGAGTGGGA |
| | | GAGCAACGGCCAGCCTGAGAACAACTACAAGACCACCC |
| | | CCCCTGTGCTGGACAGCGACGGCAGCTTCTTCCTGTACT |
| | | CCAAACTGACCGTGGACAAGAGCCGGTGGCAGCAGGGC |
| | | AACGTGTTCAGCTGCAGCGTGATGCACGAGGCCCTGCA |
| | | CAACCACTACACCCAGAAGTCCCTGAGCCTGAGCCCCG |
| | | GCGGAGGCGGCGGAAGCGGAGGAGGAGGATCCAGAGA |
| | | GGGCCCTGAGCTGAGCCCCGATGATCCTGCTGGACTGCT |
| | | GGACCTGCGGCAGGGCATGTTTGCTCAGCTGGTGGCCCA |
| | | GAACGTGCTGCTGATCGATGGCCCCCTGTCCTGGTACAG |
| | | CGATCCTGGACTGGCTGGCGTGTCACTGACAGGCGGCCT |
| | | GAGCTACAAAGAGGACACCAAAGAACTGGTGGTGGCCA |
| | | AGGCCGGCGTGTACTACGTGTTCTTTCAGCTGGAACTGC |
| | | GGAGAGTGGTGGCCGGCGAAGGATCTGGCTCTGTGTCTC |
| | | TGGCCCTGCATCTGCAGCCTCTGAGAAGCGCTGCTGGCG |
| | | CTGCAGCTCTGGCACTGACAGTGGATCTGCCTCCTGCCA |
| | | GCTCCGAGGCCCGGAATAGCGCATTTGGGTTTCAAGGCA |
| | | GGCTGCTGCACCTGTCTGCCGGCCAGAGGCTGGGAGTG |
| | | CATCTGCACACAGAGGCCAGGGCTAGACACGCCTGGCA |
| | | GCTGACACAGGGCGCTACAGTGCTGGGCCTGTTCAGAGT |
| | | GACCCCCGAGATTCCAGCCGGCCTGCCTTCTCCAAGAAG |
| | | CGAAGGCGGAGGCGGATCTGGCGGCGGAGGATCTAGAG |
| | | AGGGACCCGAACTGTCCCCTGACGATCCAGCCGGGCTG |
| | | CTGGATCTGAGACAGGGAATGTTCGCCCAGCTGGTGGCT |
| | | CAGAATGTGCTGCTGATTGACGGACCTCTGAGCTGGTAC |
| | | TCCGACCCAGGGCTGGCAGGGGTGTCCCTGACTGGGGG |
| | | ACTGTCCTACAAAGAAGATACAAAAGAACTGGTGGTGG |
| | | CTAAAGCTGGGGTGTACTATGTGTTTTTTCAGCTGGAAC |
| | | TGAGGCGGGTGGTGGCTGGGGAGGGCTCAGGATCTGTG |
| | | TCCCTGGCTCTGCATCTGCAGCCACTGCGCTCTGCTGCT |
| | | GGCGCAGCTGCACTGGCTCTGACTGTGGACCTGCCACCA |
| | | GCCTCTAGCGAGGCCAGAAACAGCGCCTTCGGGTTCCA |
| | | AGGACGCCTGCTGCATCTGAGCGCCGGACAGCGCCTGG |
| | | GAGTGCATCTGCATACTGAAGCCAGAGCCCGGCATGCTT |
| | | GGCAGCTGACTCAGGGGGCAACTGTGCTGGGACTGTTTC |
| | | GCGTGACACCTGAGATCCCTGCCGGACTGCCAAGCCCTA |
| | | GATCAGAA |
| 103 | nucleotide | See Table 1 |
| | sequence of anti- | |
| | FAP(4B9) light | |
| | chain | |
| 211 | nucleotide | see Table 42 |
| | sequence of DP47 | |
| | (VL-CH1) light | |
| | chain | |
| 217 | anti-FAP(4B9) Fc | EVQLLESGGGLVQPGGSLRLSCAASGFTFSSYAMSWVRQA |
| | hole monomeric | PGKGLEWVSAIIGSGASTYYADSVKGRFTISRDNSKNTLYL |
| | 4-1BBL (71-254) | QMNSLRAEDTAVYYCAKGWFGGFNYWGQGTLVTVSSAS |
| | chain | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVCTLPPSRDELTKN |
| | | QVSLSCAVKGFYPSDIAVEWESNGQPENNYKTTPPVLDSD |
| | | GSFFLVSKLTVDKSRWQQGNVFSCSVMHEALHNHYTQKS |
| | | LSLSPGGGGGSGGGGSREGPELSPDDPAGLLDLRQGMFAQ |
| | | LVAQNVLLIDGPLSWYSDPGLAGVSLTGGLSYKEDTKELV |
| | | VAKAGVYYVFFQLELRRVVAGEGSGSVSLALHLQPLRSAA |
| | | GAAALALTVDLPPASSEARNSAFGFQGRLLHLSAGQRLGV |
| | | HLHTEARARHAWQLTQGATVLGLFRVTPEIPAGLPSPRSE |
| 218 | DP47 (VH-CL) | EVQLLESGGGLVQPGGSLRLSCAASGFTFSSYAMSWVRQA |
| | Fc knob dimeric | PGKGLEWVSAISGSGGSTYYADSVKGRFTISRDNSKNTLYL |
| | 4-1BB ligand (71- | QMNSLRAEDTAVYYCAKGSGFDYWGQGTLVTVSSASVA |
| | 254) chain | APSVFIFPPSDEQLKSGTASVVCLLNNFYPREAKVQWKVD |
| | | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVYTLPPCRDE |
| | | LTKNQVSLWCLVKGFYPSDIAVEWESNGQPENNYKTTPPV |
| | | LDSDGSFFLYSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGLPS |
| | | PRSEGGGGSGGGGSREGPELSPDDPAGLLDLRQGMFAQLV |
| | | AQNVLLIDGPLSWYSDPGLAGVSLTGGLSYKEDTKELVVA |
| | | KAGVYYVFFQLELRRVVAGEGSGSVSLALHLQPLRSAAGA |
| | | AALALTVDLPPASSEARNSAFGFQGRLLHLSAGQRLGVHL |
| | | HTEARARHAWQLTQGATVLGLFRVTPEIPAGLPSPRSE |
| 106 | anti-FAP(4B9) | See Table 1 |
| | light chain | |
| 214 | DP47 (VL-CH1) | see Table 42 |
| | light chain | |

4.2 Preparation of Monovalent FAP(4B9)-Targeted/DP47 4-1BB Ligand Trimer-containingFc (Kih) Fusion Antigen Binding Molecule, Wherein the 4-1BB Ligands are C-Terminally Fused (Constructs 2.17 and 2.18) (FAP on the Knob Chain)

The DNA sequence encoding part of the ectodomain (amino acids 71-254) of human 4-BB ligand was synthetized according to the P41273 sequence of Uniprot database.

A polypeptide containing two ectodomains of 4-1BB ligand, separated by (G4S)2 linkers was subcloned in frame to the C-terminus of human IgG1 Fc hole or knob chain, as depicted in FIG. 1A: human IgG1 Fc, (G4S)2 connector, human 4-1BB ligand, (G4S)2 connector, human 4-1BB ligand. A polypeptide containing one ectodomain of 4-1BB ligand was subcloned in frame to the C-terminus of human IgG1 Fc knob or hole chain as described in FIG. 1B: human IgG1 Fc, (G4S)2 connector, human 4-1BB ligand.

The variable region of heavy and light chain DNA sequences encoding a binder specific for fibroblast activation protein (FAP), clone 4B9, were subcloned in frame with either the constant heavy chain of the knob or the constant light chain of human IgG1. The variable region of heavy and light chain DNA sequences encoding a non-binding clone, clone DP47, were subcloned in frame with either the constant heavy chain of the hole or the constant light chain of human IgG1. The crossmab technology was applied to reduce the formation of wrongly paired light chains (International patent application No. WO 2010/145792 A1). In this example a crossed Fab unit (VHCL) of the binder DP47 was fused to the knob chain.

The Pro329Gly, Leu234Ala and Leu235Ala mutations have been introduced in the constant region of the knob and hole heavy chains to abrogate binding to Fc gamma receptors (International Patent Appl. Publ. No. WO 2012/130831 A1).

Combination of the DP47 huIgG1 Fc hole chain containing the Y349C/T366S/L368A/Y407V mutations, the anti-FAP huIgG1 Fc knob chain containing the S354C/T366W mutations and the two light chains, anti-FAP and DP47, allows generation of a heterodimer, which includes an assembled trimeric 4-1BB ligand, one FAP binding Fab and one non binding DP47 Fab (FIGS. 5C and 5D).

Table 46 shows the cDNA and amino acid sequences of the monovalent DP47/FAP(4B9)-targeted 4-1BB ligand (71-254) trimer-containing Fc (kih) fusion antigen binding molecule, wherein the dimeric 4-1BBL is fused to Fc hole chain (Construct 2.17).

TABLE 46
Sequences of FAP(4B9)-targeted/DP47 human 4-1BB ligand trimer containing Fc
(kih) fusion antigen binding molecule Construct 2.17

| SEQ ID | | |
|---|---|---|
| NO: | Description | Sequence |
| 219 | nucleotide | GAGGTGCAATTGTTGGAGTCTGGGGGAGGCTTGGTACA |
| | sequence of | GCCTGGGGGGTCCCTGAGACTCTCCTGTGCAGCCTCCGG |
| | DP47 (VH-C L) Fc | ATTCACCTTTAGCAGTTATGCCATGAGCTGGGTCCGCCA |
| | hole dimeric 4- | GGCTCCAGGGAAGGGGCTGGAGTGGGTCTCAGCTATTA |
| | 1BB ligand (71- | GTGGTAGTGGTGGTAGCACATACTACGCAGACTCCGTGA |
| | 254) chain | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAAACTCACACATGCCCACCGTG |
| | | CCCAGCACCTGAAGCTGCAGGGGGACCGTCAGTCTTCCT |
| | | CTTCCCCCCAAAACCCAAGGACACCCTCATGATCTCCCG |
| | | GACCCCTGAGGTCACATGCGTGGTGGTGGACGTGAGCC |
| | | ACGAAGACCCTGAGGTCAAGTTCAACTGGTACGTGGAC |
| | | GGCGTGGAGGTGCATAATGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTGCACCCTGCCCCCATCCCGGGAT |
| | | GAGCTGACCAAGAACCAGGTCAGCCTCTCGTGCGCAGT |
| | | CAAAGGCTTCTATCCCAGCGACATCGCCGTGGAGTGGG |
| | | AGAGCAATGGGCAGCCGGAGAACAACTACAAGACCACG |
| | | CCTCCCGTGCTGGACTCCGACGGCTCCTTCTTCCTCGTG |
| | | AGCAAGCTCACCGTGGACAAGAGCAGGTGGCAGCAGGG |
| | | GAACGTCTTCTCATGCTCCGTGATGCATGAGGCTCTGCA |
| | | CAACCACTACACGCAGAAGAGCCTCTCCCTGTCTCCGGG |
| | | TGGAGGCGGCGGAAGCGGAGGAGGAGGATCCAGAGAG |
| | | GGCCCTGAGCTGAGCCCCGATGATCCTGCTGGACTGCTG |
| | | GACCTGCGGCAGGGCATGTTTGCTCAGCTGGTGGCCCAG |
| | | AACGTGCTGCTGATCGATGGCCCCCTGTCCTGGTACAGC |
| | | GATCCTGGACTGGCTGGCGTGTCACTGACAGGCGGCCTG |
| | | AGCTACAAAGAGGACACCAAAGAACTGGTGGTGGCCAA |
| | | GGCCGGCGTGTACTACGTGTTCTTTCAGCTGGAACTGCG |
| | | GAGAGTGGTGGCCGGCGAAGGATCTGGCTCTGTGTCTCT |
| | | GGCCCTGCATCTGCAGCCTCTGAGAAGCGCTGCTGGCGC |
| | | TGCAGCTCTGGCACTGACAGTGGATCTGCCTCCTGCCAG |
| | | CTCCGAGGCCCGGAATAGCGCATTTGGGTTTCAAGGCAG |
| | | GCTGCTGCACCTGTCTGCCGGCCAGAGGCTGGGAGTGC |
| | | ATCTGCACACAGAGGCCAGGGCTAGACACGCCTGGCAG |
| | | CTGACACAGGGCGCTACAGTGCTGGGCCTGTTCAGAGTG |
| | | ACCCCCGAGATTCCAGCCGGCCTGCCTTCTCCAAGAAGC |
| | | GAAGGCGGAGGCGGATCTGGCGGCGGAGGATCTAGAGA |
| | | GGGACCCGAACTGTCCCCTGACGATCCAGCCGGGCTGCT |
| | | GGATCTGAGACAGGGAATGTTCGCCCAGCTGGTGGCTC |
| | | AGAATGTGCTGCTGATTGACGGACCTCTGAGCTGGTACT |
| | | CCGACCCAGGGCTGGCAGGGGTGTCCCTGACTGGGGGA |
| | | CTGTCCTACAAAGAAGATACAAAAGAACTGGTGGTGGC |
| | | TAAAGCTGGGGTGTACTATGTGTTTTTTCAGCTGGAACT |
| | | GAGGCGGGTGGTGGCTGGGGAGGGCTCAGGATCTGTGT |
| | | CCCTGGCTCTGCATCTGCAGCCACTGCGCTCTGCTGCTG |
| | | GCGCAGCTGCACTGGCTCTGACTGTGGACCTGCCACCAG |
| | | CCTCTAGCGAGGCCAGAAACAGCGCCTTCGGGTTCCAA |
| | | GGACGCCTGCTGCATCTGAGCGCCGGACAGCGCCTGGG |
| | | AGTGCATCTGCATACTGAAGCCAGAGCCCGGCATGCTTG |
| | | GCAGCTGACTCAGGGGGCAACTGTGCTGGGACTGTTTCG |
| | | CGTGACACCTGAGATCCCTGCCGGACTGCCAAGCCCTAG |
| | | ATCAGAA |
| 220 | nucleotide | GAGGTGCAGCTGCTCGAAAGCGGCGGAGGACTGGTGCA |
| | sequence of FAP | GCCTGGCGGCAGCCTGAGACTGTCTTGCGCCGCCAGCG |
| | (4B9) Fc knob | GCTTCACCTTCAGCAGCTACGCCATGAGCTGGGTCCGCC |
| | monomeric 4- | AGGCCCCTGGCAAGGGACTGGAATGGGTGTCCGCCATC |
| | 1BB (71-254) | ATCGGCTCTGGCGCCAGCACCTACTACGCCGACAGCGTG |
| | ligand | AAGGGCCGGTTCACCATCAGCCGGGACAACAGCAAGAA |
| | | CACCCTGTACCTGCAGATGAACAGCCTGCGGGCCGAGG |
| | | ACACCGCCGTGTACTACTGCGCCAAGGGATGGTTCGGC |
| | | GGCTTCAACTACTGGGGACAGGGCACCCTGGTCACAGT |
| | | GTCCAGCGCTAGCACCAAGGGCCCCTCCGTGTTCCCCCT |
| | | GGCCCCCAGCAGCAAGAGCACCAGCGGCGGCACAGCCG |
| | | CTCTGGGCTGCCTGGTCAAGGACTACTTCCCCGAGCCCG |
| | | TGACCGTGTCCTGGAACAGCGGAGCCCTGACCTCCGGC |
| | | GTGCACACCTTCCCCGCCGTGCTGCAGAGTTCTGGCCTG |
| | | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAGACCCACACCTGTCCCCCTTGTCCTG |
| | | CCCCTGAAGCTGCTGGCGGCCCTTCTGTGTTCCTGTTCCC |
| | | CCCAAAGCCCAAGGACACCCTGATGATCAGCCGGACCC |
| | | CCGAAGTGACCTGCGTGGTGGTGGATGTGTCCCACGAG |
| | | GACCCTGAAGTGAAGTTCAATTGGTACGTGGACGGCGT |
| | | GGAAGTGCACAACGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTACACCCTGCCCCCCTGCAGAGATGAGCTG |
| | | ACCAAGAACCAGGTGTCCCTGTGGTGTCTGGTCAAGGGC |
| | | TTCTACCCCAGCGATATCGCCGTGGAGTGGGAGAGCAA |
| | | CGGCCAGCCTGAGAACAACTACAAGACCACCCCCCCTG |
| | | TGCTGGACAGCGACGGCAGCTTCTTCCTGTACTCCAAAC |
| | | TGACCGTGGACAAGAGCCGGTGGCAGCAGGGCAACGTG |
| | | TTCAGCTGCAGCGTGATGCACGAGGCCCTGCACAACCA |
| | | CTACACCCAGAAGTCCCTGAGCCTGAGCCCCGGCGGAG |
| | | GCGGCGGAAGCGGAGGAGGAGGATCCAGAGAGGGCCC |
| | | TGAGCTGAGCCCCGATGATCCTGCTGGACTGCTGGACCT |
| | | GCGGCAGGGCATGTTTGCTCAGCTGGTGGCCCAGAACGT |
| | | GCTGCTGATCGATGGCCCCCTGTCCTGGTACAGCGATCC |
| | | TGGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGAAGCGCTGCTGGCGCTGCAG |
| | | CTCTGGCACTGACAGTGGATCTGCCTCCTGCCAGCTCCG |
| | | AGGCCCGGAATAGC GCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCAGCCGGCCTGCCTTCTCCAAGAAGCGAA |
| 103 | nucleotide | see Table 1 |
| | sequence of anti- | |
| | FAP(4B9) light | |
| | chain | |
| 211 | nucleotide | see Table 42 |
| | sequence of DP47 | |
| | (VL-CH1) light | |
| | chain | |
| 221 | DP47(VH-CL) Fc | EVQLLESGGGLVQPGGSLRLSCAASGFTFSSYAMSWVRQA |
| | hole dimeric 4- | PGKGLEWVSAISGSGGSTYYADSVKGRFTISRDNSKNTLYL |
| | 1BB ligand (71- | QMNSLRAEDTAVYYCAKGSGFDYWGQGTLVTVSSASVA |
| | 254) chain | APSVFIFPPSDEQLKSGTASVVCLLNNFYPREAKVQWKVD |
| | | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVCTLPPSRDE |
| | | LTKNQVSLSCAVKGFYPSDIAVEWESNGQPENNYKTTPPV |
| | | LDSDGSFFLVSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGLPS |
| | | PRSEGGGGSGGGGSREGPELSPDDPAGLLDLRQGMFAQLV |
| | | AQNVLLIDGPLSWYSDPGLAGVSLTGGLSYKEDTKELVVA |
| | | KAGVYYVFFQLELRRVVAGEGSGSVSLALHLQPLRSAAGA |
| | | AALALTVDLPPASSEARNSAFGFQGRLLHLSAGQRLGVHL |
| | | HTEARARHAWQLTQGATVLGLFRVTPEIPAGLPSPRSE |
| 222 | FAP (4B9) Fc | EVQLLESGGGLVQPGGSLRLSCAASGFTFSSYAMSWVRQA |
| | knob monomenc | PGKGLEWVSAIIGSGASTYYADSVKGRFTISRDNSKNTLYL |
| | 4-1BB (71-254) | QMNSLRAEDTAVYYCAKGWFGGFNYWGQGTLVTVSSAS |
| | ligand | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGGGGGSGGGGSREGPELSPDDPAGLLDLRQGMFAQ |
| | | LVAQNVLLIDGPLSWYSDPGLAGVSLTGGLSYKEDTKELV |
| | | VAKAGVYYVFFQLELRRVVAGEGSGSVSLALHLQPLRSAA |
| | | GAAALALTVDLPPASSEARNSAFGFQGRLLHLSAGQRLGV |
| | | HLHTEARARHAWQLTQGATVLGLFRVTPEIPAGLPSPRSE |
| 106 | anti-FAP(4B9) | see Table 1 |
| | light chain | |
| 214 | DP47 (VL-CH1) | see Table 42 |
| | light chain | |

Table 47 shows the cDNA and amino acid sequences of the monovalent DP47/FAP(4B9)-targeted 4-1BB ligand (71-254)trimer-containing Fc (kih) fusion antigen binding molecule, wherein the dimeric 4-1BBL is fused to the knob chain (Construct 2.18).

TABLE 47
Sequences of FAP(4B9)-targeted human 4-1BB ligand trimer containing Fc (kih)
fusion molecule Construct 2.18

| SEQ ID | | |
|---|---|---|
| NO: | Description | Sequence |
| 223 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | (VH-CL) Fc hole | ATTCACCTTTAGCAGTTATGCCATGAGCTGGGTCCGCCA |
| | monomeric | GGCTCCAGGGAAGGGGCTGGAGTGGGTCTCAGCTATTA |
| | 4-1BBL (71-254) | GTGGTAGTGGTGGTAGCACATACTACGCAGACTCCGTGA |
| | chain | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAAACTCACACATGCCCACCGTG |
| | | CCCAGCACCTGAAGCTGCAGGGGGACCGTCAGTCTTCCT |
| | | CTTCCCCCCAAAACCCAAGGACACCCTCATGATCTCCCG |
| | | GACCCCTGAGGTCACATGCGTGGTGGTGGACGTGAGCC |
| | | ACGAAGACCCTGAGGTCAAGTTCAACTGGTACGTGGAC |
| | | GGCGTGGAGGTGCATAATGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTGCACCCTGCCCCCATCCCGGGAT |
| | | GAGCTGACCAAGAACCAGGTCAGCCTCTCGTGCGCAGT |
| | | CAAAGGCTTCTATCCCAGCGACATCGCCGTGGAGTGGG |
| | | AGAGCAATGGGCAGCCGGAGAACAACTACAAGACCACG |
| | | CCTCCCGTGCTGGACTCCGACGGCTCCTTCTTCCTCGTG |
| | | AGCAAGCTCACCGTGGACAAGAGCAGGTGGCAGCAGGG |
| | | GAACGTCTTCTCATGCTCCGTGATGCATGAGGCTCTGCA |
| | | CAACCACTACACGCAGAAGAGCCTCTCCCTGTCTCCGGG |
| | | TGGAGGCGGCGGAAGCGGAGGAGGAGGATCCAGAGAG |
| | | GGCCCTGAGCTGAGCCCCGATGATCCTGCTGGACTGCTG |
| | | GACCTGCGGCAGGGCATGTTTGCTCAGCTGGTGGCCCAG |
| | | AACGTGCTGCTGATCGATGGCCCCCTGTCCTGGTACAGC |
| | | GATCCTGGACTGGCTGGCGTGTCACTGACAGGCGGCCTG |
| | | AGCTACAAAGAGGACACCAAAGAACTGGTGGTGGCCAA |
| | | GGCCGGCGTGTACTACGTGTTCTTTCAGCTGGAACTGCG |
| | | GAGAGTGGTGGCCGGCGAAGGATCTGGCTCTGTGTCTCT |
| | | GGCCCTGCATCTGCAGCCTCTGAGAAGCGCTGCTGGCGC |
| | | TGCAGCTCTGGCACTGACAGTGGATCTGCCTCCTGCCAG |
| | | CTCCGAGGCCCGGAATAGCGCATTTGGGTTTCAAGGCAG |
| | | GCTGCTGCACCTGTCTGCCGGCCAGAGGCTGGGAGTGC |
| | | ATCTGCACACAGAGGCCAGGGCTAGACACGCCTGGCAG |
| | | CTGACACAGGGCGCTACAGTGCTGGGCCTGTTCAGAGTG |
| | | ACCCCCGAGATTCCAGCCGGCCTGCCTTCTCCAAGAAGC |
| | | GAA |
| 224 | nucleotide | GAGGTGCAGCTGCTCGAAAGCGGCGGAGGACTGGTGCA |
| | sequence of FAP | GCCTGGCGGCAGCCTGAGACTGTCTTGCGCCGCCAGCG |
| | (4B9) Fc knob | GCTTCACCTTCAGCAGCTACGCCATGAGCTGGGTCCGCC |
| | dimeric 4-1BB | AGGCCCCTGGCAAGGGACTGGAATGGGTGTCCGCCATC |
| | ligand (71-254) | ATCGGCTCTGGCGCCAGCACCTACTACGCCGACAGCGTG |
| | chain | AAGGGCCGGTTCACCATCAGCCGGGACAACAGCAAGAA |
| | | CACCCTGTACCTGCAGATGAACAGCCTGCGGGCCGAGG |
| | | ACACCGCCGTGTACTACTGCGCCAAGGGATGGTTCGGC |
| | | GGCTTCAACTACTGGGGACAGGGCACCCTGGTCACAGT |
| | | GTCCAGCGCTAGCACCAAGGGCCCCTCCGTGTTCCCCCT |
| | | GGCCCCCAGCAGCAAGAGCACCAGCGGCGGCACAGCCG |
| | | CTCTGGGCTGCCTGGTCAAGGACTACTTCCCCGAGCCCG |
| | | TGACCGTGTCCTGGAACAGCGGAGCCCTGACCTCCGGC |
| | | GTGCACACCTTCCCCGCCGTGCTGCAGAGTTCTGGCCTG |
| | | TATAGCCTGAGCAGCGTGGTCACCGTGCCTTCTAGCAGC |
| | | CTGGGCACCCAGACCTACATCTGCAACGTGAACCACAA |
| | | GCCCAGCAACACCAAGGTGGACAAGAAGGTGGAGCCCA |
| | | AGAGCTGCGACAAGACCCACACCTGTCCCCCTTGTCCTG |
| | | CCCCTGAAGCTGCTGGCGGCCCTTCTGTGTTCCTGTTCCC |
| | | CCCAAAGCCCAAGGACACCCTGATGATCAGCCGGACCC |
| | | CCGAAGTGACCTGCGTGGTGGTGGATGTGTCCCACGAG |
| | | GACCCTGAAGTGAAGTTCAATTGGTACGTGGACGGCGT |
| | | GGAAGTGCACAACGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCGGCGCCCCCATCG |
| | | AGAAAACCATCTCCAAAGCCAAAGGGCAGCCCCGAGAA |
| | | CCACAGGTGTACACCCTGCCCCCCTGCAGAGATGAGCTG |
| | | ACCAAGAACCAGGTGTCCCTGTGGTGTCTGGTCAAGGGC |
| | | TTCTACCCCAGCGATATCGCCGTGGAGTGGGAGAGCAA |
| | | CGGCCAGCCTGAGAACAACTACAAGACCACCCCCCCTG |
| | | TGCTGGACAGCGACGGCAGCTTCTTCCTGTACTCCAAAC |
| | | TGACCGTGGACAAGAGCCGGTGGCAGCAGGGCAACGTG |
| | | TTCAGCTGCAGCGTGATGCACGAGGCCCTGCACAACCA |
| | | CTACACCCAGAAGTCCCTGAGCCTGAGCCCCGGCGGAG |
| | | GCGGCGGAAGCGGAGGAGGAGGATCCAGAGAGGGCCC |
| | | TGAGCTGAGCCCCGATGATCCTGCTGGACTGCTGGACCT |
| | | GCGGCAGGGCATGTTTGCTCAGCTGGTGGCCCAGAACGT |
| | | GCTGCTGATCGATGGCCCCCTGTCCTGGTACAGCGATCC |
| | | TGGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGAAGCGCTGCTGGCGCTGCAG |
| | | CTCTGGCACTGACAGTGGATCTGCCTCCTGCCAGCTCCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCAGCCGGCCTGCCTTCTCCAAGAAGCGAA |
| | | GGCGGAGGCGGATCTGGCGGCGGAGGATCTAGAGAGGG |
| | | ACCCGAACTGTCCCCTGACGATCCAGCCGGGCTGCTGGA |
| | | TCTGAGACAGGGAATGTTCGCCCAGCTGGTGGCTCAGA |
| | | ATGTGCTGCTGATTGACGGACCTCTGAGCTGGTACTCCG |
| | | ACCCAGGGCTGGCAGGGGTGTCCCTGACTGGGGGACTG |
| | | TCCTACAAAGAAGATACAAAAGAACTGGTGGTGGCTAA |
| | | AGCTGGGGTGTACTATGTGTTTTTTCAGCTGGAACTGAG |
| | | GCGGGTGGTGGCTGGGGAGGGCTCAGGATCTGTGTCCCT |
| | | GGCTCTGCATCTGCAGCCACTGCGCTCTGCTGCTGGCGC |
| | | AGCTGCACTGGCTCTGACTGTGGACCTGCCACCAGCCTC |
| | | TAGCGAGGCCAGAAACAGCGCCTTCGGGTTCCAAGGAC |
| | | GCCTGCTGCATCTGAGCGCCGGACAGCGCCTGGGAGTG |
| | | CATCTGCATACTGAAGCCAGAGCCCGGCATGCTTGGCA |
| | | GCTGACTCAGGGGGCAACTGTGCTGGGACTGTTTCGCGT |
| | | GACACCTGAGATCCCTGCCGGACTGCCAAGCCCTAGATC |
| | | AGAA |
| 103 | nucleotide | See Table 1 |
| | sequence of anti- | |
| | FAP(4B9) light | |
| | chain | |
| 211 | nucleotide | see Table 42 |
| | sequence of DP47 | |
| | (VL-CH1) light | |
| | chain | |
| 225 | DP47 (VH-CL) | EVQLLESGGGLVQPGGSLRLSCAASGFTFSSYAMSWVRQA |
| | Fc hole | PGKGLEWVSAISGSGGSTYYADSVKGRFTISRDNSKNTLYL |
| | monomeric 4- | QMNSLRAEDTAVYYCAKGSGFDYWGQGTLVTVSSASVA |
| | 1BBL (71-254) | APSVFIFPPSDEQLKSGTASVVCLLNNFYPREAKVQWKVD |
| | chain | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVCTLPPSRDE |
| | | LTKNQVSLSCAVKGFYPSDIAVEWESNGQPENNYKTTPPV |
| | | LDSDGSFFLVSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGLPS |
| | | PRSE |
| 226 | anti-FAP(4B9) Fc | EVQLLESGGGLVQPGGSLRLSCAASGFTFSSYAMSWVRQA |
| | knob dimeric | PGKGLEWVSAIIGSGASTYYADSVKGRFTISRDNSKNTLYL |
| | 4-1BB ligand | QMNSLRAEDTAVYYCAKGWFGGFNYWGQGTLVTVSSAS |
| | (71-254) chain | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPEAAGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPCRDELTKN |
| | | QVSLWCLVKGFYPSDIAVEWESNGQPENNYKTTPPVLDSD |
| | | GSFFLYSKLTVDKSRWQQGNVFSCSVMHEALHNHYTQKS |
| | | LSLSPGGGGGSGGGGSREGPELSPDDPAGLLDLRQGMFAQ |
| | | LVAQNVLLIDGPLSWYSDPGLAGVSLTGGLSYKEDTKELV |
| | | VAKAGVYYVFFQLELRRVVAGEGSGSVSLALHLQPLRSAA |
| | | GAAALALTVDLPPASSEARNSAFGFQGRLLHLSAGQRLGV |
| | | HLHTEARARHAWQLTQGATVLGLFRVTPEIPAGLPSPRSEG |
| | | GGGSGGGGSREGPELSPDDPAGLLDLRQGMFAQLVAQNV |
| | | LLIDGPLSWYSDPGLAGVSLTGGLSYKEDTKELVVAKAGV |
| | | YYVFFQLELRRVVAGEGSGSVSLALHLQPLRSAAGAAALA |
| | | LTVDLPPASSEARNSAFGFQGRLLHLSAGQRLGVHLHTEA |
| | | RARHAWQLTQGATVLGLFRVTPEIPAGLPSPRSE |
| 106 | anti-FAP(4B9) | See Table 1 |
| | light chain | |
| 214 | DP47 (VL-CH1) | see Table 42 |
| | light chain | |

4.3 Preparation of Monovalent FAP(4B9)-Targeted/DP47 4-1BB Ligand Trimer-containingFc (Kih) Fusion Antigen Binding Molecule, Wherein the 4-1BB Ligands are C-Terminally Fused (Constructs 2.19 and 2.20) (FAP on the Hole Chain)

The DNA sequence encoding part of the ectodomain (amino acids 71-248) of human 4-1 BB ligand was synthetized according to the P41273 sequence of Uniprot database. Constructs 2.19 and 2.20 were prepared as described in Example 4.1 for Constructs 2.15 and 2.16.

Table 48 shows the cDNA and amino acid sequences of the monovalent FAP(4B9)-targeted/DP47 4-1BB ligand (71-248)trimer-containing Fc (kih) fusion antigen binding molecule, wherein the dimeric 4-1BBL is fused to the hole chain (Construct 2.19).

TABLE 48
Sequences of FAP(4B9)-targeted/DP47 human 4-1BB ligand trimer containing Fc
(kih) fusion antigen binding molecule Construct 2.19

| SEQ ID | | |
|---|---|---|
| NO: | Description | Sequence |
| 171 | nucleotide | see Table 23 |
| | sequence of anti- | |
| | FAP(4B9) Fc | |
| | hole dimeric 4- | |
| | 1BB ligand (71- | |
| | 248) chain | |
| 227 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | (VHCL) Fc knob | ATTCACCTTTAGCAGTTATGCCATGAGCTGGGTCCGCCA |
| | monomeric 4- | GGCTCCAGGGAAGGGGCTGGAGTGGGTCTCAGCTATTA |
| | 1BB (71-248) | GTGGTAGTGGTGGTAGCACATACTACGCAGACTCCGTGA |
| | ligand | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAGACCCACACCTGTCCCCCTTG |
| | | TCCTGCCCCTGAAGCTGCTGGCGGCCCTTCTGTGTTCCT |
| | | GTTCCCCCCAAAGCCCAAGGACACCCTGATGATCAGCC |
| | | GGACCCCCGAAGTGACCTGCGTGGTGGTGGATGTGTCCC |
| | | ACGAGGACCCTGAAGTGAAGTTCAATTGGTACGTGGAC |
| | | GGCGTGGAAGTGCACAACGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTACACCCTGCCCCCCTGCAGAGAT |
| | | GAGCTGACCAAGAACCAGGTGTCCCTGTGGTGTCTGGTC |
| | | AAGGGCTTCTACCCCAGCGATATCGCCGTGGAGTGGGA |
| | | GAGCAACGGCCAGCCTGAGAACAACTACAAGACCACCC |
| | | CCCCTGTGCTGGACAGCGACGGCAGCTTCTTCCTGTACT |
| | | CCAAACTGACCGTGGACAAGAGCCGGTGGCAGCAGGGC |
| | | AACGTGTTCAGCTGCAGCGTGATGCACGAGGCCCTGCA |
| | | CAACCACTACACCCAGAAGTCCCTGAGCCTGAGCCCCG |
| | | GCGGAGGCGGCGGAAGCGGAGGAGGAGGATCCAGAGA |
| | | GGGCCCTGAGCTGAGCCCTGATGATCCTGCCGGACTGCT |
| | | GGACCTGCGGCAGGGAATGTTTGCCCAGCTGGTGGCCC |
| | | AGAACGTGCTGCTGATCGATGGCCCCCTGTCCTGGTACA |
| | | GCGATCCTGGACTGGCTGGCGTGTCACTGACAGGCGGC |
| | | CTGAGCTACAAAGAGGACACCAAAGAACTGGTGGTGGC |
| | | CAAGGCCGGCGTGTACTACGTGTTCTTTCAGCTGGAACT |
| | | GCGGAGAGTGGTGGCCGGCGAAGGATCTGGCTCTGTGT |
| | | CTCTGGCCCTGCATCTGCAGCCTCTGAGATCTGCTGCTG |
| | | GCGCCGCTGCTCTGGCACTGACAGTGGATCTGCCTCCTG |
| | | CCAGCAGCGAGGCCCGGAATAGCGCATTTGGGTTTCAA |
| | | GGCAGGCTGCTGCACCTGTCTGCCGGCCAGAGGCTGGG |
| | | AGTGCATCTGCACACAGAGGCCAGGGCTAGACACGCCT |
| | | GGCAGCTGACACAGGGCGCTACAGTGCTGGGCCTGTTC |
| | | AGAGTGACCCCCGAGATTCCTGCCGGGCTC |
| 103 | nucleotide | see Table 1 |
| | sequence of anti- | |
| | FAP(4B9) light | |
| | chain | |
| 211 | nucleotide | see Table 42 |
| | sequence of DP47 | |
| | (VL-CH1) light | |
| | chain | |
| 173 | anti-FAP(4B9) Fc | see Table 23 |
| | hole dimeric 4- | |
| | 1BB ligand (71- | |
| | 248) chain | |
| 228 | DP47 (VH-CL) | EVQLLESGGGLVQPGGSLRLSCAASGFTFSSYAMSWVRQA |
| | Fc knob | PGKGLEWVSAISGSGGSTYYADSVKGRFTISRDNSKNTLYL |
| | monomeric 4- | QMNSLRAEDTAVYYCAKGSGFDYWGQGTLVTVSSASVA |
| | 1BB (71-248) | APSVFIFPPSDEQLKSGTASVVCLLNNFYPREAKVQWKVD |
| | ligand | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVYTLPPCRDE |
| | | LTKNQVSLWCLVKGFYPSDIAVEWESNGQPENNYKTTPPV |
| | | LDSDGSFFLYSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGL |
| 106 | anti-FAP(4B9) | see Table 1 |
| | light chain | |
| 214 | DP47 (VL-CH1) | see Table 42 |
| | light chain | |

Table 49 shows the cDNA and amino acid sequences of the monovalent FAP(4B9)-targeted/DP47 4-1BB ligand (71-248)trimer-containing Fc (kih) fusion antigen binding molecule, wherein the dimeric 4-1BBL is fused to the hole chain (Construct 2.20).

TABLE 49
Sequences of FAP(4B9)-targeted human 4-1BB ligand trimer containing Fc (kih)
fusion molecule Construct 2.20

| SEQ ID | | |
|---|---|---|
| NO: | Description | Sequence |
| 175 | nucleotide | see Table 24 |
| | sequence of anti- | |
| | FAP(4B9) Fc | |
| | hole monomeric | |
| | 4-1BBL (71-248) | |
| | chain | |
| 229 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | (VH-CL) Fc knob | ATTCACCTTTAGCAGTTATGCCATGAGCTGGGTCCGCCA |
| | dimeric 4-1BB | GGCTCCAGGGAAGGGGCTGGAGTGGGTCTCAGCTATTA |
| | ligand (71-248) | GTGGTAGTGGTGGTAGCACATACTACGCAGACTCCGTGA |
| | chain | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAGACCCACACCTGTCCCCCTTG |
| | | TCCTGCCCCTGAAGCTGCTGGCGGCCCTTCTGTGTTCCT |
| | | GTTCCCCCCAAAGCCCAAGGACACCCTGATGATCAGCC |
| | | GGACCCCCGAAGTGACCTGCGTGGTGGTGGATGTGTCCC |
| | | ACGAGGACCCTGAAGTGAAGTTCAATTGGTACGTGGAC |
| | | GGCGTGGAAGTGCACAACGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTACACCCTGCCCCCCTGCAGAGAT |
| | | GAGCTGACCAAGAACCAGGTGTCCCTGTGGTGTCTGGTC |
| | | AAGGGCTTCTACCCCAGCGATATCGCCGTGGAGTGGGA |
| | | GAGCAACGGCCAGCCTGAGAACAACTACAAGACCACCC |
| | | CCCCTGTGCTGGACAGCGACGGCAGCTTCTTCCTGTACT |
| | | CCAAACTGACCGTGGACAAGAGCCGGTGGCAGCAGGGC |
| | | AACGTGTTCAGCTGCAGCGTGATGCACGAGGCCCTGCA |
| | | CAACCACTACACCCAGAAGTCCCTGAGCCTGAGCCCCG |
| | | GCGGAGGCGGCGGAAGCGGAGGAGGAGGATCCAGAGA |
| | | GGGCCCTGAGCTGAGCCCTGATGATCCTGCCGGACTGCT |
| | | GGACCTGCGGCAGGGAATGTTTGCCCAGCTGGTGGCCC |
| | | AGAACGTGCTGCTGATCGATGGCCCCCTGTCCTGGTACA |
| | | GCGATCCTGGACTGGCTGGCGTGTCACTGACAGGCGGC |
| | | CTGAGCTACAAAGAGGACACCAAAGAACTGGTGGTGGC |
| | | CAAGGCCGGCGTGTACTACGTGTTCTTTCAGCTGGAACT |
| | | GCGGAGAGTGGTGGCCGGCGAAGGATCTGGCTCTGTGT |
| | | CTCTGGCCCTGCATCTGCAGCCTCTGAGATCTGCTGCTG |
| | | GCGCCGCTGCTCTGGCACTGACAGTGGATCTGCCTCCTG |
| | | CCAGCAGCGAGGCCCGGAATAGCGCATTTGGGTTTCAA |
| | | GGCAGGCTGCTGCACCTGTCTGCCGGCCAGAGGCTGGG |
| | | AGTGCATCTGCACACAGAGGCCAGGGCTAGACACGCCT |
| | | GGCAGCTGACACAGGGCGCTACAGTGCTGGGCCTGTTC |
| | | AGAGTGACCCCCGAGATTCCAGCAGGCCTGGGAGGCGG |
| | | CGGATCTGGCGGCGGAGGATCTAGAGAAGGACCCGAGC |
| | | TGTCCCCCGACGATCCCGCTGGGCTGCTGGATCTGAGAC |
| | | AGGGCATGTTCGCTCAGCTGGTGGCTCAGAATGTGCTGC |
| | | TGATTGACGGACCTCTGAGCTGGTACTCCGACCCAGGGC |
| | | TGGCAGGGGTGTCCCTGACTGGGGGACTGTCCTACAAA |
| | | GAAGATACAAAAGAACTGGTGGTGGCTAAAGCTGGGGT |
| | | GTACTATGTGTTTTTTCAGCTGGAACTGAGGCGGGTGGT |
| | | GGCTGGGGAGGGCTCAGGATCTGTGTCCCTGGCTCTGCA |
| | | TCTGCAGCCACTGCGCTCTGCAGCAGGGGCTGCAGCACT |
| | | GGCCCTGACTGTGGACCTGCCCCCAGCTTCTTCCGAGGC |
| | | CAGAAACAGCGCCTTCGGGTTCCAAGGACGCCTGCTGC |
| | | ATCTGAGCGCCGGACAGCGCCTGGGAGTGCATCTGCAT |
| | | ACTGAAGCCAGAGCCCGGCATGCTTGGCAGCTGACTCA |
| | | GGGGGCAACTGTGCTGGGACTGTTTCGCGTGACACCTGA |
| | | GATCCCAGCCGGGCTC |
| 103 | nucleotide | See Table 1 |
| | sequence of anti- | |
| | FAP(4B9) light | |
| | chain | |
| 211 | nucleotide | see Table 42 |
| | sequence of DP47 | |
| | (VL-CH1) light | |
| | chain | |
| 177 | anti-FAP(4B9) Fc | see Table 24 |
| | hole monomeric | |
| | 4-1BBL (71-248) | |
| | chain | |
| 230 | DP47 (VH-CL) | EVQLLESGGGLVQPGGSLRLSCAASGFTFSSYAMSWVRQA |
| | Fc knob dimeric | PGKGLEWVSAISGSGGSTYYADSVKGRFTISRDNSKNTLYL |
| | 4-1BB ligand (71- | QMNSLRAEDTAVYYCAKGSGFDYWGQGTLVTVSSASVA |
| | 248) chain | APSVFIFPPSDEQLKSGTASVVCLLNNFYPREAKVQWKVD |
| | | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVYTLPPCRDE |
| | | LTKNQVSLWCLVKGFYPSDIAVEWESNGQPENNYKTTPPV |
| | | LDSDGSFFLYSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGLGG |
| | | GGSGGGGSREGPELSPDDPAGLLDLRQGMFAQLVAQNVL |
| | | LIDGPLSWYSDPGLAGVSLTGGLSYKEDTKELVVAKAGVY |
| | | YVFFQLELRRVVAGEGSGSVSLALHLQPLRSAAGAAALAL |
| | | TVDLPPASSEARNSAFGFQGRLLHLSAGQRLGVHLHTEAR |
| | | ARHAWQLTQGATVLGLFRVTPEIPAGL |
| 106 | anti-FAP(4B9) | See Table 1 |
| | light chain | |
| 214 | DP47 (VL-CH1) | see Table 42 |
| | light chain | |

4.4 Preparation of Monovalent DP47/FAP(4B9)-Targeted 4-1BB Ligand Trimer-Containing Fc(Kih) Fusion Antigen Binding Molecules, Wherein the 4-1BB Ligands are C-Terminally Fused (Constructs 2.21 and 2.22) (FAP on the Knob Chain)

The DNA sequence encoding part of the ectodomain (amino acids 71-248) of human 4-1 BB ligand was synthetized according to the P41273 sequence of Uniprot database. Constructs 2.21 and 2.22 were prepared as described in Example 4.2 for Constructs 2.17 and 2.18.

Table 50 shows the cDNA and amino acid sequences of the monovalent DP47/FAP(4B9)-targeted 4-1BB ligand (71-254)trimer-containing Fc (kih) fusion antigen binding molecule, wherein the dimeric 4-1BBL is fused to Fc hole chain (Construct 2.21).

TABLE 50
Sequences of FAP(4B9)-targeted/DP47 human 4-1BB ligand trimer containing Fc
(kih) fusion antigen binding molecule Construct 2.21

| SEQ ID | | |
|---|---|---|
| NO: | Description | Sequence |
| 231 | nucleotide | GAGGTGCAATTGTTGGAGTCTGGGGGAGGCTTGGTACA |
| | sequence of | GCCTGGGGGGTCCCTGAGACTCTCCTGTGCAGCCTCCGG |
| | DP47(VH-CL) Fc | ATTCACCTTTAGCAGTTATGCCATGAGCTGGGTCCGCCA |
| | hole dimeric 4- | GGCTCCAGGGAAGGGGCTGGAGTGGGTCTCAGCTATTA |
| | 1BB ligand (71- | GTGGTAGTGGTGGTAGCACATACTACGCAGACTCCGTGA |
| | 248) chain | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAAACTCACACATGCCCACCGTG |
| | | CCCAGCACCTGAAGCTGCAGGGGGACCGTCAGTCTTCCT |
| | | CTTCCCCCCAAAACCCAAGGACACCCTCATGATCTCCCG |
| | | GACCCCTGAGGTCACATGCGTGGTGGTGGACGTGAGCC |
| | | ACGAAGACCCTGAGGTCAAGTTCAACTGGTACGTGGAC |
| | | GGCGTGGAGGTGCATAATGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTGCACCCTGCCCCCATCCCGGGAT |
| | | GAGCTGACCAAGAACCAGGTCAGCCTCTCGTGCGCAGT |
| | | CAAAGGCTTCTATCCCAGCGACATCGCCGTGGAGTGGG |
| | | AGAGCAATGGGCAGCCGGAGAACAACTACAAGACCACG |
| | | CCTCCCGTGCTGGACTCCGACGGCTCCTTCTTCCTCGTG |
| | | AGCAAGCTCACCGTGGACAAGAGCAGGTGGCAGCAGGG |
| | | GAACGTCTTCTCATGCTCCGTGATGCATGAGGCTCTGCA |
| | | CAACCACTACACGCAGAAGAGCCTCTCCCTGTCTCCGGG |
| | | TGGAGGCGGCGGAAGCGGAGGAGGAGGATCCAGAGAG |
| | | GGCCCTGAGCTGAGCCCTGATGATCCTGCCGGACTGCTG |
| | | GACCTGCGGCAGGGAATGTTTGCCCAGCTGGTGGCCCA |
| | | GAACGTGCTGCTGATCGATGGCCCCCTGTCCTGGTACAG |
| | | CGATCCTGGACTGGCTGGCGTGTCACTGACAGGCGGCCT |
| | | GAGCTACAAAGAGGACACCAAAGAACTGGTGGTGGCCA |
| | | AGGCCGGCGTGTACTACGTGTTCTTTCAGCTGGAACTGC |
| | | GGAGAGTGGTGGCCGGCGAAGGATCTGGCTCTGTGTCTC |
| | | TGGCCCTGCATCTGCAGCCTCTGAGATCTGCTGCTGGCG |
| | | CCGCTGCTCTGGCACTGACAGTGGATCTGCCTCCTGCCA |
| | | GCAGCGAGGCCCGGAATAGCGCATTTGGGTTTCAAGGC |
| | | AGGCTGCTGCACCTGTCTGCCGGCCAGAGGCTGGGAGT |
| | | GCATCTGCACACAGAGGCCAGGGCTAGACACGCCTGGC |
| | | AGCTGACACAGGGCGCTACAGTGCTGGGCCTGTTCAGA |
| | | GTGACCCCGAGATTCCTGCC |
| 102 | nucleotide | see Table 1 |
| | sequence of FAP | |
| | (4B9) Fc knob | |
| | monomeric 4- | |
| | 1BB (71-248) | |
| | ligand | |

| | | |
|---|---|---|
| 103 | nucleotide | see Table 1 |
| | sequence of anti- | |
| | FAP(4B9) light | |
| | chain | |
| 211 | nucleotide | see Table 42 |
| | sequence of DP47 | |
| | (VL-CH1) light | |
| | chain | |
| 232 | DP47(VH-CL) Fc | EVQLLESGGGLVQPGGSLRLSCAASGFTFSSYAMSWVRQA |
| | hole dimeric 4- | PGKGLEWVSAISGSGGSTYYADSVKGRFTISRDNSKNTLYL |
| | 1BB ligand (71- | QMNSLRAEDTAVYYCAKGSGFDYWGQGTLVTVSSASVA |
| | 248) chain | APSVFIFPPSDEQLKSGTASVVCLLNNFYPREAKVQWKVD |
| | | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVCTLPPSRDE |
| | | LTKNQVSLSCAVKGFYPSDIAVEWESNGQPENNYKTTPPV |
| | | LDSDGSFFLVSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGL |
| 105 | FAP (4B9) Fc | see Table 1 |
| | knob monomeric | |
| | 4-1BB (71-248) | |
| | ligand | |
| 106 | anti-FAP(4B9) | see Table 1 |
| | light chain | |
| 214 | DP47 (VL-CH1) | see Table 42 |
| | light chain | |

Table 51 shows the cDNA and amino acid sequences of the monovalent DP47/FAP(4B9)-targeted 4-1BB ligand (71-254) trimer-containing Fc (kih) fusion antigen binding molecule, wherein the dimeric 4-1BBL is fused to the knob chain (Construct 2.22).

TABLE 51
Sequences of FAP(4B9)-targeted human 4-1BB ligand trimer containing Fc (kih)
fusion molecule Construct 2.22

| SEQ ID | | |
|---|---|---|
| NO: | Description | Sequence |
| 233 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | (VH-CL) Fc hole | ATTCACCTTTAGCAGTTATGCCATGAGCTGGGTCCGCCA |
| | monomeric 4- | GGCTCCAGGGAAGGGGCTGGAGTGGGTCTCAGCTATTA |
| | 1BBL (71-248) | GTGGTAGTGGTGGTAGCACATACTACGCAGACTCCGTGA |
| | chain | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGCG |
| | | CTAGTGTGGCCGCTCCCTCCGTGTTTATCTTTCCCCCATC |
| | | CGATGAACAGCTGAAAAGCGGCACCGCCTCCGTCGTGT |
| | | GTCTGCTGAACAATTTTTACCCTAGGGAAGCTAAAGTGC |
| | | AGTGGAAAGTGGATAACGCACTGCAGTCCGGCAACTCC |
| | | CAGGAATCTGTGACAGAACAGGACTCCAAGGACAGCAC |
| | | CTACTCCCTGTCCTCCACCCTGACACTGTCTAAGGCTGA |
| | | TTATGAGAAACACAAAGTCTACGCCTGCGAAGTCACCC |
| | | ATCAGGGCCTGAGCTCGCCCGTCACAAAGAGCTTCAAC |
| | | AGGGGAGAGTGTGACAAAACTCACACATGCCCACCGTG |
| | | CCCAGCACCTGAAGCTGCAGGGGGACCGTCAGTCTTCCT |
| | | CTTCCCCCCAAAACCCAAGGACACCCTCATGATCTCCCG |
| | | GACCCCTGAGGTCACATGCGTGGTGGTGGACGTGAGCC |
| | | ACGAAGACCCTGAGGTCAAGTTCAACTGGTACGTGGAC |
| | | GGCGTGGAGGTGCATAATGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCGGCGCCCC |
| | | CATCGAGAAAACCATCTCCAAAGCCAAAGGGCAGCCCC |
| | | GAGAACCACAGGTGTGCACCCTGCCCCCATCCCGGGAT |
| | | GAGCTGACCAAGAACCAGGTCAGCCTCTCGTGCGCAGT |
| | | CAAAGGCTTCTATCCCAGCGACATCGCCGTGGAGTGGG |
| | | AGAGCAATGGGCAGCCGGAGAACAACTACAAGACCACG |
| | | CCTCCCGTGCTGGACTCCGACGGCTCCTTCTTCCTCGTG |
| | | AGCAAGCTCACCGTGGACAAGAGCAGGTGGCAGCAGGG |
| | | GAACGTCTTCTCATGCTCCGTGATGCATGAGGCTCTGCA |
| | | CAACCACTACACGCAGAAGAGCCTCTCCCTGTCTCCGGG |
| | | TGGAGGCGGCGGAAGCGGAGGAGGAGGATCCAGAGAG |
| | | GGCCCTGAGCTGAGCCCTGATGATCCTGCCGGACTGCTG |
| | | GACCTGCGGCAGGGAATGTTTGCCCAGCTGGTGGCCCA |
| | | GAACGTGCTGCTGATCGATGGCCCCCTGTCCTGGTACAG |
| | | CGATCCTGGACTGGCTGGCGTGTCACTGACAGGCGGCCT |
| | | GAGCTACAAAGAGGACACCAAAGAACTGGTGGTGGCCA |
| | | AGGCCGGCGTGTACTACGTGTTCTTTCAGCTGGAACTGC |
| | | GGAGAGTGGTGGCCGGCGAAGGATCTGGCTCTGTGTCTC |
| | | TGGCCCTGCATCTGCAGCCTCTGAGATCTGCTGCTGGCG |
| | | CCGCTGCTCTGGCACTGACAGTGGATCTGCCTCCTGCCA |
| | | GCAGCGAGGCCCGGAATAGCGCATTTGGGTTTCAAGGC |
| | | AGGCTGCTGCACCTGTCTGCCGGCCAGAGGCTGGGAGT |
| | | GCATCTGCACACAGAGGCCAGGGCTAGACACGCCTGGC |
| | | AGCTGACACAGGGCGCTACAGTGCTGGGCCTGTTCAGA |
| | | GTGACCCCCGAGATTCCTGCCGGGCTC |
| 108 | nucleotide | see Table 2 |
| | sequence of FAP | |
| | (4B9) Fc knob | |
| | dimeric 4-1BB | |
| | ligand (71-248) | |
| | chain | |
| 103 | nucleotide | See Table 1 |
| | sequence of anti- | |
| | FAP (4B9) light | |
| | chain | |
| 211 | nucleotide | see Table 42 |
| | sequence of DP47 | |
| | (VL-CH1) light | |
| | chain | |
| 234 | DP47 (VH-CL) | EVQLLESGGGLVQPGGSLRLSCAASGFTFSSYAMSWVRQA |
| | Fc hole | PGKGLEWVSAISGSGGSTYYADSVKGRFTISRDNSKNTLYL |
| | monomeric 4- | QMNSLRAEDTAVYYCAKGSGFDYWGQGTLVTVSSASVA |
| | 1BBL (71-248) | APSVFIFPPSDEQLKSGTASVVCLLNNFYPREAKVQWKVD |
| | chain | NALQSGNSQESVTEQDSKDSTYSLSSTLTLSKADYEKHKV |
| | | YACEVTHQGLSSPVTKSFNRGECDKTHTCPPCPAPEAAGG |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALGAPIEKTISKAKGQPREPQVCTLPPSRDE |
| | | LTKNQVSLSCAVKGFYPSDIAVEWESNGQPENNYKTTPPV |
| | | LDSDGSFFLVSKLTVDKSRWQQGNVFSCSVMHEALHNHY |
| | | TQKSLSLSPGGGGGSGGGGSREGPELSPDDPAGLLDLRQG |
| | | MFAQLVAQNVLLIDGPLSWYSDPGLAGVSLTGGLSYKEDT |
| | | KELVVAKAGVYYVFFQLELRRVVAGEGSGSVSLALHLQPL |
| | | RSAAGAAALALTVDLPPASSEARNSAFGFQGRLLHLSAGQ |
| | | RLGVHLHTEARARHAWQLTQGATVLGLFRVTPEIPAGL |
| 110 | anti-FAP(4B9) | Fc |
| | knob dimeric 4- | see Table 2 |
| | 1BB ligand (71- | |
| | 248) chain | |
| 106 | anti-FAP(4B9) | See Table 1 |
| | light chain | |
| 214 | DP47 (VL-CH1) | see Table 42 |
| | light chain | |

### Example 5

### Production of Monovalent FAP-Targeted Human DP47/4-1BB Ligand Trimer-Containing Fc Fusion Antigen Binding Molecules

The monovalent FAP-targeted trimeric 4-1BB ligand C-terminal Fc (kih) fusion antigen binding molecule encoding sequences were cloned into a plasmid vector, which drives expression of the insert from an MPSV promoter and contains a synthetic polyA sequence located at the 3′ end of the CDS. In addition, the vector contains an EBV OriP sequence for episomal maintenance of the plasmid.

The split trimeric 4-1BB ligand Fc (kih) fusion molecules were produced by co-transfecting HEK293-EBNA cells with the mammalian expression vectors using polyethylenimine. The cells were transfected with the corresponding expression vectors. For constructs 2.15 to 2.22, at a 1:1:1:1 ratio (“vector Fc hole chain”: “vector light chain 1”: “vector Fe knob chain”: “vector light chain 2”). The production and purification was cared out as described in Example 2.

Table 52 summarizes the yield and final monomer content of an exemplary DP47/FAP(4B9)-targeted split trimeric C-terminal 4-1BB ligand Fe(kih) fusion antigen binding molecule.

TABLE 52
Biochemical analysis of an exemplary targeted split trimeric
C-terminal 4-1BB ligand Fc(kih) fusion antigen binding molecule

| | Monomer | |
|---|---|---|
| | [%] | Yield |
| Construct | (SEC) | [mg/l] |
| monovalent FAP(4B9) targeted split trimeric | 98.9 | 14.5 |
| 4-1BB ligand C-terminal Fc (kih) fusion | | |
| (construct 2.15) | | |

### Example 5

### Determination of Simultaneous Binding of DP47/FAP(4B9)-Targeted C-Terminal 4-1BB Split Trimeric Ligand Fc Fusion Antigen Binding Molecules by Surface Plasmon Resonance

The capacity of binding simultaneously to human 4-1BB Fc(kih) and human FAP was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany).

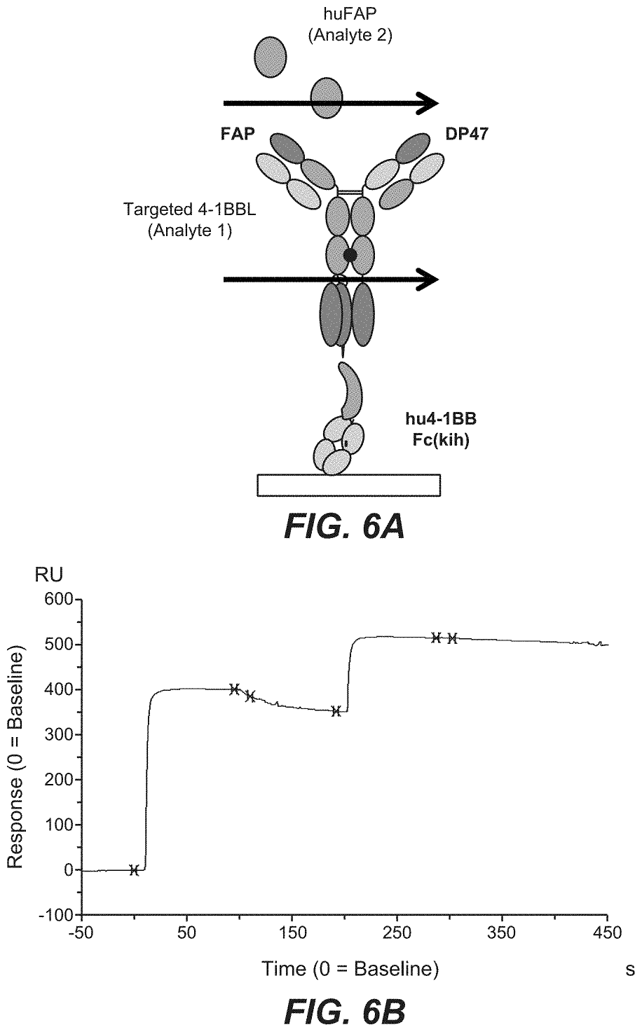

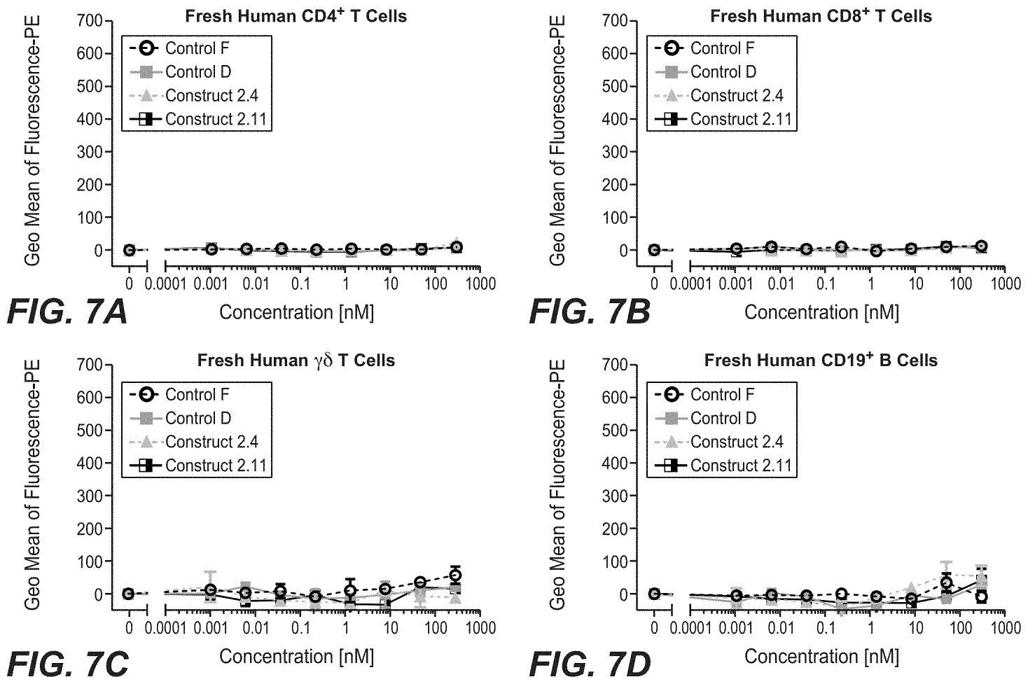

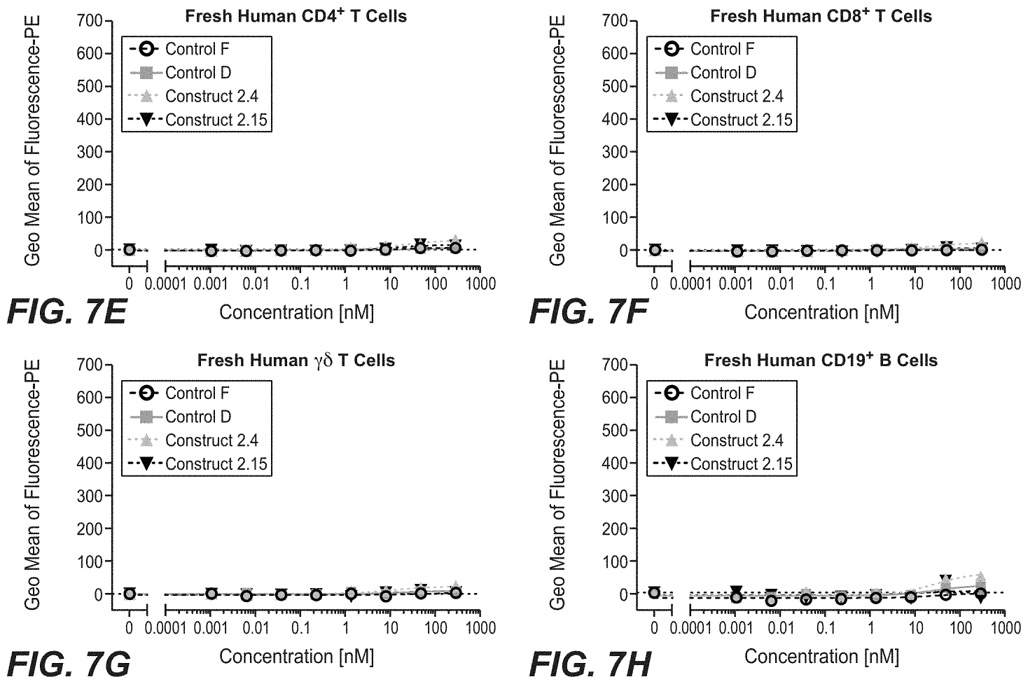

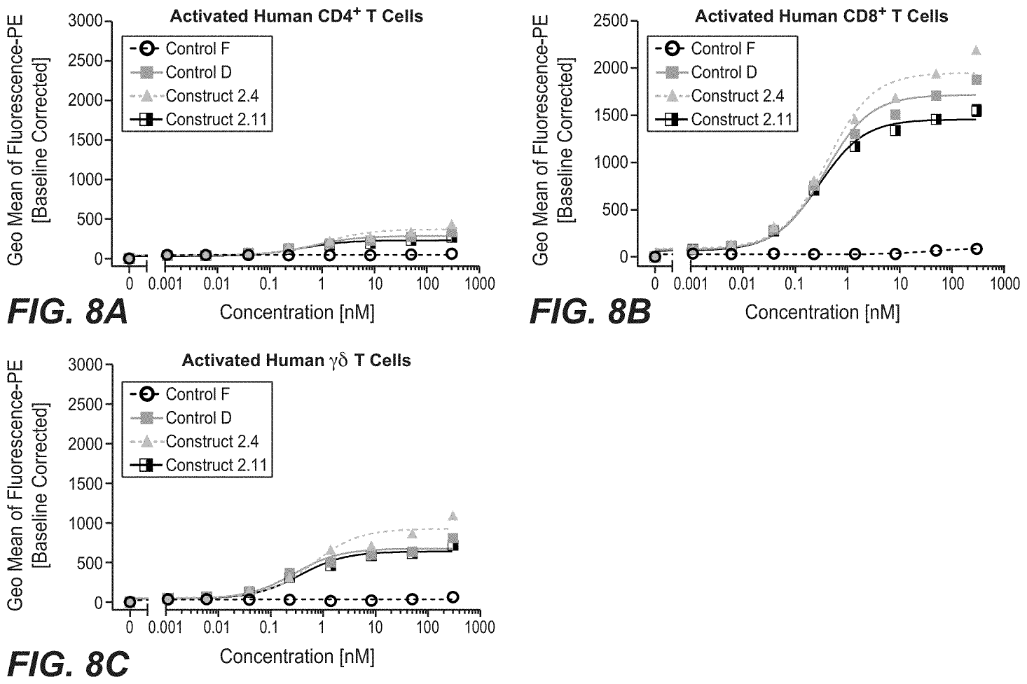

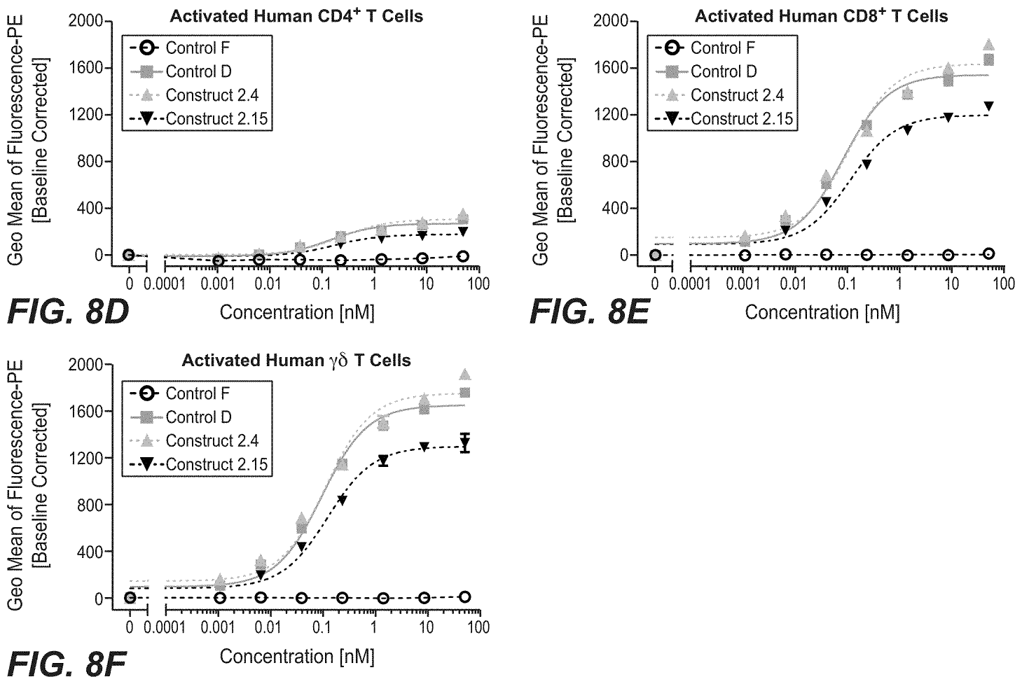

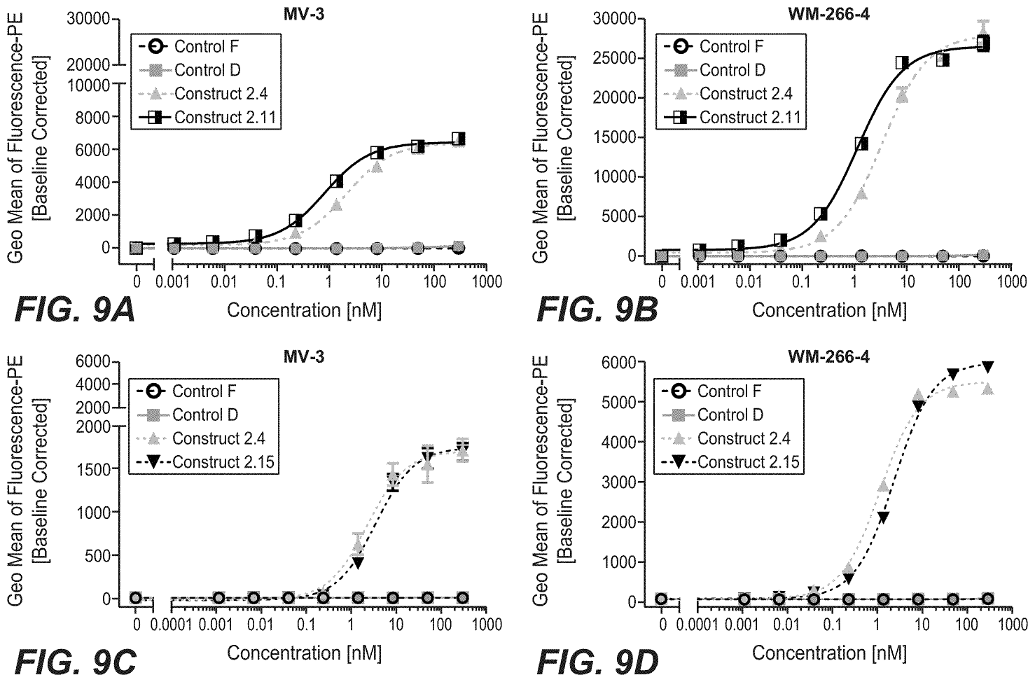

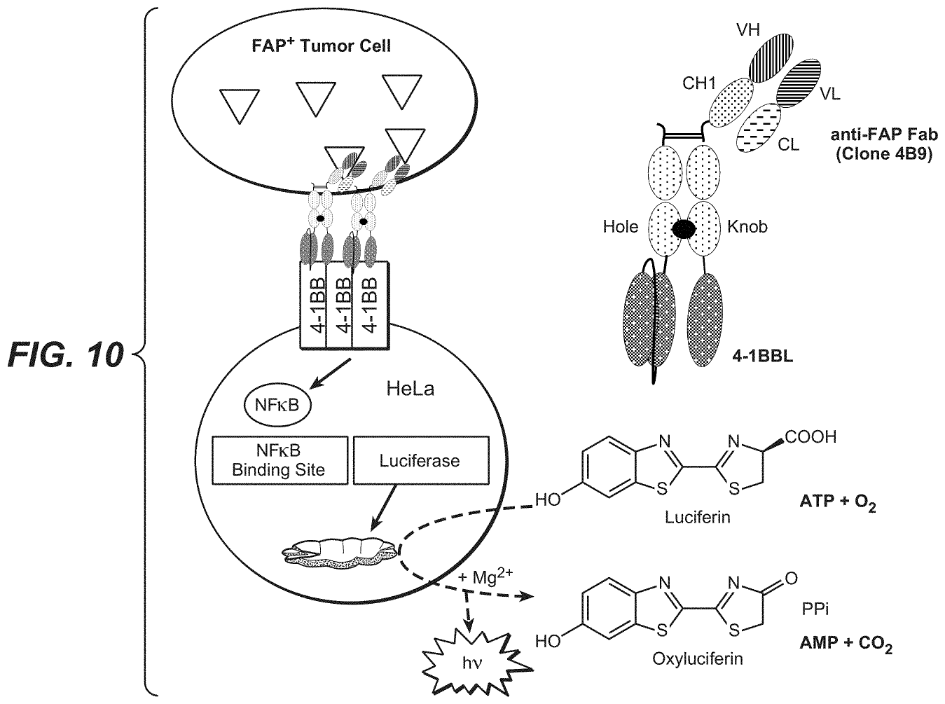

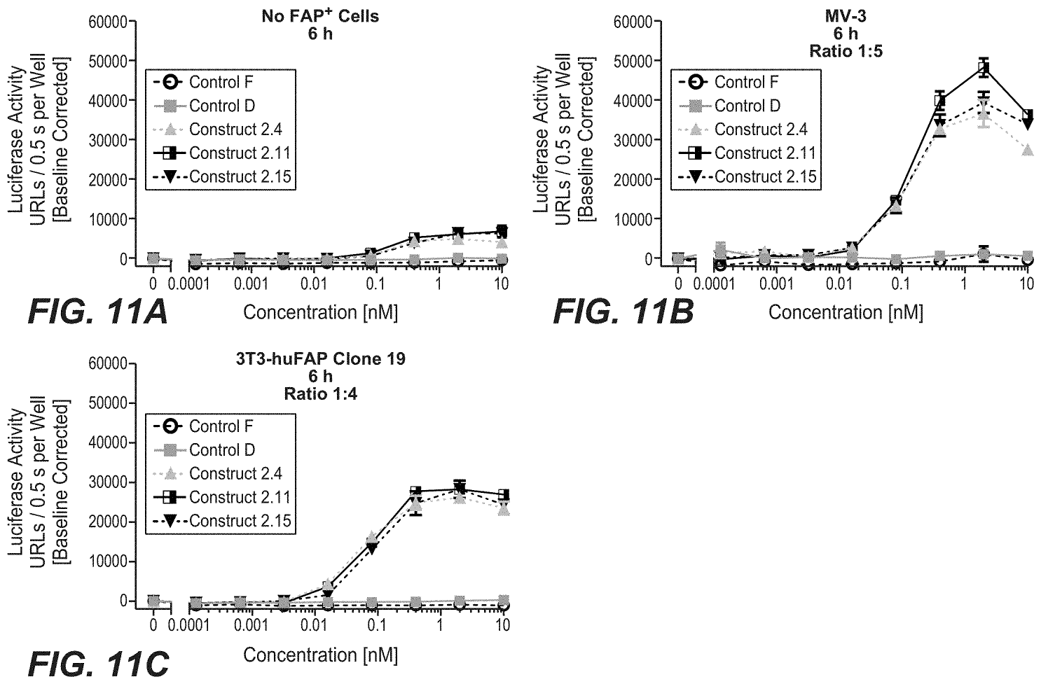

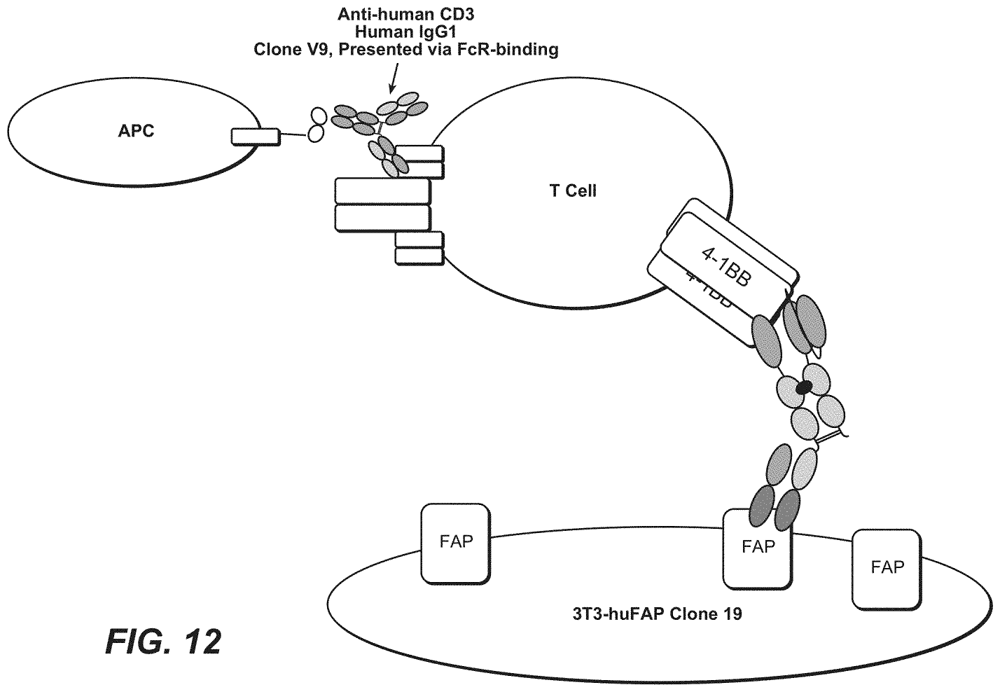

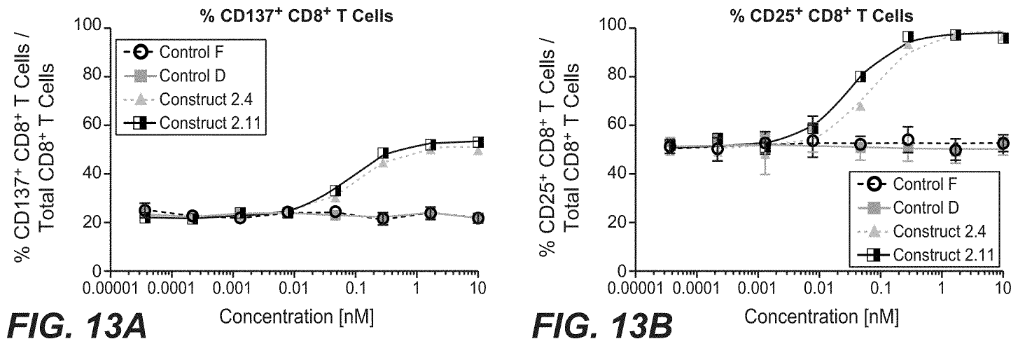

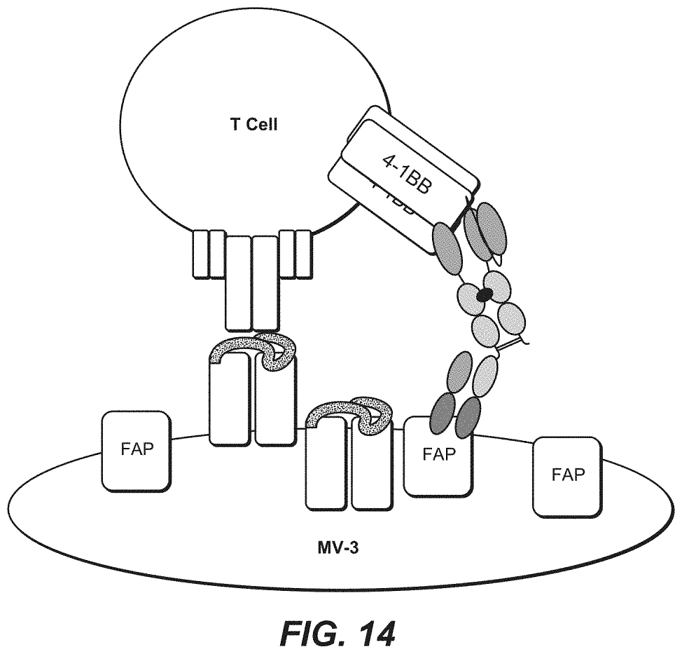

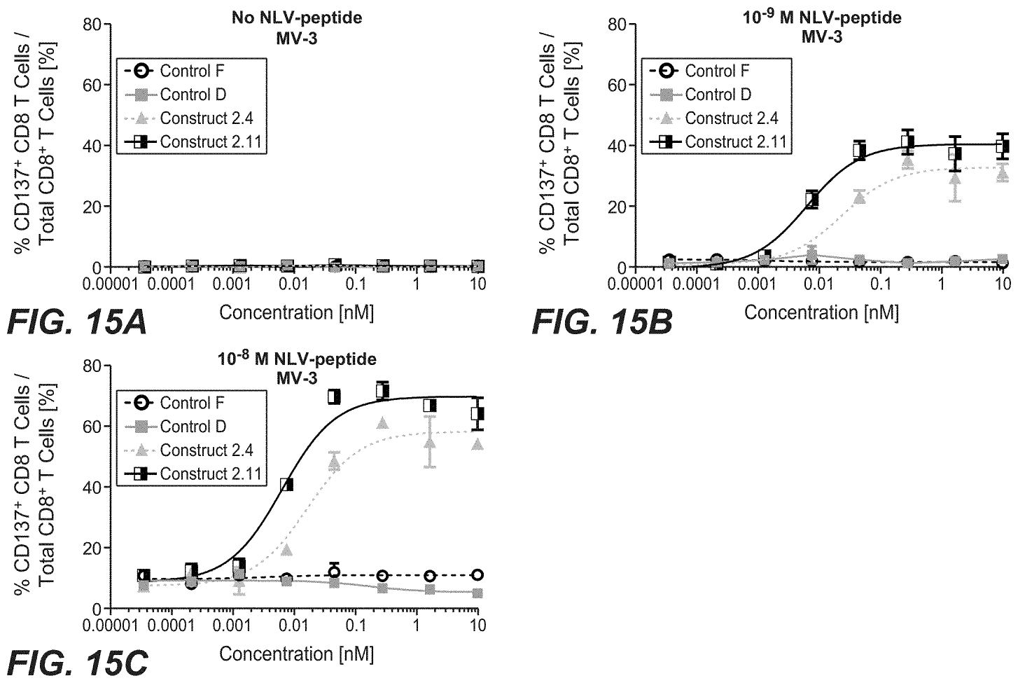

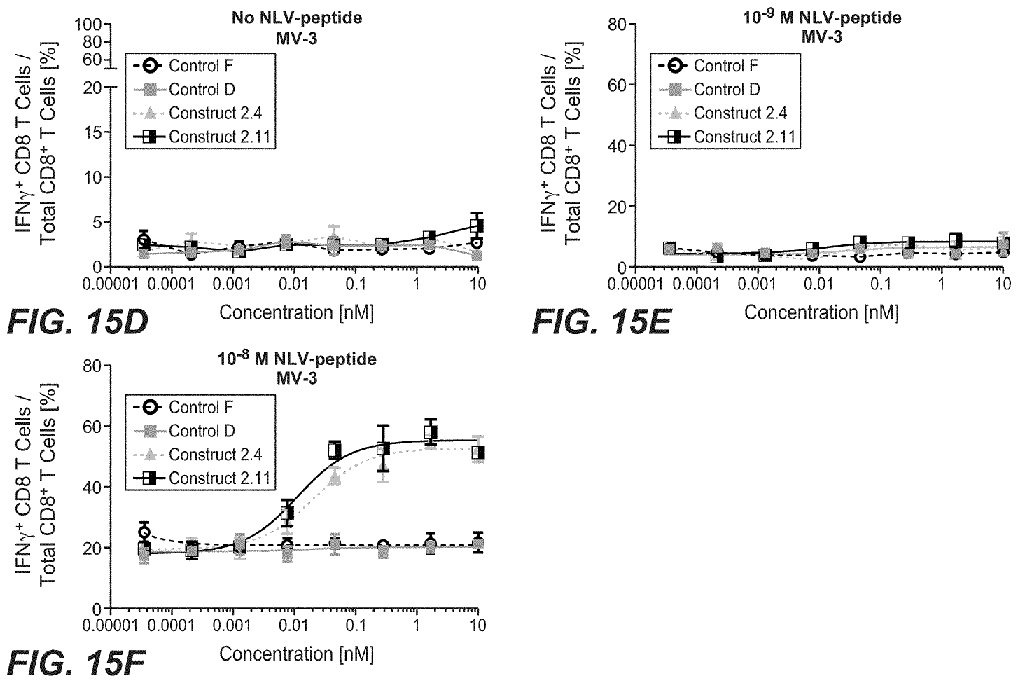

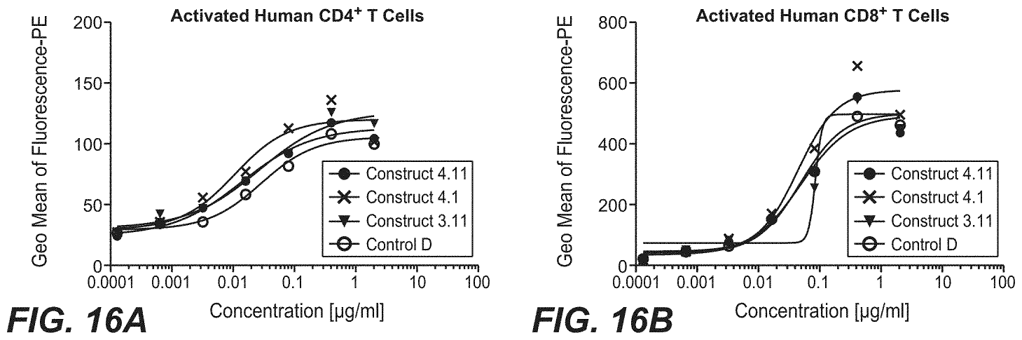

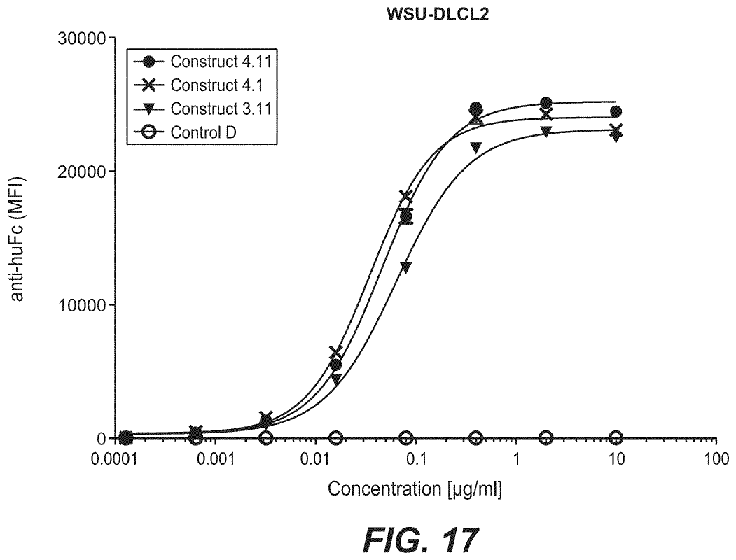

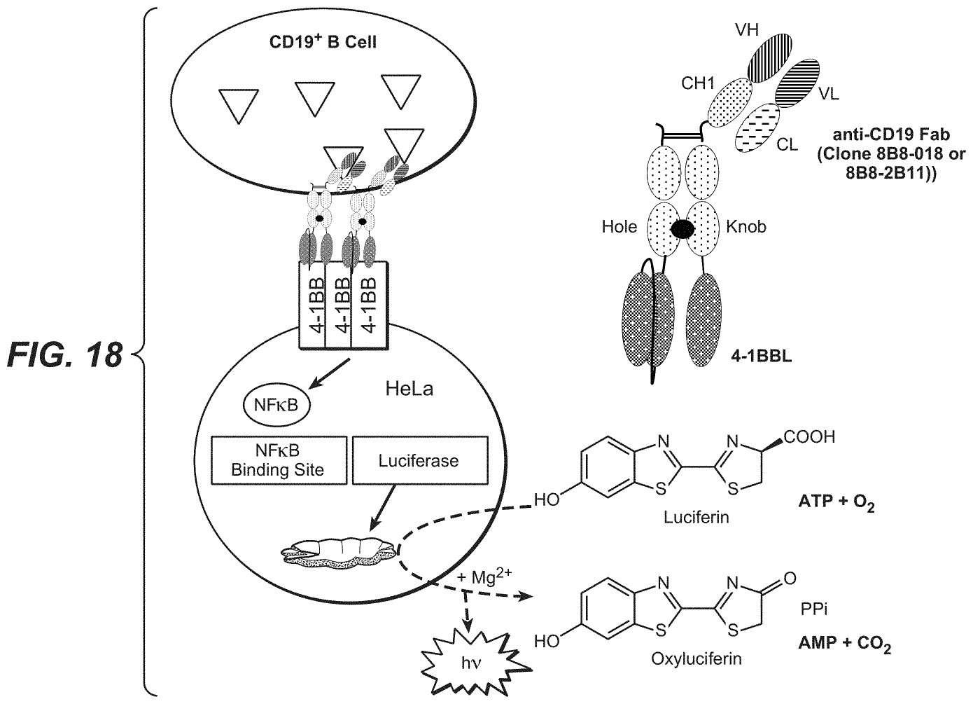

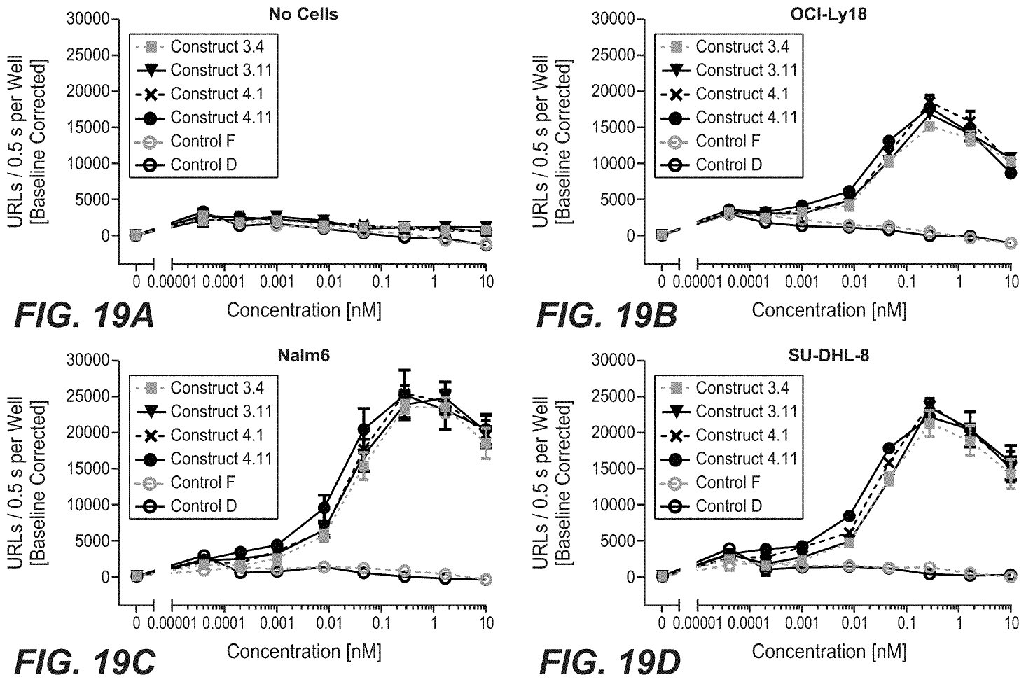

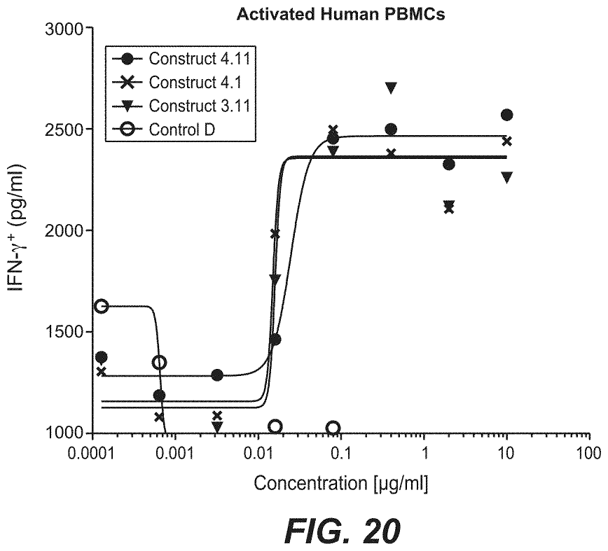

Biotinylated human 4-1BB Fc(kih) was directly coupled to a flow cell of a streptavidin (SA) sensor chip. Immobilization levels up to 200 resonance units (RU) were used. The targeted trimeric C-terminal 4-1BB ligand Fc (kih) fusion molecules were passed at a concentration range of 200 nM with a flow of 30 μL/minute through the flow cells over 90 seconds and dissociation was set to zero sec. Human FAP was injected as second analyte with a flow of 30 μL/minute through the flow cells over 90 seconds at a concentration of 500 nM (FIG. 6A). The dissociation was monitored for 120 sec. Bulk refractive index differences were corrected for by subtracting the response obtained in a reference flow cell, where no protein was immobilized. All bispecific constructs could bind simultaneously human 4-1BB and human FAP (FIG. 6B).

### Example 6

### Functional Characterization of Monovalent FAP-Targeted C-Terminal 4-1BB Split Trimeric Ligand Fc Fusion Antigen Binding Molecules

6.1 Binding on Fresh and Activated Human PBMCs

Buffy coats were obtained from the Zurich blood donation center. To isolate fresh peripheral blood mononuclear cells (PBMCs) the buffy coat was diluted with the same volume of DPBS (Gibco by Life Technologies, Cat. No. 14190 326). 50 mL polypropylene centrifuge tubes (TPP, Cat.-No. 91050) were supplied with 15 mL Histopaque 1077 (SIGMA Life Science, Cat.-No. 10771, polysucrose and sodium diatrizoate, adjusted to a density of 1.077 g/mL) and the diluted buffy coat contents were layered above the Histopaque 1077. The tubes were centrifuged for 30 min at 450×g. PBMCs were then collected from the interface, washed three times with DPBS and resuspended in T cell medium consisting of RPMI 1640 medium (Gibco by Life Technology, Cat. No. 42401-042) supplied with 10% (v/v) Fetal Bovine Serum (FBS, US-origin, PAN biotech, P30-2009, Lot P150307GI, gamma irradiated mycoplasma free, heat inactivated 35 min 56° C.), 1% (v/v) GlutaMAX I (GIBCO by Life Technologies, Cat. No. 35050 038), 1 mM Sodium-Pyruvat (SIGMA, Cat. No. S8636), 1% (v/v) MEM non-essential amino acids (SIGMA, Cat.-No. M7145) and 50 μM β-Mercaptoethanol (SIGMA, M3148).

PBMCs were used directly after isolation (binding on fresh human PBMCs) or they were stimulated to induce 4-1BB expression on the cell surface of T cells (binding on activated human PBMCs). To induce 4-1BB upregulation on human CD8 T cells, 6-well tissue culture plates (TTP, Cat.-No. 92006) were coated with 10 ug/mL anti-human CD3 (clone OKT3, Mouse IgG2a, BioLegend, Cat.-No. 317315) and 2 ug/mL anti-human CD28 (clone CD28.2, Mouse IgG1, BioLegend, Cat.-No. 302923) in DPBS at 4° C. overnigh; shortly before further use plates were washed twice with DPBS to remove not coated antibodies. Freshly isolated PBMCs were set to 1×10⁶ cells/mL in T cell medium (same medium as described above), seeded to the aCD3/aCD28 coated 6-well plates and cultured for 3 days at 37° C. and 5% CO₂.

For binding assay, 0.1×10⁶ fresh or activated PBMCs were added to each well of a round-bottom suspension cell 96-well plates (greiner bio-one, cellstar, Cat. No. 650185). Plates were centrifuged 5 minutes with 350×g and at 4° C. and supernatant were discarded. Afterwards cells were stained in 100 μL/well DPBS containing 1:5000 diluted fixable viability dye eF660 (eBioscience, Cat.-No. 65-0864-18) for 30 minutes at 4° C. in the dark. Cells were washed once with 200 μL ice cold cold DPBS buffer. Next, 50 μL/well of 4° C. cold FACS buffer (DPBS supplied with 2% FBS, 5 mM EDTA pH8 (Amresco, Cat. No. E177) and 7.5 mM Sodium azide (Sigma-Aldrich, Cat.-No. S2002)) containing monovalent FAP-targeted C-terminal 4-1BB split trimeric ligand Fc fusion antigen binding molecules (in particular construct 2.11 or construct 2.15), as positive control construct 2.4 and as negative controls Control D and Control F (prepared as described in Example 1.11) were added and cells were incubated for 60 minutes at 4° C., washed three times with 200 μL/well 4° C. FACS buffer. Cells were further resuspended in 50 μL/well of 4° C. cold FACS buffer containing 0.67 μg/mL anti-human CD45-AF488 (clone H130, mouse igG1k, BioLegend, Cat.-No. 304019), 0.33 μg/mL anti-human CD8a-BV510 (clone SKI, mouse IgG1k, BioLegend, Cat.-No. 344732), 0.23 μg/mL anti-human CD4-BV421 (clone OKT4, mouse IgG2bx, BioLegend, Cat.-No. 317434), 0.67 μg/mL anti-human CD3-PerCP-Cy5.5 (clone UCHT1, mouse IgG1k, BioLegend, Cat.-No. 300430), 0.67 μg/mL anti-human CD19-PE/Cy7 (clone HIB19, mouse IgG1k, BioLegend, Cat.-No. 302216), 5 μg/mL PE-conjugated AffiniPure anti-human IgG F(ab′)2-fragment-specific goat F(ab′)2 fragment (Jackson ImmunoResearch, Cat. No. 109 116 098) and incubated for 30 min at 4° C. Cells were washed twice with 200 μL/well 4° C. FACS buffer and then fixed with DPBS supplied with 1% Formaldehyde (Sigma, HT501320-9.5L). Cells were resuspended in 100 μL/well FACS-buffer and acquired using the MACS Quant Analyzer 10 (Miltenyi Biotech). Data was analyzed using FlowJo V10 (FlowJo, LLC) and GraphPad Prism 6.04 (GraphPad Software, Inc).

Gates were set on living CD45⁺ CD3⁺ CD19^{neg} CD8^{neg} CD4⁺ T cells, CD45⁺ CD3⁺ CD19^{neg} CD4^{neg} CD8⁺ T cells, CD45⁺ CD3⁺ CD4_{neg} CD8^{neg} γδ T cells and CD45⁺ CD3^{neg} CD19⁺ B cells and Geo Mean of fluorescence intensity of PE-conjugated AffiniPure anti-human IgG IgG Fcγ-fragment-specific goat F(ab′)2 fragment was blotted against the titrated concentration of FAP-targeted or untargeted C-terminal 4-1BB split trimeric ligand Fc fusion antigen binding molecules. As shown in FIGS. 7A-H and FIGS. 8A-F, only after activation of human PBMCs 4-1BB was upregulated on human CD4+ and CD8⁺ T cells. In Tables 53a and 53b the EC₅₀ values are summarized for the binding curves to activated human PBMCs.

TABLE 53a
EC₅₀ values of binding curves to activated human PBMCs
(construct 2.11) (see FIGS. 8A-C)

| | control | construct | construct |
|---|---|---|---|
| | D | 2.4 | 2.11 |
| EC₅₀ [nM] on activated CD4⁺ T cells | 0.37 | 0.41 | 0.28 |
| EC₅₀ [nM] on activated CD8⁺ T cells | 0.63 | 1.13 | 0.41 |
| EC₅₀ [nM] on activated γδ T cells | 0.28 | 0.64 | 0.36 |

TABLE 53b
EC₅₀ values of binding curves to activated human PBMCs
(construct 2.15) (see FIGS. 8D-F)

| | control | construct | construct |
|---|---|---|---|
| | D | 2.4 | 2.15 |
| EC₅₀ [nM] on activated CD4⁺ T cells | 0.08 | 0.10 | 0.11 |
| EC₅₀ [nM] on activated CD8⁺ T cells | 0.15 | 0.23 | 0.17 |
| EC₅₀ [nM] on activated γδ T cells | 0.09 | 0.11 | 0.12 |

6.2 Binding of FAP-Expressing Tumor Cells

For binding assays on FAP expressing cells different FAP-expressing tumor cells were used: human melanoma cell line MV-3 (first published: van Muijen G N, Jansen K F, Cornelissen I M, Smeets D F, Beck J L, Ruiter D J. Establishment and characterization of a human melanoma cell line (MV3) which is highly metastatic in nude mice. Int J Cancer. 1991 Apr. 22; 48(1):85-91) and the human melanoma cell line WM-266-4 (ATCC CRL-1676).

0.1×10⁶ tumor cells resuspended in DPBS (Gibco by Life Technologies, Cat. No. 14190 326) were added to each well of a round-bottom suspension cell 96-well plates (greiner bio-one, cellstar, Cat. No. 650185). Cells were washed once with 200 μL DPBS. Cells were resuspended in 100 μL/well of 4° C. cold DPBS buffer containing 1:5000 diluted Fixable Viability Dye eFluor 660 (eBioscience, Cat. No. 65-0864-18) and plates were incubated for 30 minutes at 4° C. Cells were washed once with 200 μL/well 4° C. cold DPBS buffer and resuspended in 50 μL/well of 4° C. cold FACS buffer (DPBS supplied with 2% FBS, 5 mM EDTA pH8 (Amresco, Cat. No. E177) and 7.5 mM Sodium azide (Sigma-Aldrich S2002)) containing construct 2.11 or construct 2.15, as positive control construct 2.4 and as negative controls control D and control F at a series of concentrations, followed by incubation for 1 hour at 4° C. After extensive washing, cells were further stained with 50 μL/well of 4° C. cold FACS buffer containing 5 μg/mL PE-conjugated AffiniPure anti-human IgG F(ab′)2-fragment-specific goat F(ab′)2 fragment (Jackson ImmunoResearch, Cat. No. 109 116 098) for 30 minutes at 4° C. Cells were washed twice with 200 μL/well 4° C. FACS buffer and cells were fixed in 50 μL/well DPBS containing 1% Formaldehyde (Sigma, HT501320-9.5L). Cells were resuspended in 100 μL/well FACS-buffer and acquired using the MACS Quant Analyzer 10 (Miltenyi Biotech). Data was analyzed using FlowJo V10 (FlowJo, LLC) and GraphPad Prism 6.04 (GraphPad Software, Inc).

Gates were set on living tumor cells and geo means of fluorescence intensity of PE-conjugated AffiniPure anti-human IgG IgG Fcγ-fragment-specific goat F(ab′)2 fragment were blotted against the titrated concentration of FAP-targeted or untargeted C-terminal 4-1BB split trimeric ligand Fc fusion antigen binding molecules. As shown in FIGS. 9A and B, only the FAP-targeted split trimeric 4-1BB ligand fusion molecules construct 2.4 and construct 2.11 bound to FAP-expressing tumor cells MV-3 amd WM-266-4 whereas the untargeted Isotype control control F and the non-targeted split trimeric 4-1BB ligand fusion molecule control D did not bind to the tumor cells. In FIGS. 9C and 9D it is shown that the trispecific DP47/FAP-targeted split trimeric 4-1BB ligand fusion molecule construct 2.15 also bound to FAP-expressing tumor cells MV-3 amd WM-266-4. In Tables 54a and 54b the EC₅₀ values of the FAP⁺ tumor binding curves are listed.

TABLE 54a
EC₅₀ values of binding curves to FAP-expressing tumor cells
(FIGS. 9A and B)

| | Construct 2.4 | Construct 2.11 |
|---|---|---|
| EC₅₀ [nM] on MV-3 | 2.16 | 0.82 |
| EC₅₀ [nM] on WM-266-4 | 3.43 | 1.16 |

TABLE 54a
EC₅₀ values of binding curves to FAP-expressing tumor cells
(FIGS. 9C and D)

| | Construct 2.4 | Construct 2.11 |
|---|---|---|
| EC₅₀ [nM] on MV-3 | 2.21 | 3.42 |
| EC₅₀ [nM] on WM-266-4 | 1.18 | 2.4 |

6.3 Biological Activity: NF-κB Activation of Human 4-1BB Expressing HeLa Reporter Cell Line

6.3.1 Generation of HeLa Reporter Cells Expressing Human 4-1BB and NF-icB-Luciferase

The human-papilloma-virus-18-induced cervix carcinoma cell line HeLa (ATCC CCL-2) was transduced with a plasmid based on the expression vector pETR10829, which contains the sequence of human 4-1BB (Uniprot accession Q07011) under control of a CMV-promoter and a puromycin resistance gene. Cells were cultured in DMEM-medium (Gibco by Life Technologies Cat. No. 42430-025) supplied with 10% Fetal Bovine Serum (FBS, GIBCO by Life Technologies, Cat. No. 16000-044, Lot. No. 941273, gamma-irradiated mycoplasma free, heat inactivated at 56° C. for 35 minutes), 1% GutaMAX-I (GIBCO by Life Technologies, Cat.-No. 35050-038) and 3 μg/mL Puromycin (InvivoGen, Cat-No. ant-pr). 4-1BB-transduced HeLa cells were tested for 4-1BB expression by flow cytometry: 0.2×106 living cells were resuspended in 100 L FACS buffer (DPBS supplied with 2% FBS, 5 mM EDTA pH 8 (Amresco, Cat. No. E177) and 7.5 mM Sodium Azide (Sigma-Aldrich, Cat. No. S2002)) containing 0.1 μg PerCP/Cy5.5 conjugated anti-human 4-1BB mouse IgG1κ clone 4B4-1 (BioLegend Cat. No. 309814) or its isotype control (PerCP/Cy5.5 conjugated mouse IgG1κ isotype control antibody clone MOPC 21, BioLegend Cat. No. 400150) and incubated for 30 minutes at 4° C. Cells were washed twice with FACS buffer, resuspended in 300 L FACS buffer containing 0.06 μg DAPI (Santa Cruz Biotec, Cat. No. Sc-3598) and acquired using a 5-laser LSR-Fortessa (BD Bioscience, DIVA software). Limited dilutions were performed to generate single clones as following: Human-4-1BB transduced HeLa cells were resuspended in medium to a concentration of 10, 5 and 2.5 cells/mL and 200 uL of cell suspensions were transferred to round bottom tissue-culture treated 96-well plates (6 plates/cell concentration, TPP Cat. No. 92697). Single clones were harvested, expanded and tested for 4-1BB expression as described above. The clone with the highest expression of 4-1BB (clone 5) was chosen for subsequent transfection with the NFκB-luciferase expression-vector 5495p Tranlucent HygB. The vector confers transfected cells both with resistance to hygromycin B and capacity to express luciferase under control of NFκB-response element (Panomics, Cat. No. LR0051). Human-4-1BB HeLa clone 5 cells were cultured to 70% confluence. 50 μg (40 μL) linearized (restriction enzymes AseI and SalI) 5495p Tranlucent HygB expression vector were added to a sterile 0.4 cm Gene Pulser/MicroPulser Cuvette (Biorad, Cat.-No, 165-2081). 2.5×10⁶ human-4-1BB HeLa clone 5 cells in 400 μl supplement-free DMEM were added and mixed carefully with the plasmid solution. Transfection of cells was performed using a Gene Pulser Xcell total system (Biorad, Cat No. 165 2660) under the following settings: exponential pulse, capacitance 500 μF, voltage 160 V, resistance oo. Immediately after the pulse transfected cells were transferred to a tissue culture flask 75 cm² (TPP, Cat. No. 90075) with 15 mL 37° C. warm DMEM-Medium (Gibco by Life Technologies Cat. No. 42430-025) supplied with 10% FBS and 1% GutaMAX I (GIBCO by Life Technologies, Cat. No. 35050 038). Next day, culture medium containing 3 μg/mL Puromycin (InvivoGen, Cat No. ant pr) and 200 μg/mL Hygromycin B (Roche, Cat.-No. 10843555001) was added. Surviving cells were expanded and limited dilution was performed as described above to generate single clones. Clones were tested for 4-1BB expression as described above and for NFκB-Luciferase activity as following: Clones were harvested in selection medium and counted using a Cell Counter Vi-cell xr 2.03 (Beckman Coulter, Cat. No. 731050). Cells were set to a cell density of 0.33×10⁶ cells/mL and 150 μL of this cell suspension were transferred to each well of a sterile white 96-well flat bottom tissue culture plate with lid (greiner bio one, Cat. No. 655083) and as a control to normal 96 well flat bottom tissue culture plate (TPP Cat. No. 92096) to test survival and cell density the next day. Cells were incubated at 37° C. and 5% CO₂ overnight. The next day 50 μL of medium containing different concentrations of recombinant human tumor necrosis factor alpha (rhTNFa, PeproTech, Cat.-No. 300 01A) were added to each well of a 96-well plate resulting in final concentration of rhTNFa of 100, 50, 25, 12.5, 6.25 and 0 ng/well. Cells were incubated for 6 hours at 37° C. and 5% CO₂ and then washed three times with 200 μL/well DPBS. Reporter Lysis Buffer (Promega, Cat-No: E3971) was added to each well (30 μl) and the plates were stored over night at −20° C. The next day frozen cell plates and Detection Buffer (Luciferase 1000 Assay System, Promega, Cat. No. E4550) were thawed to room temperature. 100 L of detection buffer were added to each well and the plate was measured as fast as possible using a SpectraMax M5/M5e microplate reader and the SoftMax Pro Software (Molecular Devices). Measured URLs above control (no rhTNFa added) were taken as luciferase activity. The NFκB-luc-4-1BB-HeLa clone 26 was chosen for further use exhibiting the highest luciferase activity and a considerable level of 4-1BB-expression.

6.3.2 NF-κB activation of HeLa reporter cells expressing human 4-1BB co-cultured with FAP-expressing tumor cells

NFκB-luc-4-1BB-HeLa clone 26 cells were washed with DPBS (Gibco by Life Technologies, Cat. No. 14190 326) and treated with 0.05% Trypsin EDTA (Gibco by Life Technologies Cat. No. 25300 054) for 5 min at 37° C. Cells were harvested and resuspended in DMEM Medium (Gibco by Life Technologies Cat. No. 42430 025) supplied with 10% Fetal Calf Serum (FBS, US-origin, PAN biotech, P30-2009, Lot P150307GI, gamma irradiated mycoplasma free, heat inactivated 35 min 56° C.) and 1% GutaMAX-I (GIBCO by Life Technologies, Cat. No. 35050 038). Cells were counted using Cell Counter Vi-cell xr 2.03 (Beckman Coulter, Cat. No. 731050) and set to a cell density of 0.2×10⁶ cells/mL. 100 μL (2×10⁴ cells) of this cell suspension were transferred to each well of a sterile white 96-well flat bottom tissue culture plate with lid (greiner bio one, Cat. No. 655083) and the plates were incubated at 37° C. and 5% CO₂ overnight. The next day 50 μL of medium containing different titrated concentrations of construct 2.11 were added. As positive control construct 2.4 and as negative controls control D and control F (as described in Example 1.11) were added. For FAP-binding-mediated crosslinking the FAP-expressing line MV-3 (first published: van Muijen G N, Jansen K F, Comelissen I M, Smeets D F, Beck J L, Ruiter D J. Establishment and characterization of a human melanoma cell line (MV3) which is highly metastatic in nude mice. Int J Cancer. 1991 Apr. 22; 48(1):85-91), human melanoma cell line WM-266-4 (ATCC CRL-1676) or FAP-expressing mouse fibroblast cell lines NIH/3T3 transfected with human FAP (NIH/3T3-huFAP clone 19) were used.

Therefore FAP expressing tumor cell lines were washed with DPBS (Gibco by Life Technologies, Cat. No. 14190 326) and treated with enzyme-free, PBS-based Cell Dissociation Buffer (Gibco by Life Technologies, Cat.-No. 13151-014) for 10 minutes at 37° C. Afterwards cell were harvested and counted using Cell Counter Vi-cell xr 2.03. Cells were resuspended in DEM supplied with 10% FBS and 1% L-GlutaMAX-I to 2×10⁶ cells/mL and 50 μL were added/well. After adding crosslinking FAP-expressing tumor cells plates were incubated for 6 hours at 37° C. and 5% CO₂. White flat bottom 96-well plates were washed three times with 200 μL/well DPBS. 40 μl fresh prepared Reporter Lysis Buffer (Promega, Cat-No: E3971) were added to each well and the plate were stored over night at −20° C. The next day frozen plates and detection buffer (Luciferase 1000 Assay System, Promega, Cat. No. E4550) were thawed to room temperature. 100 uL of detection buffer were added to each well and plates were measured as fast as possible using the SpectraMax M5/M5e microplate reader and SoftMax Pro Software (Molecular Devices) with following settings: for luciferase (RLUs), 500 ms integration time, no filter, collecting all wave length and top reading. The setup of the assay is shown in FIG. 10 and the activities as measured for Constructs 2.11, 2.15 and 2.4 and Controls are shown in FIG. 11. In Table 55 the EC₅₀ values of the NFκB-activation-induced Luciferase activity-curves are listed.

TABLE 55
EC₅₀ values of NFκB-activation-induced Luciferase activity-curves

| | Construct 2.4 | Construct 2.11 | Construct 2.15 |
|---|---|---|---|
| EC₅₀ [nM] on MV-3 | 0.15 | 0.18 | 0.16 |
| EC₅₀ [nM] on 3T3- | 0.06 | 0.08 | 0.10 |
| huFAP | | | |

6.4 Activation Assay of Human PBMCs in the Presence of FAP-Expressing Cells

For FAP-binding-mediated crosslinking the FAP-expressing mouse fibroblast cell lines NIH/3T3 transfected with human FAP (NIH/3T3-huFAP clone 19) was used. The cells were generated by transfection of mouse embryonic fibroblast NIH/3T3 cells (ATCC CRL-1658) with the expression pETR4921 plasmid encoding human FAP under a CMV promoter. Cells were maintained DMEM (Gibco by Life Technologies Cat.-No. 42430-025) supplied with 10% Calf Serum (CS, Iron supplemented from formula-fed calves, Sigma-Aldrich, C8056-500 mL) in the presence of 1.5 μg/mL puromycin (InvivoGen, Cat.-No.: ant-pr-5).

NIH/3T3-huFAP clone 19 cells were resuspended in T cell medium consisting of RPMI 1640 (GIBCO by Life Technologies, Cat.-No. 42401-042) supplied with 10% fetal bovine serum (FBS, US-origin, PAN biotech, P30-2009, Lot P150307GI, gamma irradiated mycoplasma free, heat inactivated 35 min 56° C.), 1% L-GlutaMAX-I (GIBCO by Life Technologies, Cat-No. 35050-038), 1 mM Sodium-Pyruvat (SIGMA-Aldrich, Cat.-No. S8636), 1% MEM-Non essential Aminoacid Solution (SIGMA-Aldrich, Cat.-No. M7145), 50 uM β-Mercaptoethanol (Sigma-Aldrich, Cyt.-No. M3148) and irradiated with 50 Gy (X-Ray Irradiator RS 2000, Rad source). 2×10⁴ NIH/3T3-huFAP clone 19 cells in 50 L T cell medium were seeded to each well of a round bottom tissue culture 96-well plate (TTP, Cat.-No. 92697). 50 μL of T cell medium containing different titrated concentrations of construct 2.11 were added. As positive control construct 2.4 and as negative controls control D and control F as described in Example 1.11 were added. Human PBMCs were labeled in 37° C. warm DPBS containing 40 nM CFDA-SE (SIGMA-Aldrich, Cat.-No. 21888-25MG-F) for 15 min at 37° C. CFSE-labeling was stopped by adding FBS, PBMCs were washed twice and resuspended in T cell medium to a final concentration of 1.5×10⁶ cells/mL. 50 L of this PBMC cell solution were seeded to each well to add 7.5×10⁴ CFSE-labeled PBMCs well. Finally a stock solution of T cell medium containing 2 mM agonistic anti-human CD3 antibody (clone V9, human IgG1, described in Rodrigues et al., Int J Cancer Suppl 7, 45-50 (1992) and U.S. Pat. No. 6,054,297) was prepared and 50 μL/well were added to each well giving a final concentration of 0.5 mM anti-human CD3 human IgG1 clone V9.

Plates were incubated for 4 days at 37° C. Cells were washed with DPBS and stained with 100 μL/well DPBS containing 1:1000 diluted LIVE/DEAD® Fixable Aqua Dead Cell Stain Kit (Molecular Probes by Life Technology, Cat.-No. L34957) for 30 min at 4° C. Cells were washed once with 200 μL/well DPBS and stained with 50 L FACS buffer (DPBS supplied with 2% FBS, 5 mM EDTA pH8 (Amresco, Cat. No. E177) and 7.5 mM Sodium azide (Sigma-Aldrich S2002)) containing 0.1 μg/mL PerCP-Cy5.5-conjugated anti-human CD137 mouse IgG1×(clone 4B4-1, BioLegend, Cat.-No. 309814), 0.1 μg/mL PE/Cy7-conjugated anti-human PD-1 mouse IgG1×(clone EH12.2H7, BioLegend, Cat.-No. 329918), 0.03 μg/mL APC-conjugated anti-human CD25 mouse IgG1κ (clone BC96, BioLegend, Cat.-No. 302610), 0.06 μg/mL APC/Cy7-conjugated anti-human CD8 Mouse IgG1 κ (clone RPA-T8, BioLegend, Cat.-No. 3301016), BV421-conjugated anti-human CD4 Mouse IgG1 κ (clone RPA-T4, BioLegend, Cat.-No. 300532) for 30 min at 4° C. Cells were washed twice with 200 μL/well DPBS and incubated for 30 min at 4° C. with 50 μL/well freshly prepared FoxP3 Perm/Fix buffer (eBioscience Cat.-No. 00-5123). Cells were washed twice with 200 μL/well DPBS, resuspended in 50 L/well freshly prepared Perm-buffer (eBioscience Cat.-No 00-8333) supplied with PE-conjugated 1:250 diluted anti-human Granzyme B mouse IgG1 κ (clone GB11, Lot 4269803, BD Pharmingen, Cat.-No. 561142) and incubated for 1 h at 4° C. Plates were washed twice with 200 μL/well DPBS and cells were fixed for 15 min with DPBS containing 1% Formaldehyde (Sigma, HT501320-9.5L). Cells were resuspended in 100 μL/well FACS-buffer and acquired using the MACS Quant Analyzer 10 (Miltenyi Biotech). Data was analyzed using FlowJo V10 (FlowJo, LLC) and GraphPad Prism 6.04 (GraphPad Software, Inc).

As shown in FIG. 13, only the FAP (4B9)-targeted split trimeric 4-1BBL fusion molecules Construct 2.11 and the positive control Construct 2.4 induced a concentration-dependent increase of surface expressed CD25 and 4-1BB (CD137) on CD8⁺ T cells. The negative control molecules Control F and D did not induce this up-regulation of activation markers. The EC₅₀ values of CD25 and 4-1BB up-regulation on CD8⁺ T cells are shown in Table 56.

TABLE 56
EC₅₀ values of induced up-regulation of activation markers 4-1BB
(CD137) and CD25 on CD8⁺ T cells

| | Construct 2.4 | Construct 2.11 |
|---|---|---|
| EC₅₀ [nM] of CD25-upregulation | 0.03 | 0.07 |
| on CD8+ T cells | | |
| EC₅₀ [nM] of CD137 (4-1BB)- | 0.08 | 0.11 |
| upregulating on CD8+ T cells | | |

6.5 Activation Assay of CMV-NLV-Specific CD8⁺ T Cells in the Presence of FAP-Expressing Cells

6.5.1 Isolation and Culture of NLV-Antigen-Specific CD8 T Cells

Fresh blood was obtained from a HLA-A2⁺ CMV-infected volunteer. PBMCs were isolated by Ficoll density centrifugation and CD8⁺ T cells were purified from PBMCs using a negative selection human CD8 T cell isolation Kit according to manufacturer's recommendations (Miltenyi Biotec, Cat. No. 130-094-156). Ten million of isolated CD8 T cells were resuspended in 1 mL sterile DPBS supplemented with 1% (v/v) FBS along with 50 μL of PE-labeled HLA-A2-pentamer containing the CMV-derived NLVPMVATV peptide (ProImmune, Cat. No. F008-2B) and incubated for 10 min at room temperature. Cells were washed twice with 3 mL sterile DPBS supplied with 1% (v/v) FBS. Cells were resuspended in 1 mL cells DPBS supplied with 1% (v/v) FBS containing 1 μg/mL anti-human CD8-FITC (clone LT8, Abcam, Cat. No. Ab28010) and incubated for 30 minutes at 4° C. Cells were washed twice, resupended to a concentration of 5×10⁶ cells/mL in DPBS supplied with 1% (v/v) FBS, and filtrated through a 30 μm pre-separation nylon-net cell strainer (Miltenyi Biotec, Cat. No. 130-041-407). NLV-peptide-specific CD8⁺ T cells were isolated by FACS sorting using an ARIA cell sorter (BD Bioscience with DIVA software) with the following settings: 100 μm nozzle and purity sort mask. Sorted cells were collected in a 15 ml polypropylene centrifuge tube (TPP, Cat. No. 91015) containing 5 ml RPMI 1640 medium supplied with 10% (v/v) FBS, 1% (v/v) GlutaMAX-I and 400 U/mL Proleukin. Sorted cells were centrifuged for 7 minutes at 350×g at room temperature and resuspended in same medium to a concentration of 0.53×10⁶ cells/mL. 100 μL/well of this cell suspension were added to each well of a previously prepared feeder plate. PHA-L-activated irradiated allogeneic feeder cells were prepared from PBMCs as previously described (Levitsky et al., 1998) and distributed to 96 well culture plates at 2×10⁵ feeder cells per well. After one day of culturing 100 μL medium/well were removed from well containing sorted CD8⁺ T-cells and replaced by new RPMI 1640 medium supplemented with 10% (v/v) FBS and 1% (v/v) GlutaMAX-I and 400 U/mL Proleukin, this was repeated during culture on a regular basis (every 2-4 days). As soon as cells start to proliferate, they were transferred to 24-well flat-bottom tissue culture plate (TPP, 92024). Cells were expanded/split and reactivated with new feeder cell preparation on a regular basis.

6.5.2 Activation Assay of NLV-Antigen-Specific CD8⁺ T Cells

MV-3 cells (already described in Example 6.2) were harvested and resuspended to 1×10⁶ cells/mL in T cell medium consisting of RPMI 1640 medium supplemented with 10% (v/v) FBS, 1% (v/v) GlutaMAX I, 1 mM Sodium-Pyruvate, 1% (v/v) MEM non-essential amino acids and 50 μM β-Mercaptoethanol and separated into three tubes. Synthetic NLVPMVATV peptide (obtained from thinkpeptides) was added to a final concentration of 1×10⁻⁹ M or 1×10⁻⁸ M and cells were incubated for 90 min. NLV-peptide pulsed MV-3 cells were washed once with T cell medium and resuspended to a density of 0.5×10⁶ cells/mL, distributed (100 μL/well) to a 96-well round bottom cell-suspension plate (Greiner bio-one, cellstar, Cat. No. 650185) and incubated over night at 37° C. and 5% CO₂. The next day, FAP-targeted 4-1BB ligand trimer-containing Fc fusion antigen binding molecule (construct 2.11 and as controls construct 2.4 and control F and D) were added in 50 μL of T-cell medium at a range of final concentrations (from 10 to 0.0004 nM). NLV-specific CD8 T cells were harvested, washed and added in 50 μL medium to each well (final tumor: CD8 T cell ratio 0.125). The principle of the assay is shown in FIG. 14.

Cells were incubated for 24 h and 50 μL/well were replaced with T cell medium containing 2.64 μL/mL Golgi stop (Protein Transport Inhibitor containing Monesin, B D Bioscience, Cat.-No. 554724, end concentration 0.66 l/mL) and 4 L/mL Golgi plug (Protein Transporter Inhibitor containing Brefeldin A, BD Bioscience, Cat.-No. 555029, end concentration 1 g/mL) were added to each well.

Cells were incubated for 4 h and then plates were washed with 200 μL/well DPBS and stained with 100 μL4° C. DPBS containing 1:1000 diluted LIVE/DEATH Fixable Aqua Dead Cell Stain (Molecular Probes, Cat.-No. L34957) for 30 minutes at 4° C. Cell were washed with 200 μL DPBS and stained in 50 L/well FACS buffer (DPBS containing 2% FBS, 5 mM EDTA, 7.5 mM Sodium-Azide) containing 1 g/mL anti-human CD137-PerCP-Cy5.5 (BioLegend, clone 4B4-1, Cat.-No. 309814), 0.67 μg/mL anti-human CD8-APC-Cy7 (BioLegend, clone RPA-T8, Cat.-No. 301016) and 0.67 μg/mL anti-human PD-1-PE-Cy7 (BioLegend, clone EH12.2H7, Cat.-No. 329918) for 30 min at 4° C. Cells were washed twice with 200 μL/well DBPS, resuspended in 50 μL freshly prepared Perm/Fix-buffer (eBioscience Cat.-No. 00-5123-43 and Cat.-No. 00-5223-56) and incubated for 30 min at 4° C. Cells were washed twice with 200 uL DPBS and resuspended in 50 μL freshly prepared Perm buffer (eBioscience Cat.-No. 00-8333-56) containing 6 ng/mL anti-human Eomes-eF660 (eBioscience, clone WD1928, Cat.-No. 50-4877-42), anti-human IFNγ-BV421 (BioLegend, clone 4S. B3, Cat.-No. 502532) and 4 L/mL anti-human Granzyme B-PE (BD, clone GB11, Cat.-No. 561142). Cells were incubated for 1 h at 4° C. and washed twice with 200 uL/well DPBS. For fixation, 50 μL/well DPBS containing 1% Formaldehyde were added and incubated for 15 min. Cells were resuspended in 100 μL/well FACS buffer and acquired using MACS Quant Analyzer 10 (Miltenyi Biotech). Data was analyzed with FlowJo v10.0 7 and Graph Pad Prism 6.04.

As shown in FIG. 15, only the FAP (4B9)-targeted split trimeric 4-1BBL fusion molecules construct 2.11 and the positive control construct 2.4 induced a concentration-dependent increase of expressed 4-1BB (CD137) (FIG. 15A) and IFNγ (FIG. 15B) on NLV-specific CD8⁺ T cells. The negative control molecules control F and D did not induce this up-regulation of activation markers. The EC₅₀ values of 4-1BB and IFNγ up-regulation on NLV-specific CD8⁺ T cells are shown in Table 57.

TABLE 57
EC50 values of induced upregulation of activation markers 4-1BB
(CD137) and IFNγ on NLV-activated CD8+ T cells.

| | | Construct | Construct |
|---|---|---|---|
| TCR stimulus | parameter | 2.4 | 2.11 |
| 10-9 M peptide | EC₅₀ [nM] on CD137⁺ CD8 | 0.023 | 0.006 |
| 10-8 M peptide | EC₅₀ [nM] on CD137⁺ CD8 | 0.017 | 0.006 |
| 10-8 M peptide | EC₅₀ [nM] on IFNγ⁺ CD8 | 0.021 | 0.011 |

### Example 7

### Functional Characterization of Monovalent CD19-Targeted C-Terminal 4-1BB Split Trimeric Ligand Fc Fusion Antigen Binding Molecules

7.1 Binding on Activated Human PBMCs

To check the binding of 4-1BBL to 4-1BB expressing T cells, human PBMCs were pre-activated by TCR stimulation for the upregulation of 4-1BB on T cells for 48 hours. PBMCs were purified from buffy coat by Ficoll (GE Healthcare, Cat No. 17-1440-03) gradient centrifugification, and were then diluted into a concentration of 2.8×10⁶/ml resuspended in RPMI medium (Gibco, Cat No. 72400-054)+10% FBS (Gibco, Cat No. 20012-068) and 1% penicillin-Streptomycin (Gibco, Cat No. 15070-063) and 50 uM of 2-Mercaptoethanol (Gibco, Cat No. 31350-010). 90 ul of cells were added to each well of a round-bottom 96-well plates (greiner bio-one, cellstar, Cat. No. 650185). Then additional 50 ul anti-CD3 and anti-CD28 microbeads (Life Technologies, Cat No. 11131D) at 8×10⁵ beads/ml were added to wells. Two 20 days later, cells were washed with cold PBS (Gibco, 20012-068) 1 time, and resuspended with 90 ul of cold PBS, and incubated with 10 ul of solution containing construct 4.11, construct 3.11, positive control construct 4.1 and negative control Control D for 1 hour at 4° C. After extensive washing, cells were further stained with 50 μL/well of cold FACS buffer containing 5 μg/mL PE-conjugated AffiniPure anti-human IgG F(ab′)2-fragment-specific goat F(ab′)2 fragment (Jackson ImmunoResearch, Cat. No. 109 116 098), and additionally with anti-human CD3 (Biolegend, Cat No. 300312), CD4 (Biolegend, Cat No. 317434) and CD8 (Biolegend, Cat No. 344710) Ab for 30 minutes at 4° C. Cells were washed twice with 200 μL/well 4° C. FACS buffer and cells were fixed in 50 μL/well DPBS containing 1% Formaldehyde (Sigma, HT501320-9.5L). Cells were resuspended in 100 uL/well FACS-buffer and acquired using the FACS LSR II (BD Biosciences). Data was analyzed using FlowJo V10 (FlowJo, LLC) and GraphPad Prism 6.04 (GraphPad Software, Inc).

The specific binding was gated on pure population of CD4 and CD8 cells. All the constructs showed similar binding properties to 4-1BB expressing CD4 or CD8 T cells in a dose dependent manner (FIG. 16). In Table 58 the EC₅₀ values of the binding curves are listed.

TABLE 58
EC₅₀ values of binding curves to 4-1BB⁺ human T cells

| | Construct | Construct | Construct | Control |
|---|---|---|---|---|
| | 4.11 | 3.11 | 4.1 | D |
| EC₅₀ [nM] on CD4 | 0.13 | 0.13 | 0.11 | 0.17 |
| EC₅₀ [μg/ml] on CD4 | 0.02 | 0.02 | 0.02 | 0.03 |
| EC₅₀ [nM] on CD8 | 0.32 | 0.38 | 0.28 | 0.23 |
| EC₅₀ [μg/ml] on CD8 | 0.05 | 0.06 | 0.05 | 0.04 |

7.2 Binding of CD19-Expressing Tumor Cells

To check the binding to tumor antigen CD19, the CD19⁺ WSU-DLCL2 cells (DSMZ No. ACC 575) were used. 0.1×10⁶ tumor cells resuspended in DPBS (Gibco by Life Technologies, Cat. No. 14190 326) were added to each well of a round-bottom suspension cell 96-well plates (greiner bio-one, cellstar, Cat. No. 650185). Cells were washed once with 200 μL DPBS. Cells were resuspended in 100 μL/well of 4° C. cold DPBS buffer containing 1:5000 diluted Fixable Viability Dye eFluor 660 (eBioscience, Cat. No. 65-0864-18) and plates were incubated for 30 minutes at 4° C. Cells were washed once with 200 μL/well 4° C. cold DPBS buffer and resuspended in 50 μL/well of 4° C. cold FACS buffer (DPBS supplied with 2% FBS, 5 mM EDTA pH8 (Amresco, Cat. No. E177) and 7.5 mM Sodium azide (Sigma-Aldrich S2002)) containing Construct 4.11, Construct 3.11, as positive control construct 4.1 and as negative control control D at a series of concentrations, followed by incubation for 1 hour at 4° C. After extensive washing, cells were further stained with 50 μL/well of 4° C. cold FACS buffer containing 5 μg/mL PE-conjugated AffiniPure anti-human IgG F(ab′)2-fragment-specific goat F(ab′)2 fragment (Jackson ImmunoResearch, Cat. No. 109 116 098) for 30 minutes at 4° C. Cells were washed twice with 200 μL/well 4° C. FACS buffer and cells were fixed in 50 μL/well DPBS containing 1% Formaldehyde (Sigma, HT501320-9.5L). Cells were resuspended in 100 μL/well FACS-buffer and acquired using the FACS LSR II (BD Biosciences). Data was analyzed using FlowJo V10 (FlowJo, LLC) and GraphPad Prism 6.04 (GraphPad Software, Inc).

Gates were set on living tumor cells and geo means of fluorescence intensity of PE-conjugated AffiniPure anti-human IgG IgG Fcγ-fragment-specific goat F(ab′)2 fragment were plotted against the titrated concentration of CD19-targeted or untargeted C-terminal 4-1BB split trimeric ligand Fc fusion antigen binding molecules. As shown in FIG. 17, only the CD19-targeted split trimeric 4-1BB ligand fusion molecules Construct 4.11 and Construct 3.11 and Construct 4.1 bound to CD19-expressing tumor cells WSU-DLCL2, whereas the untargeted split trimeric 4-1BB ligand fusion molecule Control D did not bind to the tumor cells. In Table 59, the EC₅₀ values of the CD19⁺ tumor binding curves are listed.

TABLE 59
EC₅₀ values of binding curves to CD19⁺ tumor cells

| | Construct 4.11 | Construct 3.11 | Construct 4.1 |
|---|---|---|---|
| EC₅₀ [nM] | 0.26 | 0.38 | 0.17 |
| EC₅₀ [μg/ml] | 0.04 | 0.06 | 0.03 |

7.3 Biological Activity: NF-κB Activation of HeLa Reporter Cells Expressing Human 4-1BB Co-Cultured with CD19-Expressing B Cells Lymphoma Cells

7.3.1 Generation of HeLa Reporter Cells Expressing Human 4-1BB and NF-κB-Luciferase

The HeLa-human-4-1BB-NFκN-luc reporter cell line was generated as described already in Example 6.3.1.

7.3.2 NF-κB Activation of HeLa Reporter Cells Expressing Human 4-1BB Co-Cultured with FAP-Expressing Tumor Cells

NFκB-luc-4-1BB-HeLa clone 26 cells were washed with DPBS (Gibco by Life Technologies, Cat. No. 14190 326) and treated with 0.05% Trypsin EDTA (Gibco by Life Technologies Cat. No. 25300 054) for 5 min at 37° C. Cells were harvested and resuspended in DMEM Medium (Gibco by Life Technologies Cat. No. 42430 025) supplied with 10% Fetal Calf Serum (FBS, US-origin, PAN biotech, P30-2009, Lot P150307GI, gamma irradiated mycoplasma free, heat inactivated 35 min 56° C.) and 1% GutaMAX-I (GIBCO by Life Technologies, Cat. No. 35050 038). Cells were counted using Cell Counter Vi-cell xr 2.03 (Beckman Coulter, Cat. No. 731050) and set to a cell density of 0.2×10⁶ cells/mL. 100 μL (2×10⁴ cells) of this cell suspension were transferred to each well of a sterile white 96-well flat bottom tissue culture plate with lid (greiner bio one, Cat. No. 655083) and the plates were incubated at 37° C. and 5% CO₂ overnight. The next day 50 μL of medium containing different titrated concentrations of Construct 3.11 and Construct 4.11 were added. As positive control Constructs 3.4 and 4.1 and as negative controls Control D and Control F were added, which have been described Example 1.11. For CD19-binding-mediated crosslinking the following CD19-expressing cell lines were used: diffuse large B-cell lymphoma cell line OCI-Ly18 (DMSZ ACC 699), acute lymphoblastic leukemia (ALL) cell line Nalm6 (DSMZ ACC-128) and non-Hodgkin's B cell lymphoma (B-NHL) cell line SU-DHL-8 (DSMZ ACC-573). Therefore CD19-expressing suspension cell lines OCI-Ly18, Nalm6 and SU-DHL-8 and were harvested, counted using Cell Counter Vi-cell xr 2.03 and resuspended in DEM supplied with 10% FBS and 1% L-GlutaMAX-I to 2×10⁶ cells/mL and 50 μL were added/well. After adding crosslinking CD19-expressing tumor cells lines plates were incubated for 6 hours at 37° C. and 5% CO₂. White flat bottom 96-well plates were washed three times with 200 μL/well DPBS. 40 μl fresh prepared Reporter Lysis Buffer (Promega, Cat-No: E3971) were added to each well and the plate were stored over night at −20° C. The next day frozen plates and detection buffer (Luciferase 1000 Assay System, Promega, Cat. No. E4550) were thawed to room temperature. 100 uL of detection buffer were added to each well and plates were measured as fast as possible using the SpectraMax M5/M5e microplate reader and SoftMax Pro Software (Molecular Devices) with following settings: for luciferase (RLUs), 500 ms integration time, no filter, collecting all wave length and top reading. The setup of the assay is shown in FIG. 18 and the activities as measured for Constructs 3.4, 4.1, 3.11 and 4.11 and Controls D and F are shown in FIG. 19. In Table 60 the EC₅₀ values of the binding curves to CD19-expressing tumor cell lines are listed.

TABLE 60
EC₅₀ values of binding curves to CD19-expressing
tumor cell lines

| | Construct | Construct | Construct |
|---|---|---|---|
| | 3.4 | 3.11 | 4.1 |
| OCI-Ly18 EC₅₀ [nM] | 0.03 | 0.03 | 0.03 |
| Nalm6 EC₅₀ [nM] | 0.03 | 0.03 | 0.02 |
| SU-DHL-8 EC₅₀ [nM] | 0.03 | 0.03 | 0.03 |

7.4 Biological Activity: Human PBMC Activation Assay

To determine the biological activities in physiological relevant settings, we measured the release of effector function molecule IFNγ in activated human PBMCs by costimluating T cells and NK cells via 4-1BBL. PBMCs were purified from buffy coat by Ficoll (GE Healthcare, Cat No. 17-1440-03) gradient centrifugification, and were then diluted into a concentration of 2.2×10⁶/ml resuspended in RPMI medium (Gibco, Cat No. 72400-054)+10% FBS (Gibco, Cat No. 20012-068) and 1% penicillin-Streptomycin (Gibco, Cat No. 15070-063) and 50 uM of 2-Mercaptoethanol (Gibco, Cat No. 31350-010). 90 μl of cells were added to each well of round-bottom 96-well plates (greiner bio-one, cellstar, Cat. No. 650185). Then 10 l of solution containing Construct 4.11, Construct 3.11, positive control construct 4.1 and negative control control D were added to the wells at a series of concentrations, and provided with additional 50 μl anti-CD3 and anti-CD28 microbeads (Life Technologies, Cat No. 11131D) at 8×10⁵ beads/ml. After 48 hour incubation, the supernatant were collected for the measurement of IFN-γ by ELISA (DuoSet Human IFNg ELISA kit, R&D Systems, Cat No. DY285).

FIG. 20 shows that Construct 4.11, Construct 3.11 and the positive control Construct 4.1 stimulated PBMCs to produce a similar amount of IFNγ, whereas the negative control D did not activate T or NK cells due to the lack of cross-linking. In Table 61 the EC₅₀ values of the IFNγ curves on activated human PBMCs are listed.

TABLE 61
EC₅₀ values of of the IFNγ curves on activated human PBMCs

| | Construct 4.11 | Construct 3.11 | Construct 4.1 |
|---|---|---|---|
| EC₅₀ [nM] | 0.19 | 0.13 | 0.06 |
| EC₅₀ [μg/ml] | 0.03 | 0.02 | 0.01 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### CITATIONS

Ascierto, P. A., E. Simeone, M. Sznol, Y. X. Fu, and I. Melero (2010), Clinical experiences with anti-CD137 and anti-PD1 therapeutic antibodies. Semin Oncol 37:508-516.
Aggarwal B. B. (2003), Signalling pathways of the TNF superfamily: a double-edged sword. Nat. Rev. Immunol.3(9), 745-56.
Banner D. et al (1993), Crystal structure of the soluble human 55 kd TNF receptor-human TNF beta complex: implications for TNF receptor activation. Cell 73, 431-445.
Bodmer J., Schneider P. and Tschopp, J. (2002), The molecular architecture of the TNF superfamily. Trends in Biochemical Sciences 27(1), 19-26.
Broll, K., Richter, G., Pauly, S., Hofstaedter, F., and Schwarz, H. (2001). CD137 expression in tumor vessel walls. High correlation with malignant tumors. Am J Clin Pathol 115, 543-549.
Buechele, C., Baessler, T., Schmiedel, B. J., Schumacher, C. E., Grosse-Hovest, L., Rittig, K., and Salih, H. R. (2012). 4-1BB ligand modulates direct and Rituximab-induced NK-cell reactivity in chronic lymphocytic leukemia. Eur J Immunol 42, 737-748.
Choi, B. K., Kim, Y. H., Kwon, P. M., Lee, S. C., Kang, S. W., Kim, M. S., Lee, M. J., and Kwon, B. S. (2009). 4-1BB functions as a survival factor in dendritic cells. J Immunol 182, 4107-4115.
Cuadros, C., Dominguez, A. L., Lollini, P. L., Croft, M., Mittler, R. S., Borgstrom, P., and Lustgarten, J. (2005). Vaccination with dendritic cells pulsed with apoptotic tumors in combination with anti-OX40 and anti-4-1BB monoclonal antibodies induces T cell-mediated protective immunity in Her-2/neu transgenic mice. Int J Cancer 116, 934-943.
Curran, M. A., Kim, M., Montalvo, W., Al-Shamkhani, A., and Allison, J. P. (2011). Combination CTLA-4 blockade and 4-1BB activation enhances tumor rejection by increasing T-cell infiltration, proliferation, and cytokine production. PLoS One 6, e19499.
Diehl, L., van Mierlo, G. J., den Boer, A. T., van der Voort, E., Fransen, M., van Bostelen, L., Krimpenfort, P., Melief, C. J., Mittler, R., Toes, R. E., and Offringa, R. (2002). In vivo triggering through 4-1BB enables Th-independent priming of CTL in the presence of an intact CD28 costimulatory pathway. J Immunol 168, 3755-3762.
Dubrot, J., Milheiro, F., Alfaro, C., Palazon, A., Martinez-Forero, I., Perez-Gracia, J. L., Morales-Kastresana, A., Romero-Trevejo, J. L., Ochoa, M. C., Hervas-Stubbs, S., et al. (2010). Treatment with anti-CD137 mAbs causes intense accumulations of liver T cells without selective antitumor immunotherapeutic effects in this organ. Cancer Immunol Immunother 59, 1223-1233.
Futagawa, T., Akiba, H., Kodama, T., Takeda, K., Hosoda, Y., Yagita, H., and Okumura, K. (2002). Expression and function of 4-1BB and 4-1BB ligand on murine dendritic cells. Int Immunol 14,275-286.
Guo, Z., Cheng, D., Xia, Z., Luan, M., Wu, L., Wang, G., and Zhang, S. (2013). Combined TIM-3 blockade and CD137 activation affords the long-term protection in a murine model of ovarian cancer. J Transl Med 11, 215.
Heinisch, I. V., Daigle, I., Knopfli, B., and Simon, H. U. (2000). CD137 activation abrogates granulocyte-macrophage colony-stimulating factor-mediated anti-apoptosis in neutrophils. Eur J Immunol 30, 3441-3446.
Hornig, N., Kermer, V., Frey, K., Diebolder, P., Kontermann, R E., Mueller, D. (2012), Combination of a bispecific antibody and costimulatory antibody-ligand fusion proteins for targeted cancer immunotherapy. J. Immunother. 35, 418-429.
Ju, S. A., Cheon, S. H., Park, S. M., Tam, N. Q., Kim, Y. M., An, W. G., and Kim, B. S. (2008). Eradication of established renal cell carcinoma by a combination of 5-fluorouracil and anti-4-1BB monoclonal antibody in mice. Int J Cancer 122, 2784-2790.
Kienzle, G., and von Kempis, J. (2000). CD137 (ILA/4-1BB), expressed by primary human monocytes, induces monocyte activation and apoptosis of B lymphocytes. Int Immunol 12, 73-82.
Kim, D. H., Chang, W. S., Lee, Y. S., Lee, K. A., Kim, Y. K., Kwon, B. S., and Kang, C. Y. (2008). 4-1BB engagement costimulates NKT cell activation and exacerbates NKT cell ligand-induced airway hyperresponsiveness and inflammation. J Immunol 180, 2062-2068.
Kim, Y. H., Choi, B. K., Oh, H. S., Kang, W. J., Mittler, R. S., and Kwon, B. S. (2009). Mechanisms involved in synergistic anticancer effects of anti-4-1BB and cyclophosphamide therapy. Mol Cancer Ther 8, 469-478.
Kwon, B. S., and Weissman, S. M. (1989). cDNA sequences of two inducible T-cell genes. Proc Natl Acad Sci USA 86, 1963-1967.
Lee, H., Park, H. J., Sohn, H. J., Kim, J. M., and Kim, S. J. (2011). Combinatorial therapy for liver metastatic colon cancer: dendritic cell vaccine and low-dose agonistic anti-4-1BB antibody co-stimulatory signal. J Surg Res 169, e43-50.
Levitsky, V., de Campos-Lima, P. O., Frisan, T., and Masucci, M. G. (1998). The clonal composition of a peptide-specific oligoclonal CTL repertoire selected in response to persistent EBV infection is stable over time. J Immunol 161, 594-601.
Li, F., and Ravetch, J. V. (2011). Inhibitory Fcgamma receptor engagement drives adjuvant and anti-tumor activities of agonistic CD40 antibodies. Science 333, 1030-1034.
Lin, W., Voskens, C. J., Zhang, X., Schindler, D. G., Wood, A., Burch, E., Wei, Y., Chen, L., Tian, G., Tamada, K., et al. (2008). Fc-dependent expression of CD137 on human NK cells: insights into “agonistic” effects of anti-CD137 monoclonal antibodies. Blood 112, 699-707.
Melero, I., Johnston, J. V., Shufford, W. W., Mittler, R. S., and Chen, L. (1998). NK1.1 cells express 4-1BB (CDw137) costimulatory molecule and are required for tumor immunity elicited by anti-4-1BB monoclonal antibodies. Cell Immunol 190, 167-172.
Melero, I., Shuford, W. W., Newby, S. A., Aruffo, A., Ledbetter, J. A., Hellstrom, K. E., Mittler, R. S., and Chen, L. (1997). Monoclonal antibodies against the 4-1BB T-cell activation molecule eradicate established tumors. Nat Med 3, 682-685.
Merchant, A. M., Zhu, Z., Yuan, J. Q., Goddard, A., Adams, C. W., Presta, L. G., and Carter, P. (1998). An efficient route to human bispecific IgG. Nat Biotechnol 16, 677-681.
Morales-Kastresana, A., Sanmamed, M. F., Rodriguez, I., Palazon, A., Martinez-Forero, I., Labiano, S., Hervas-Stubbs, S., Sangro, B., Ochoa, C., Rouzaut, A., et al. (2013). Combined immunostimulatory monoclonal antibodies extend survival in an aggressive transgenic hepatocellular carcinoma mouse model. Clin Cancer Res 19, 6151-6162.
Mueller, D., Frey, K., Kontermann, R. E. (2008), A novel antibody-4-1BBl fusion protein for targeted costimulation in cancer immunotherapy, J. Immunother. 31, 714-722.
Murillo, O., Dubrot, J., Palazon, A., Arina, A., Azpilikueta, A., Alfaro, C., Solano, S., Ochoa, M. C., Berasain, C., Gabari, I., et al. (2009). In vivo depletion of DC impairs the anti-tumor effect of agonistic anti-CD137 mAb. Eur J Immunol 39, 2424-2436.
Narazaki, H., Zhu, Y., Luo, L., Zhu, G., and Chen, L. (2010). CD137 agonist antibody prevents cancer recurrence: contribution of CD137 on both hematopoietic and nonhematopoietic cells. Blood 115, 1941-1948.
Nishimoto, H., Lee, S. W., Hong, H., Potter, K. G., Maeda-Yamamoto, M., Kinoshita, T., Kawakami, Y., Mittler, R. S., Kwon, B. S., Ware, C. F., et al. (2005). Costimulation of mast cells by 4-1BB, a member of the tumor necrosis factor receptor superfamily, with the high-affinity IgE receptor. Blood 106, 4241-4248.
Olofsson, P. S., Soderstrom, L. A., Wagsater, D., Sheikine, Y., Ocaya, P., Lang, F., Rabu, C., Chen, L., Rudling, M., Aukrust, P., et al. (2008). CD137 is expressed in human atherosclerosis and promotes development of plaque inflammation in hypercholesterolemic mice. Circulation 117, 1292-1301.
Palazon, A., Teijeira, A., Martinez-Forero, I., Hervas-Stubbs, S., Roncal, C., Penuelas, I., Dubrot, J., Morales-Kastresana, A., Perez-Gracia, J. L., Ochoa, M. C., et al. (2011). Agonist anti-CD137 mAb act on tumor endothelial cells to enhance recruitment of activated T lymphocytes. Cancer Res 71, 801-811.
Schwarz, H., Valbracht, J., Tuckwell, J., von Kempis, J., and Lotz, M. (1995). ILA, the human 4-1BB homologue, is inducible in lymphoid and other cell lineages. Blood 85, 1043-1052.
Shao, Z., and Schwarz, H. (2011). CD137 ligand, a member of the tumor necrosis factor family, regulates immune responses via reverse signal transduction. J Leukoc Biol 89, 21-29.
Shi, W., and Siemann, D. W. (2006). Augmented antitumor effects of radiation therapy by 4-1BB antibody (BMS-469492) treatment. Anticancer Res 26, 3445-3453.
Simeone, E., and Ascierto, P. A. (2012). Immunomodulating antibodies in the treatment of metastatic melanoma: the experience with anti-CTLA-4, anti-CD137, and anti-PD1. J Immunotoxicol 9, 241-247.
Snell, L. M., Lin, G. H., McPherson, A. J., Moraes, T. J., and Watts, T. H. (2011). T-cell intrinsic effects of GITR and 4-1BB during viral infection and cancer immunotherapy. Immunol Rev 244, 197-217.
Stagg, J., Loi, S., Divisekera, U., Ngiow, S. F., Duret, H., Yagita, H., Teng, M. W., and Smyth, M. J. (2011). Anti-ErbB-2 mAb therapy requires type I and II interferons and synergizes with anti-PD-1 or anti-CD137 mAb therapy. Proc Natl Acad Sci USA 108, 7142-7147.
Teng, M. W., Sharkey, J., McLaughlin, N. M., Exley, M. A., and Smyth, M. J. (2009). CDld-based combination therapy eradicates established tumors in mice. J Immunol 183, 1911-1920.
von Kempis, J., Schwarz, H., and Lotz, M. (1997). Differentiation-dependent and stimulus-specific expression of ILA, the human 4-1BB-homologue, in cells of mesenchymal origin. Osteoarthritis Cartilage 5, 394-406.
Wei, H., Zhao, L., Li, W., Fan, K., Qian, W., Hou, S., Wang, H., Dai, M., Hellstrom, I., Hellstrom, K. E., and Guo, Y. (2013). Combinatorial PD-1 blockade and CD137 activation has therapeutic efficacy in murine cancer models and synergizes with cisplatin. PLoS One 8, e84927.
Wilcox, R. A., Chapoval, A. I., Gorski, K. S., Otsuji, M., Shin, T., Flies, D. B., Tamada, K., Mittler, R. S., Tsuchiya, H., Pardoll, D. M., and Chen, L. (2002). Cutting edge: Expression of functional CD137 receptor by dendritic cells. J Immunol 168, 4262-4267.
Wilcox, R. A., Tamada, K., Flies, D. B., Zhu, G., Chapoval, A. I., Blazar, B. R., Kast, W. M., and Chen, L. (2004). Ligation of CD137 receptor prevents and reverses established anergy of CD8+ cytolytic T lymphocytes in vivo. Blood 103, 177-184.
Zhang, N., Sadun, R. E., Arias, R. S., Flanagan, M. L., Sachsman, S. M., Nien, Y, Khawli, L. A., Hu, P., Epstein, A. L. (2007). Targeted and untargeted CD137L fusion proteins for the immunotherapy of experimental solid tumors. Clin. Cancer Res. 13, 2758-2767.
Zhang, X., Voskens, C. J., Sallin, M., Maniar, A., Montes, C. L., Zhang, Y., Lin, W., Li, G., Burch, E., Tan, M., et al. (2010). CD137 promotes proliferation and survival of human B cells. J Immunol 184, 787-795.

1. A TNF family ligand trimer-containing antigen binding molecule comprising
(a) one Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to one of the subunits of the Fc domain and wherein the second polypeptide is fused at its N-terminus to the C-terminus of the other subunit of the Fc domain,
wherein the TNF family ligand trimer-containing antigen binding molecule is monovalent for the binding to the target cell antigen.

2. The TNF family ligand trimer-containing antigen binding molecule of claim 1, wherein the TNF ligand family member costimulates human T-cell activation.

3. The TNF family ligand trimer-containing antigen binding molecule of claim 1 or 2, wherein the TNF ligand family member is selected from 4-1BBL and OX40L.

4. The TNF family ligand trimer-containing antigen binding molecule of any one of claims 1 to 3, wherein the TNF ligand family member is 4-1BBL.

5. The TNF family ligand trimer-containing antigen binding molecule of any one of claims 1 to 4, wherein the ectodomain of a TNF ligand family member comprises the amino acid sequence selected from the group consisting of SEQ ID NO:1, SEQ ID NO: 2, SEQ ID NO:3, SEQ ID NO:4, SEQ ID NO:5, SEQ ID NO:6, SEQ ID NO:7 and SEQ ID NO:8, particularly the amino acid sequence of SEQ ID NO:1 or SEQ ID NO:5.

6. The TNF family ligand trimer-containing antigen binding molecule of any one of claims 1 to 5, wherein the first polypeptide comprising two ectodomains of a TNF ligand family member or fragments thereof comprises an amino acid sequence selected from the group consisting of SEQ ID NO:9 and SEQ ID NO:10 and the second polypeptide comprising one ectodomain of said TNF ligand family member or a fragment thereof comprises the amino acid sequence selected from the group consisting of SEQ ID NO:1 and SEQ ID NO:5.

7. The TNF family ligand trimer-containing antigen binding molecule of any one of claims 1 to 3, wherein the TNF ligand family member is OX40L.

8. The TNF family ligand trimer-containing antigen binding molecule of any one of claims 1 to 3 or 7, wherein the ectodomain of a TNF ligand family member comprises the amino acid sequence selected from the group consisting of SEQ ID NO:11 and SEQ ID NO:12.

9. The TNF family ligand trimer-containing antigen binding molecule of any one of claims 1 to 8, wherein the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.

10. The TNF family ligand trimer-containing antigen binding molecule of any one of claims 1 to 9, wherein the target cell antigen is Fibroblast Activation Protein (FAP).

11. The TNF family ligand trimer-containing antigen binding molecule of any one of claims 1 to 10, wherein the Fab domain capable of specific binding to FAP comprises
(a) a VH domain comprising (i) CDR-H1 comprising the amino acid sequence of SEQ ID NO:13, (ii) CDR-H2 comprising the amino acid sequence of SEQ ID NO:14 and (iii) CDR-H3 comprising the amino acid sequence of SEQ ID NO:15, and a VL domain comprising (iv) CDR-L1 comprising the amino acid sequence of SEQ ID NO:16, (v) CDR-L2 comprising the amino acid sequence of SEQ ID NO:17 and (vi) CDR-L3 comprising the amino acid sequence of SEQ ID NO:18 or
(b) a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.

12. The TNF family ligand trimer-containing antigen binding molecule of any one of claims 1 to 11, wherein the Fab domain capable of specific binding to FAP comprises a variable heavy chain comprising an amino acid sequence of SEQ ID NO:25 and a variable light chain comprising an amino acid sequence of SEQ ID NO:26 or wherein the Fab domain capable of specific binding to FAP comprises a variable heavy chain comprising an amino acid sequence of SEQ ID NO:27 and a variable light chain comprising an amino acid sequence of SEQ ID NO:28.

13. The TNF family ligand trimer-containing antigen binding molecule of any one of claims 1 to 9, wherein the target cell antigen is CD19.

14. The TNF family ligand trimer-containing antigen binding molecule of any one of claims 1 to 9 or 13, wherein Fab domain capable of specific binding to CD19 comprises
(a) a VH domain comprising (i) CDR-H1 comprising the amino acid sequence of SEQ ID NO:29, (ii) CDR-H2 comprising the amino acid sequence of SEQ ID NO:30 and (iii) CDR-H3 comprising the amino acid sequence of SEQ ID NO:31, and a VL domain comprising (iv) CDR-L1 comprising the amino acid sequence of SEQ ID NO:32, (v) CDR-L2 comprising the amino acid sequence of SEQ ID NO:33 and (vi) CDR-L3 comprising the amino acid sequence of SEQ ID NO:34 or
(b) a VH domain comprising (i) CDR-H1 comprising the amino acid sequence of SEQ ID NO:35, (ii) CDR-H2 comprising the amino acid sequence of SEQ ID NO:36 and (iii) CDR-H3 comprising the amino acid sequence of SEQ ID NO:37, and a VL domain comprising (iv) CDR-L1 comprising the amino acid sequence of SEQ ID NO:38, (v) CDR-L2 comprising the amino acid sequence of SEQ ID NO:39 and (vi) CDR-L3 comprising the amino acid sequence of SEQ ID NO:40.

15. The TNF family ligand trimer-containing antigen binding molecule of any one of claims 1 to 9, 13 or 14, wherein the Fab domain capable of specific binding to CD19 comprises a variable heavy chain comprising an amino acid sequence of SEQ ID NO:41 and a variable light chain comprising an amino acid sequence of SEQ ID NO:42 or wherein the Fab domain capable of specific binding to FAP comprises a variable heavy chain comprising an amino acid sequence of SEQ ID NO:43 and a variable light chain comprising an amino acid sequence of SEQ ID NO:44.

16. The TNF family ligand trimer-containing antigen binding molecule of any one of claims 1 to 9, wherein the target cell antigen is CEA.

17. The TNF family ligand trimer-containing antigen binding molecule of any one of points 1 to 9 or 16, wherein Fab domain capable of specific binding to CEA comprises a VH domain comprising (i) CDR-H1 comprising the amino acid sequence of SEQ ID NO:45, (ii) CDR-H2 comprising the amino acid sequence of SEQ ID NO:46 and (iii) CDR-H3 comprising the amino acid sequence of SEQ ID NO:47, and a VL domain comprising (iv) CDR-L1 comprising the amino acid sequence of SEQ ID NO:48, (v) CDR-L2 comprising the amino acid sequence of SEQ ID NO:49 and (vi) CDR-L3 comprising the amino acid sequence of SEQ ID NO:50.

18. The TNF family ligand trimer-containing antigen binding molecule of any one of claims 1 to 9, 16 or 17, wherein the Fab domain capable of specific binding to CEA comprises a variable heavy chain comprising an amino acid sequence of SEQ ID NO:51 and a variable light chain comprising an amino acid sequence of SEQ ID NO:52.

19. The TNF family ligand trimer-containing antigen binding molecule of any one of claims 1 to 18, wherein the Fc domain composed of a first and a second subunit capable of stable association is an IgG domain, in particular an IgG1 domain.

20. The TNF family ligand trimer-containing antigen binding molecule of any one of claims 1 to 19, wherein the Fc domain comprises one or more amino acid substitution that reduces binding to an Fc receptor, in particular towards Fcγ receptor.

21. The TNF family ligand trimer-containing antigen binding molecule of any one of claims 1 to 20, wherein the Fc domain is an IgG1 Fc domain comprising amino acid substitutions at positions 234 and 235 (numbering according to Kabat EU index) and/or 329 (numbering according to Kabat EU index).

22. The TNF family ligand trimer-containing antigen binding molecule of any one of claims 1 to 21, wherein the first subunit of the Fc domain comprises the amino acid substitutions S354C and T366W (numbering according to Kabat EU index) and the second subunit of the Fc domain comprises the amino acid substitutions Y349C, T366S, L368A and Y407V (numbering according to Kabat EU index).

23. The TNF family ligand trimer-containing antigen binding molecule of any one of claims 1 to 22, comprising
(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fc domain comprising the hole mutations and wherein the VH and CH1 domain of the Fab domain capable of specific binding to a target cell antigen is fused at the C-terminus to the N-terminus of the second subunit of the Fc domain comprising the knob mutations.

24. The TNF family ligand trimer-containing antigen binding molecule of any one of claims 1 to 22, comprising
(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the second subunit of the Fc domain comprising the knob mutations and wherein the second polypeptide is fused at its N-terminus to the C-terminus of the first subunit of the Fc domain comprising the hole mutations and wherein the VH and CH1 domain of the Fab domain capable of specific binding to a target cell antigen is fused at the C-terminus to the N-terminus of the first subunit of the Fc domain comprising the hole mutations.

25. The TNF family ligand trimer-containing antigen binding molecule of any one of claims 1 to 22, comprising
(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fc domain comprising the hole mutations and wherein the second polypeptide is fused at its N-terminus to the C-terminus of the second subunit of the Fc domain comprising the knob mutations and wherein the VH and CH1 domain of the Fab domain capable of specific binding to a target cell antigen is fused at the C-terminus to the N-terminus of the second subunit of the Fc domain comprising the knob mutations.

26. The TNF family ligand trimer-containing antigen binding molecule of any one of claims 1 to 22, comprising
(a) a Fab domain capable of specific binding to a target cell antigen,
(b) a Fc domain composed of a first and a second subunit capable of stable association, and
(c) a first polypeptide comprising two ectodomains of a TNF ligand family member or fragments thereof that are connected to each other by a peptide linker and a second polypeptide comprising one ectodomain of said TNF ligand family member or a fragment thereof, wherein the first polypeptide is fused at its N-terminus to the C-terminus to the first subunit of the Fc domain comprising the hole mutations and wherein the second polypeptide is fused at its N-terminus to the C-terminus of the second subunit of the Fc domain comprising the knob mutations and wherein the VH and CH1 domain of the Fab domain capable of specific binding to a target cell antigen is fused at the C-terminus to the N-terminus of the first subunit of the Fe domain comprising the hole mutations.

27. The TNF family ligand trimer-containing antigen binding molecule of any one of claims 1 to 26 further comprising
(d) a Fab domain that is not capable of specific binding to a target cell antigen.

28. An isolated polynucleotide encoding the TNF family ligand trimer-containing antigen binding molecule of any one of claims 1 to 27.

29. A vector, particularly an expression vector, comprising the isolated polynucleotide of claim 28.

30. A host cell comprising the isolated polynucleotide of claim 28 or the vector of claim 29.

31. A method for producing the TNF family ligand trimer-containing antigen binding molecule of any one of claims 1 to 27, comprising culturing the host cell of claim 30 under conditions suitable for expression of the antigen binding molecule and recovering the antigen binding molecule.

32. A pharmaceutical composition comprising the TNF family ligand trimer-containing antigen binding molecule of any one of points 1 to 27 and at least one pharmaceutically acceptable excipient.

33. The TNF family ligand trimer-containing antigen binding molecule of any one of claims 1 to 27, or the pharmaceutical composition of claim 32, for use as a medicament.

34. The TNF family ligand trimer-containing antigen binding molecule of any one of claims 1 to 27, or the pharmaceutical composition of claim 32, for use in the treatment of cancer.

35. Use of the TNF family ligand trimer-containing antigen binding molecule of any one of claims 1 to 27 for the manufacture of a medicament for the treatment of cancer.

36. A method of treating a disease in an individual, comprising administering to said individual a therapeutically effective amount of a composition comprising a TNF family ligand trimer-containing antigen binding molecule of any one of claims 1 to 27 in a pharmaceutically acceptable form.

37. The method of claim 36, wherein said disease is cancer.
